(19) United States
(12) Patent Application Publication
(10) Pub. No.: US 2022/0227745 A1
(43) Pub. Date: Jul. 21, 2022

(54) Compounds For Modulating FXR

(71) Applicant: NANJING RUIJIE PHARMA TECH CO., LTD., Nanjing, Jiangsu (CN)

(72) Inventors: Junbo Zhang, Nanjing, Jiangsu (CN); Shuhao Zhu, Nanjing, Jiangsu (CN); Xiaoxin Qi, Nanjing, Jiangsu (CN)

(21) Appl. No.: 17/618,878

(22) PCT Filed: Jun. 12, 2020
(86) PCT No.: PCT/CN2020/095754

§ 371 Date: Dec. 13, 2021

(30) Foreign Application Priority Data
Jun. 14, 2019 (CN) PCT/CN2019/091253

(51) Int. Cl. C07D 413/12; C07D 261/08; C07D 471/08

(52) U.S. Cl. CPC C07D 413/12; C07D 471/08; C07D 261/08

(57) ABSTRACT

Provided herein are compounds of Formula (I), a stereoisomer, enantiomer or a pharmaceutically acceptable salt thereof; wherein variables are as defined herein; and their pharmaceutical compositions, which are useful as modulators of the activity of Farnesoid X receptors (FXR).

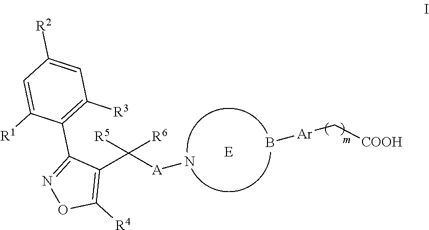

### CROSS-REFERENCE TO RELATED APPLICATION(S)

This application is a 371 application of International Application No. PCT/CN2020/095754, filed on Jun. 12, 2020, which claims priority to International Application No. PCT/CN2019/091253 filed on Jun. 14, 2019, the entire disclosures of both of which are hereby incorporated by reference.

### TECHNICAL FIELD

The current disclosure relates to the fields of medicinal chemistry, pharmacology, and medicine. Specifically, the disclosure relates to novel compounds useful for modulating the activity of farnesoid X receptors (FXRs).

### BACKGROUND

The farnesoid X receptor is a member of the nuclear hormone receptor superfamily and is primarily expressed in the liver, kidney and intestine (see, e.g., Seol et al. (1995) Mol. Endocrinol. 9:72-85 and Forman et al. (1995) Cell 81:687-693). It functions as a heterodimer with the retinoid X receptor (RXR) and binds to response elements in the promoters of target genes to regulate gene transcription. The FXR-RXR heterodimer binds with highest affinity to an inverted repeat-1 (IR-1) response element, in which consensus receptor-binding hexamers are separated by one nucleotide. FXR is part of an interrelated process, in that FXR is activated by bile acids (the end product of cholesterol metabolism) (see, e.g., Makishima et al. (1999) Science 284: 1362-1365, Parks et al. (1999) Science 284: 1365-1368, Wang et al. (1999) Mol. Cell. 3:543-553), which serve to inhibit cholesterol catabolism. See also, Urizar et al. (2000) J. Biol. Chem. 275:39313-39317.

FXR is a key regulator of cholesterol homeostasis, triglyceride synthesis and lipogenesis. (Crawley, Expert Opinion Ther. Patents (2010), 20(8): 1047-1057). In addition to the treatment of dyslipidemia, multiple indications for FXR have been described, including treatment of liver disease, diabetes, vitamin D-related diseases, drug-induced side effects and hepatitis. (Crawley, supra).

Obeticholic Acid (6α-ethyl-chenodeoxycholic acid) developed by Intercept Co., (abbreviated to OCA and also known as INT-747) is the first FXR agonist approved by FDA on May 31, 2016. It's the analogue to the natural bile acid chenodeoxycholic acid. In clinical studies, OCA showed efficacy in both Primary Biliary Cirrhosis (PBC) and non-alcoholic steatohepatitis (NASH) subjects; however, OCA treatment may be associated with increased pruritus. OCA was tested at doses between 5 mg and 50 mg in PBC subjects or NASH subjects. GW4604 (WO2000037077) developed by GSK is an isoxazole FXR agonist with strong agonistic activity to FXR, but it's unstable to light and has low bioavailability. LY-2562175 (WO2009012125A1) is a novel potent, selective, partial FXR agonist originally developed by Eli Lilly and later licensed to TERN and renumbered as TERN-101, it didn't promote transcriptional activation of other nuclear receptor but lowered LDL and triglycerides while raising HDL in preclinical species. PX-I04 (W02011020615A1) is also an isoxazole FXR agonist, it's originally developed by Phenex and later licensed to Gilead. It's currently in clinical phase II. Tropifexor, also known as LJN-452 (WO2012087519A1), is a non-steroidal FXR agonist currently in clinical phase II for the treatment of NASH, fatty liver and primary biliary cholangitis, and is expected to be completed in 2019. It was originally developed by Novartis Pharmaceuticals and later licensed to Pfizer for collaborative research and development. In 2016, Novartis released the first clinical data (95 people) of LJN452, and the results were gratifying. LJN452 performed well in safety and tolerability at a single dose of up to 3 mg. No drug-related adverse reactions were observed. No drug-related pruritus was observed after multiple doses. ALT/AST increased in individual subjects, but did not cause clinical sequelae. Other FXR agonist in development included LMB-763, GS-9674, TERN-101, MET-409 and so on.

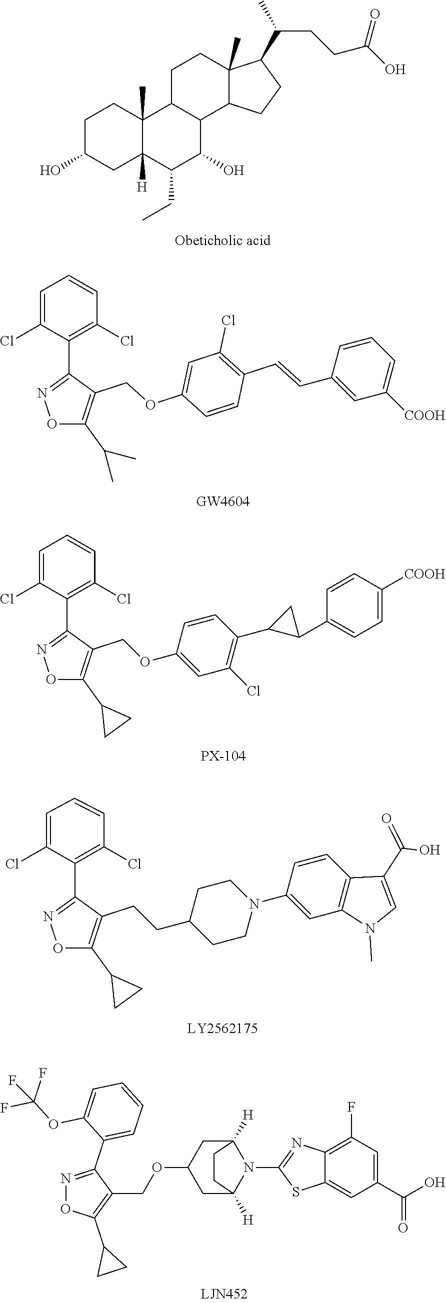 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12 ; CC(C)c1onc(-c2c(Cl)cccc2Cl)c1COc1ccc(/C=C/c2cccc(C(=O)O)c2)c(Cl)c1 ; O=C(O)c1ccc(C2CC2c2ccc(OCc3c(-c4c(Cl)cccc4Cl)noc3C3CC3)cc2Cl)cc1 ; Cn1cc(C(=O)O)c2ccc(N3CCC(CCc4c(-c5c(Cl)cccc5Cl)noc4C4CC4)CC3)cc21 ; O=C(O)c1cc(F)c2nc(N3[C@@H]4CC[C@H]3CC(OCc3c(-c5ccccc5OC(F)(F)F)noc3C3CC3)C4)sc2c1]

Although advances have been made in the development of novel FXR agonists, significant room for improvement remains. It is the object of the present disclosure to provide novel compounds that are agonists or partial agonists of FXR exhibiting physicochemical, in vitro and/or in vivo ADME (adsorption, distribution, metabolism and excretion) properties superior to known agonists of FXR and/or superior pharmacokinetics in vivo.

### SUMMARY

The present disclosure provides a compound having Formula (I)

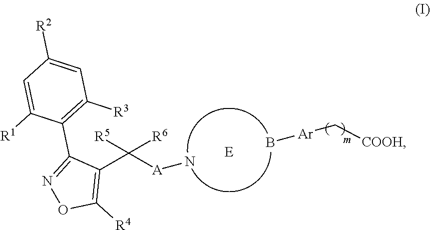

or a stereoisomer, enantiomer or a pharmaceutically acceptable salt thereof;

R¹, R² and R³ are independently selected from H, C₁₋₆ alkyl, haloC₁₋₆ alkyl, C₁₋₆ alkoxy, haloC₁₋₆ alkoxy, or cyclopropyl;

R⁴ is selected from C₁₋₃ alkyl, haloC₁₋₃ alkyl or cyclopropyl optionally substituted with C₁₋₃ alkyl or haloC₁₋₃ alkyl;

R⁵ and R⁶ are independently selected from H, C₁₋₃ alkyl or haloC₁₋₃ alkyl;

A is selected from C═O or CR⁷R⁸;

R⁷ and R⁸ are independently selected from H, C₁₋₃ alkyl or C₁₋₃ alkoxy;

B is CH or N;

ring E is a substituted or unsubstituted 6-8 membered heteroring or bridged-heteroring;

Ar is phenylene, C₅₋₇ cycloalkylene or 5-14 membered monocyclic or bicyclic heteroaryl containing 1-2 heteroatoms selected from N, O and S; each of which is optionally substituted with R¹⁰ and R¹¹,

R¹⁰ and R¹¹ are independently selected from H, halogen, C₁₋₆ alkyl, haloC₁₋₆ alkyl, C₁₋₆ alkoxy, haloC₁₋₆ alkoxy, or cyclopropyl;

m is 0 or 1.

In some embodiments, the compounds of the disclosure are defined by formula (I) wherein R¹, R² and R³ are independently selected from H, Cl, F, CH₃, OCF₃, CF₃ and OMe.

In some embodiments, the compounds of the disclosure are defined by formula (I) wherein R⁴ is C₁₋₃ alkyl or cyclopropyl.

More particularly, wherein R⁴ is cyclopropyl, methyl or i-Pr.

In some embodiments, the compounds of the disclosure are defined by formula (I) wherein R⁵ and R⁶ are independently selected from hydrogen or Me.

In some embodiments, the compounds of the disclosure are defined by formula (I), wherein R⁷ and R⁸ are independently selected from H or Me.

In some embodiments, wherein Ar is phenylene, pyridylene, pyrimidinylene, pyrazinylene, pyridazinylene, thiazolylene, benzothiazolyl, benzo[d]isothiazolyl, imidazo[1,2-a]pyridinyl, quinolinyl, 1H-indolyl, pyrrolo[1,2-b]pyridazinyl, benzofuranyl, benzo[b]thiophenyl, 1H-indazolyl, benzo[d]isoxazolyl, quinazolinyl, 1H-pyrrolo[3,2-c]pyridinyl, pyrazolo[1,5-a]pyrimidinyl, imidazo[1,2-b]pyridazinyl, pyrazolo[1,5-a]pyridinyl; each of which is optionally substituted with R¹⁰ and R¹¹, R¹⁰ and R¹¹ are independently selected from H, halogen, C₁₋₆ alkyl, haloC₁₋₆ alkyl, C₁₋₆alkoxy, haloC₁₋₆alkoxy or cyclopropyl.

More particularly, wherein Ar is selected from phenylene, benzothiazolyl, quinolinyl, 1H-indolyl, 1H-indazolyl, each of which is optionally substituted with 0˜2 groups of Me or F.

More particularly, wherein Ar is phenylene or selected from the following structures:

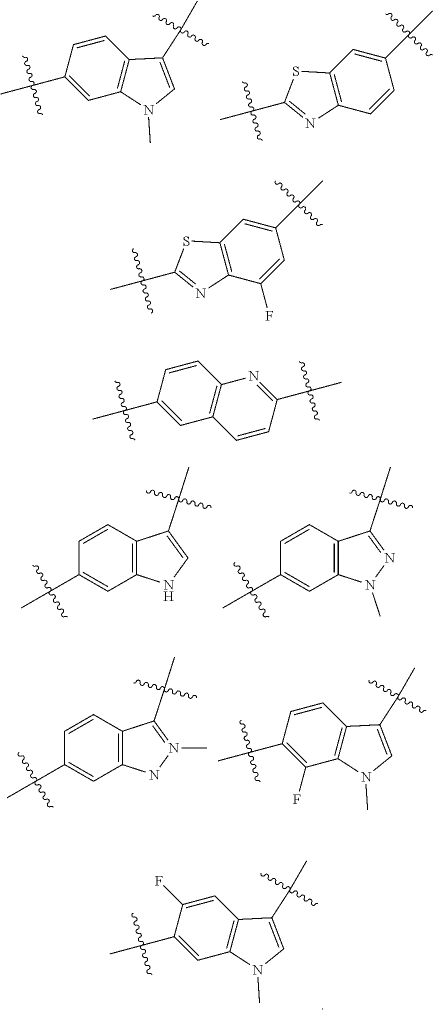

In some embodiments, wherein ring E is selected from the following structures, which is optionally substituted with 0˜2 groups of Me

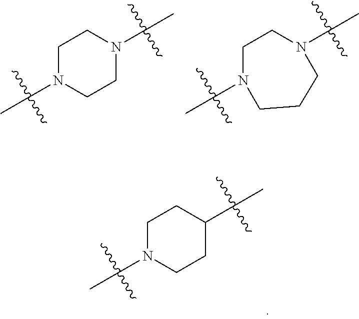 [SMILES: *N1CCN(*)CC1 ; *N1CCCN(*)CC1 ; *C1CCN(*)CC1]

More particularly, wherein ring E is selected from the following structures

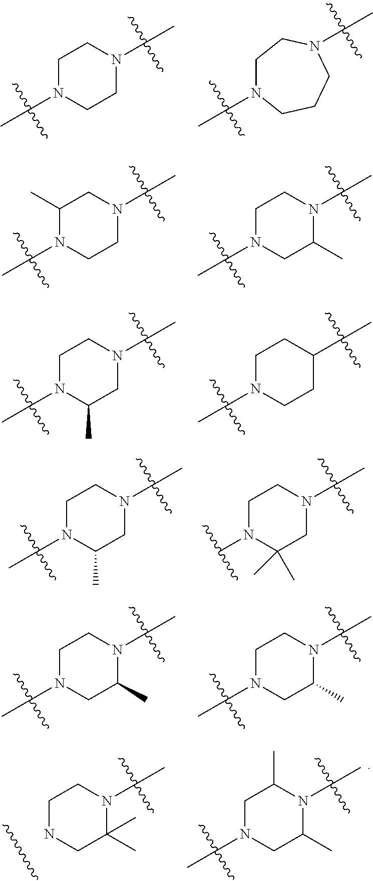

In some embodiments, wherein ring E is selected from the following structures

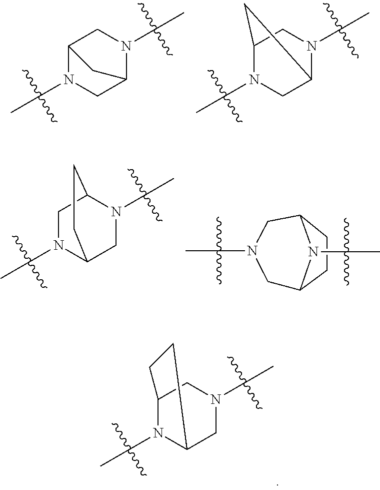 [SMILES: *N1CC2CC1CN2* ; *N1CC2CC1CN2* ; *N1CC2CCC1CN2* ; *N1CC2CCC(C1)N2* ; *N1CC2CCC(C1)N2*]

In some embodiments, wherein said compound is selected from the group consisting of

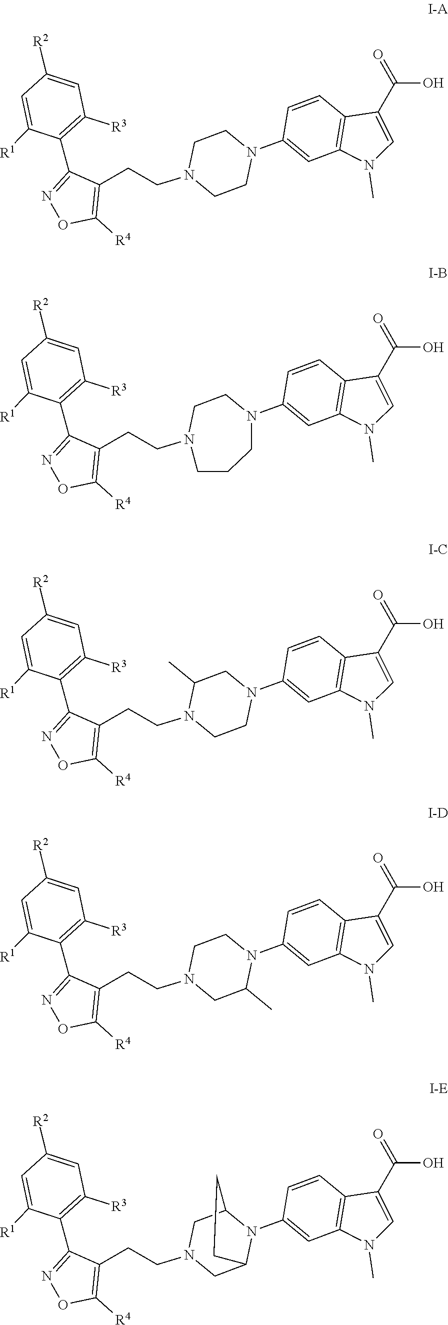

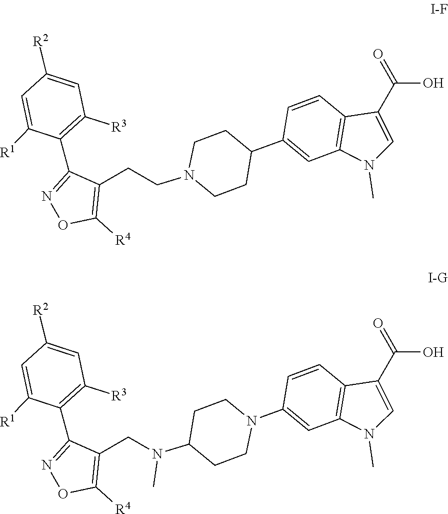

Wherein R¹, R² and R³ are independently selected from H, Cl, F, CH₃, OCF₃, CF₃ and OMe; W is cyclopropyl or i-Pr;

or a stereoisomer, enantiomer or a pharmaceutically acceptable salt thereof.

A particularly preferred compound of formula (I), as defined above is that selected from one of the following structure

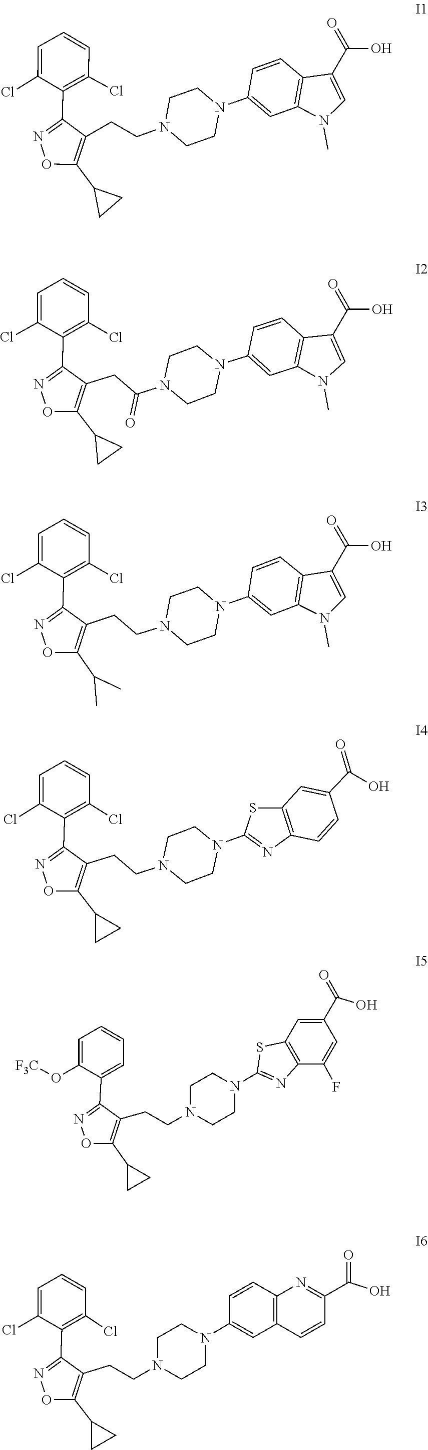

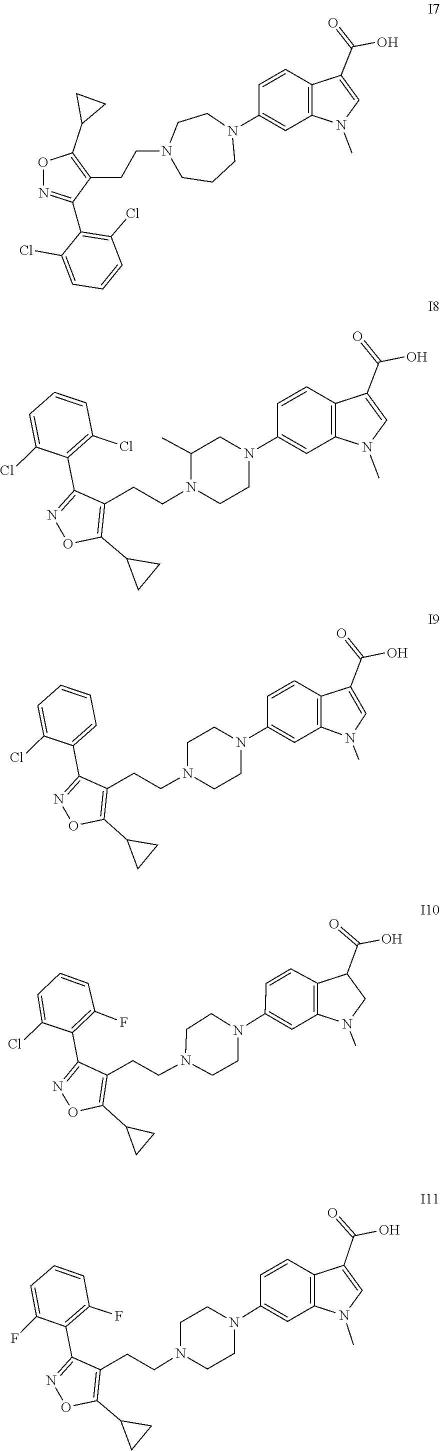 [SMILES: Cn1cc(C(=O)O)c2ccc(N3CCCN(CCc4c(-c5c(Cl)cccc5Cl)noc4C4CC4)CC3)cc21 ; CC1CN(c2ccc3c(C(=O)O)cn(C)c3c2)CCN1CCc1c(-c2c(Cl)cccc2Cl)noc1C1CC1 ; Cn1cc(C(=O)O)c2ccc(N3CCN(CCc4c(-c5ccccc5Cl)noc4C4CC4)CC3)cc21 ; CN1CC(C(=O)O)c2ccc(N3CCN(CCc4c(-c5c(F)cccc5Cl)noc4C4CC4)CC3)cc21 ; Cn1cc(C(=O)O)c2ccc(N3CCN(CCc4c(-c5c(F)cccc5F)noc4C4CC4)CC3)cc21]

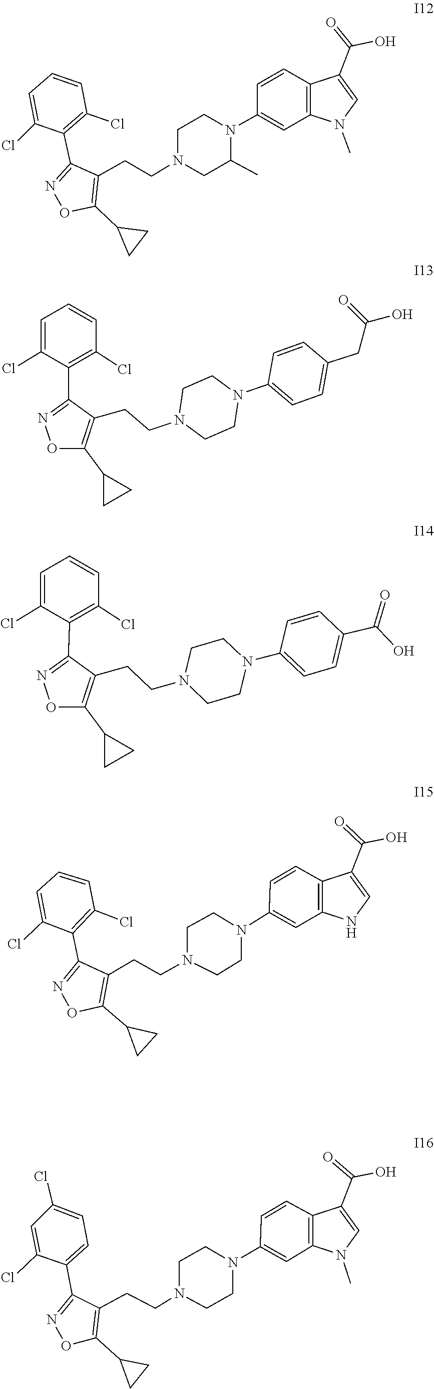 [SMILES: CC1CN(CCc2c(-c3c(Cl)cccc3Cl)noc2C2CC2)CCN1c1ccc2c(C(=O)O)cn(C)c2c1 ; O=C(O)Cc1ccc(N2CCN(CCc3c(-c4c(Cl)cccc4Cl)noc3C3CC3)CC2)cc1 ; O=C(O)c1ccc(N2CCN(CCc3c(-c4c(Cl)cccc4Cl)noc3C3CC3)CC2)cc1 ; O=C(O)c1c[nH]c2cc(N3CCN(CCc4c(-c5c(Cl)cccc5Cl)noc4C4CC4)CC3)ccc12 ; Cn1cc(C(=O)O)c2ccc(N3CCN(CCc4c(-c5ccc(Cl)cc5Cl)noc4C4CC4)CC3)cc21]

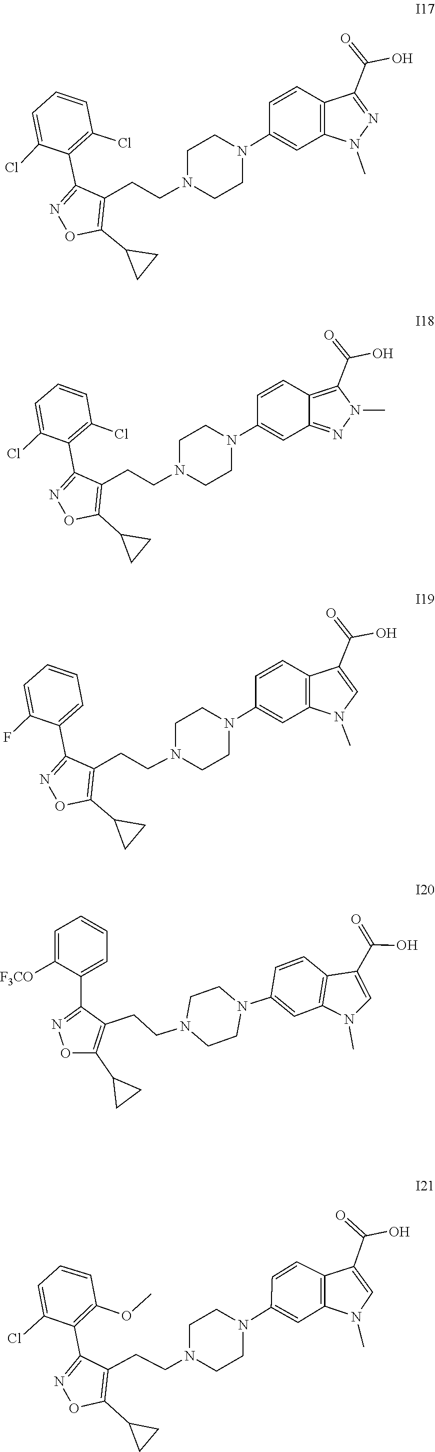 [SMILES: Cn1nc(C(=O)O)c2ccc(N3CCN(CCc4c(-c5c(Cl)cccc5Cl)noc4C4CC4)CC3)cc21 ; Cn1nc2cc(N3CCN(CCc4c(-c5c(Cl)cccc5Cl)noc4C4CC4)CC3)ccc2c1C(=O)O ; Cn1cc(C(=O)O)c2ccc(N3CCN(CCc4c(-c5ccccc5F)noc4C4CC4)CC3)cc21 ; Cn1cc(C(=O)O)c2ccc(N3CCN(CCc4c(-c5ccccc5OC(F)(F)F)noc4C4CC4)CC3)cc21 ; COc1cccc(Cl)c1-c1noc(C2CC2)c1CCN1CCN(c2ccc3c(C(=O)O)cn(C)c3c2)CC1]

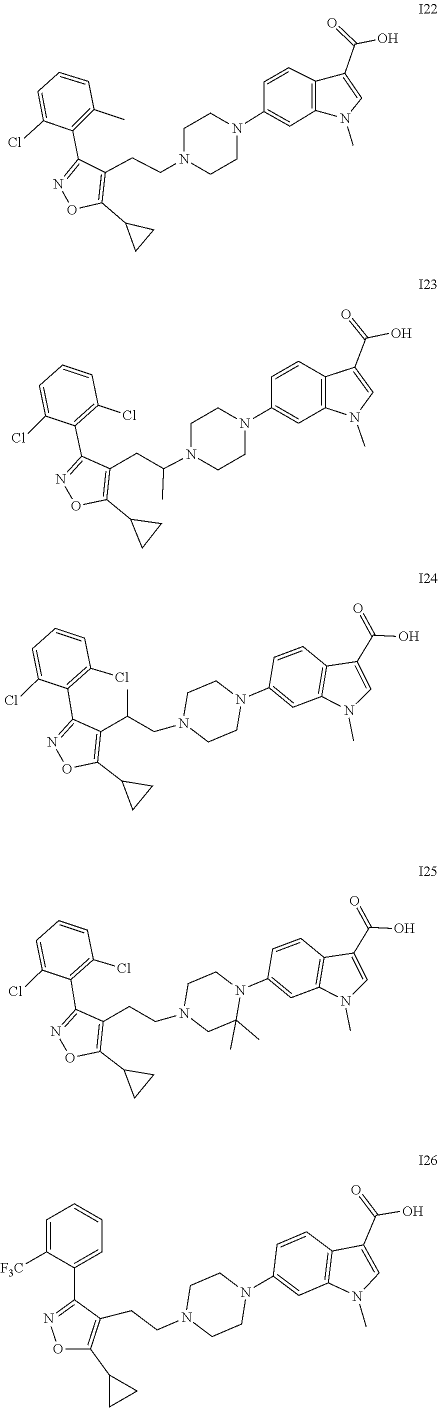 [SMILES: Cc1cccc(Cl)c1-c1noc(C2CC2)c1CCN1CCN(c2ccc3c(C(=O)O)cn(C)c3c2)CC1 ; CC(Cc1c(-c2c(Cl)cccc2Cl)noc1C1CC1)N1CCN(c2ccc3c(C(=O)O)cn(C)c3c2)CC1 ; CC(CN1CCN(c2ccc3c(C(=O)O)cn(C)c3c2)CC1)c1c(-c2c(Cl)cccc2Cl)noc1C1CC1 ; Cn1cc(C(=O)O)c2ccc(N3CCN(CCc4c(-c5c(Cl)cccc5Cl)noc4C4CC4)CC3(C)C)cc21 ; Cn1cc(C(=O)O)c2ccc(N3CCN(CCc4c(-c5ccccc5C(F)(F)F)noc4C4CC4)CC3)cc21]

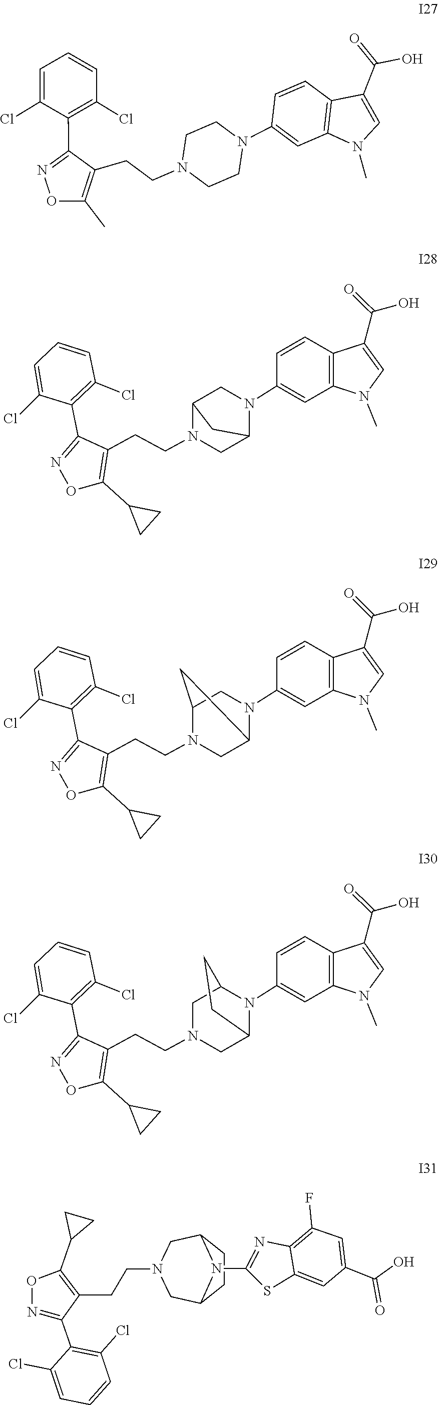 [SMILES: Cc1onc(-c2c(Cl)cccc2Cl)c1CCN1CCN(c2ccc3c(C(=O)O)cn(C)c3c2)CC1 ; Cn1cc(C(=O)O)c2ccc(N3CC4CC3CN4CCc3c(-c4c(Cl)cccc4Cl)noc3C3CC3)cc21 ; Cn1cc(C(=O)O)c2ccc(N3CC4CC3CN4CCc3c(-c4c(Cl)cccc4Cl)noc3C3CC3)cc21 ; Cn1cc(C(=O)O)c2ccc(N3C4CCC3CN(CCc3c(-c5c(Cl)cccc5Cl)noc3C3CC3)C4)cc21 ; O=C(O)c1cc(F)c2nc(N3C4CCC3CN(CCc3c(-c5c(Cl)cccc5Cl)noc3C3CC3)C4)sc2c1]

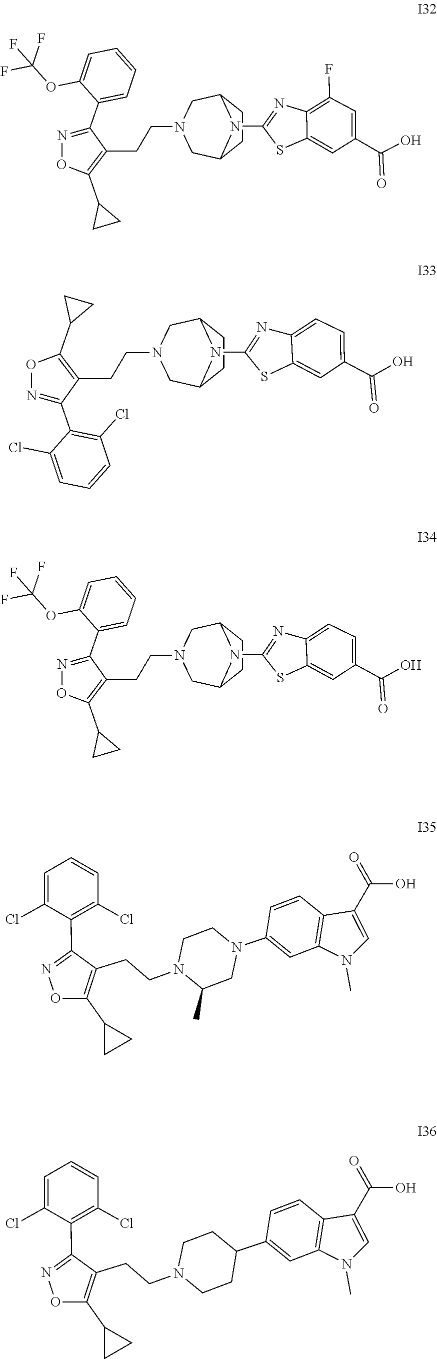 [SMILES: O=C(O)c1cc(F)c2nc(N3C4CCC3CN(CCc3c(-c5ccccc5OC(F)(F)F)noc3C3CC3)C4)sc2c1 ; O=C(O)c1ccc2nc(N3C4CCC3CN(CCc3c(-c5c(Cl)cccc5Cl)noc3C3CC3)C4)sc2c1 ; O=C(O)c1ccc2nc(N3C4CCC3CN(CCc3c(-c5ccccc5OC(F)(F)F)noc3C3CC3)C4)sc2c1 ; C[C@@H]1CN(c2ccc3c(C(=O)O)cn(C)c3c2)CCN1CCc1c(-c2c(Cl)cccc2Cl)noc1C1CC1 ; Cn1cc(C(=O)O)c2ccc(C3CCN(CCc4c(-c5c(Cl)cccc5Cl)noc4C4CC4)CC3)cc21]

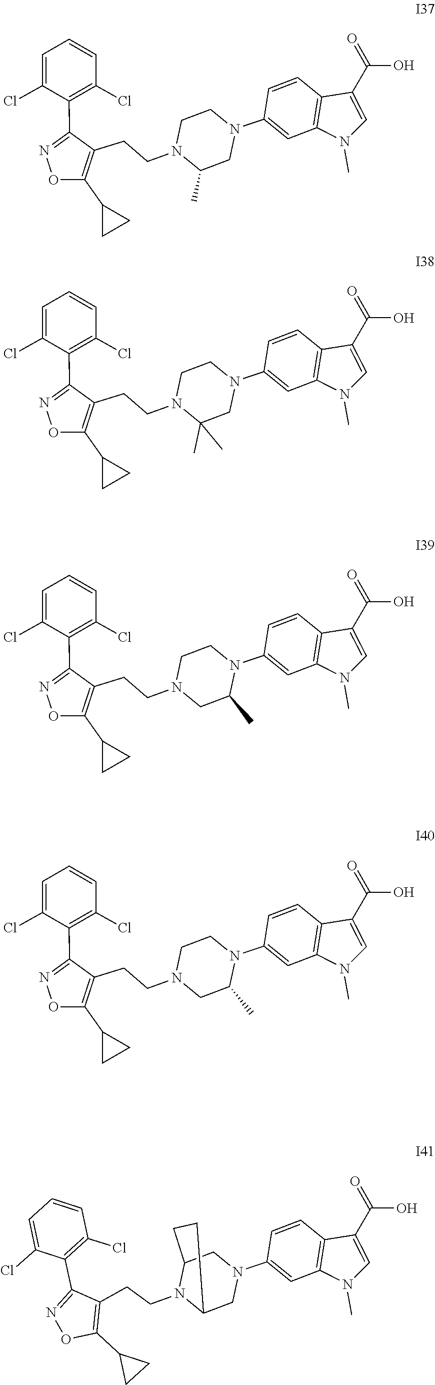 [SMILES: C[C@H]1CN(c2ccc3c(C(=O)O)cn(C)c3c2)CCN1CCc1c(-c2c(Cl)cccc2Cl)noc1C1CC1 ; Cn1cc(C(=O)O)c2ccc(N3CCN(CCc4c(-c5c(Cl)cccc5Cl)noc4C4CC4)C(C)(C)C3)cc21 ; C[C@H]1CN(CCc2c(-c3c(Cl)cccc3Cl)noc2C2CC2)CCN1c1ccc2c(C(=O)O)cn(C)c2c1 ; C[C@@H]1CN(CCc2c(-c3c(Cl)cccc3Cl)noc2C2CC2)CCN1c1ccc2c(C(=O)O)cn(C)c2c1 ; Cn1cc(C(=O)O)c2ccc(N3CC4CCC(C3)N4CCc3c(-c4c(Cl)cccc4Cl)noc3C3CC3)cc21]

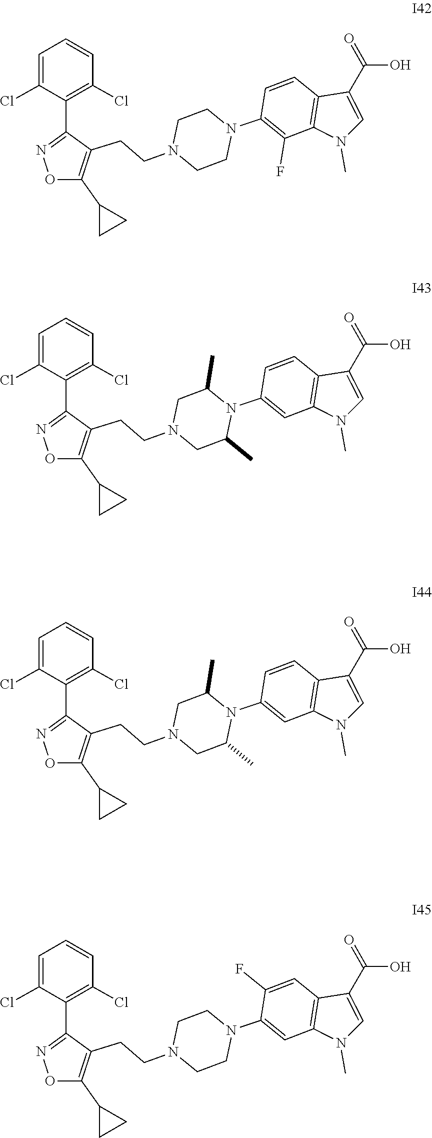 [SMILES: Cn1cc(C(=O)O)c2ccc(N3CCN(CCc4c(-c5c(Cl)cccc5Cl)noc4C4CC4)CC3)c(F)c21 ; C[C@@H]1CN(CCc2c(-c3c(Cl)cccc3Cl)noc2C2CC2)C[C@H](C)N1c1ccc2c(C(=O)O)cn(C)c2c1 ; C[C@@H]1CN(CCc2c(-c3c(Cl)cccc3Cl)noc2C2CC2)C[C@@H](C)N1c1ccc2c(C(=O)O)cn(C)c2c1 ; Cn1cc(C(=O)O)c2cc(F)c(N3CCN(CCc4c(-c5c(Cl)cccc5Cl)noc4C4CC4)CC3)cc21]

or a stereoisomer, enantiomer or a pharmaceutically acceptable salt thereof.

In another aspect, the present disclosure provides a compound having Formula (I′):

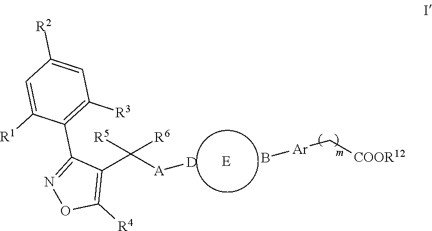

or a stereoisomer, enantiomer or a pharmaceutically acceptable salt thereof;

R¹, R² and R³ are independently selected from H, C₁₋₆alkyl, haloC₁₋₆alkyl, C₁₋₆ alkoxy, haloC₁₋₆alkoxy, or cyclopropyl;

R⁴ is selected from C₁₋₃alkyl, haloC₁₋₃alkyl or cyclopropyl optionally substituted with C₁₋₃ alkyl or haloC₁₋₃alkyl;

R⁵ and R⁶ are independently selected from H, C₁₋₃alkyl or haloC₁₋₃alkyl;

A is selected from C═O, CR⁷R⁸, 0 or NR⁹;

R⁷ and R⁸ are independently selected from H, C₁₋₃alkyl or C₁₋₃alkoxy;

R⁹ is selected from H, C₁₋₃alkyl or C₁₋₃alkoxy;

B is CR¹³ or N;

D is CR¹⁴ or N;

ring E is a substituted or unsubstituted 6-8 membered heteroring or bridged-heteroring; D and B are atoms or groups on ring E.

Ar is phenylene, C₅₋₇ cycloalkylene or 5-14 membered monocyclic or bicyclic heteroaryl containing 1-2 heteroatoms selected from N, O and S; each of which is optionally substituted with R¹⁰ and R¹¹,

R¹⁰ and R¹¹ are independently selected from H, halogen, C₁₋₆ alkyl, haloC₁₋₆ alkyl, C₁₋₆ alkoxy, haloC₁₋₆ alkoxy, or cyclopropyl;

R¹² is selected from H, C₁₋₃alkyl or C₁₋₃alkoxy;

R¹³ is selected from H, OH, C₁₋₃alkyl or C₁₋₃alkoxy;

R¹⁴ is selected from H, OH, C₁₋₃alkyl or C₁₋₃alkoxy.

m is 0 or 1.

In some embodiments, the compounds of the disclosure are defined by formula (I′), wherein

A is selected from C═O or CR⁷R⁸;

B is CR¹³ or N;

D is N;

R¹² is H;

R is H.

In some embodiments, the compounds of the disclosure are defined by formula (I′), wherein

A is selected from O or NMe;

B is CR¹³ or N;

D is N or CH;

R¹² is H or Me;

R¹³ is H or OH.

In some embodiments, the compounds of the disclosure are defined by formula (I′), wherein R¹, R² and R³ are independently selected from H, Cl, F, CH₃, OCF₃, CF₃ and OMe.

In some embodiments, the compounds of the disclosure are defined by formula (I′), wherein R⁴ is C₁₋₃ alkyl or cyclopropyl, more particularly, wherein R⁴ is cyclopropyl, methyl or i-Pr.

In some embodiments, the compounds of the disclosure are defined by formula (I′), wherein R⁵ and R⁶ are independently selected from hydrogen or Me.

In some embodiments, the compounds of the disclosure are defined by formula (I′), wherein R⁷ and R⁸ are independently selected from H or Me.

In some embodiments, the compounds of the disclosure are defined by formula (I′), wherein R⁹ is selected from H, Me, Et, n-Pr or i-Pr; R¹² and R¹³ are independently selected from H, Me, Et, n-Pr or i-Pr.

In some embodiments, the compounds of the disclosure are defined by formula (I′), wherein Ar is selected from substituted or unsubstituted phenylene, pyridylene, pyrimidinylene, pyrazinylene, pyridazinylene, thiazolylene, benzothiazolyl, benzo[d]isothiazolyl, imidazo[1,2-a]pyridinyl, quinolinyl, 1H-indolyl, pyrrolo[1,2-b]pyridazinyl, benzofuranyl, benzo[b]thiophenyl, 1H-indazolyl, benzo[d]isoxazolyl, quinazolinyl, 1H-pyrrolo[3,2-c]pyridinyl, pyrazolo[1,5-a]pyrimidinyl, imidazo[1,2-b]pyridazinyl, pyrazolo[1,5-a]pyridinyl; each of which is optionally substituted with R¹⁰ and R¹¹ selected from H, halogen, C₁₋₆ alkyl, haloC₁₋₆ alkyl, C₁₋₆ alkoxy, haloC₁₋₆ alkoxy, or cyclopropyl.

More particularly, wherein Ar is selected from phenylene, benzothiazolyl, quinolinyl, 1H-indolyl, 1H-indazolyl, each of which is optionally substituted with 0˜2 groups of Me or F.

More particularly, wherein Ar is phenylene or selected from the following structure:

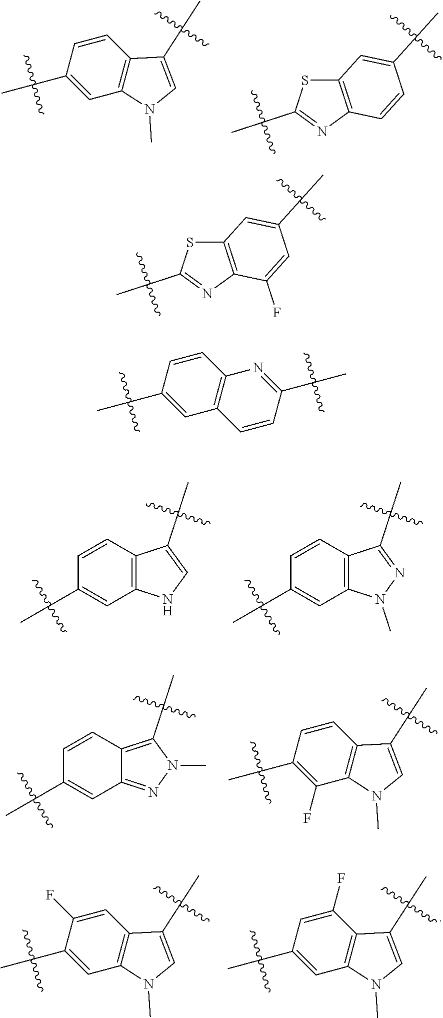

In some embodiments, the compounds of the disclosure are defined by formula (I′), wherein ring E is selected from the following structure, which is optionally substituted with 0˜2 groups of OH or Me

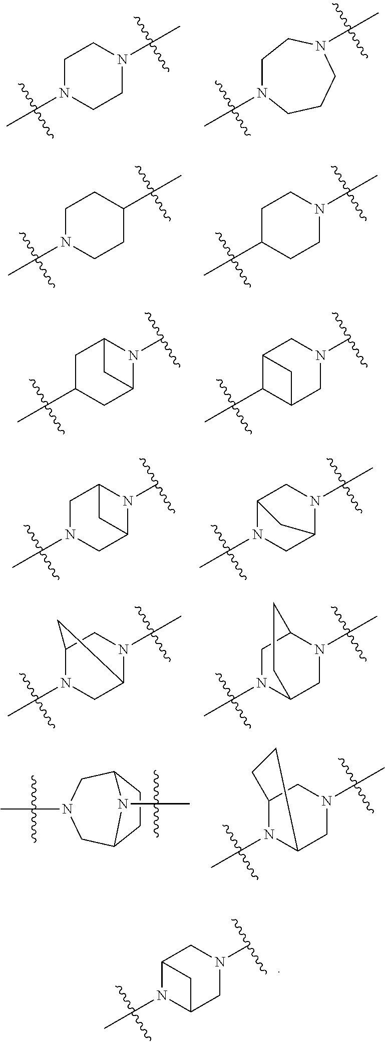

More particularly, wherein ring E is selected from the following structure

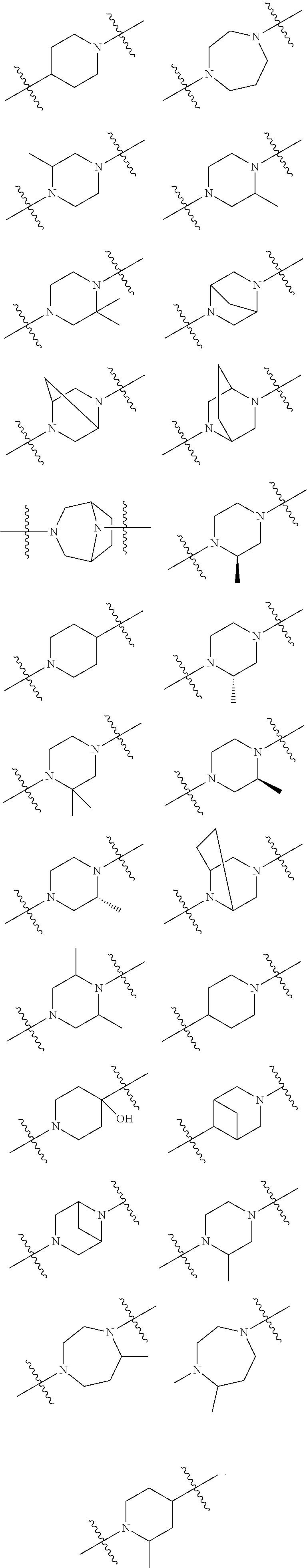

In some embodiments, the compounds of the disclosure are defined by formula (I′), wherein said compound is selected from the following structure:

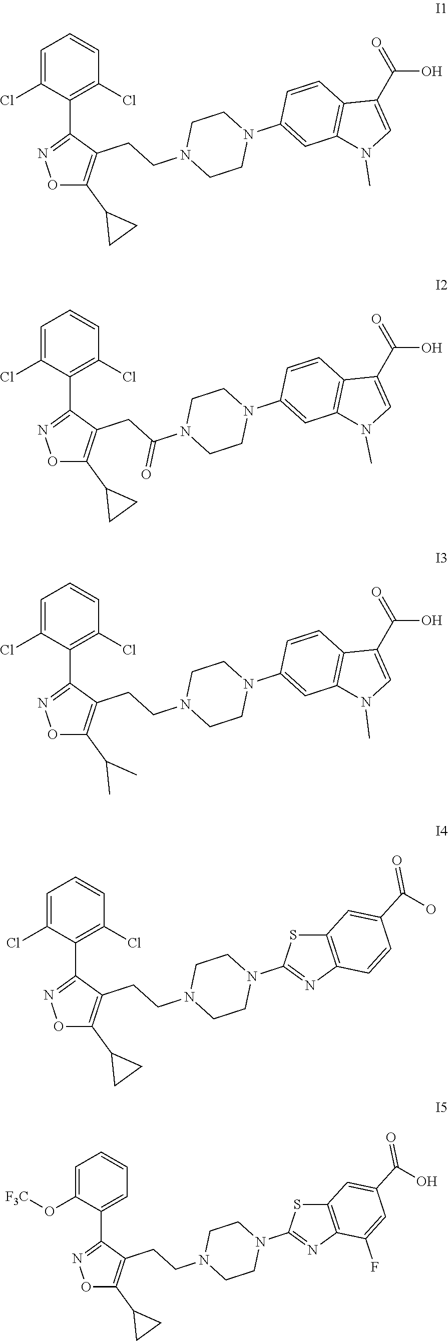 [SMILES: Cn1cc(C(=O)O)c2ccc(N3CCN(CCc4c(-c5c(Cl)cccc5Cl)noc4C4CC4)CC3)cc21 ; Cn1cc(C(=O)O)c2ccc(N3CCN(C(=O)Cc4c(-c5c(Cl)cccc5Cl)noc4C4CC4)CC3)cc21 ; CC(C)c1onc(-c2c(Cl)cccc2Cl)c1CCN1CCN(c2ccc3c(C(=O)O)cn(C)c3c2)CC1 ; O=C(O)c1ccc2nc(N3CCN(CCc4c(-c5c(Cl)cccc5Cl)noc4C4CC4)CC3)sc2c1 ; O=C(O)c1cc(F)c2nc(N3CCN(CCc4c(-c5ccccc5OC(F)(F)F)noc4C4CC4)CC3)sc2c1]

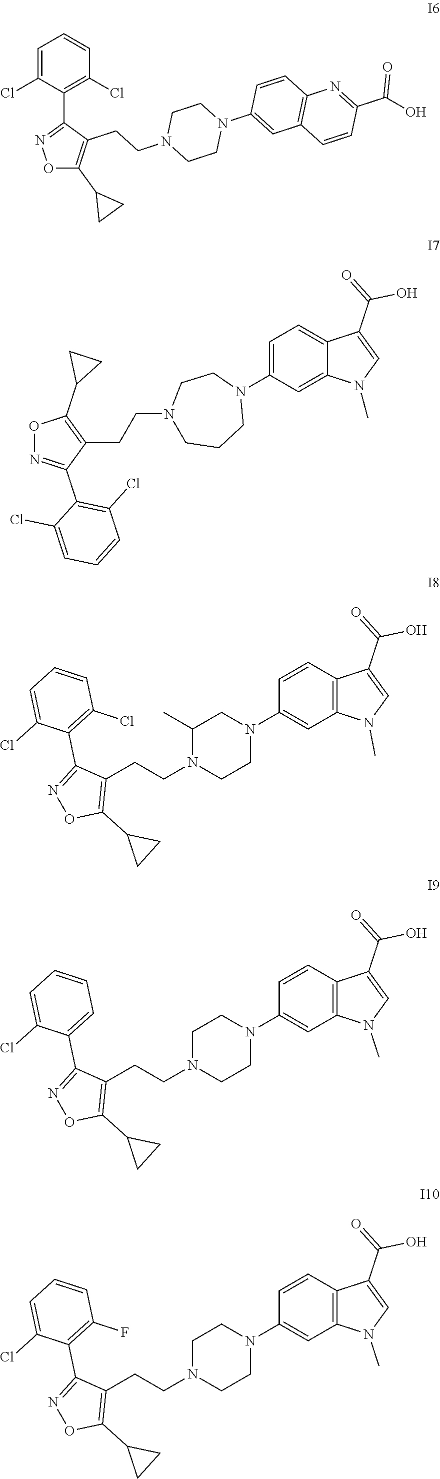 [SMILES: O=C(O)c1ccc2cc(N3CCN(CCc4c(-c5c(Cl)cccc5Cl)noc4C4CC4)CC3)ccc2n1 ; Cn1cc(C(=O)O)c2ccc(N3CCCN(CCc4c(-c5c(Cl)cccc5Cl)noc4C4CC4)CC3)cc21 ; CC1CN(c2ccc3c(C(=O)O)cn(C)c3c2)CCN1CCc1c(-c2c(Cl)cccc2Cl)noc1C1CC1 ; Cn1cc(C(=O)O)c2ccc(N3CCN(CCc4c(-c5ccccc5Cl)noc4C4CC4)CC3)cc21 ; Cn1cc(C(=O)O)c2ccc(N3CCN(CCc4c(-c5c(F)cccc5Cl)noc4C4CC4)CC3)cc21]

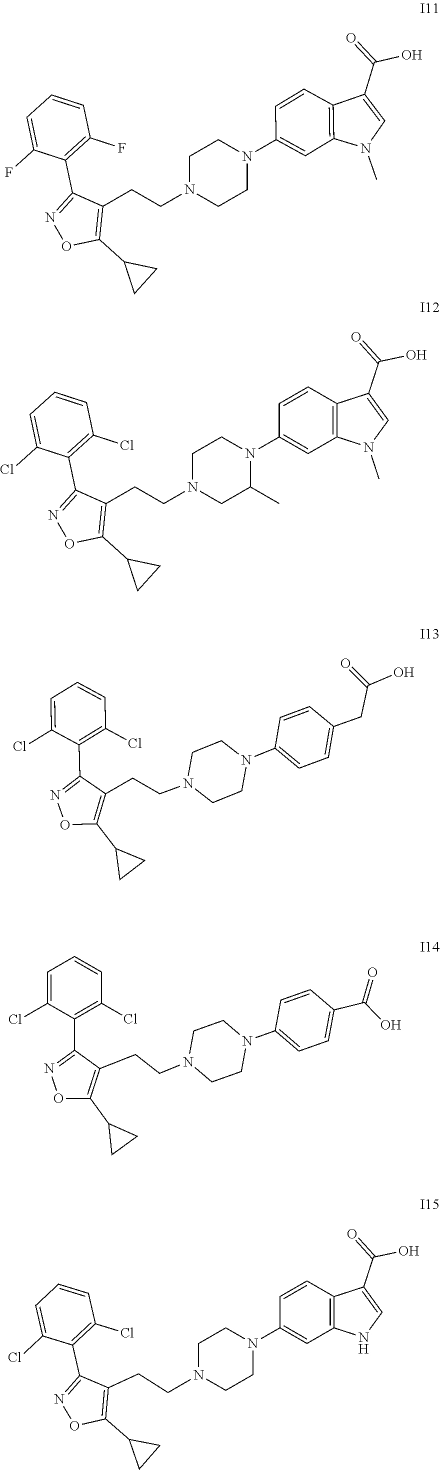 [SMILES: Cn1cc(C(=O)O)c2ccc(N3CCN(CCc4c(-c5c(F)cccc5F)noc4C4CC4)CC3)cc21 ; CC1CN(CCc2c(-c3c(Cl)cccc3Cl)noc2C2CC2)CCN1c1ccc2c(C(=O)O)cn(C)c2c1 ; O=C(O)Cc1ccc(N2CCN(CCc3c(-c4c(Cl)cccc4Cl)noc3C3CC3)CC2)cc1 ; O=C(O)c1ccc(N2CCN(CCc3c(-c4c(Cl)cccc4Cl)noc3C3CC3)CC2)cc1 ; O=C(O)c1c[nH]c2cc(N3CCN(CCc4c(-c5c(Cl)cccc5Cl)noc4C4CC4)CC3)ccc12]

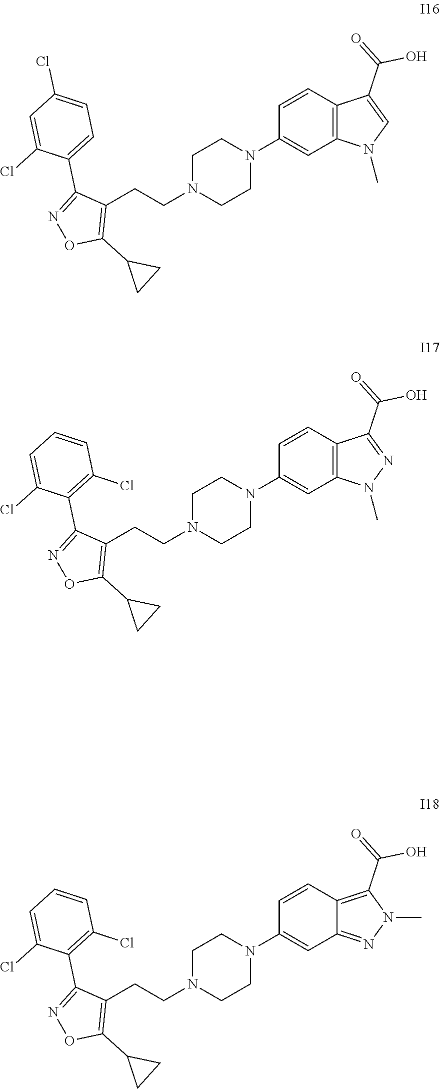 [SMILES: Cn1cc(C(=O)O)c2ccc(N3CCN(CCc4c(-c5ccc(Cl)cc5Cl)noc4C4CC4)CC3)cc21 ; Cn1nc(C(=O)O)c2ccc(N3CCN(CCc4c(-c5c(Cl)cccc5Cl)noc4C4CC4)CC3)cc21 ; Cn1nc2cc(N3CCN(CCc4c(-c5c(Cl)cccc5Cl)noc4C4CC4)CC3)ccc2c1C(=O)O]

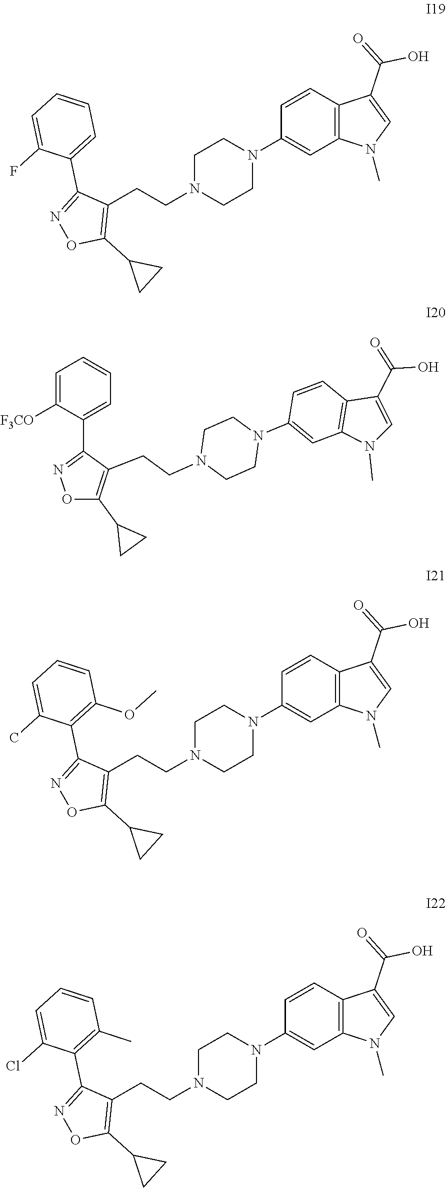 [SMILES: Cn1cc(C(=O)O)c2ccc(N3CCN(CCc4c(-c5ccccc5F)noc4C4CC4)CC3)cc21 ; Cn1cc(C(=O)O)c2ccc(N3CCN(CCc4c(-c5ccccc5OC(F)(F)F)noc4C4CC4)CC3)cc21 ; COc1cccc(C)c1-c1noc(C2CC2)c1CCN1CCN(c2ccc3c(C(=O)O)cn(C)c3c2)CC1 ; Cc1cccc(Cl)c1-c1noc(C2CC2)c1CCN1CCN(c2ccc3c(C(=O)O)cn(C)c3c2)CC1]

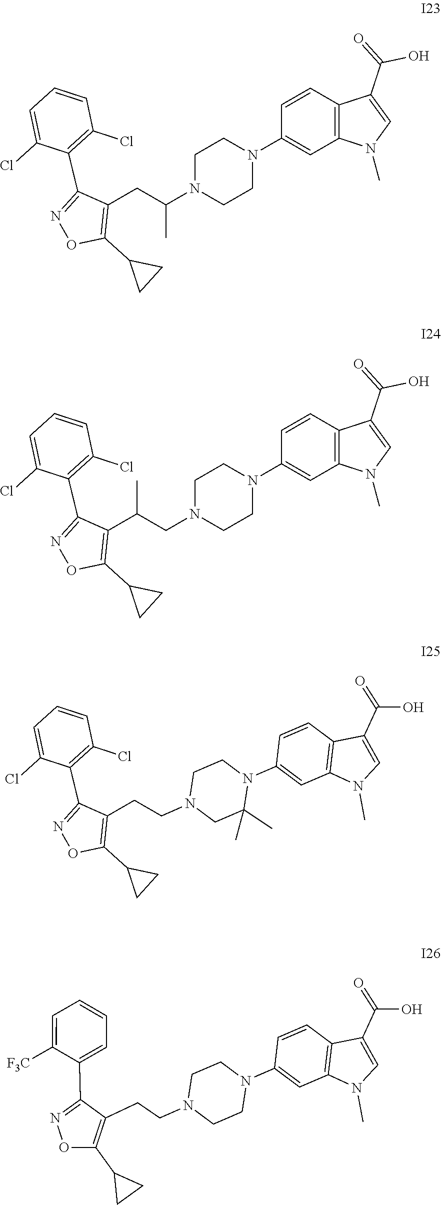 [SMILES: CC(Cc1c(-c2c(Cl)cccc2Cl)noc1C1CC1)N1CCN(c2ccc3c(C(=O)O)cn(C)c3c2)CC1 ; CC(CN1CCN(c2ccc3c(C(=O)O)cn(C)c3c2)CC1)c1c(-c2c(Cl)cccc2Cl)noc1C1CC1 ; Cn1cc(C(=O)O)c2ccc(N3CCN(CCc4c(-c5c(Cl)cccc5Cl)noc4C4CC4)CC3(C)C)cc21 ; Cn1cc(C(=O)O)c2ccc(N3CCN(CCc4c(-c5ccccc5C(F)(F)F)noc4C4CC4)CC3)cc21]

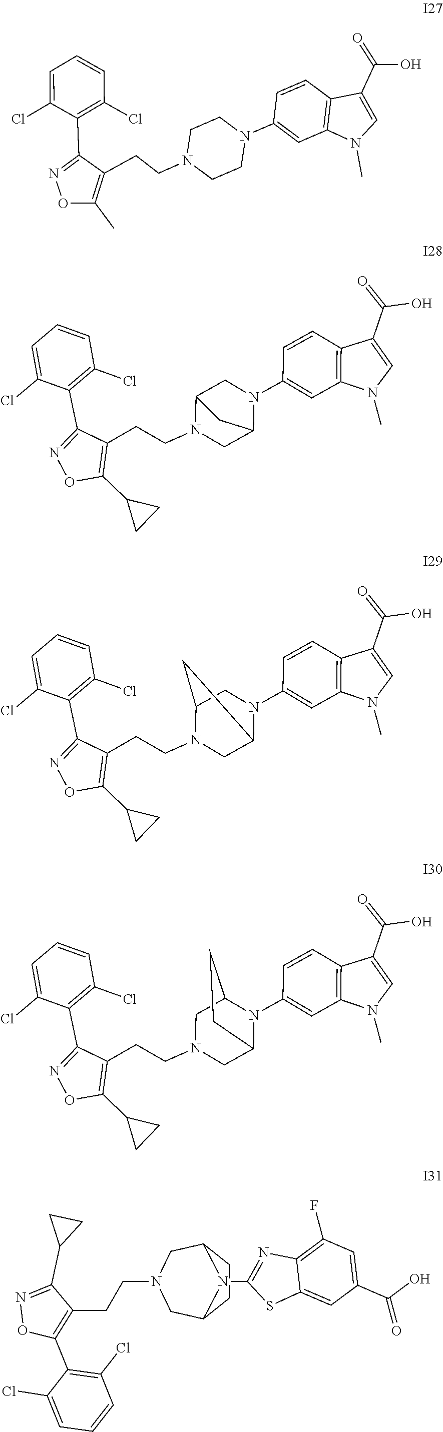 [SMILES: Cc1onc(-c2c(Cl)cccc2Cl)c1CCN1CCN(c2ccc3c(C(=O)O)cn(C)c3c2)CC1 ; Cn1cc(C(=O)O)c2ccc(N3CC4CC3CN4CCc3c(-c4c(Cl)cccc4Cl)noc3C3CC3)cc21 ; Cn1cc(C(=O)O)c2ccc(N3CC4CC3CN4CCc3c(-c4c(Cl)cccc4Cl)noc3C3CC3)cc21 ; Cn1cc(C(=O)O)c2ccc(N3C4CCC3CN(CCc3c(-c5c(Cl)cccc5Cl)noc3C3CC3)C4)cc21 ; O=C(O)c1cc(F)c2nc(N3C4CCC3CN(CCc3c(C5CC5)noc3-c3c(Cl)cccc3Cl)C4)sc2c1]

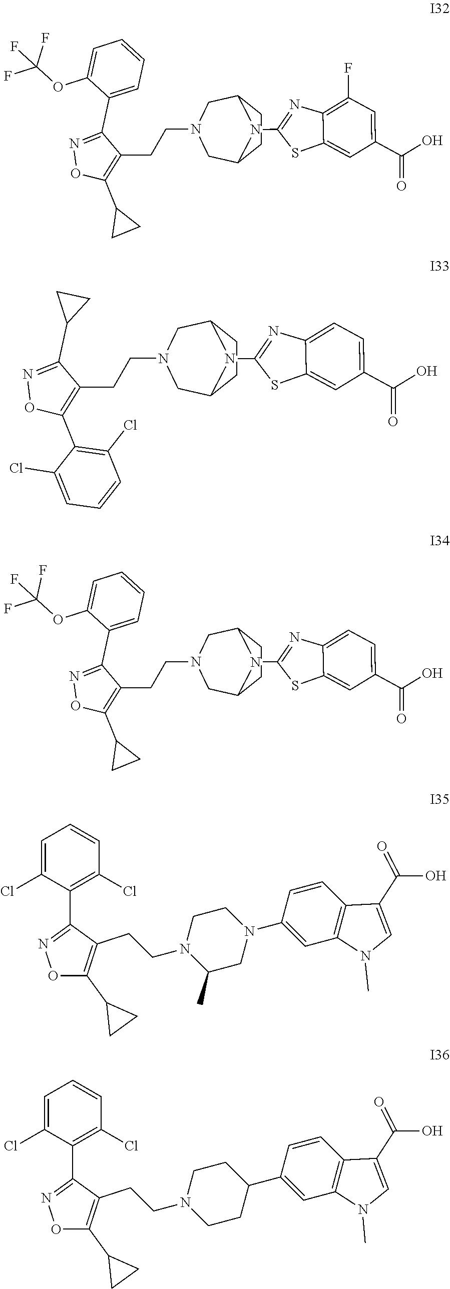 [SMILES: O=C(O)c1cc(F)c2nc(N3C4CCC3CN(CCc3c(-c5ccccc5OC(F)(F)F)noc3C3CC3)C4)sc2c1 ; O=C(O)c1ccc2nc(N3C4CCC3CN(CCc3c(C5CC5)noc3-c3c(Cl)cccc3Cl)C4)sc2c1 ; O=C(O)c1ccc2nc(N3C4CCC3CN(CCc3c(-c5ccccc5OC(F)(F)F)noc3C3CC3)C4)sc2c1 ; C[C@@H]1CN(c2ccc3c(C(=O)O)cn(C)c3c2)CCN1CCc1c(-c2c(Cl)cccc2Cl)noc1C1CC1 ; Cn1cc(C(=O)O)c2ccc(C3CCN(CCc4c(-c5c(Cl)cccc5Cl)noc4C4CC4)CC3)cc21]

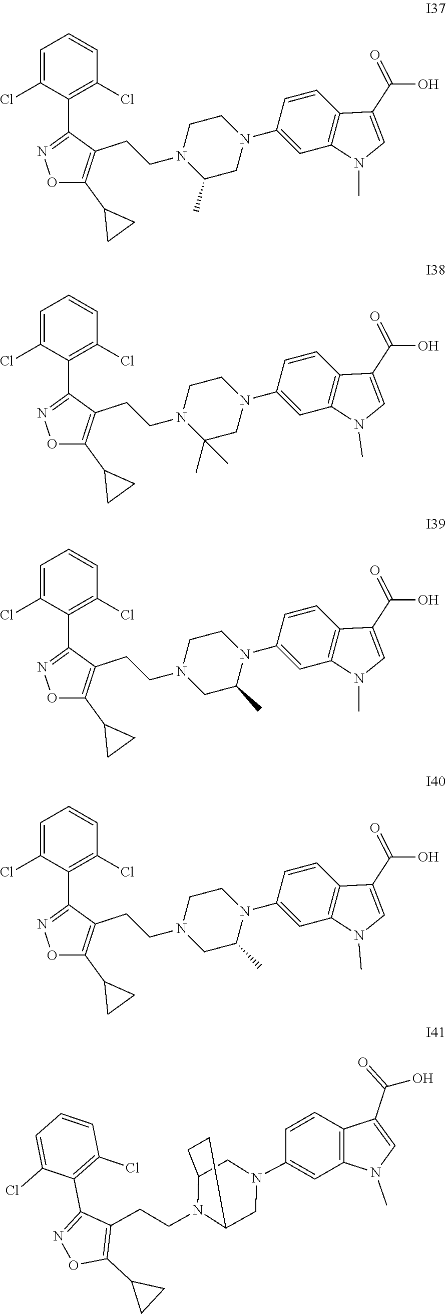 [SMILES: C[C@H]1CN(c2ccc3c(C(=O)O)cn(C)c3c2)CCN1CCc1c(-c2c(Cl)cccc2Cl)noc1C1CC1 ; Cn1cc(C(=O)O)c2ccc(N3CCN(CCc4c(-c5c(Cl)cccc5Cl)noc4C4CC4)C(C)(C)C3)cc21 ; C[C@H]1CN(CCc2c(-c3c(Cl)cccc3Cl)noc2C2CC2)CCN1c1ccc2c(C(=O)O)cn(C)c2c1 ; C[C@@H]1CN(CCc2c(-c3c(Cl)cccc3Cl)noc2C2CC2)CCN1c1ccc2c(C(=O)O)cn(C)c2c1 ; Cn1cc(C(=O)O)c2ccc(N3CC4CCC(C3)N4CCc3c(-c4c(Cl)cccc4Cl)noc3C3CC3)cc21]

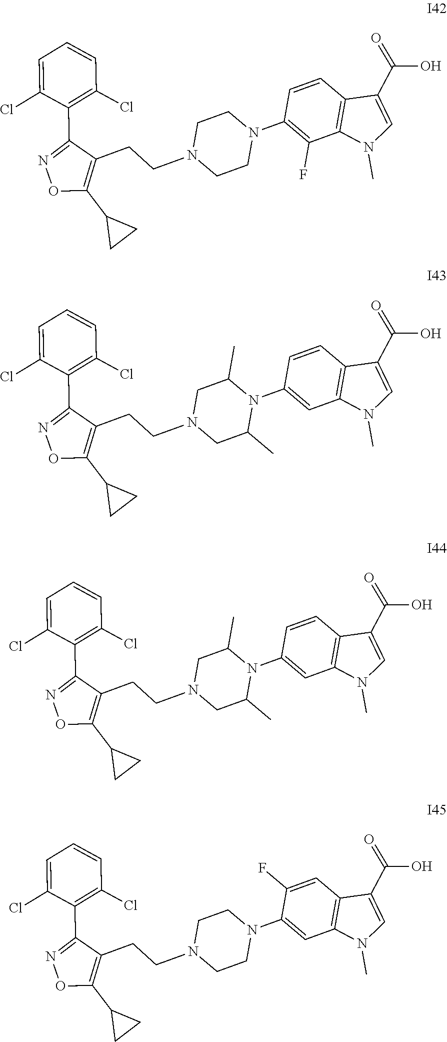 [SMILES: Cn1cc(C(=O)O)c2ccc(N3CCN(CCc4c(-c5c(Cl)cccc5Cl)noc4C4CC4)CC3)c(F)c21 ; CC1CN(CCc2c(-c3c(Cl)cccc3Cl)noc2C2CC2)CC(C)N1c1ccc2c(C(=O)O)cn(C)c2c1 ; CC1CN(CCc2c(-c3c(Cl)cccc3Cl)noc2C2CC2)CC(C)N1c1ccc2c(C(=O)O)cn(C)c2c1 ; Cn1cc(C(=O)O)c2cc(F)c(N3CCN(CCc4c(-c5c(Cl)cccc5Cl)noc4C4CC4)CC3)cc21]

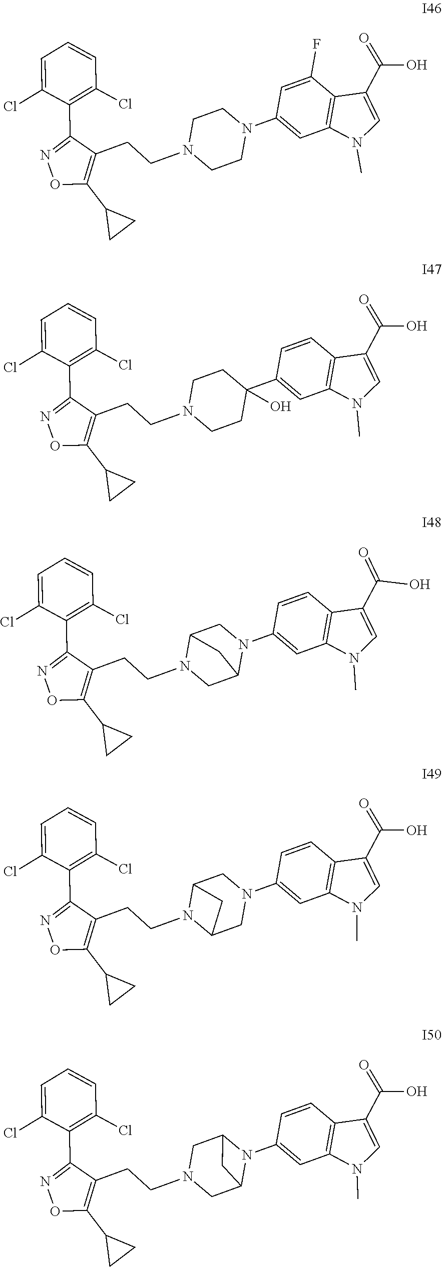 [SMILES: Cn1cc(C(=O)O)c2c(F)cc(N3CCN(CCc4c(-c5c(Cl)cccc5Cl)noc4C4CC4)CC3)cc21 ; Cn1cc(C(=O)O)c2ccc(C3(O)CCN(CCc4c(-c5c(Cl)cccc5Cl)noc4C4CC4)CC3)cc21 ; Cn1cc(C(=O)O)c2ccc(N3CC4CC3CN4CCc3c(-c4c(Cl)cccc4Cl)noc3C3CC3)cc21 ; Cn1cc(C(=O)O)c2ccc(N3CC4CC(C3)N4CCc3c(-c4c(Cl)cccc4Cl)noc3C3CC3)cc21 ; Cn1cc(C(=O)O)c2ccc(N3C4CC3CN(CCc3c(-c5c(Cl)cccc5Cl)noc3C3CC3)C4)cc21]

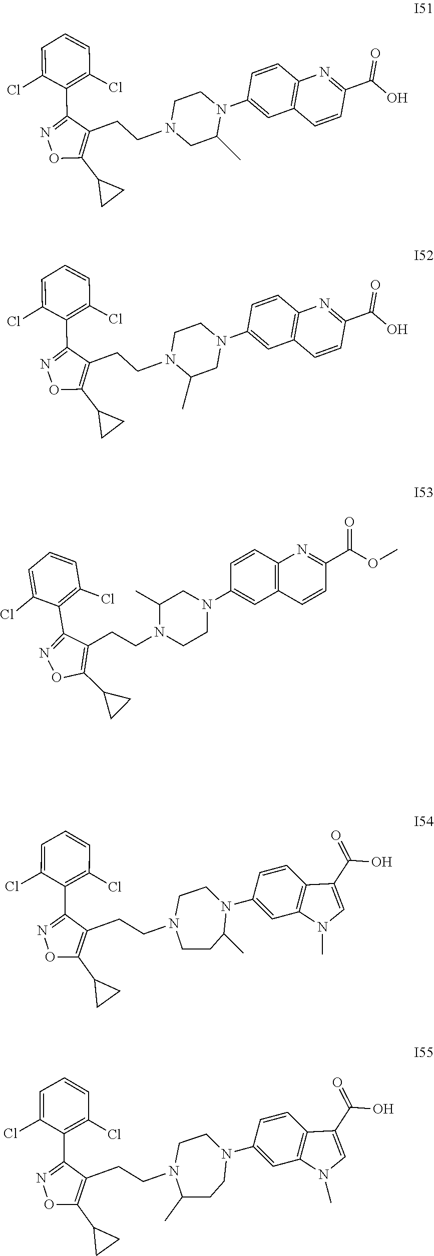 [SMILES: CC1CN(CCc2c(-c3c(Cl)cccc3Cl)noc2C2CC2)CCN1c1ccc2nc(C(=O)O)ccc2c1 ; CC1CN(c2ccc3nc(C(=O)O)ccc3c2)CCN1CCc1c(-c2c(Cl)cccc2Cl)noc1C1CC1 ; COC(=O)c1ccc2cc(N3CCN(CCc4c(-c5c(Cl)cccc5Cl)noc4C4CC4)C(C)C3)ccc2n1 ; CC1CCN(CCc2c(-c3c(Cl)cccc3Cl)noc2C2CC2)CCN1c1ccc2c(C(=O)O)cn(C)c2c1 ; CC1CCN(c2ccc3c(C(=O)O)cn(C)c3c2)CCN1CCc1c(-c2c(Cl)cccc2Cl)noc1C1CC1]

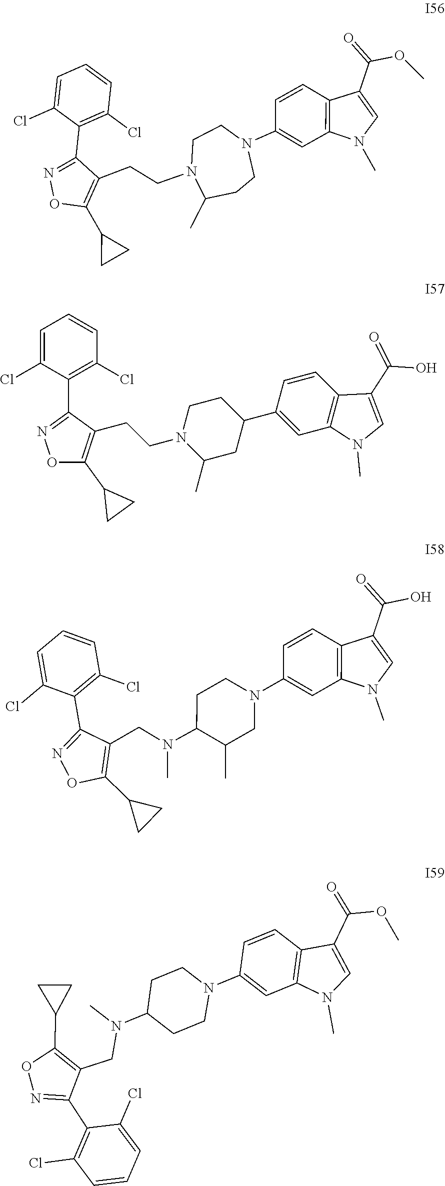 [SMILES: COC(=O)c1cn(C)c2cc(N3CCC(C)N(CCc4c(-c5c(Cl)cccc5Cl)noc4C4CC4)CC3)ccc12 ; CC1CC(c2ccc3c(C(=O)O)cn(C)c3c2)CCN1CCc1c(-c2c(Cl)cccc2Cl)noc1C1CC1 ; CC1CN(c2ccc3c(C(=O)O)cn(C)c3c2)CCC1N(C)Cc1c(-c2c(Cl)cccc2Cl)noc1C1CC1 ; COC(=O)c1cn(C)c2cc(N3CCC(N(C)Cc4c(-c5c(Cl)cccc5Cl)noc4C4CC4)CC3)ccc12]

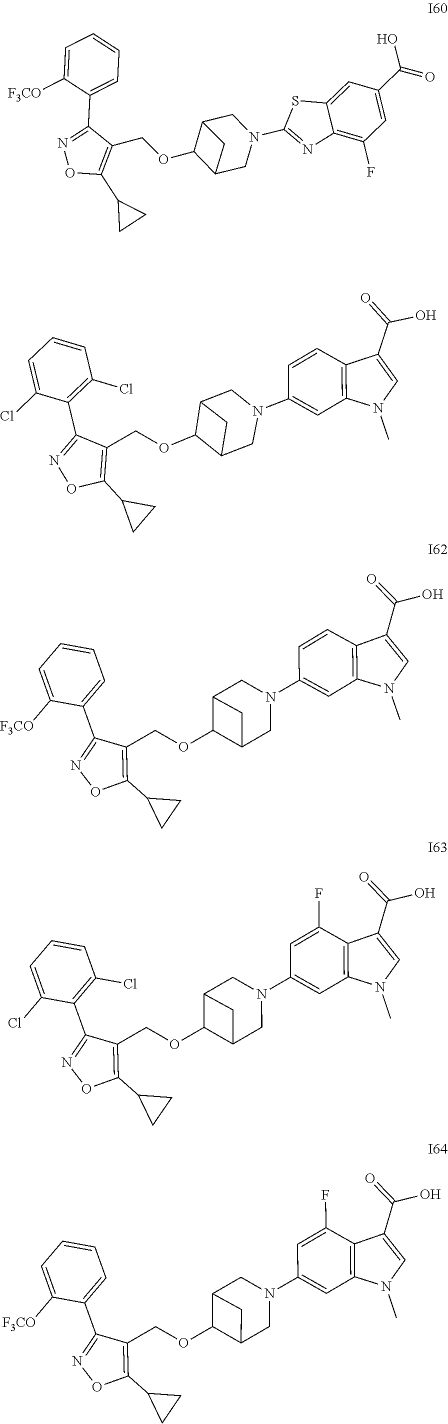 [SMILES: O=C(O)c1cc(F)c2nc(N3CC4CC(C3)C4OCc3c(-c4ccccc4OC(F)(F)F)noc3C3CC3)sc2c1 ; Cn1cc(C(=O)O)c2ccc(N3CC4CC(C3)C4OCc3c(-c4c(Cl)cccc4Cl)noc3C3CC3)cc21 ; Cn1cc(C(=O)O)c2ccc(N3CC4CC(C3)C4OCc3c(-c4ccccc4OC(F)(F)F)noc3C3CC3)cc21 ; Cn1cc(C(=O)O)c2c(F)cc(N3CC4CC(C3)C4OCc3c(-c4c(Cl)cccc4Cl)noc3C3CC3)cc21 ; Cn1cc(C(=O)O)c2c(F)cc(N3CC4CC(C3)C4OCc3c(-c4ccccc4OC(F)(F)F)noc3C3CC3)cc21]

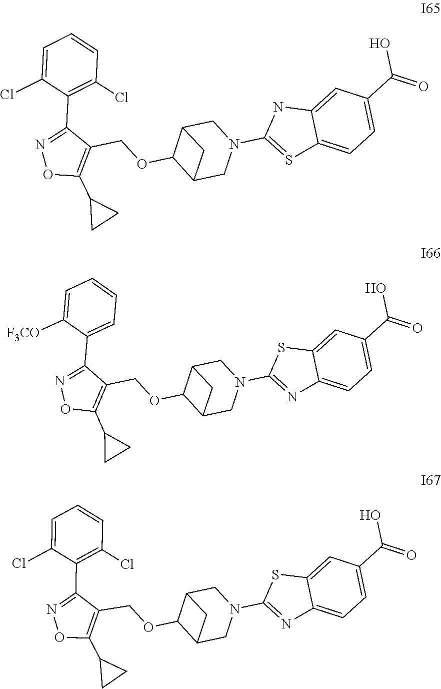

or a stereoisomer, enantiomer or a pharmaceutically acceptable salt thereof.

The compounds of the present disclosure are agonists of FXRs. The present disclosure also provides a pharmaceutical composition comprising a therapeutically effective amount of a compound of present disclosure and a pharmaceutically acceptable carrier.

The present disclosure also provides a combination comprising a therapeutically effective amount of present disclosure in the treatment of cholestasis, intrahepatic cholestatis, estrogen-induced cholestasis, drug-induced cholestasis, cholestasis of pregnancy, parenteral nutrition-associated cholestasis, primary biliary cirrhosis (PBC), primary sclerosing cholangistis (PSC), progressive familiar cholestatis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, dyslipidemia, atherosclerosis, diabetes, diabetic nephropathy, colitis, newborn jaundice, prevention of kernicterus, venocclusive disease, portal hypertension, metabolic syndrome, hypercholesterolemia, intestinal bacterial overgrowth, or erectile dysfunction.

The present disclosure also provides a method for treating a condition mediated by FXR in a subject suffering therefrom, comprising administering to the subject a therapeutically effective amount of present disclosure, or a pharmaceutical composition thereof.

A pharmaceutical composition comprising a compound according to the present disclosure for use in the treatment of a condition mediated by FXR.

Use of any compound of the present disclosure, or a pharmaceutical composition thereof, for the preparation of a medicament for the treatment of a condition mediated by FXR in a subject.

Wherein said condition is cholestasis, intrahepatic cholestatis, estrogen-induced cholestasis, drug-induced cholestasis, cholestasis of pregnancy, parenteral nutrition-associated cholestasis, primary biliary cirrhosis (PBC), primary sclerosing cholangistis (PSC), progressive familiar cholestatis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, dyslipidemia, atherosclerosis, diabetes, diabetic nephropathy, colitis, newborn jaundice, prevention of kernicterus, venocclusive disease, portal hypertension, metabolic syndrome, hypercholesterolemia, intestinal bacterial overgrowth, or erectile dysfunction.

### Definitions

As used herein, “C₁₋₆ alkyl” denotes an alkyl radical having from 1 up to 6, particularly up to 4 carbon atoms, the radicals being either linear or branched with single or multiple branching; for example, butyl, such as n-butyl, sec-butyl, isobutyl, tert-butyl; propyl, such as n-propyl or isopropyl; ethyl or methyl; more particularly, methyl, propyl or tert-butyl. “C₁₋₃ alkyl” refers to an alkyl radical as defined herein, containing one to three carbon atoms.

As used herein, the term “alkylene” refers to divalent alkyl group as defined herein above having a specified number of carbon atoms. Representative examples of alkylene include, but are not limited to, methylene, ethylene, n-propylene, iso-propylene, n-butylene, sec-butylene, iso-butylene, tert-butylene, and the like.

As used herein, “aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic substituent which can be a single aromatic ring, or multiple aromatic rings that are fused together; and may encompass monovalent and divalent aryls, which will be apparent to those skilled in the art. Non-limiting examples include phenyl, phenylene, naphthyl, naphthylene, tetrahydronaphthyl or tetrahydronaphthylene.

As used herein, “heteroaryl” refers to a 5-14 membered monocyclic- or bicyclic- or tricyclic-aromatic ring system having 1 to 8 heteroatoms. Typically, the heteroaryl is a 5-10 membered ring system (e.g., 5-7 membered monocycle or an 8-10 membered bicycle) or a 5-7 membered ring system. Furthermore, the term “heteroaryl” as used herein may encompass monovalent or divalent heteroaryls, which will be apparent to those skilled in the art. Typical monocyclic heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, 2-, 4-, or 5-pyrimidinyl, and monovalent or divalent forms thereof. Typical bicyclic heteroaryl groups include benzofuranyl, benzo[d]isothiazolyl, benzo[d]isoxazolyl, benzothiazolyl, benzo[b]thiophenyl, imidazo[1,2-a]pyridinyl, imidazo[1,2-b]pyridazinyl, 1H-indolyl, 1H-indazolyl, pyrazolo[1,5-a]pyridinyl, pyrrolo[1,2-b]pyridazinyl, 1H-pyrrolo[3,2-c]pyridinyl, pyrazolo[1,5-a]pyrimidinyl, quinazolinyl and the like, and monovalent or divalent forms thereof.

As used herein, “C₁₋₆ alkoxy” refers to C₁₋₆ alkyl-O—, and is particularly methoxy, ethoxy, isopropyloxy, or tert-butoxy.

As used herein, “halogen” or “halo” refers to fluoro, chloro, bromo, and iodo; and more particularly, fluoro or chloro.

As used herein, “haloC₁₋₆ alkyl” refers to an alkyl radical, as defined above, that is substituted by one or more halo radicals, as defined above, and is particularly fluoroC₁₋₆ alkyl, more particularly trifluoromethyl.

As used herein, “haloC₁₋₆ alkoxy” refers to an alkoxy radical, as defined above, that is substituted by one or more halo radicals, as defined above, and is particularly fluoroC₁₋₆ alkoxy, more particularly, trifluoromethoxy or difluoromethoxy.

disclosureAs used herein, the term “therapeutically effective amount” refers to an amount of the compound of Formula I, Formula I′ and (I-A) to (I-G) which is sufficient to achieve the stated effect.

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

### DISCLOSURE

### Detailed Description

disclosuredisclosureIn one aspect, compounds of the disclosure are defined by Formula (I)

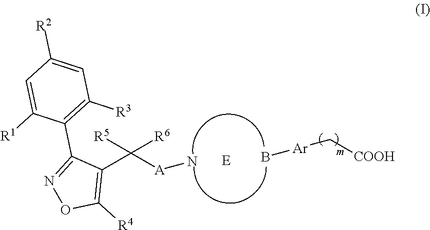

or a stereoisomer, enantiomer, a pharmaceutically acceptable salt or an amino acid conjugate thereof;

R¹, R² and R³ are independently selected from H, C₁₋₆ alkyl, haloC₁₋₆ alkyl, C₁₋₆ alkoxy, haloC₁₋₆ alkoxy, or cyclopropyl;

R⁴ is selected from C₁₋₃ alkyl, haloC₁₋₃ alkyl or cyclopropyl optionally substituted with C₁₋₃ alkyl or haloC₁₋₃ alkyl;

R⁵ and R⁶ are independently selected from H, C₁₋₃ alkyl or haloC₁₋₃ alkyl;

A is selected from C═O or CR⁷R⁸;

R⁷ and R⁸ are independently selected from H, C₁₋₃ alkyl or C₁₋₃ alkoxy;

B is CH or N;

ring E is a substituted or unsubstituted 6-8 membered heteroring or bridged-heteroring;

Ar is phenylene, C₅₋₇ cycloalkylene or 5-14 membered monocyclic or bicyclic heteroaryl containing 1-2 heteroatoms selected from N, O and S; each of which is optionally substituted with R¹⁰ and R¹¹,

R¹⁰ and R¹¹ are independently selected from H, halogen, C₁₋₆ alkyl, haloC₁₋₆ alkyl, C₁₋₆ alkoxy, haloC₁₋₆ alkoxy, or cyclopropyl;

m is 0 or 1.

In another aspect, compounds of the disclosure are defined by Formula (I′)

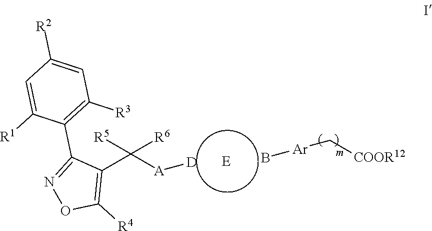

or a stereoisomer, enantiomer or a pharmaceutically acceptable salt thereof;

R¹, R² and R³ are independently selected from H, C₁₋₆alkyl, haloC₁₋₆alkyl, C₁₋₆ alkoxy, haloC₁₋₆ alkoxy, or cyclopropyl;

R⁴ is selected from C₁₋₃alkyl, haloC₁₋₃alkyl or cyclopropyl optionally substituted with C₁₋₃ alkyl or haloC₁₋₃alkyl;

R⁵ and R⁶ are independently selected from H, C₁₋₃alkyl or haloC₁₋₃alkyl;

A is selected from C═O, CR⁷R⁸, O or NR⁹;

R⁷ and R⁸ are independently selected from H, C₁₋₃alkyl or C₁₋₃alkoxy;

R⁹ is selected from H, C₁₋₃alkyl Or C₁₋₃alkoxy;

B is CR¹³ or N;

D is CR¹⁴ or N:

ring E is a substituted or unsubstituted 6-8 membered heteroring or bridged-heteroring;

Ar is phenylene, C₅₋₇ cycloalkylene or 5-14 membered monocyclic or bicyclic heteroaryl containing 1-2 heteroatoms selected from N, O and S; each of which is optionally substituted with R¹⁰ and R¹¹,

R¹⁰ and R¹¹ are independently selected from H, halogen, C₁₋₆ alkyl, haloC₁₋₆ alkyl, C₁₋₆ alkoxy, haloC₁₋₆ alkoxy, or cyclopropyl;

R¹² is selected from H, C₁₋₃alkyl or C₁₋₃alkoxy:

R¹³ is selected from H, OH, C₁₋₃alkyl or C₁₋₃alkoxy:

R¹⁴ is selected from H, OH, C₁₋₃alkyl or C₁₋₃alkoxy;

m is 0 or 1.

In some embodiments, wherein said compound is selected from the group consisting of:

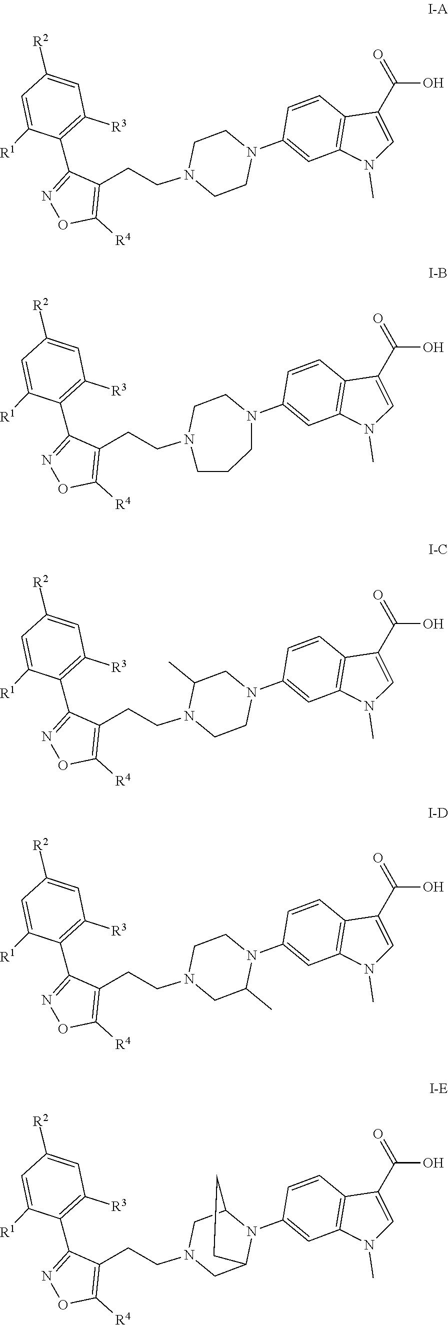

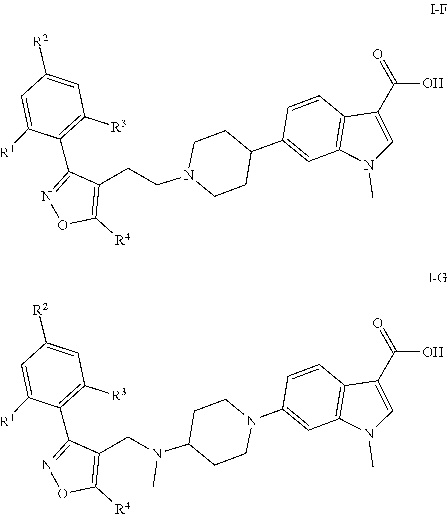

Wherein R¹, R² and R³ are independently selected from H, Cl, F, CH₃, OCF₃, CF₃ and OMe, R⁴ is cyclopropyl or i-Pr,

or a stereoisomer, enantiomer or a pharmaceutically acceptable salt thereof.

In another aspect, the present disclosure provides pharmaceutical compositions comprising a compound having Formula I, Formula I′ and (I-A) to (I-G) and a pharmaceutically acceptable carrier. The present disclosure also provides a pharmaceutical composition comprising a compound of Formula I and (I-A) to (I-G) for use in the treatment of a condition mediated by FXR.

The compounds of Formula I, Formula I′ and (I-A) to (I-G) and their pharmaceutically acceptable salts exhibit valuable pharmacological properties when tested in vitro in cell-free kinase assays and in cellular assays, and are therefore useful as pharmaceuticals. disclosuredisclosure

In another aspect, the disclosure provides methods for modulating FXR in a cell, comprising contacting the cell with an effective amount of a compound of Formula I, Formula I′ and (I-A) to (I-G) or a pharmaceutical composition thereof.

In another aspect, the disclosure provides methods to treat, ameliorate or prevent a FXR-mediated disorder in a subject suffering there from, comprising administering to the subject a therapeutically effective amount of a compound of Formula I, Formula I′ and (I-A) to (I-G), or a pharmaceutical composition thereof, and optionally in combination with a second therapeutic agent. The present disclosure also provides for the use of a compound of Formula I, Formula I′ and (I-A) to (I-G), and optionally in combination with a second therapeutic agent, in the manufacture of a medicament for treating a FXR-mediated disorder such as cholestasis, intrahepatic cholestatis, estrogen-induced cholestasis, drug-induced cholestasis, cholestasis of pregnancy, parenteral nutrition-associated cholestasis, PBC, PSC, PFIC, NAFLD, NASH, drug-induced bile duct injury, gallstones, liver cirrhosis, alcohol-induced cirrhosis, cystic fibrosis, bile duct obstruction, cholelithiasis, liver fibrosis, dyslipidemia, atherosclerosis, diabetes, diabetic nephropathy, colitis, newborn jaundice, prevention of kernicterus, venocclusive disease, portal hypertension, metabolic syndrome, hypercholesterolemia, intestinal bacterial overgrowth, or erectile dysfunction.

In yet another aspect, the present disclosure provides a combination comprising a therapeutically effective amount of a compound of Formula I, Formula I′ and (I-A) to (I-G), and a second therapeutic agent being useful in the treatment of FXR-mediated conditions disorder described above.

Unless specified otherwise, the term “compounds of the present disclosure” refers to compounds of Formula I, Formula I′ and (I-A) to (I-G), prodrugs thereof, salts of the compound and/or prodrugs, hydrates or solvates of the compounds, salts and/or prodrugs, as well as all stereoisomers (including diastereoisomers and enantiomers), tautomers and isotopically labeled compounds (including deuterium substitutions), as well as inherently formed moieties (e.g., polymorphs, solvates and/or hydrates).

disclosuredisclosuredisclosuredisclosuredisclosurePharmaceutically acceptable solvates in accordance with the disclosure include those wherein the solvent of crystallization may be isotopically substituted.

Compounds of the disclosure, i.e. compounds of Formula I, Formula I′ and (I-A) to (I-G) that contain groups capable of acting as donors and/or acceptors for hydrogen bonds may be capable of forming co-crystals with suitable co-crystal formersdisclosure.

disclosuredisclosuredisclosurePHARMACOLOGY AND UTILITY

In one embodiment, said compounds and pharmaceutical compositions are used for the preparation of a medicament for the treatment of chronic intrahepatic and some forms of extrahepatic cholestatic conditions, such as primary biliary cirrhosis (PBC), primary sclerosing cholangitis (PSC), progressive familiar cholestasis (PFIC), alcohol-induced cirrhosis and associated cholestasis, or liver fibrosis resulting from chronic cholestatic conditions or acute intraheptic cholestatic conditions such as estrogen or drug induced cholestasis.

In another embodiment, the compounds according to the disclosure and pharmaceutical compositions comprising said compounds are used in the treatment of Type II Diabetes which can be overcome by FXR-mediated upregulation of systemic insulin sensitivity and intracellular insulin signalling in liver, increased peripheral glucose uptake and metabolisation, increased glycogen storage in liver, decreased output of glucose into serum from liver-borne gluconeogenesis.

The disclosure also relates to a compound of Formula I, Formula I′ and (I-A) to (I-G), or to a pharmaceutical composition comprising said compound, for the treatment of gastrointestinal conditions with a reduced uptake of dietary fat and fat-soluble dietary vitamins which can be overcome by increased intestinal levels of bile acids and phospholipids.

In another embodiment, the compounds according to the disclosure are useful for beneficially altering lipid profiles, including but not limited to lowering total cholesterol levels, lowering LDL cholesterol levels, lowering VLDL cholesterol levels, raising HDL cholesterol levels, and/or lowering triglyceride levels. Thus, the present disclosure provides a method for treating FXR mediated conditions such as dyslipidemia and diseases related to dyslipidemia comprising administering a therapeutically effective amount of a compound of the present disclosure to a subject in need thereof.

In a further embodiment, said compound or pharmaceutical composition is used for treating a disease selected from the group consisting of lipid and lipoprotein disorders such as hypercholesterolemia, hypertriglyceridemia, and atherosclerosis as a clinically manifest condition which can be ameliorated by FXR's beneficial effect on raising HDL cholesterol, lowering serum triglycerides, increasing conversion of liver cholesterol into bile acids and increased clearance and metabolic conversion of VLDL and other lipoproteins in the liver.

In one further embodiment, said compound and pharmaceutical composition are used for the preparation of a medicament where the combined lipid lowering, anti-cholestatic and antI-Gibrotic effects of FXR-targeted medicaments can be exploited for the treatment of liver steatosis and associated syndromes such as non-alcoholic steatohepatitis (“NASH”), or for the treatment of cholestatic and fibrotic effects that are associated with alcohol-induced cirrhosis, or with viral-borne forms of hepatitis.

In conjunction with the hypolipidemic effects, it was also shown that loss of functional FXR leads to increased atherosclerosis in ApoE knockout mice (Hanniman et al., J. Lipid Res. 2005, 46(12), 2595-2604). Therefore, FXR agonists might have clinical utility as anti-atherosclerotic and cardioprotective drugs. The downregulation of Endothelin-1 in Vascular Smooth Muscle Cells might also contribute to such beneficial therapeutic effects (He et al., Circ. Res. 2006, 98(2), 192-9).

The disclosure also relates to a compound according to Formula I, Formula I′ and (I-A) to (I-G) or a pharmaceutical composition comprising said compound, for preventive and posttraumatic treatment of cardiovascular disorders such as acute myocardial infarction, acute stroke, or thrombosis which occur as an endpoint of chronic obstructive atherosclerosis. In a few selected publications, the effects of FXR and FXR agonists on proliferation of cancer and non-malignant cells and apoptosis have been assessed. From these preliminary results it seems as if FXR agonists might also influence apoptosis in cancer cell lines (Niesor et al., Curr. Pharm. Des. 2001, 7(4), 231-59) and in Vascular Smooth Muscle Cells (VSMCs) (Bishop-Bailey et al., Proc. Natl. Acad. Sci. USA. 2004, 101(10), 3668-3673).

Furthermore, FXR seems to be expressed in metastasizing breast cancer cells and in colon cancer (Silva, J. Lipid Res. 2006, 47(4), 724-733; De Gottardi et al., Dig. Dis. Sci. 2004, 49(6), 982-989). Other publications that focus primarily on FXR's effect on metabolism draw a line to intracellular signaling from FXR via the Forkhead/Wingless (FOXO) family of transcriptional modulators to the Phosphatidylinositol-trisphosphat (PI3)-Kinase/Akt signal transduction pathway (Duran-Sandoval et al., J. Biol. Chem. 2005, 280(33), 29971-29979; Zhang et al., Proc. Natl. Acad. Sci. USA. 2006, 103(4), 1006-1011) that is similarly employed by insulin intracellular signaling as well as neoplastically transformed cells. Thus, FXR may also be a potential target for the treatment of proliferative diseases, especially metastasizing cancer forms that overexpress FXR or those where the FOXO/PI3-Kinase/Akt Pathway is responsible for driving proliferation. Therefore, the compounds according to Formula I, Formula I′ and (I-A) to (I-G), or pharmaceutical composition comprising said compounds are suitable for treating non-malignant hyperproliferative disorders such as increased neointima formation after balloon vessel dilatation and stent application due to increased proliferation of vascular smooth muscle cells (VSMCs) or Bening Prostate Hyperplasia (BPH), a pre-neoplastic form of hyperproliferation, other forms of scar tissue formation and fibrotisation which can be overcome by e.g. FXR-mediated intervention into the PI-3Kinase/AKT/mTOR intracellular signalling pathway, reduction in Matrix-Metalloproteinase activity and alpha-Collagen deposition.

In a further embodiment, said compounds and pharmaceutical compositions are used for the treatment of malignant hyperproliferative disorders such as cancer (e.g. certain forms of breast or prostate cancer) where interference with PI-3-Kinase/AKT/mTOR signalling and/or induction of p27kip and/or induction of apoptosis will have a beneficial impact.

FXR seems also to be involved in the control of antibacterial defense in the intestine (Inagaki et al., Proc. Natl. Acad. Sci. USA. 2006, 103(10), 3920-3905) although an exact mechanism is not provided. From these published data, however, one can conclude that treatment with FXR agonists might have a beneficial impact in the therapy of Inflammatory Bowel Disorders (IBD), in particular those forms where the upper (ileal) part of the intestine is affected (e.g. ileal Crohn's disease) because this seems to be the site of action of FXR's control on bacterial growth. In IBD, the desensitization of the adaptive immune response is somehow impaired in the intestinal immune system. Bacterial overgrowth might then be the causative trigger towards establishment of a chronic inflammatory response. Hence, dampening of bacterial growth by FXR-borne mechanisms might be a key mechanism to prevent acute inflammatory episodes. Thus, the disclosure also relates to a compound according to formula I and Formula I′ or a pharmaceutical composition comprising said compound for treating a disease related to Inflammatory Bowel Diseases such as Crohn's disease or Colitis ulcerosa. FXR-mediated restoration of intestinal barrier function and reduction in non-commensal bacterial load is believed to be helpful in reducing the exposure of bacterial antigens to the intestinal immune system and can therefore reduce inflammatory responses.

The disclosure further relates to a compound or pharmaceutical composition for the treatment of obesity and associated disorders such as metabolic syndrome (combined conditions of dyslipidemias, diabetes and abnormally high body-mass index) which can be overcome by FXR-mediated lowering of serum triglycerides, blood glucose and increased insulin sensitivity and FXR-mediated weight loss.

In one embodiment, said compound or pharmaceutical composition is for treating persistent infections by intracellular bacteria or parasitic protozoae such as Mycobacterium spec. (Treatment of Tuberculosis or Lepra), Listeria monocytogenes (Treatment of Listeriosis), Leishmania spec. (Leishmaniosis), Trypanosoma spec. (Chagas Disease; Trypanosomiasis; Sleeping Sickness).

In a further embodiment, the compounds or pharmaceutical composition of the present disclosure are useful in the preparation of a medicament for treating clinical complications of Type I and Type II Diabetes. Examples of such complications include Diabetic Nephropathy, Diabetic Retinopathy, Diabetic Neuropathies, Peripheral Arterial Occlusive Disease (PAOD). Other clinical complications of Diabetes are also encompassed by the present disclosure.

Furthermore, conditions and diseases which result from chronic fatty and fibrotic degeneration of organs due to enforced lipid and specifically triglyceride accumulation and subsequent activation of profibrotic pathways may also be treated by applying the compounds or pharmaceutical composition of the present disclosure. Such conditions and diseases encompass Non-Alcoholic Steatohepatitis (NASH) and chronic cholestatic conditions in the liver, Glomerulosclerosis and Diabetic Nephropathy in the kidney, Macula Degeneration and Diabetic Retinopathy in the eye and Neurodegenerative diseases such as Alzheimer's Disease in the brain or Diabetic Neuropathies in the peripheral nervous system.

Administration and Pharmaceutical Compositions

In another aspect, the present disclosure provides a pharmaceutical composition comprising a compound of the present disclosure and a pharmaceutically acceptable carrier. The pharmaceutical composition can be formulated for particular routes of administration such as oral administration, parenteral administration, and rectal administration, etc. In addition, the pharmaceutical compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Suitable compositions for oral administration include an effective amount of a compound of the disclosure in the form of tablets, lozenges, aqueous or oily suspensions, dispersible powders or granules, emulsion, hard or soft capsules, or syrups or elixirs.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

disclosuredisclosuredisclosuredisclosuredisclosuredisclosureThe present disclosure further provides anhydrous pharmaceutical compositions and dosage forms comprising the compounds of the present disclosure as active ingredients, since water may facilitate the degradation of certain compounds. Anhydrous pharmaceutical compositions and dosage forms of the disclosure can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

The disclosure further provides pharmaceutical compositions and dosage forms that comprise one or more agents that reduce the rate by which the compound of the present disclosure as an active ingredient will decompose. Such agents, which are referred to herein as “stabilizers,” include, but are not limited to, antioxidants such as ascorbic acid, pH buffers, or salt buffers, etc.

The pharmaceutical composition or combination of the present disclosure can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present disclosure can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations. A therapeutically effective amount in vivo may range depending on the route of administration, between about 0.1-500 mg/kg, or between about 1-100 mg/kg.

The compound of the present disclosure may be administered either simultaneously with, or before or after, one or more other therapeutic agent. The compound of the present disclosure may be administered separately, by the same or different route of administration, or together in the same pharmaceutical composition as the other agents.

In one embodiment, the disclosure provides a product comprising a compound of Formula I, Formula I′ and (TA) to (I-G) and at least one other therapeutic agent as a combined preparation for simultaneous, separate or sequential use in therapy. In one embodiment, the therapy is the treatment of a disease or condition mediated by FXR. Products provided as a combined preparation include a composition comprising a compound of Formula I, Formula I′ and (TA) to (I-G), and the other therapeutic agent(s) together in the same pharmaceutical composition, or the compound of Formula I, Formula I′, (TA) to (I-Y), (Γ), II, and (I A)-(II-K) and the other therapeutic agent(s) in separate form, e.g. in the form of a kit.

In one embodiment, the disclosure provides a pharmaceutical composition comprising a compound of Formula I, Formula I′ and (I-A) to (I-G) and another therapeutic agent(s). It is contemplated that the disclosure provides a pharmaceutical composition comprising a compound of Formula I, Formula I′ and (I-A) to (I-G) in combination with a naturally occurring non-toxic bile acid, such as ursodeoxycholic acid, as an aid in preventing possible depletion of fat-soluble vitamins secondary to treatment with an FXR agonist. Accordingly, the compounds of the disclosure may be administered concurrently with the naturally occurring non-toxic bile acid, either as separate entities or as a single formulation comprising a compound of Formula I, Formula I′ and (I-A) to (I-G) and naturally occurring bile acid.

Optionally, the pharmaceutical composition may comprise a pharmaceutically acceptable excipient, as described above.

In one embodiment, the disclosure provides a kit comprising two or more separate pharmaceutical compositions, at least one of which contains a compound of Formula I, Formula I′ and (I-A) to (I-G). In one embodiment, the kit comprises means for separately retaining said compositions, such as a container, divided bottle, or divided foil packet. An example of such a kit is a blister pack, as typically used for the packaging of tablets, capsules and the like.

The kit of the disclosure may be used for administering different dosage forms, for example, oral and parenteral, for administering the separate compositions at different dosage intervals, or for titrating the separate compositions against one another. To assist compliance, the kit of the disclosure typically comprises directions for administration.

In the combination therapies of the disclosure, the compound of the disclosure and the other therapeutic agent may be manufactured and/or formulated by the same or different manufacturers. Moreover, the compound of the disclosure and the other therapeutic may be brought together into a combination therapy: (i) prior to release of the combination product to physicians (e.g. in the case of a kit comprising the compound of the disclosure and the other therapeutic agent); (ii) by the physician themselves (or under the guidance of the physician) shortly before administration; (iii) in the patient themselves, e.g. during sequential administration of the compound of the disclosure and the other therapeutic agent.

Accordingly, the disclosure provides the use of a compound of Formula I, Formula I′ and (I-A) to (I-G) for treating a disease or condition mediated by FXR, wherein the medicament is prepared for administration with another therapeutic agent. The disclosure also provides the use of another therapeutic agent for treating a disease or condition mediated by FXR, wherein the medicament is administered with a compound of Formula I, Formula I′ and (I-A) to (I-G).

The disclosure also provides a compound of Formula I, Formula I′ and (I-A) to (I-G) for use in a method of treating a disease or condition mediated by FXR, wherein the compound of Formula I, Formula I′ and (I-A) to (I-G) is prepared for administration with another therapeutic agent. The disclosure also provides another therapeutic agent for use in a method of treating a disease or condition mediated by FXR, wherein the other therapeutic agent is prepared for administration with a compound of Formula I, Formula I′ and (I-A) to (I-G). The disclosure also provides a compound of Formula I, Formula I′ and (I-A) to (I-G) for use in a method of treating a disease or condition mediated by FXR, wherein the compound of Formula I, Formula I′ and (I-A) to (I-G) is administered with another therapeutic agent. The disclosure also provides another therapeutic agent for use in a method of treating a disease or condition mediated by FXR, wherein the other therapeutic agent is administered with a compound of Formula I, Formula I′ and (I-A) to (I-G).

The disclosure also provides the use of a Formula I, Formula I′ and (I-A) to (I-G) for treating a disease or condition mediated by FXR, wherein the patient has previously (e.g. within 24 hours) been treated with another therapeutic agent. The disclosure also provides the use of another therapeutic agent for treating a disease or condition mediated by FXR, wherein the patient has previously (e.g. within 24 hours) been treated with a compound of Formula I, Formula I′ and (I-A) to (I-G).

In one embodiment, the other therapeutic agent is useful in the treatment of dyslipidemia, cholestasis, estrogen-induced cholestasis, drug-induced cholestasis, PBC, PSC, PFIC, alcohol-induced cirrhosis, cystic fibrosis, cholelithiasis, liver fibrosis, atherosclerosis or diabetes, particularly type II diabetes.

Processes for Making Compounds of the Disclosure

When Pg is H, the compounds of Formula I, Formula I′ and (I-A) to (I-G) can be prepared by coupling of compounds of Formula II and Formula III; otherwise, another deprotection step was required to afford the compounds (Schemes I).

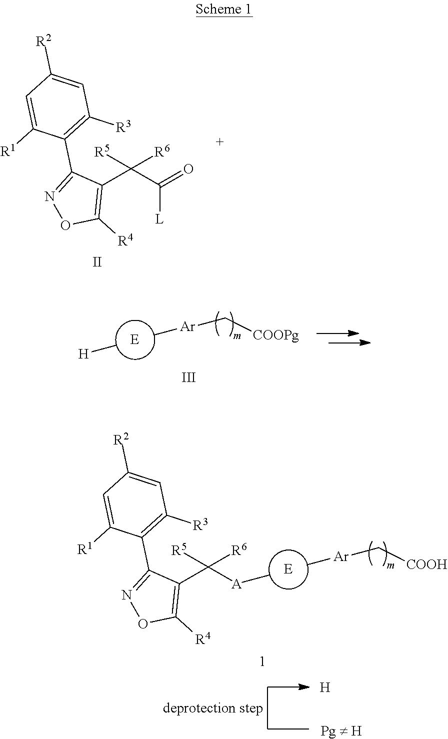

Wherein R¹˜R⁶, Ar and ring E are as defined in Formula I or Formula I′; L is H, Me or OH; Pg is H or carboxyl protecting group such as methyl, m=0 or 1.

Optionally, the present disclosure included converting a compound of Formula I, wherein the substituents have the meaning as defined, into another compound of Formula I as defined; and recovering the resulting compound of Formula I in free form or as a salt; and optionally converting the compound of Formula I obtained in free form into a desired salt, or an obtained salt into the free form.

disclosuredisclosuredisclosuredisclosuredisclosuredisclosuredisclosuredisclosuredisclosuredisclosuredisclosuredisclosuredisclosuredisclosuredisclosureThe present disclosure also provides pro-drugs of the compounds of the present disclosure that converts in vivo to the compounds of the present disclosure. A pro-drug is an active or inactive compound that is modified chemically through in vivo physiological action, such as hydrolysis, metabolism and the like, into a compound of this disclosure following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compounds that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the co-(amino, mono- or di-lower alkylamino, carboxy, lower alkoxycarbonyl)-lower alkyl esters, the cc-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Furthermore, the compounds of the present disclosure, including their salts, may also be obtained in the form of hydrates, or their crystals may, for example, include the solvent used for crystallization. Different crystalline forms may be present. The compounds of the present disclosure may inherently or by design form solvates with pharmaceutically acceptable solvents (including water); therefore, it is intended that the disclosure embrace both solvated and unsolvated forms. The term “solvate” refers to a molecular complex of a compound of the present disclosure (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water. The compounds of the present disclosure, including salts, hydrates and solvates thereof, may inherently or by design form polymorphs.

Compounds of the disclosure in unoxidized form may be prepared from N-oxides of compounds of the disclosure by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

All starting materials, building blocks, reagents, acids, bases, dehydrating agents, solvents and catalysts utilized to synthesize the compounds of the present disclosure are either commercially available or can be produced by organic synthesis methods known to one of ordinary skill in the art (Houben-Weyl 4th Ed. 1952, Methods of Organic Synthesis, Thieme, Volume 21). All methods described herein can be performed in any suitable order unless otherwise indicated herein or otherwise clearly contradicted by context. The use of any and all examples, or exemplary language (e.g. “such as”) provided herein is intended merely to better illuminate the disclosure and does not pose a limitation on the scope of the disclosure otherwise claimed.

### EXAMPLES

### Example 1

### 6-(4-(2-(5-cyclopropyl-3-(2,6-dichlorophenyl)isoxazol-4-yl)ethyl)piperazin-1-yl)-1-methyl-1H-indole-3-carboxylic Acid (I1)

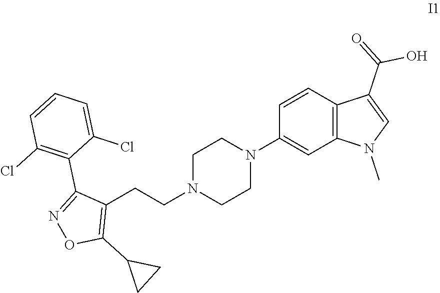 [SMILES: Cn1cc(C(=O)O)c2ccc(N3CCN(CCc4c(-c5c(Cl)cccc5Cl)noc4C4CC4)CC3)cc21]

### Step 1: Synthesis of 4

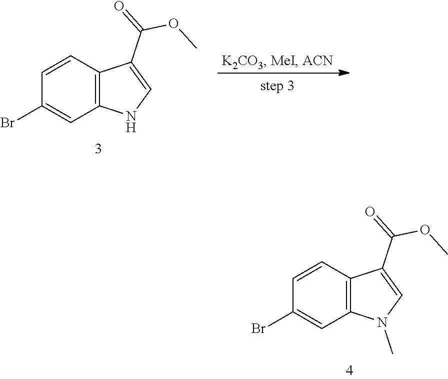

To a solution of compound 3 (31 g, 122.0 mmol) in ACN (300 mL), K₂CO₃ (33.7 g, 244.0 mmol) was added followed by the MeI (43.3 g, 305.0 mmol) at rt. The reaction mixture was stirred overnight was and then concentrated. The residue was diluted with water, extracted with EA, and the combined organic layers were washed with brine, dried, concentrated and purified by silica column with heptanes/EA (5:1) to give the desired product compound 4 (12.1 g, white solid. Three steps totally yield 15%). LCMS: (ESI-MS): [M+H]⁺=268.0, 270.0; ¹H NMR (400 MHz, DMSO) δ ppm: 8.15 (s, 1H), 7.92 (d, J=8.8 Hz, 1H), 8.84 (s, 1H), 7.38 (s, 1H), 3.85 (s, 3H), 3.80 (s, 3H).

### Step 2: Synthesis of 5

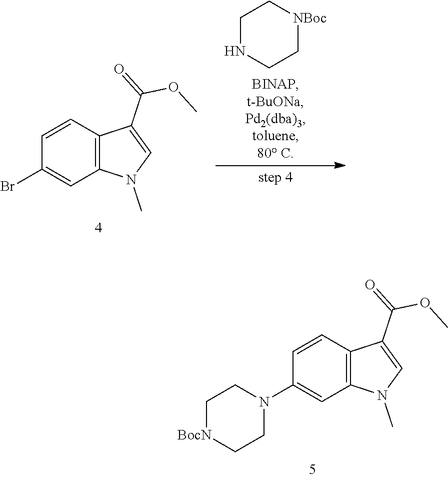 [SMILES: CC(C)(C)OC(=O)N1CCNCC1 ; COC(=O)c1cn(C)c2cc(Br)ccc12 ; COC(=O)c1cn(C)c2cc(N3CCN(C(=O)OC(C)(C)C)CC3)ccc12]

Under nitrogen, a mixture of compound 4 (9.4 g, 35.1 mmol), tert-butyl piperazine-1-carboxylate (7.8 g, 42.1 mmol), t-BuONa (5.1 g, 52.6 mmol), BINAP (2.2 g, 3.5 mmol), Pd₂(dba)₃ (1.6 g, 1.7 mmol) were successively added to toluene (110 mL). The mixture was stirred at 80° C. for overnight then filtered. The filter cake was washed with EA, the filtrate is concentrated, dried and purified by silica column with heptanes/EA (5:1) to give the desired product 5 (4 g, yellow solid, yield 30.1%). LCMS: (ESI-MS): [M+H]⁺=374.2; ¹H NMR (400 MHz, CDCl₃) δ ppm: 8.04 (d, J=8.8 Hz, 1H), 7.68 (s, 1H), 7.04-7.02 (m, 1H), 6.80 (s, 1H), 3.90 (s, 3H), 3.79 (s, 3H), 3.65-3.64 (m, 4H), 3.18-3.17 (m, 4H), 7.68 (s, 9H).

### Step 3: Synthesis of 6

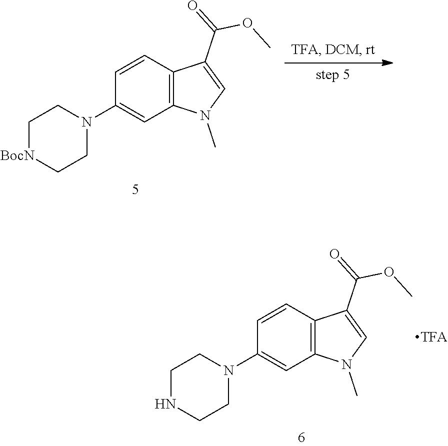 [SMILES: COC(=O)c1cn(C)c2cc(N3CCN(C(=O)OC(C)(C)C)CC3)ccc12 ; COC(=O)c1cn(C)c2cc(N3CCNCC3)ccc12]

To a solution of compound 5 (4 g, 10.7 mmol) in DCM (40 mL), TFA (10 mL) were added. The reaction was stirred for 2 h at rt. After completion of the reaction, the reaction solution was concentrated; the residue was diluted with MTBE (20 mL) and filtered. The filter cake was washed with MTBE (10 mL×2), dried to get compound 6. LCMS: (ESI-MS): [M+H]⁺=274.1; ¹H NMR (300 MHz, DMSO) δ ppm: 7.99 (s, 1H), 7.86 (d, J=8.4 Hz, 1H), 7.05-7.02 (m, 2H), 3.81 (s, 3H), 3.78 (s, 3H), 3.36-3.29 (m, 8H).

### Step 4: Synthesis of 8

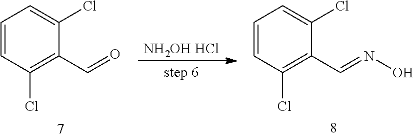

To a solution of sodium hydroxide (7 g, 175 mmol) in water (120 mL) was added NH₂OH.HCl (11.8 g, 170 mmol) in water (120 mL) at 0° C. The resulting solution was stirred for 10 min at 0° C. then a solution of compound 7 (25.8 g, 147.3 mmol) in ethanol (120 mL) was added, and stirred for an additional 1 h at rt. The reaction was diluted with water, extracted with EA, the combined organic layers were washed with brine, dried, concentrated to give the desired product compound 8 and which was used directly in the next step.

### Step 5: Synthesis of 9

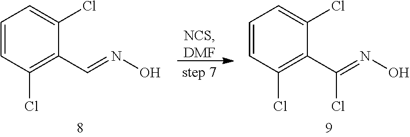

NCS (23.9 g, 179 mmol) was slowly added to a stirred solution of compound 8 (28.3 g, 149 mmol) in DMF (300 mL) at <25° C. After the reaction mixture was stirred for 1 h at rt. It was diluted with water, extracted with EA, the combined organic layers were washed with brine, dried, concentrated to give the desired product compound 9 used directly in the next step. ¹H NMR (DMSO-d6) δ ppm: 12.68 (br, 1H), 7.67-7.55 (m, 3H).

### Step 6: Synthesis of 10

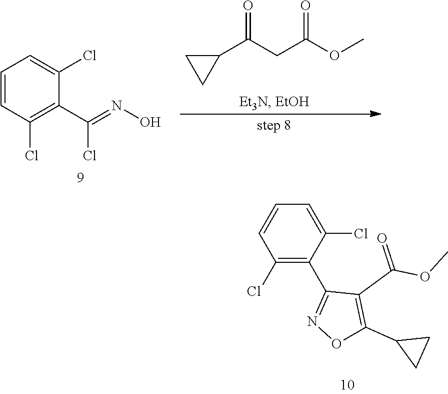

Triethylamine (24.1 g, 240.6 mmol) was added to methyl 3-cyclopropyl-3-oxopropanoate (17.2 g, 120.3 mmol) and the mixture was stirred at rt for 30 min. Then, the mixture is cooled to about 10° C. and a suspension of compound 9 (27 g, 120.3 mmol) in EtOH (550 mL) is added slowly below 24° C. After the reaction was stirred overnight at rt. It was diluted with water, extracted with EA, the combined organic layers were washed with brine, dried, filtered and concentrated to about 10% of its total volume. The precipitate formed is filtered, triturated with ether (200 mL) and dried under vacuum to obtain compound 10. LCMS: (ESI-MS): [M+H]⁺=312.0; ¹H NMR (300 MHz, CDCl₃) δ ppm: 7.44-7.35 (m, 3H), 3.87 (s, 3H), 2.95-2.90 (m, 1H), 1.45-1.40 (m, 2H), 1.37-1.28 (m, 2H).

### Step 7: Synthesis of 11

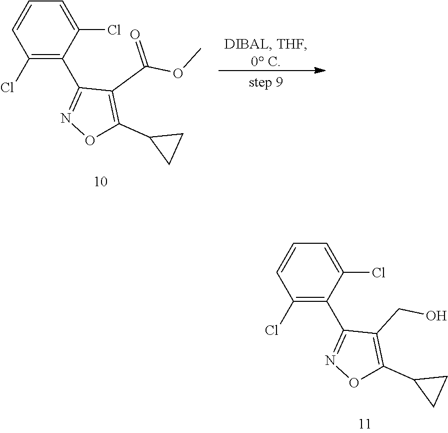 [SMILES: COC(=O)c1c(-c2c(Cl)cccc2Cl)noc1C1CC1 ; "DIBAL, THF,0° C." ; OCc1c(-c2c(Cl)cccc2Cl)noc1C1CC1]

DIBAL-H 1.5M (90.8 mL, 136.2 mmol) is added dropwise to a stirred solution of compound 10 (17 g, 54.5 mmol) in THF (150 mL) at 10° C. The mixture is stirred at rt for 2 h and then quenched with MeOH. The reaction was diluted with water, extracted with EA. The combined organic layers were dried and purified by silica column with heptanes/EA (5:1) to give the desired product compound 11 (13.2 g, white solid, yield 84.2%). LCMS: (ESI-MS): [M+H]+=284.0; 1H NMR (400 MHz, CDCl3) δ ppm: 7.44-7.33 (m, 3H), 4.41 (s, 2H), 2.22-2.15 (m, 1H), 1.30-1.26 (m, 2H), 1.17-1.13 (m, 2H).

### Step 8: Synthesis of 12

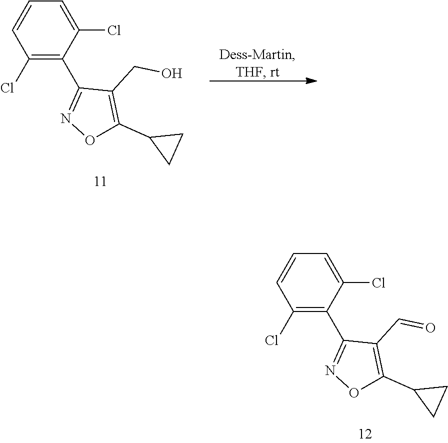 [SMILES: OCc1c(-c2c(Cl)cccc2Cl)noc1C1CC1 ; "Dess-Martin,THF, rt" ; O=Cc1c(-c2c(Cl)cccc2Cl)noc1C1CC1]

To a solution of compound 11 (5.68 g, 20 mmol) in THF (60 mL) at 0° C., the Dess-martin (12.7 g, 30 mol) was partly added. Then the reaction mixture was stirred for 3 h at rt. After the reaction completed, a saturated aqueous solution of NaHCO₃ and Na₂S₂O₃ was added to the reaction and stirred for 30 minutes. Then the solution extracted with EA and the combined organic layers were dried, concentrated and purified by silica column with heptanes/EA (20:1) to give the desired product compound 12 (4.4 g, white solid, yield 78%). LCMS: (ESI-MS): [M+H]+=282.0. 1H NMR (400 MHz, CDCl3) δ ppm: 9.67 (s, 1H), 7.47-7.40 (m, 3H), 2.84-2.80 (m, 1H), 1.50-1.47 (m, 2H), 1.39-1.34 (m, 2H).

### Step 9: Synthesis of 13

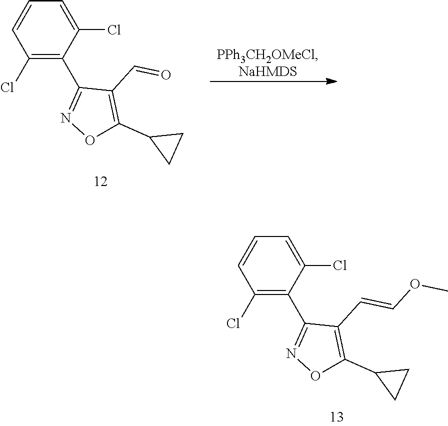

Under nitrogen, a solution of CH₂OMeCl (3.43 g, 10 mmol) in THF (35 mL) was cooled to −10° C., then 2N NaHMDS (5 mL, 10 mmol) was added dropwise. The reaction mixture was stirred at −10° C. for 20 min then compound 12 (1.41 g, 5 mmol) in THF (15 mL) was added dropwise to the reaction mixture. The reaction mixture was stirred at rt overnight, quenched with water, extracted with EA. The combined organic layers were washed with brine, dried, concentrated and purified by silica column with heptanes/EA (50:1) to give compound 13 (1.45 g, yellow solid, yield: 93%). LCMS: (ESI-MS): [M+H]+=310.1. 1H NMR (300 MHz, CDCl₃) δ ppm: 7.45-7.32 (m, 3H), 6.44 (d, J=13.2 Hz, 1H), 6.44 (d, J=13.2 Hz, 1H), 3.56 (s, 3H), 2.10-2.03 (m, 1H), 1.24-1.18 (m, 2H), 1.16-1.08 (m, 2H).

### Step 10: Synthesis of 14

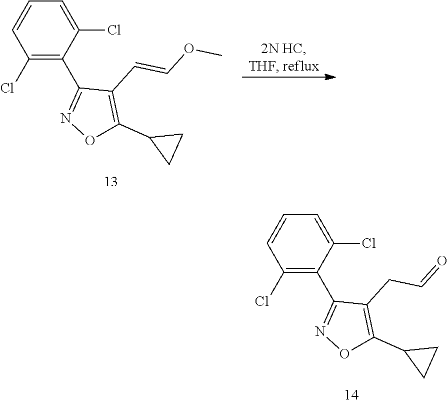 [SMILES: CO/C=C/c1c(-c2c(Cl)cccc2Cl)noc1C1CC1 ; "2N HC,THF, reflux" ; O=CCc1c(-c2c(Cl)cccc2Cl)noc1C1CC1]

To a solution of compound 13 (500 mg, 1.61 mmol) in THF (6 mL) was added 2N HCl (3 mL) at rt. The reaction mixture was heated to reflux overnight. After completion of the reaction, it was extracted with EA, the combined organic layers were washed with saturated NaHCO₃ and brine, dried, concentrated and purified by silica gel column with heptanes/EA (5:1) to give compound 14 (320 mg, oily liquid, yield: 67%). LCMS: [M+H]+=296.0. ¹H NMR (CDCl₃) δ ppm: 9.59 (s, 1H), 7.46-7.34 (m, 3H), 3.35 (s, 2H), 2.06-1.92 (m, 1H), 1.27-1.25 (m, 2H), 1.20-1.13 (m, 2H).

### Step 11: Synthesis of 15

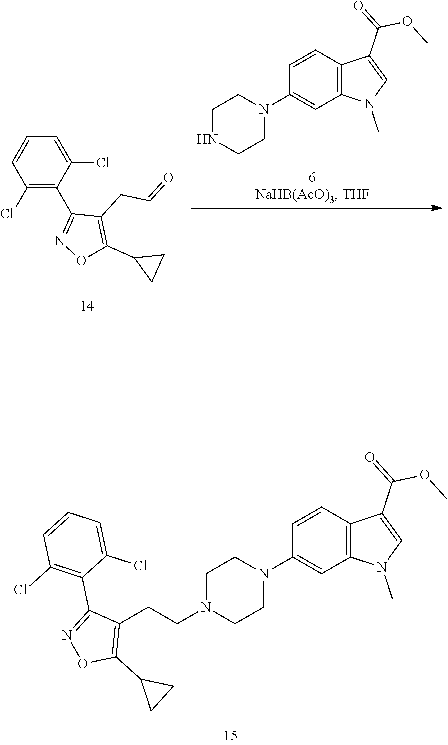 [SMILES: O=CCc1c(-c2c(Cl)cccc2Cl)noc1C1CC1 ; COC(=O)c1cn(C)c2cc(N3CCNCC3)ccc12 ; COC(=O)c1cn(C)c2cc(N3CCN(CCc4c(-c5c(Cl)cccc5Cl)noc4C4CC4)CC3)ccc12]

To a solution of compound 14 (320 mg, 1.1 mmol) in THF (5 mL) was added compound 6 (426 mg, 1.1 mmol) at rt. Then the reaction mixture was stirred at rt for 30 min, then treated with NaHB (AcO)₃ (700 mg, 3.3 mmol), stirred at rt for another 30 min. After the reaction was completed, the reaction was quenched with water, extracted with EA, and the combined organic layers were washed with brine, dried, concentrated and purified by silica gel column with heptanes/EA (2:1) to give compound 15 (150 mg, white solid, yield: 25%). LCMS: (ESI-MS): [M+H]+=553.1. 1H NMR (400 MHz, CDCl₃) δ ppm: 7.93 (d, J=8.8 Hz, 1H), 7.59 (s, 1H), 7.37-7.35 (m, 2H), 7.30-7.28 (m, 1H), 7.28 (s, 1H), 6.91-6.89 (m, 1H), 6.67 (s, 1H), 3.81 (s, 3H), 3.69 (s, 3H), 3.15 (s, 4H), 2.56-2.47 (m, 8H), 2.02-1.95 (m, 1H), 1.21-1.15 (m, 2H), 1.05-1.03 (m, 2H).

### Step 12: Synthesis of I1

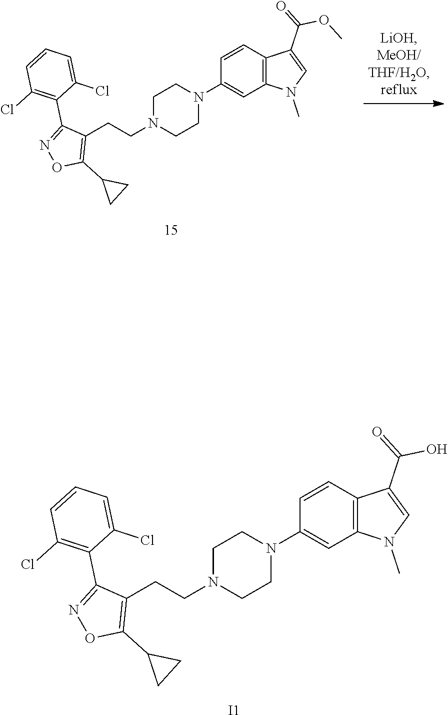 [SMILES: COC(=O)c1cn(C)c2cc(N3CCN(CCc4c(-c5c(Cl)cccc5Cl)noc4C4CC4)CC3)ccc12 ; Cn1cc(C(=O)O)c2ccc(N3CCN(CCc4c(-c5c(Cl)cccc5Cl)noc4C4CC4)CC3)cc21 ; "I1"]

To a solution of compound 15 (150 mg, 0.27 mmol) in MeOH/THF/H₂O=1:1:1 (3 mL), LiOH (97 mg, 4.1 mmol) was added at rt. Then the reaction mixture was heated to reflux and stirred for overnight. After completion of the reaction, the reaction was acidified with 2N aqueous and a precipitate formed. Then the reaction was diluted with water (5 mL), extracted with EA, the combined organic layers were washed with brine, dried and purified by pre-TLC with EA to give compound I1 (22 mg, white solid, yield 15%). LCMS: (ESI-MS): [M+H]+=539.1.1H NMR (300 MHz, DMSO-d6) δ ppm: 11.80 (br, 1H), 7.83-7.77 (m, 2H), 7.69-7.57 (m, 3H), 6.92-6.88 (m, 2H), 3.76 (s, 3H), 3.06 (s, 4H), 2.54-2.50 (m, 2H), 2.50-2.43 (m, 4H), 2.33-2.28 (m, 3H), 1.13-1.05 (m, 4H).

### Example 2

### 6-(4-(2-(5-cyclopropyl-3-(2,6-dichlorophenyl)isoxazol-4-yl)acetyl)piperazin-1-yl)-1-methyl-1H-indole-3-carboxylic Acid (I2)

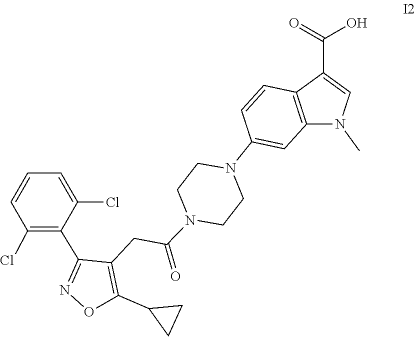 [SMILES: Cn1cc(C(=O)O)c2ccc(N3CCN(C(=O)Cc4c(-c5c(Cl)cccc5Cl)noc4C4CC4)CC3)cc21]

### Step 1: Synthesis of 16

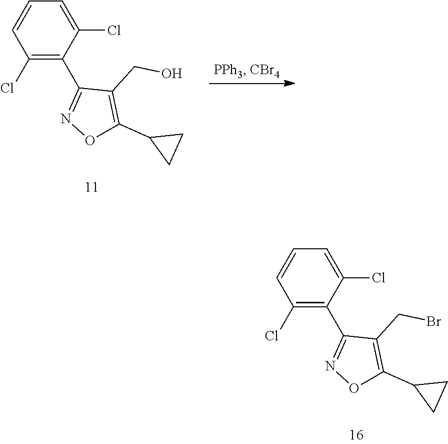 [SMILES: OCc1c(-c2c(Cl)cccc2Cl)noc1C1CC1 ; Clc1cccc(Cl)c1-c1noc(C2CC2)c1CBr]

To a solution of compound 11 (460 mg, 1.62 mmol) in DCM (10 mL), PPh3 (637 mg, 2.43 mmol) was added. Then CBr₄ (805 mg, 2.43 mmol) was added dropwise to the mixture and stirred for 1 h at rt. After completion of the reaction, the reaction solution was concentrated, purified by silica column with heptanes/EA (10:1) to give the desired product compound 16 (360 mg, white solid, yield 64%). LCMS: (ESI-MS): [M+H]+=347.9; 1H NMR (300 MHz, CDCl₃) δ ppm: 7.47-7.37 (m, 3H), 4.24 (s, 2H), 2.17-2.12 (m, 1H), 1.32-1.29 (m, 2H), 1.24-1.20 (m, 2H).

### Step 2: Synthesis of 17

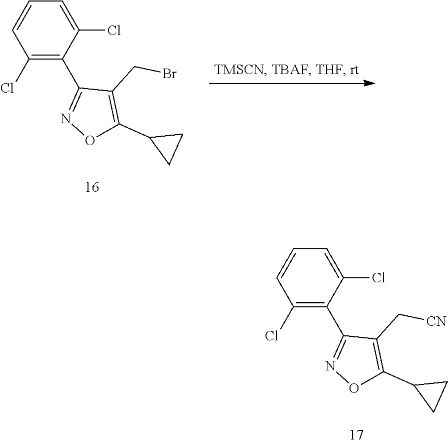 [SMILES: Clc1cccc(Cl)c1-c1noc(C2CC2)c1CBr ; N#CCc1c(-c2c(Cl)cccc2Cl)noc1C1CC1]

To a solution of compound 16 (320 mg, 0.92 mmol) in THF (5 mL), TBAF 1M/THF (1.84 mL, 1.84 mmol) was added. The TMSCN (182.5 mg, 1.84 mmol) was slowly added dropwise to mixture while keeping temperature below 25° C. and stirred overnight at rt. The reaction was diluted with water, extracted with EA, the combined organic layers were washed with brine, dried and purified by silica column with heptanes/EA (5:1) to give the desired product compound 17 (320 mg, white solid, yield 90%). LCMS: (ESI-MS): [M+H]+=293.0; 1H NMR (400 MHz, CDCl₃) δ ppm: 7.47-7.37 (m, 3H), 3.39 (s, 2H), 2.14-2.09 (m, 1H), 1.31-1.28 (m, 2H), 1.26-1.1 (m, 2H).

### Step 3: Synthesis of 18

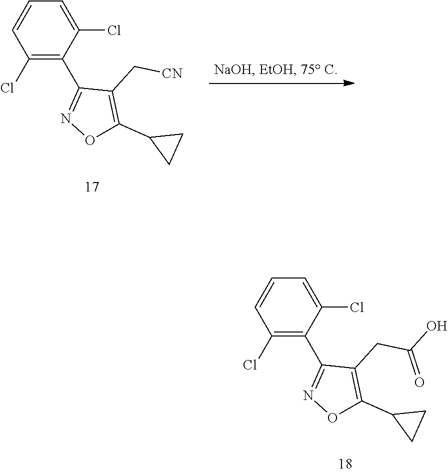 [SMILES: N#CCc1c(-c2c(Cl)cccc2Cl)noc1C1CC1 ; O=C(O)Cc1c(-c2c(Cl)cccc2Cl)noc1C1CC1]

To a solution of compound 17 (300 mg, 1.02 mmol) in EtOH, NaOH 4M/H₂O (1 mL, 4.08 mmol) was added at rt. Then the reaction mixture was heated to 75° C. for 3 h. The reaction was acidified with 2N aqueous and a precipitate formed. Then the reaction was diluted with water, extracted with EA, the combined organic layers were washed with brine, dried and purified by silica column with EA to give the desired product compound 18 (200 mg, white solid, yield 62.8%). LCMS: (ESI-MS): [M+H]+=312.0; ¹H NMR (400 MHz, DMSO-d6) δ ppm: 7.64-7.54 (m, 3H), 4.06 (s, 2H), 3.28 (br, 1H), 2.30-2.24 (m, 1H), 1.14-1.04 (m, 4H).

### Step 4: Synthesis of 19

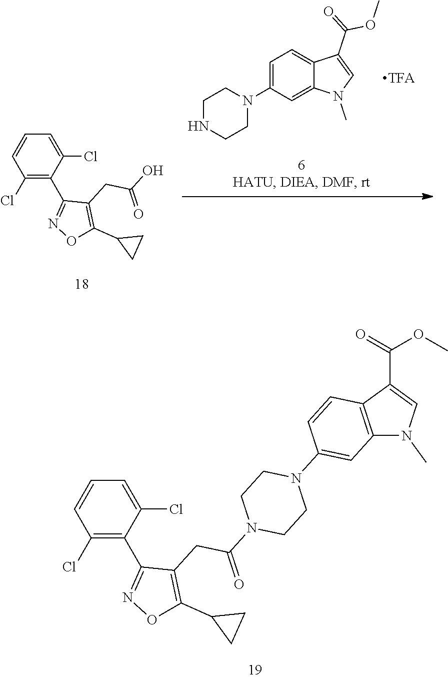 [SMILES: O=C(O)Cc1c(-c2c(Cl)cccc2Cl)noc1C1CC1 ; COC(=O)c1cn(C)c2cc(N3CCNCC3)ccc12 ; COC(=O)c1cn(C)c2cc(N3CCN(C(=O)Cc4c(-c5c(Cl)cccc5Cl)noc4C4CC4)CC3)ccc12]

Compound 18 (200 mg, 0.6 mmol), Compound 6 (256 g, 0.6 mmol), HATU (368 mg, 0.9 mmol) and DIEA (332 mg, 2.4 mmol) were successively added to DMF (7 mL). The mixture is stirred at rt for 4 h. After completion of the reaction, the reaction was diluted with water, extracted with EA. The combined organic layers were washed with brine, dried and purified by silica column with heptanes/EA (2:1) to give the desired product compound 19 (240 mg, white solid, yield 70%). LCMS: (ESI-MS): [M+H]+=567.1; ¹H NMR (300 MHz, CDCl₃) δ ppm: 8.06 (d, J=7.5 Hz, 1H), 7.71 (s, 1H), 7.47-7.34 (m, 4H), 6.99 (d, J=8.4 Hz, 1H), 3.91 (s, 3H), 3.81-3.80 (m, 5H), 3.70-3.45 (m, 4H), 3.30-2.99 (m, 4H), 2.20-2.19 (m, 1H), 1.28-1.20 (m, 2H), 1.15-1.12 (m, 2H).

### Step 5: Synthesis of I2

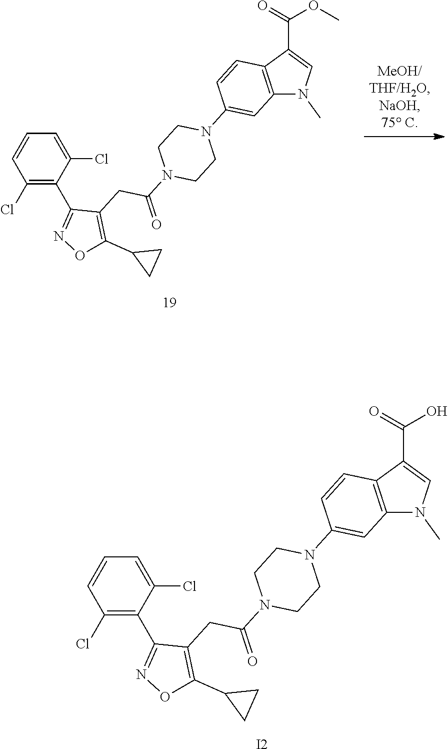

Follow the procedure of Synthesis of I1 to give the compound I2. LCMS: [M+H]=553.1; ¹H NMR (400 MHz, DMSO) δ ppm: 11.83 (br, 1H), 7.86-7.81 (m, 2H), 7.62-7.51 (m, 3H), 6.95-6.92 (m, 2H), 3.78 (s, 3H), 3.49-3.48 (m, 6H), 3.02-3.01 (m, 4H), 2.08-2.07 (m, 1H), 1.13-1.08 (m, 2H).

### Example 3

### 6-(4-(2-(3-(2,6-dichlorophenyl)-5-isopropylisoxazol-4-yl)ethyl)piperazin-1-yl)-1-methyl-1H-indole-3-carboxylic Acid (I3)

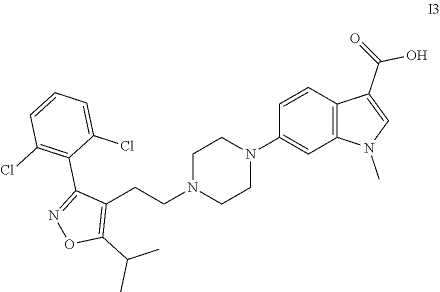 [SMILES: CC(C)c1onc(-c2c(Cl)cccc2Cl)c1CCN1CCN(c2ccc3c(C(=O)O)cn(C)c3c2)CC1]

### Step 1: Synthesis of 22

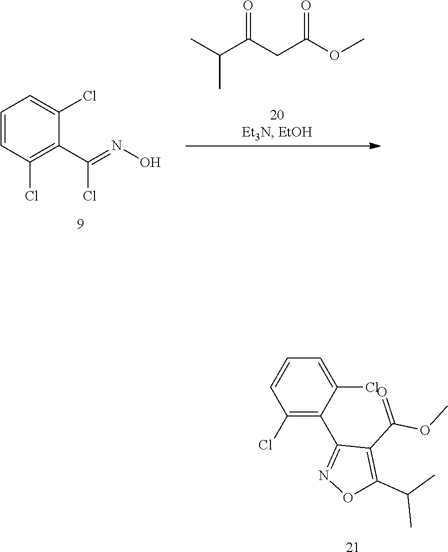

Follow the procedure of Synthesis of 10 to obtain compound 21. ¹H NMR (400 MHz, DMSO-d6) δ ppm: 7.73-7.58 (m, 3H), 3.93-3.81 (m, 1H), 3.69 (s, 3H), 1.43 (d, J=6.8 Hz, 6H).

### Step 2: Synthesis of 22

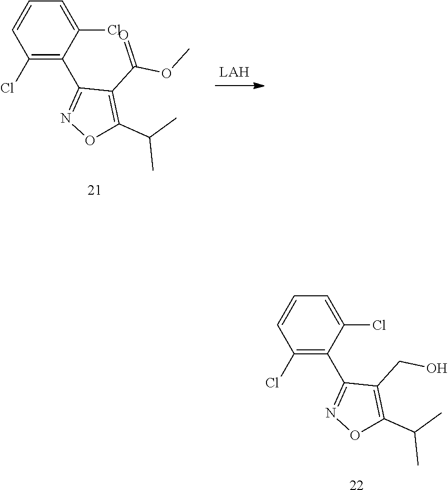 [SMILES: COC(=O)c1c(-c2c(Cl)cccc2Cl)noc1C(C)C ; "21" ; CC(C)c1onc(-c2c(Cl)cccc2Cl)c1CO ; "22"]

To a solution of compound 21 (6.0 g, 22 mmol) in THF (40 mL), LAH (88 mL, 88 mmol, 1M in THF) was added by dropwise at 0° C. The reaction is stirred at room temperature for 2 h then 100 mL of 1N NaOH aq was added. The precipitate formed was filtered through celite and all the solvents were removed in vacuum. The residue was purified with flash chromatography (PE:EA=1:2) to give product 22 (2.11 g as white solid, yield: 33.5%). 1HNMR (400 MHz, DMSO-d6): δ ppm: 7.71-7.51 (m, 3H), 4.96-4.91 (m, 1H), 4.22 (d, J=4.8 Hz, 2H), 3.41-3.35 (m, 1H), 1.31 (d, J=6.4 Hz, 6H).

### Step 3: Synthesis of 23

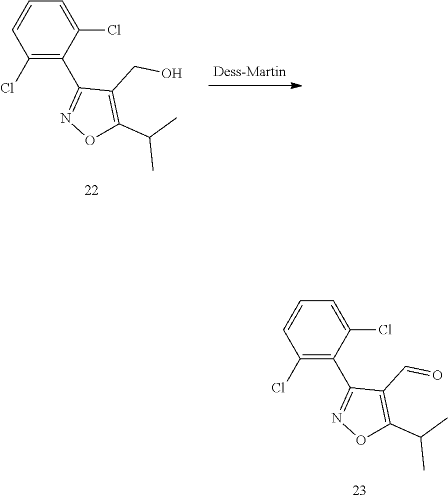 [SMILES: CC(C)c1onc(-c2c(Cl)cccc2Cl)c1CO ; "22" ; CC(C)c1onc(-c2c(Cl)cccc2Cl)c1C=O ; "23"]

Follow the procedure of Synthesis of 12 to give product 23. ¹H NMR (400 MHz, DMSO-d6): δ ppm 9.97 (s, 1H), 7.73-7.58 (m, 3H), 3.93-3.82 (m, 1H), 3.41-3.35 (m, 1H), 1.42 (d, J=6.4 Hz, 6H).

### Step 4: Synthesis of 24

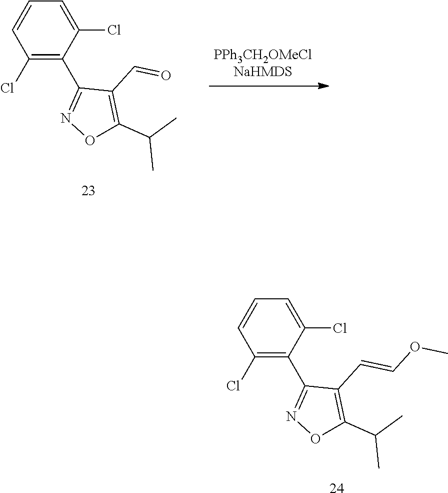 [SMILES: CC(C)c1onc(-c2c(Cl)cccc2Cl)c1C=O ; "23" ; CO/C=C/c1c(-c2c(Cl)cccc2Cl)noc1C(C)C ; "24"]

Follow the procedure of Synthesis of 13 to give product 24 (1.3 g of desired as mixture of isomers (E:Z=2:1).

### Step 5: Synthesis of 25

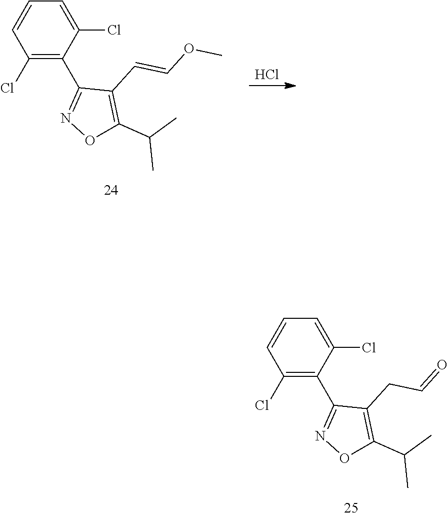 [SMILES: CO/C=C/c1c(-c2c(Cl)cccc2Cl)noc1C(C)C ; Cl ; "24" ; CC(C)c1onc(-c2c(Cl)cccc2Cl)c1CC=O ; "25"]

Follow the procedure of Synthesis of 14 to give the crude product 25, which could be used in next step without further purification.

### Step 6: Synthesis of 26

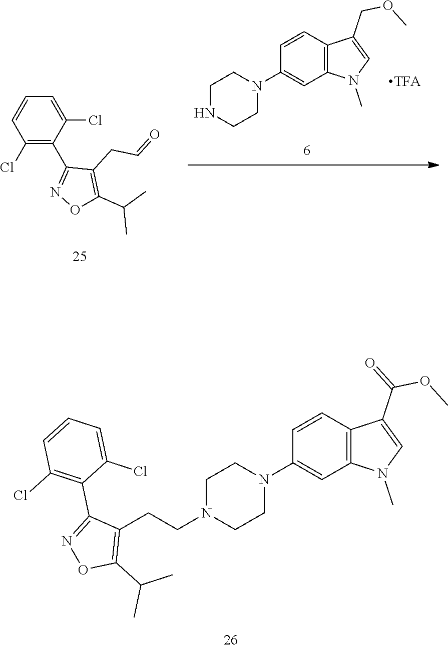 [SMILES: CC(C)c1onc(-c2c(Cl)cccc2Cl)c1CC=O ; COCc1cn(C)c2cc(N3CCNCC3)ccc12 ; COC(=O)c1cn(C)c2cc(N3CCN(CCc4c(-c5c(Cl)cccc5Cl)noc4C(C)C)CC3)ccc12]

Follow the procedure of Synthesis of 15 to give product 26.

### Step 7: Synthesis of I3

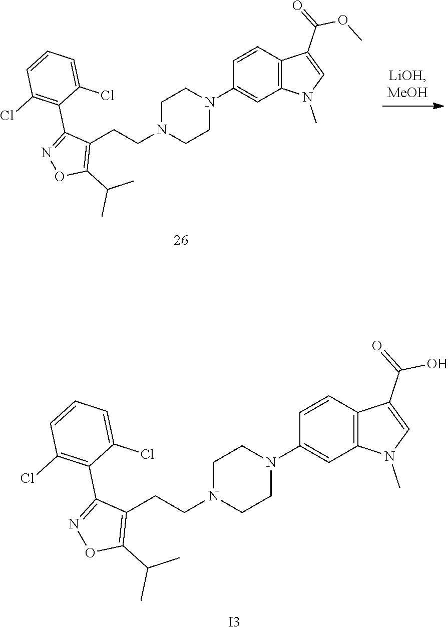 [SMILES: COC(=O)c1cn(C)c2cc(N3CCN(CCc4c(-c5c(Cl)cccc5Cl)noc4C(C)C)CC3)ccc12 ; CC(C)c1onc(-c2c(Cl)cccc2Cl)c1CCN1CCN(c2ccc3c(C(=O)O)cn(C)c3c2)CC1 ; "I3"]

Follow the procedure of Synthesis of I1 to give 61 mg of desired product 13, yield: 65.1%. LCMS: [M−1]=541.1. ¹H NMR (400 MHz, CDCl₃) δ ppm 7.98-7.96 (m, 1H), 7.69 (s, 1H), 7.39-7.37 (m, 2H), 7.34-7.32 (m, 1H), 6.87-6.85 (m, 1H), 6.75 (s, 1H), 3.71 (s, 3H), 3.66-3.33 (m, 6H), 3.16-3.15 (m, 1H), 2.91-2.65 (m, 6H), 1.31 (d, J=6.4 Hz, 6H).

### Example 4

### 2-(4-(2-(5-cyclopropyl-3-(2,6-dichlorophenyl)isoxazol-4-yl)ethyl)piperazin-1-yl)benzo[d]thiazole-6-carboxylic Acid (I4)

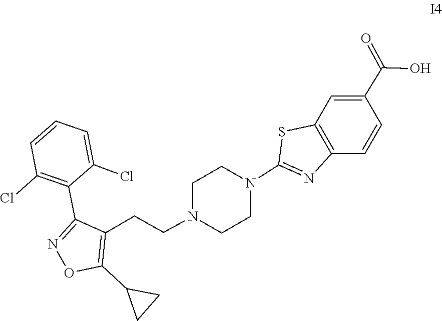 [SMILES: O=C(O)c1ccc2nc(N3CCN(CCc4c(-c5c(Cl)cccc5Cl)noc4C4CC4)CC3)sc2c1]

### Step 1: Synthesis of 29

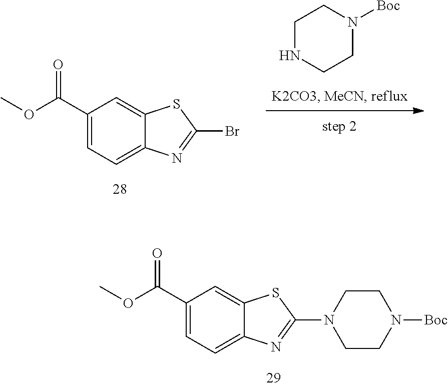 [SMILES: COC(=O)c1ccc2nc(Br)sc2c1 ; CC(C)(C)OC(=O)N1CCNCC1 ; COC(=O)c1ccc2nc(N3CCN(C(=O)OC(C)(C)C)CC3)sc2c1]

Compound 28 (450 mg, 1.65 mmol), tert-butyl piperazine-1-carboxylate (384 mg. 2.06 mmol) and K₂CO₃ (456 mg, 3.3 mmol) were suspended in acetonitrile (20 mL) and refluxed overnight. The resulting mixture was then concentrated in vacuo to approximately 2 mL, diluted with water and extracted with ethyl acetate. The organic layers were dried and concentrated in vacuo and purified by silica gel column with heptanes/EA (5:1) to give compound 29 (500 mg, white solid, yield 80%). LCMS: (ESI-MS): [M+H]+=378.1; ¹H NMR (300 MHz, CDCl₃) δ ppm: 8.33 (s, 1H), 8.02 (d, J=9.3 Hz, 1H), 7.57 (d, J=8.4 Hz, 1H), 3.92 (s, 3H), 3.70-3.68 (m, 4H), 3.62-3.61 (m, 4H), 1.50 (s, 9H).

### Step 2: Synthesis of 30

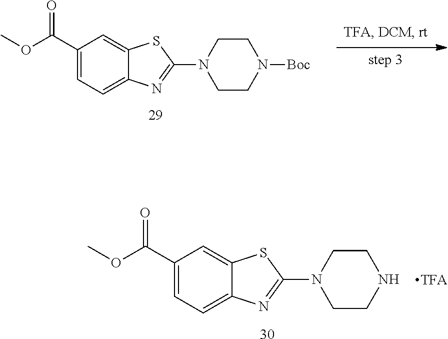

Follow the procedure of Synthesis of 6 to give product 30. LCMS: [M+H]+=278.1; ¹H NMR (400 MHz, DMSO-d6) δ ppm: 9.04 (s, 2H), 8.48 (s, 1H), 7.92-7.90 (m, 1H), 7.55 (d, J=8.8 Hz, 1H), 3.85 (s, 7H), 3.30-3.28 (m, 4H).

### Step 3: Synthesis of 31

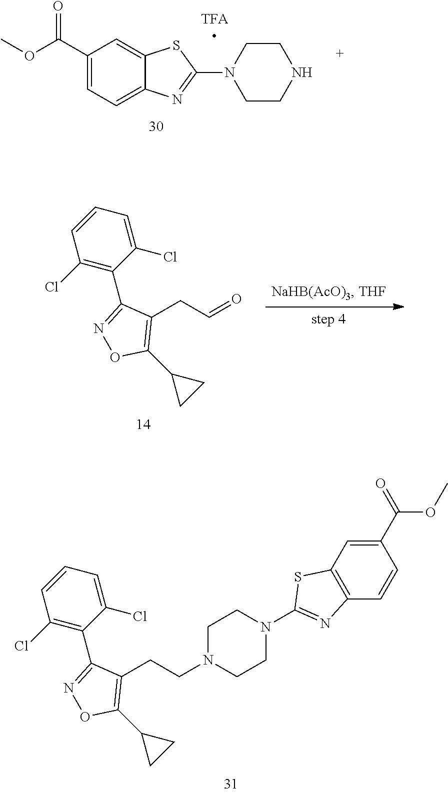

Follow the procedure of Synthesis of 15 to give product 31. LCMS: [M+H]+=557.1; ¹H NMR (400 MHz, CDCl3) δ ppm: 8.30 (s, 1H), 7.99 (d, J=8.7 Hz, 1H), 7.51 (d, J=8.1 Hz, 1H), 7.44-7.33 (m, 3H), 3.91 (s, 3H), 3.62 (s, 4H), 2.50 (s, 8H), 2.08-2.05 (m, 1H), 1.29-1.15 (m, 4H).

### Step 4: Synthesis of I4

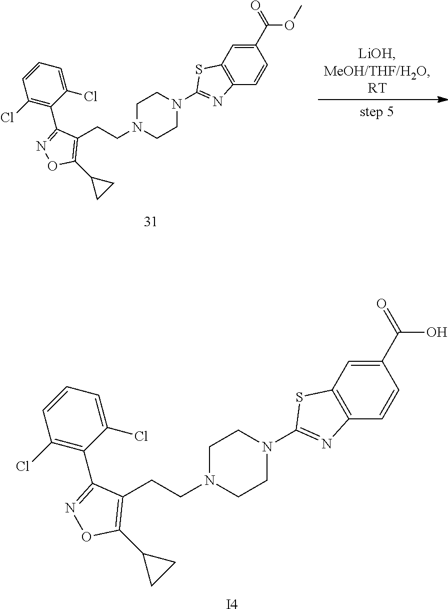

Follow the procedure of Synthesis of I1 to give the compound I4. LCMS: [M+H]+=543.1. ¹H NMR (400 MHz, MeOD) δ ppm: 8.41 (s, 1H), 8.03-8.01 (m, 1H), 7.66-7.55 (m, 4H), 3.96 (s, 4H), 3.48 (s, 4H), 3.33-3.25 (m, 2H), 2.91-2.87 (m, 2H), 2.30-2.26 (m, 1H), 1.24-1.20 (m, 4H).

### Example 5

### 2-(4-(2-(5-cyclopropyl-3-(2-(trifluoromethoxy)phenyl)isoxazol-4-yl)ethyl)piperazin-1-yl)-4-fluorobenzo[d]thiazole-6-carboxylic Acid (I5)

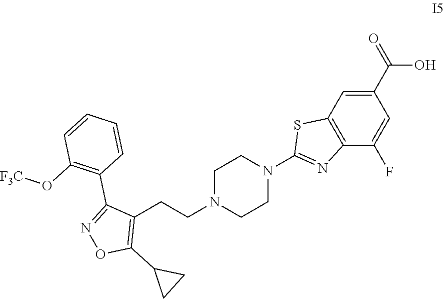 [SMILES: O=C(O)c1cc(F)c2nc(N3CCN(CCc4c(-c5ccccc5OC(F)(F)F)noc4C4CC4)CC3)sc2c1]

### Step 1: Synthesis of 33

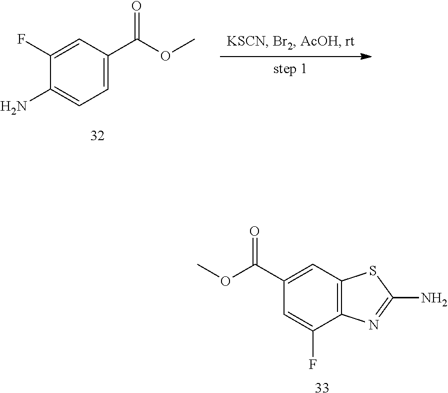

To a solution of compound 32 (5 g, 29.5 mmol) in AcOH (50 mL) was added KSCN (11.5 g, 118.2 mmol) at rt in one portion, and the resulting mixture was stirred at rt until it became a clear solution. Then, Br₂ (4.7 g, 175 mmol) in AcOH (30 mL) was added at rt over 45 min, and the whole reaction mixture was stirred at rt for 20 h. The precipitate that formed during the reaction was removed by filtration. The filtrate was poured into water and basified with cons. NH₃.H₂O to pH 8-9. The resulting precipitate was collected by suction filtration and dried at 60° C. under vacuum to give a crude product compound 33 (4.1 g) which was used directly in the next step. LCMS: [M+H]+=227.0.

### Step 2: Synthesis of 34

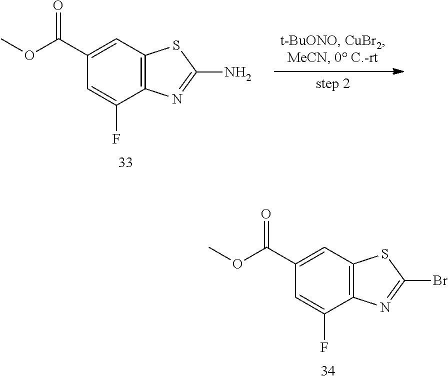

To a suspension of CuBr₂ (2.9 g, 13.3 mmol) in acetonitrile. t-BuONO (2 g, 17.7 mmol) at 0° C. dropwise over 10 min. To this solution was added 33 (2 g, 8.8 mmol) was added, and the reaction mixture was stirred at 30° C. for 48 h. The reaction mixture was then diluted with EtOAc, washed with water and brine, dried over anhydrous sodium sulfate, and concentrated under vacuum. The crude residue was purified by silica column with heptanes/EA (5:1) to give the desired product compound 34. LCMS: [M+H]+=290.0, 292.0; ¹H NMR (400 MHz, CDCl₃) δ ppm: 8.12 (s, 1H), 7.72 (d, J=6.9 Hz, 1H), 3.95 (s, 3H).

### Step 3: Synthesis of 35

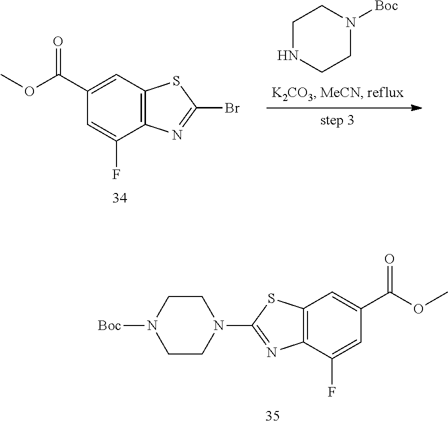

Follow the procedure of Synthesis of 29 to give 35. LCMS: [M+H]+=396.1.

### Step 4: Synthesis of Compound 36

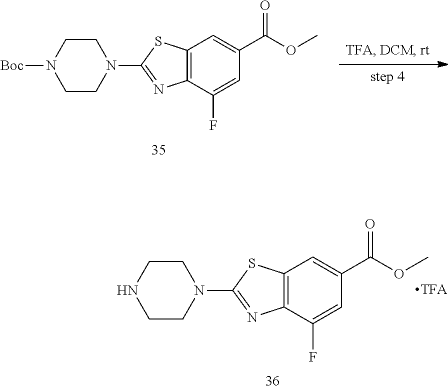 [SMILES: COC(=O)c1cc(F)c2nc(N3CCN(C(=O)OC(C)(C)C)CC3)sc2c1 ; "TFA, DCM, rt" ; "step 4" ; "35" ; COC(=O)c1cc(F)c2nc(N3CCNCC3)sc2c1 ; "36"]

Follow the procedure of Synthesis of 6 to give product 36. LCMS: [M+H]+=296.0; ¹H NMR (400 MHz, CDCl₃) δ ppm: 9.06 (br, 2H), 8.37 (s, 1H), 7.69-7.66 (m, 1H), 3.88-3.86 (m, 7H), 3.32-3.29 (m, 4H).

### Step 5: Synthesis of 38

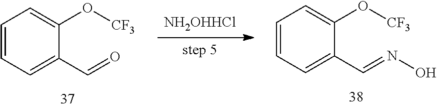 [SMILES: O=Cc1ccccc1OC(F)(F)F ; "NH2OHHCl" ; "step 5" ; O/N=C/c1ccccc1OC(F)(F)F]

Follow the procedure of Synthesis of 8 to give product 38 used directly in the next step. LCMS: [M+H]+=206.0.

### Step 6: Synthesis of 39

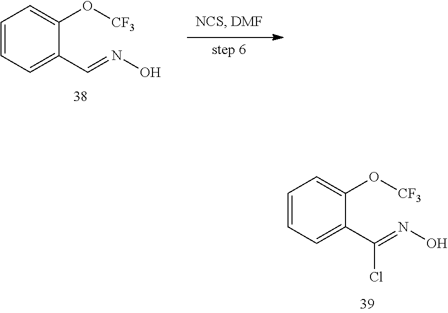

Follow the procedure of Synthesis of 9 to give product 39 used directly in the next step.

### Step 7: Synthesis of 40

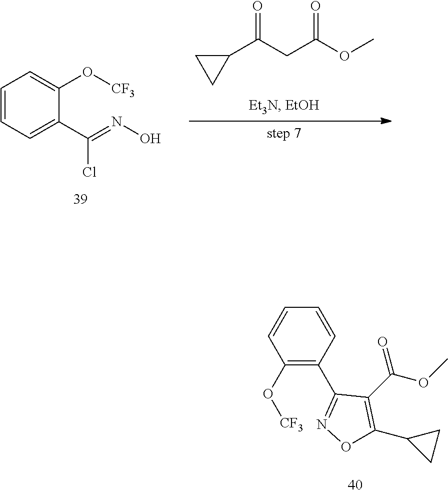

Follow the procedure of Synthesis of 10 to give product 40 (10.5 g, white solid); LCMS: [M+H]+=328.0; ¹H NMR (400 MHz, CDCl₃) δ ppm: 7.56-7.50 (m, 2H), 7.40-7.35 (m, 2H), 3.78 (s, 3H), 2.92-2.83 (m, 1H), 1.39-1.37 (m, 2H), 1.32-1.25 (m, 2H).

### Step 8: Synthesis of 41

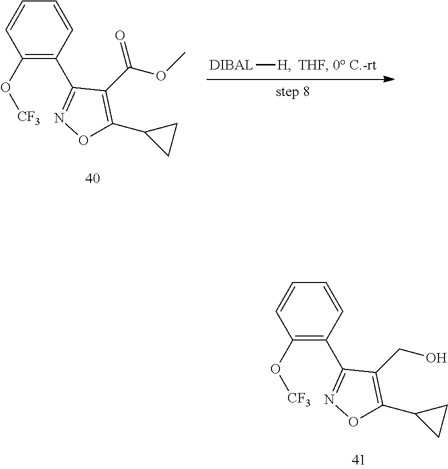

Follow the procedure of Synthesis of 11 to give product 41; LCMS: [M+H]+=300.1. ¹H NMR (400 MHz, CDCl₃) δ ppm: 7.51-7.43 (m, 2H), 7.34-7.31 (m, 2H), 4.42 (s, 2H), 2.13-2.09 (m, 1H), 1.19-1.15 (m, 2H), 1.07-1.05 (m, 2H).

### Step 9: Synthesis of 42

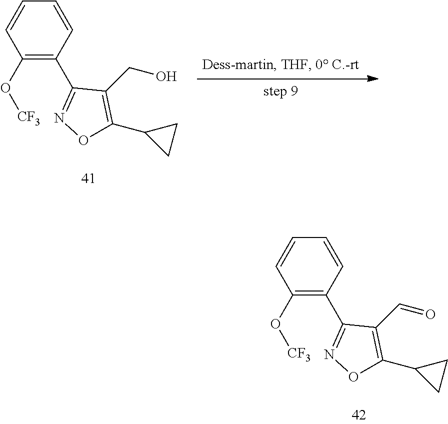 [SMILES: OCc1c(-c2ccccc2OC(F)(F)F)noc1C1CC1 ; "Dess-martin, THF, 0° C.-rt" ; "step 9" ; O=Cc1c(-c2ccccc2OC(F)(F)F)noc1C1CC1]

Follow the procedure of Synthesis of 12 to give product 42. LCMS: [M+H]+=298.1; ¹H NMR (400 MHz, CDCl₃) δ ppm: 9.75 (s, 1H), 7.61-7.57 (m, 2H), 7.46-7.41 (m, 2H), 2.90-2.85 (m, 1H), 1.44-1.41 (m, 2H), 1.38-1.29 (m, 2H).

### Step 10: Synthesis of 43

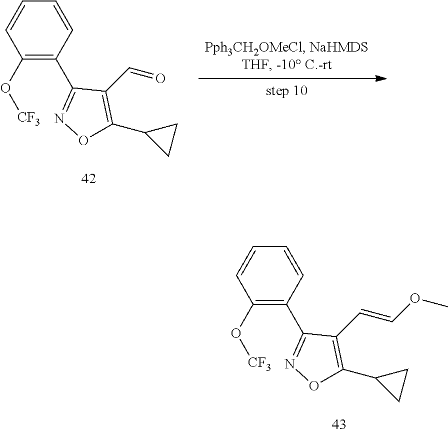 [SMILES: O=Cc1c(-c2ccccc2OC(F)(F)F)noc1C1CC1 ; "Pph3CH2OMeCl, NaHMDSTHF, -10° C.-rt" ; "step 10" ; CO/C=C/c1c(-c2ccccc2OC(F)(F)F)noc1C1CC1]

Follow the procedure of Synthesis of 13 to give product 43. LCMS: [M+H]+=310.1; ¹H NMR (400 MHz, CDCl₃) δ ppm: 7.36-7.34 (m, 2H), 7.29-7.26 (m, 2H), 6.35 (d, J=13.2 Hz, 1H), 5.26 (d, J=13.2 Hz, 1H), 3.47 (s, 3H), 2.01-1.97 (m, 1H), 1.17-1.13 (m, 2H), 1.05-1.02 (m, 2H).

### Step 11: Synthesis of 44

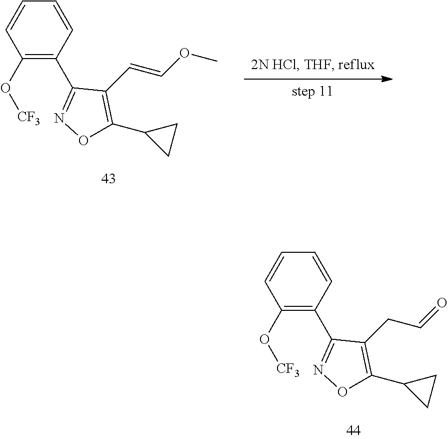 [SMILES: CO/C=C/c1c(-c2ccccc2OC(F)(F)F)noc1C1CC1 ; "2N HCl, THF, reflux" ; "step 11" ; O=CCc1c(-c2ccccc2OC(F)(F)F)noc1C1CC1]

Follow the procedure of Synthesis of 14 to give product 44. LCMS: [M+H]+=312.1; ¹H NMR (400 MHz, CDCl₃) δ ppm: 9.54 (s, 1H), 7.48-7.43 (m, 2H), 7.35-7.30 (m, 2H), 3.39 (s, 2H), 1.86-1.79 (m, 1H), 1.15-1.12 (m, 2H), 1.11-1.05 (m, 2H).

### Step 12: Synthesis of 45

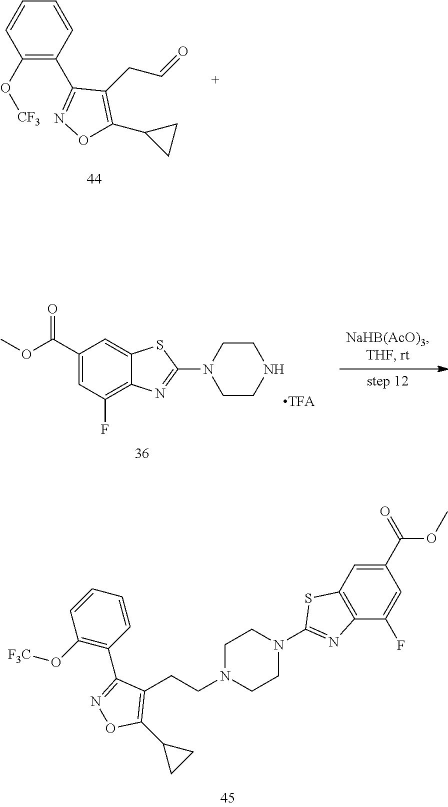

Follow the procedure of Synthesis of 15 to give product 45. LCMS: [M+H]+=591.1; ¹H NMR (400 MHz, CDCl₃) δ ppm: 8.23 (s, 1H), 7.92 (d, J=7.6 Hz, 1H), 7.45-7.42 (m, 1H), 7.37-7.31 (m, 2H), 7.29-7.27 (m, 1H), 3.84 (s, 3H), 3.54 (s, 4H), 2.44-2.41 (m, 8H), 1.98-1.94 (m, 1H), 1.21-1.15 (m, 4H).

### Step 13: Synthesis of I5

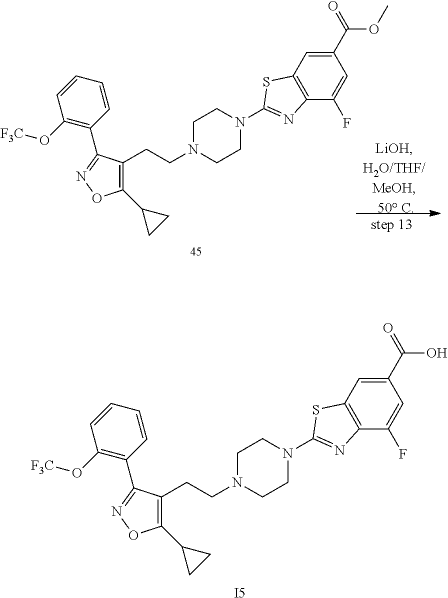

Follow the procedure of Synthesis of I1 to give compound I5. LCMS: [M+H]+=539.1; ¹H NMR (400 MHz, MeOD) δ ppm: 8.24 (s, 1H), 7.73-7.69 (m, 2H), 7.60-7.56 (m, 3H), 3.99 (s, 4H), 3.34-3.33 (m, 4H), 3.32-3.21 (m, 2H), 3.02-2.97 (m, 2H), 2.26-2.23 (m, 1H), 1.23-1.20 (m, 4H).

### Example 6

### 6-(4-(2-(5-cyclopropyl-3-(2,6-dichlorophenyl)isoxazol-4-yl)ethyl)piperazin-1-yl)quinoline-2-carboxylic Acid (I6)

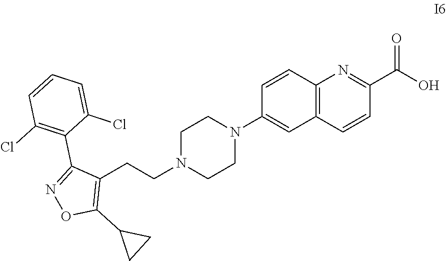 [SMILES: O=C(O)c1ccc2cc(N3CCN(CCc4c(-c5c(Cl)cccc5Cl)noc4C4CC4)CC3)ccc2n1]

### Step 1: Synthesis of 47

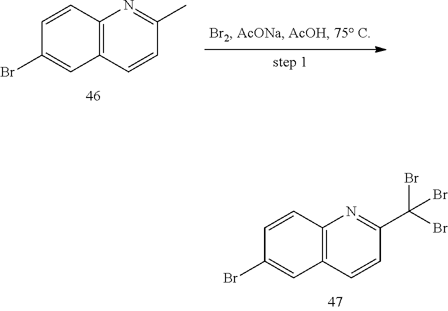

Compound 46 (10 g, 45.03 mmol) and sodium acetate (20.3 g, 247.7 mmol) in acetic acid (100 mL) was heated at 75° C. and stirred until a solution formed. A solution of Br₂ (7.5 mL, 148.6 mmol) in acetic acid (25 mL) was added over 15 min during which time the reaction temperature rose to 86° C. The resulting suspension was heated at 120° C. for 1 h. The suspension was cooled to 80° C. and added to ice-water (200 mL) with stirring. The resulting white solid was collected by filtration, washed with water and dried to give compound 47 (18 g, white solid, yield 87%). LCMS: (ESI-MS): [M+H]+=459; (400 MHz, DMSO-d6) δ ppm: 8.57 (d, J=8.8 Hz, 1H), 8.42 (s, 1H), 8.34 (d, J=8.8 Hz, 1H), 8.05-7.99 (m, 2H).

### Step 2: Synthesis of 48

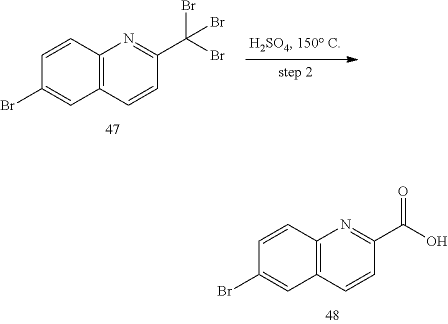 [SMILES: Brc1ccc2nc(C(Br)(Br)Br)ccc2c1 ; O=C(O)c1ccc2cc(Br)ccc2n1]

Concentrated sulfuric acid (38 mL) was added during 15 min to a stirred suspension of 47 (18 g, 39.2 mmol) in water (90 mL). The resulting suspension was heated at 150° C. for 5 h. The mixture was cooled and the precipitate was collected by filtration, washed with water and dried to give compound 48 (4.9 g, white solid, yield 58%). LCMS: (ESI-MS): [M+H]+=251.9, 253.9; (400 MHz, DMSO-d6) δ ppm: 8.53 (d, J=8.8 Hz, 1H), 8.42 (s, 1H), 8.15 (d, J=8.4 Hz, 1H), 8.10 (d, J=8.8 Hz, 1H), 7.80-7.97 (m, 1H).

### Step 3: Synthesis of 49

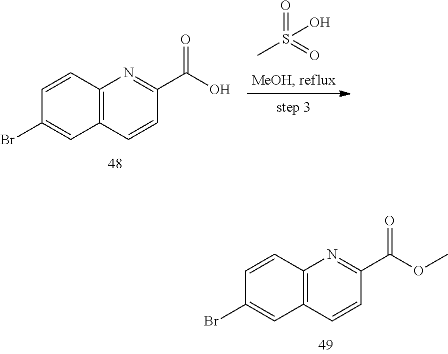

Compound 48 (4.9 g, 19.4 mmol) and methane sulfonic acid (490 mg, 5.05 mmol) in methanol (40 mL) was refluxed for 6 h. After completion of the reaction, the reaction mixture was cooled to rt, then diluted with saturated sodium bicarbonate solution, extracted with EA, and the combined organic layers were washed with brine, dried, concentrated and purified by silica column with heptanes/EA (5:1) to give compound 49 (4.2 g, white solid, yield 81%). LCMS: (ESI-MS): [M+H]+=265.9, 267.9; ¹H NMR (400 MHz, CDCl₃) δ ppm: 8.15-8.09 (m, 3H), 7.99 (s, 1H), 7.80-7.78 (m, 1H), 4.02 (s, 3H).

### Step 4: Synthesis of 50

Under nitrogen, a mixture of compound 49 (4.2 g, 15.8 mmol), tert-butyl piperazine-1-carboxylate (3.53 g, 18.9 mmol), t-BuONa (2.3 g, 23.7 mmol), BINAP (983 mg, 1.6 mmol), Pd2(dba)3 (7.23 mg, 0.8 mmol) were successively added to toluene (55 mL). Then the mixture was heated to 80° C. and stirred overnight. After completion of the reaction, the suspension was filtered, the filter cake was washed with EA. The filtrate was concentrated, dried and purified by silica column with heptanes/EA (2:1) to give compound 50 (1.2 g, yellow solid, yield 14%), and 51 (800 mg, white solid, yield 13%); LCMS: [M+H]+=526.3; ¹H NMR (300 MHz, CDCl₃) δ ppm: 8.09 (d, J=8.1 Hz, 1H), 7.97 (d, J=9.6 Hz, 1H), 7.70 (d, J=8.4 Hz, 1H), 7.53 (d, J=9.9 Hz, 1H), 7.05 (s, 1H), 6.80 (s, 1H), 3.84 (s, 2H), 3.76 (s, 2H), 3.66-3.64 (m, 4H), 3.60 (s, 2H), 3.52 (s, 2H), 3.34-3.33 (m, 4H), 1.52-1.50 (m, 18H).

### Step 5: Synthesis of 52

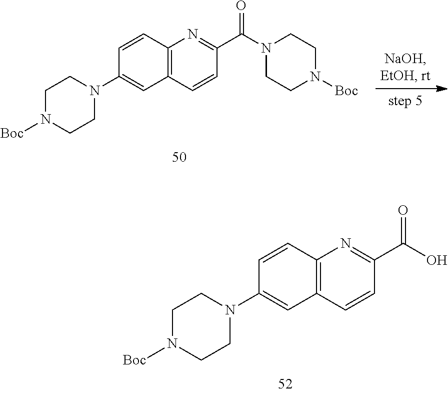 [SMILES: CC(C)(C)OC(=O)N1CCN(C(=O)c2ccc3cc(N4CCN(C(=O)OC(C)(C)C)CC4)ccc3n2)CC1 ; CC(C)(C)OC(=O)N1CCN(c2ccc3nc(C(=O)O)ccc3c2)CC1]

To a solution of compound 50 (742 mg, 1.4 mmol) in EtOH (15 mL), NaOH (560 mg, 14 mmol) was added at rt for overnight. After completion of the reaction, the reaction was acidified with 2 N HCl and purified by pre-HPLC to give compound 52 (200 mg, yellow solid, yield 40%). LCMS: [M+H]+=358.2.

### Step 6: Synthesis of 53

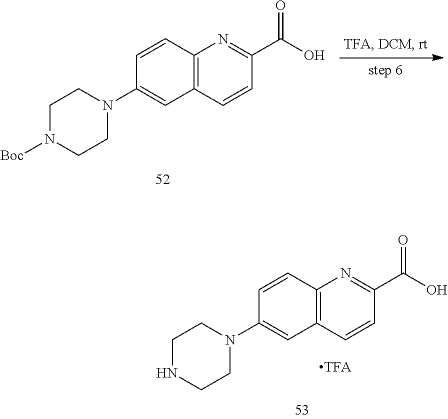 [SMILES: CC(C)(C)OC(=O)N1CCN(c2ccc3nc(C(=O)O)ccc3c2)CC1 ; O=C(O)c1ccc2cc(N3CCNCC3)ccc2n1]

Follow the procedure of Synthesis of 6 to give product 53. LCMS: [M+H]+=258.1.

### Step 7: Synthesis of I6

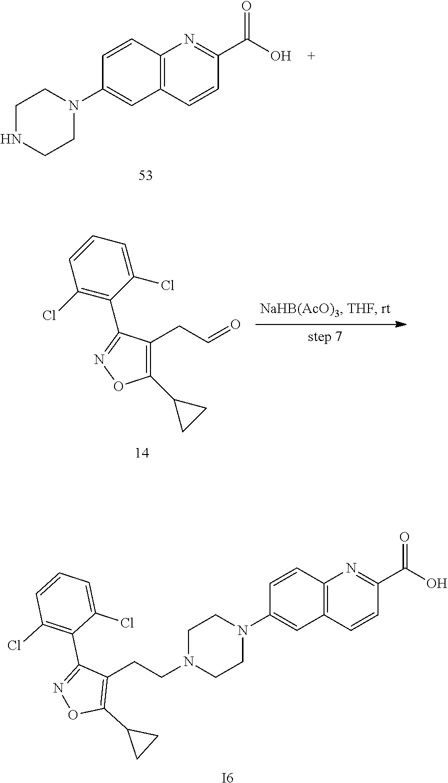

Follow the procedure of Synthesis of 15 to give product to give crude product and purified by pre-HPLC to give compound I6. LCMS: [M+H]+=537.1; ¹H NMR (400 MHz, MeOD) δ ppm: 8.37 (d, J=8.0 Hz, 1H), 8.17-8.15 (m, 2H), 7.75 (d, J=8.8 Hz, 1H), 7.64-7.58 (m, 3H), 7.35 (s, 1H), 3.67-3.53 (m, 8H), 3.33 (s, 2H), 2.94-2.89 (m, 2H), 2.31-2.29 (m, 1H), 1.26-1.23 (m, 4H).

### Example 7

### 6-(4-(2-(5-cyclopropyl-3-(2,6-dichlorophenyl)isoxazol-4-yl)ethyl)-1,4-diazepan-1-yl)-1-methyl-1H-indole-3-carboxylic Acid (I7)

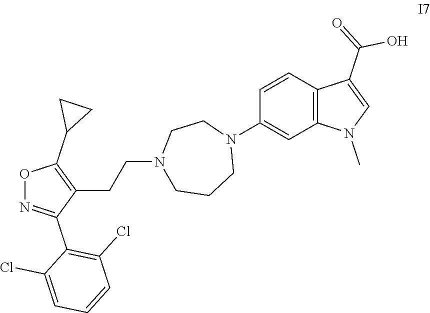 [SMILES: Cn1cc(C(=O)O)c2ccc(N3CCCN(CCc4c(-c5c(Cl)cccc5Cl)noc4C4CC4)CC3)cc21]

### Step 1: Synthesis of 54

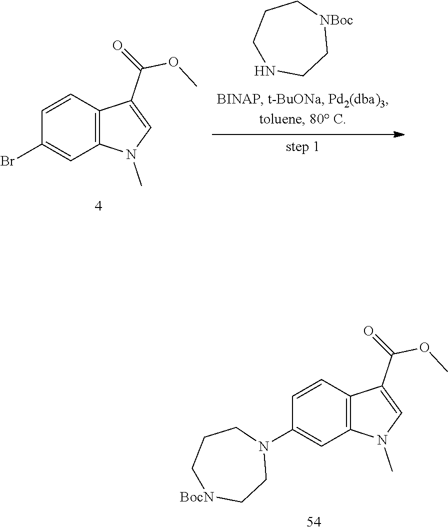 [SMILES: COC(=O)c1cn(C)c2cc(Br)ccc12 ; CC(C)(C)OC(=O)N1CCCNCC1 ; COC(=O)c1cn(C)c2cc(N3CCCN(C(=O)OC(C)(C)C)CC3)ccc12]

Follow the procedure of Synthesis of 5 to give product 54. ¹H NMR (400 MHz, CDCl₃) δ 7.88 (d, J=8.7 Hz, 1H), 7.51 (s, 1H), 6.73 (d, J=8.4 Hz, 1H), 6.43 (s, 1H), 3.81 (s, 3H), 3.66 (s, 3H), 3.58 (t, J=7.0 Hz, 6H), 3.26 (s, 1H), 3.15 (s, 1H), 1.96 (d, J=10.2 Hz, 2H), 1.37 (s, 5H), 1.28 (s, 4H). LCMS: [M+H]+=388.3.

### Step 2: Synthesis of 55

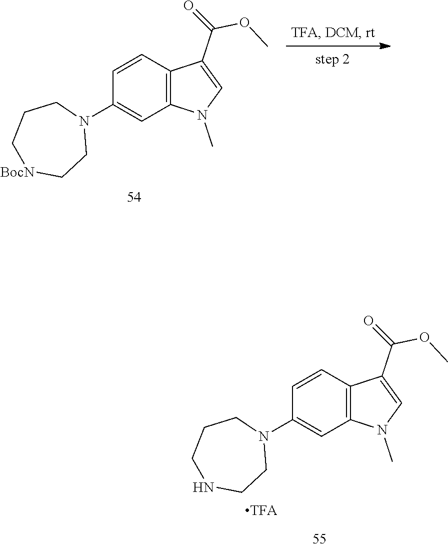 [SMILES: COC(=O)c1cn(C)c2cc(N3CCCN(C(=O)OC(C)(C)C)CC3)ccc12 ; COC(=O)c1cn(C)c2cc(N3CCCNCC3)ccc12]

Follow the procedure of Synthesis of 6 to give product 55. ¹H NMR (400 MHz, CDCl₃) δ 9.51 (s, 1H), 8.01-7.95 (m, 1H), 7.61 (d, J=9.3 Hz, 1H), 6.68-6.76 (m, 1H), 3.82 (s, 3H), 3.70 (s, 3H), 3.68-3.56 (m, 2H), 3.43 (d, J=16.0 Hz, 1H), 3.36-3.17 (m, 1H), 2.68 (s, 4H), 2.31 (s, 3H). LCMS: [M+H]+=288.2.

### Step 3: Synthesis of 56

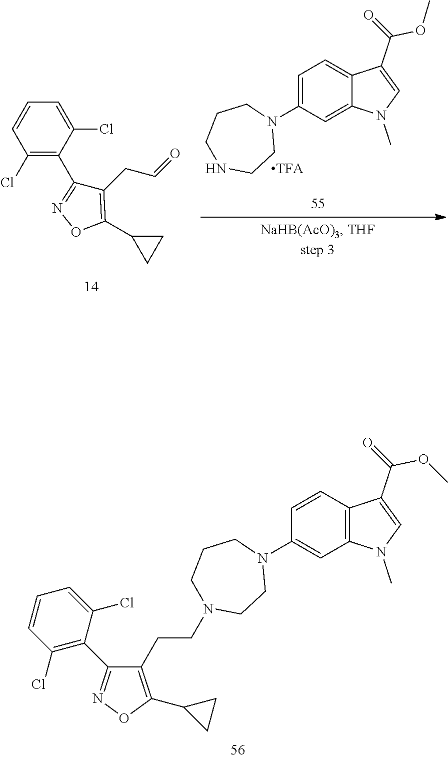

Follow the procedure of Synthesis of 15 to give product 56. ¹H NMR (400 MHz, CDCl₃) δ 7.87 (s, 1H), 7.51 (s, 1H), 7.29 (s, 2H), 7.20 (s, 1H), 6.67 (s, 1H), 6.36 (s, 1H), 3.81 (s, 3H), 3.65 (s, 3H), 3.47 (d, J=32.5 Hz, 4H), 2.77 (s, 2H), 2.16 (s, 3H), 1.97 (s, 4H), 1.19-1.12 (m, 6H); LCMS: [M+H]+=569.2.

### Step 4: Synthesis of 17

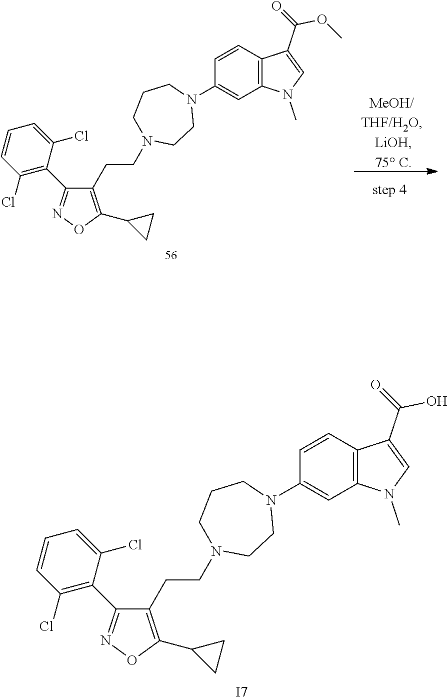

Follow the procedure of Synthesis of I1 to give compound I7. ¹H NMR (400 MHz, CDCl₃) δ 7.96 (d, J=8.8 Hz, 1H), 7.65 (s, 1H), 7.37 (d, J=7.5 Hz, 2H), 7.33-7.28 (m, 1H), 6.73 (d, J=8.0 Hz, 1H), 6.45 (s, 1H), 3.73 (s, 3H), 3.63 (s, 2H), 3.51 (t, J=6.0 Hz, 2H), 2.91 (s, 2H), 2.67 (s, 6H), 2.08 (s, 4H), 1.18 (d, J=4.2 Hz, 2H), 1.06 (d, J=5.9 Hz, 2H). LCMS: [M+H]+=553.2.

### Example 8

### 6-(4-(2-(5-cyclopropyl-3-(2,6-dichlorophenyl)isoxazol-4-yl)ethyl)-3-methylpiperazin-1-yl)-1-methyl-1H-indole-3-carboxylic Acid (I8)

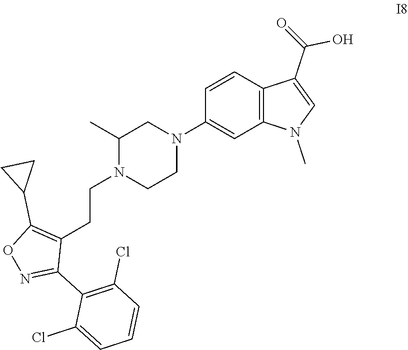 [SMILES: CC1CN(c2ccc3c(C(=O)O)cn(C)c3c2)CCN1CCc1c(-c2c(Cl)cccc2Cl)noc1C1CC1]

### Step 1: Synthesis of 57

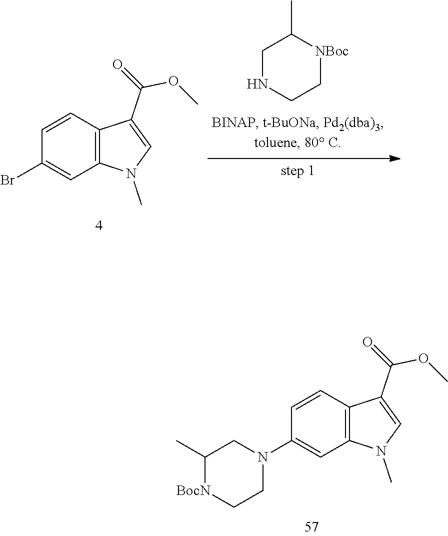

Under nitrogen, a mixture of compound 4 (1.5 g, 5.59 mmol, 1.0 eq), tert-butyl 2-methylpiperazine-1-carboxylate (1.34 g, 6.71 mmol, 1.2 eq), t-BuONa (5.1 g, 8.39 mmol, 1.5 eq), BINAP (348 mg, 0.55 mmol, 0.1 eq) and Pd2(dba)3 (256 mg, 0.28 mmol, 0.05 eq) were successively added to toluene (15 mL). Then the mixture was heated to 80° C. and stirred overnight. After completion of the reaction, the suspension is filtered, the filter cake was washed with EA (50 mL×2), the filtrate is concentrated, dried and purified by silica column with PE/EA (10:1) to give the desired product compound 57 (360 mg, yellow solid, yield 16.5%). 1HNMR (400 MHz, CDCl3) δ 7.95 (d, J=8.7 Hz, 1H), 7.60 (s, 1H), 6.92 (d, J=8.5 Hz, 1H), 6.68 (s, 1H), 4.27 (d, J=27.6 Hz, 1H), 3.92 (d, J=12.9 Hz, 1H), 3.82 (s, 3H), 3.70 (s, 3H), 3.45 (d, J=11.3 Hz, 1H), 3.31 (t, J=12.0 Hz, 2H), 1.97 (s, 1H), 1.41 (d, J=8.5 Hz, 1H), 1.30 (d, J=6.5 Hz, 9H), 1.19 (t, J=7.1 Hz, 3H). LCMS: (ESI-MS): [M+H]+=388.3.

### Step 2: Synthesis of 58

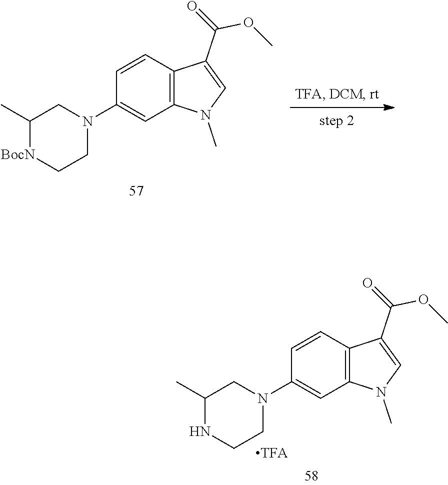

To a solution of compound 57 (360 mg, 0.927 mmol, 1.0 eq) in dichloromethane (4 mL), TFA (1 mL) were added. Then the reaction was stirred 1 h at room temperature. After completion of the reaction, the reaction solution was concentrated, the residue diluted with MTBE (5 mL), the suspension is filtered, the filter cake was washed with MTBE (5 mL×2), dried to get compound 58 (260 mg, white solid, yield 69.7%).

1H NMR (400 MHz, CDCl3) δ 9.97 (s, 1H), 9.28 (s, 1H), 8.00 (d, J=8.7 Hz, 1H), 7.65 (s, 1H), 6.93 (d, J=8.4 Hz, 1H), 6.80 (s, 1H), 3.83 (s, 3H), 3.73 (s, 3H), 3.55-3.42 (m, 4H), 3.30-3.15 (m, 2H), 3.00 (t, J=12 Hz, 1H), 1.40 (d, J=6.1 Hz, 3H). LCMS: (ESI-MS): [M+H]+=288.2.

### Step 3: Synthesis of 59

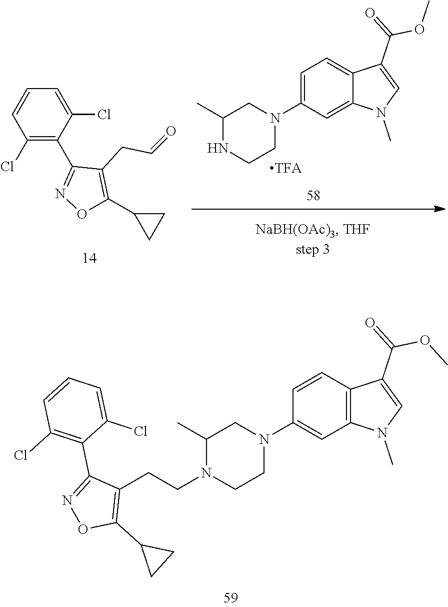

To a solution of compound 14 (192 mg, 0.65 mmol, 1.0 eq) in THF (5 ml), compound 58 (260 mg, 0.65 mmol, 1.0 eq) was added at rt. Then the reaction mixture was stirred at rt for 30 min and treated with NaBH(OAc)3 (412 mg, 1.94 mmol, 3.0 eq), allowed to stirred at rt for another 30 min. After the reaction completed, quenched with water, extracted with EA (10 mL×2), and the combined organic layers were washed with brine (20 mL), dried, concentrated and purified by silica column with PE/EA (2:1) to give compound 59 (180 mg, white solid, yield 48.7%). 1H NMR (400 MHz, CDCl3) δ 7.92 (s, 1H), 7.58 (s, 1H), 7.42-7.32 (m, 1H), 7.29-7.23 (m, 1H), 6.90 (dd, J=8.8, 2.1 Hz, 1H), 6.65 (d, J=1.9 Hz, 1H), 3.81 (s, 3H), 3.63 (s, 3H), 3.41-3.26 (m, 3H), 2.62-2.34 (m, 6H), 2.14 (d, J=9.8 Hz, 3H), 2.03-1.96 (m, 2H), 1.19-1.12 (m, 6H). LCMS: (ESI-MS): [M+H]+=567.2.

### Step 4: Synthesis of I8

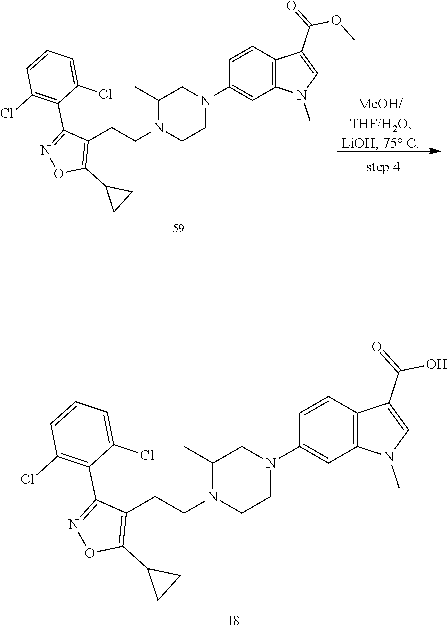

To a solution of compound 59 (180 mg, 0.32 mmol, 1.0 eq) in MeOH/THF/H₂O=1:1:1 (5 mL), LiOH (200 mg, 4.77 mmol, 15.0 eq) was added at rt. Then the reaction mixture was heated to 75° C. for overnight. After completion of the reaction, the reaction was acidified with 2 N aqueous and a precipitate formed as the solution cooled to RT. Then the reaction was diluted with water (5 mL), extracted with EA (15 mL×2), the combined organic layers were washed with brine, dried and purified by Prep-TLC with DCM:MEOH=10:1 to give the compound I8 (27 mg, white solid, yield 15.4%). 1NMR (400 MHz, CDCl3) δ 7.96 (d, J=8.7 Hz, 1H), 7.67 (s, 1H), 7.37 (d, J=7.8 Hz, 2H), 7.33-7.24 (m, 1H), 6.94-6.82 (m, 1H), 6.68 (s, 1H), 3.71 (s, 3H), 3.37 (s, 3H), 2.88 (s, 4H), 2.55 (s, 6H), 2.03 (s, 3H), 1.17 (d, J=11.8 Hz, 4H), 1.05 (s, 5H). LCMS: (ESI-MS): [M+H]+=553.2.

### Example 9

### 6-(4-(2-(3-(2-chlorophenyl)-5-cyclopropylisoxazol-4-yl)ethyl)piperazin-1-yl)-1-methyl-1H-indole-3-carboxylic Acid (19)

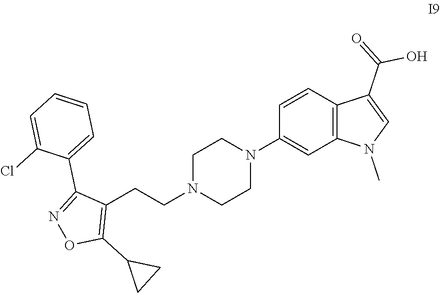 [SMILES: Cn1cc(C(=O)O)c2ccc(N3CCN(CCc4c(-c5ccccc5Cl)noc4C4CC4)CC3)cc21]

### Step 1: Synthesis of 62

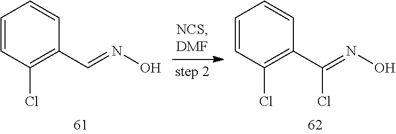 [SMILES: O/N=C/c1ccccc1Cl ; O/N=C(\Cl)c1ccccc1Cl]

Follow the procedure of Synthesis of 9 to give product 62 used directly in the next step. ¹H NMR (400 MHz, DMSO-d6) δ ppm: 12.68 (br, 1H), 7.60-7.47 (m, 3H), 7.50-7.43 (m, 1H).

### Step 2: Synthesis of 63

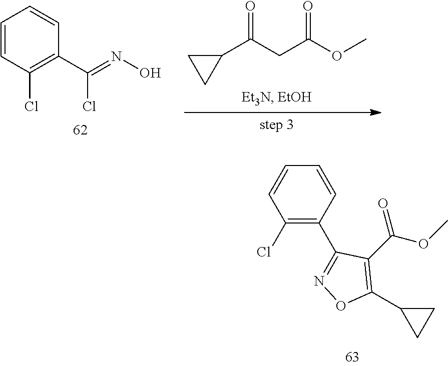

Follow the procedure of Synthesis of 10 to give 63. LCMS: [M+H]+=278.1; ¹H NMR (400 MHz, CDCl₃) δ ppm: 7.50-7.45 (m, 1H), 7.45-7.35 (m, 3H), 3.72 (s, 3H), 2.93-2.87 (m, 1H), 1.41-1.37 (m, 2H), 1.35-1.28 (m, 2H).

### Step 3: Synthesis of 64

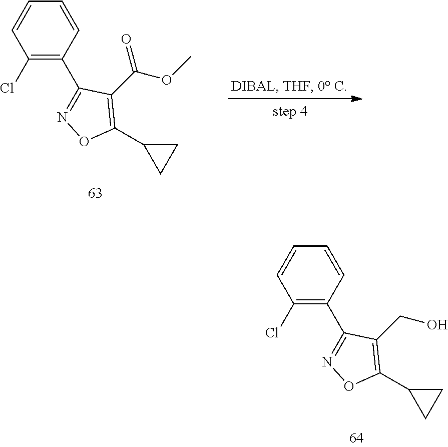 [SMILES: COC(=O)c1c(-c2ccccc2Cl)noc1C1CC1 ; OCc1c(-c2ccccc2Cl)noc1C1CC1]

Follow the procedure of Synthesis of 11 to give 64. LCMS: [M+H]+=250.1; ¹H NMR (CDCl₃) δ ppm: 7.43-7.26 (m, 4H), 4.40 (s, 2H), 2.13-2.10 (m, 1H), 1.20-1.15 (m, 2H), 1.07-1.03 (m, 2H).

### Step 4: Synthesis of 65

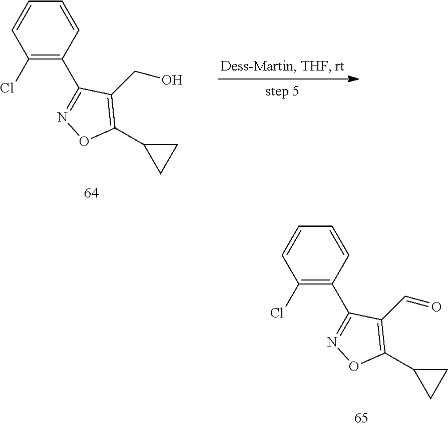 [SMILES: OCc1c(-c2ccccc2Cl)noc1C1CC1 ; O=Cc1c(-c2ccccc2Cl)noc1C1CC1]

Follow the procedure of Synthesis of 12 to give 65. LCMS: [M+H]+=248.1; ¹H NMR (400 MHz, CDCl₃) δ ppm: 9.63 (s, 1H), 7.47-7.45 (m, 1H), 7.42-7.39 (m, 2H), 7.35-7.31 (m, 1H), 2.84-2.80 (m, 1H), 1.38-1.34 (m, 2H), 1.28-1.24 (m, 2H).

### Step 5: Synthesis of 66

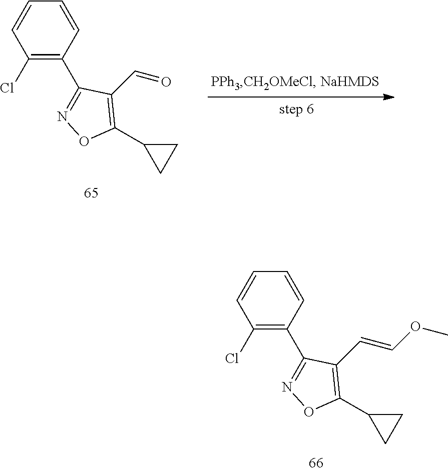 [SMILES: O=Cc1c(-c2ccccc2Cl)noc1C1CC1 ; CO/C=C/c1c(-c2ccccc2Cl)noc1C1CC1]

Follow the procedure of Synthesis of 13 to give 66. LCMS: [M+H]+=276.1; ¹H NMR (400 MHz, CDCl₃) δ ppm: 7.41-7.25 (m, 4H), 6.43 (d, J=13.2 Hz, 1H), 5.29 (d, J=13.2 Hz, 1H), 3.48 (s, 3H), 2.04-1.93 (m, 1H), 1.13-1.10 (m, 2H), 1.03-0.99 (m, 2H).

### Step 6: Synthesis of 67

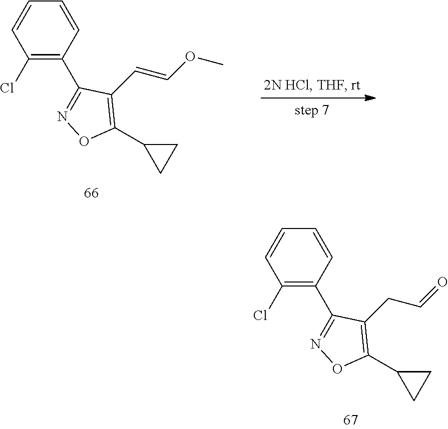 [SMILES: CO/C=C/c1c(-c2ccccc2Cl)noc1C1CC1 ; O=CCc1c(-c2ccccc2Cl)noc1C1CC1]

Follow the procedure of Synthesis of 14 to give 67. LCMS: [M+H]+=262.1; ¹H NMR (400 MHz, CDCl₃) δ ppm: 9.53 (s, 1H), 7.43-7.40 (m, 3H), 7.37-7.28 (m, 3H), 3.39 (s, 2H), 1.85-1.81 (m, 1H), 1.14-1.11 (m, 2H), 1.05-1.01 (m, 2H).

### Step 7: Synthesis of 68

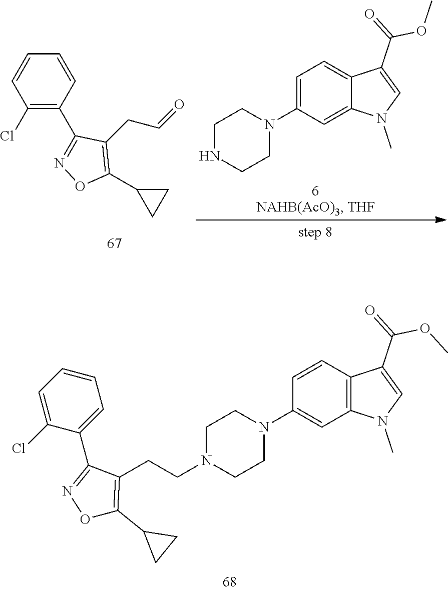

Follow the procedure of Synthesis of 15 to give 68. LCMS: [M+H]+=519.2.

### Step 8: Synthesis of I9

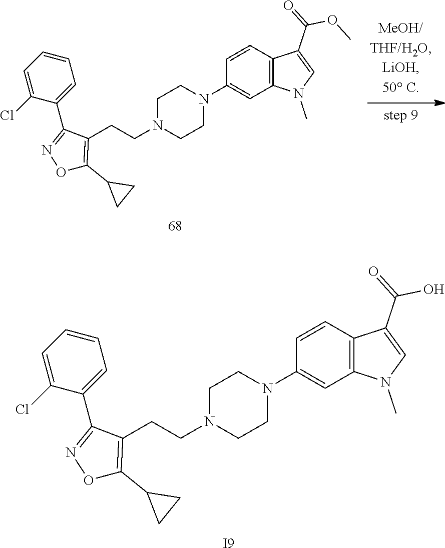

Follow the procedure of Synthesis of I1 to give compound I9. LCMS: [M+H]+=505.1; ¹H NMR (400 MHz, DMSO-d6) δ ppm: 11.78 (br, 1H), 7.83-7.80 (m, 2H), 7.77-7.67 (m, 1H), 7.64-7.50 (m, 3H), 6.92-6.87 (m, 2H), 3.76 (s, 3H), 3.04 (s, 4H), 2.56 (s, 2H), 2.39 (s, 4H), 2.29-2.25 (m, 3H), 1.11-1.04 (m, 4H).

### Example 10

### 6-(4-(2-(3-(2-chloro-6-fluorophenyl)-5-cyclopropylisoxazol-4-yl)ethyl)piperazin-1-yl)-1-methyl-1H-indole-3-carboxylic Acid (I10)

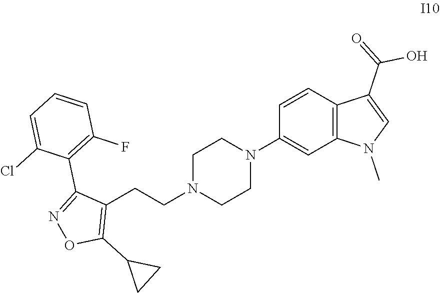 [SMILES: Cn1cc(C(=O)O)c2ccc(N3CCN(CCc4c(-c5c(F)cccc5Cl)noc4C4CC4)CC3)cc21]

### Step 1: Synthesis of 71

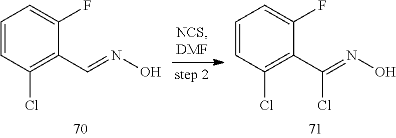 [SMILES: O/N=C/c1c(F)cccc1Cl ; O/N=C(\Cl)c1c(F)cccc1Cl]

Follow the procedure of Synthesis of 9 to give 71 used directly in the next step. ¹H NMR (400 MHz, DMSO-d6) δ ppm: 12.82 (br, 1H), 7.61-7.60 (m, 1H), 6.48 (d, J=8.0 Hz, 1H), 7.41-7.39 (m, 1H).

### Step 2: Synthesis of 72

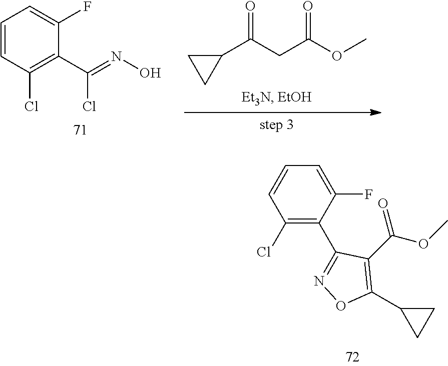

Follow the procedure of Synthesis of 10 to give 72. LCMS: [M+H]+=296.0; ¹H NMR (400 MHz, CDCl₃) δ ppm: 7.44-7.38 (m, 1H), 7.32 (d, J=8.0 Hz, 1H), 7.14-7.10 (m, 1H), 3.87 (s, 3H), 2.95-2.90 (m, 1H), 1.45-1.40 (m, 2H), 1.37-1.28 (m, 2H).

### Step 3: Synthesis of 73

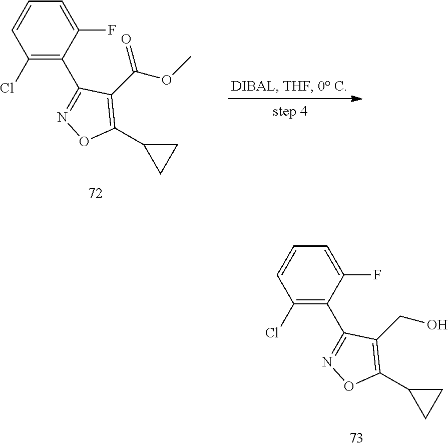 [SMILES: COC(=O)c1c(-c2c(F)cccc2Cl)noc1C1CC1 ; OCc1c(-c2c(F)cccc2Cl)noc1C1CC1]

Follow the procedure of Synthesis of 11 to give 73. LCMS: [M+H]+=268.0; ¹H NMR (CDCl₃) δ ppm: 7.35-7.29 (m, 1H), 7.24 (d, J=8.0 Hz, 1H), 7.06-7.02 (m, 1H), 4.35 (s, 2H), 2.14-2.07 (m, 1H), 1.19-1.15 (m, 2H), 1.13-1.11 (m, 2H).

### Step 4: Synthesis of 74

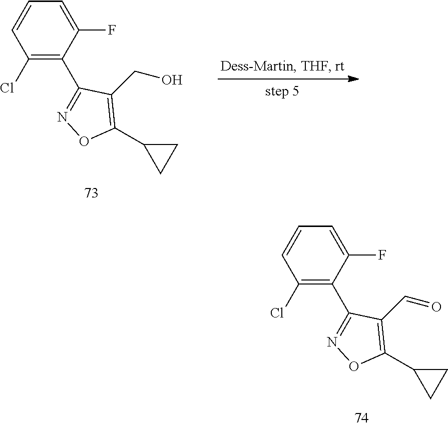 [SMILES: OCc1c(-c2c(F)cccc2Cl)noc1C1CC1 ; O=Cc1c(-c2c(F)cccc2Cl)noc1C1CC1]

Follow the procedure of Synthesis of 12 to give 74. LCMS: [M+H]+=266.0; ¹H NMR (CDCl₃) δ ppm: 9.74 (s, 1H), 7.50-7.47 (m, 1H), 7.38 (d, J=8.0 Hz, 1H), 7.24-7.16 (m, 1H), 2.87-2.82 (m, 1H), 1.51-1.47 (m, 2H), 1.40-1.35 (m, 2H).

### Step 5: Synthesis of 75

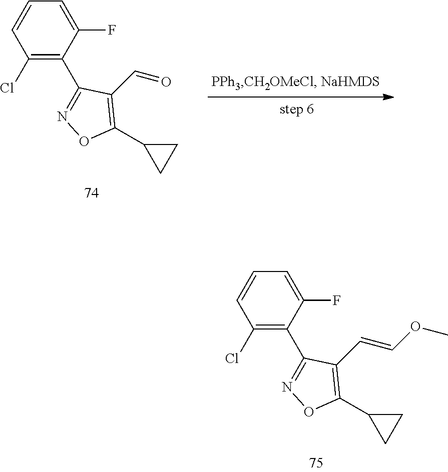 [SMILES: O=Cc1c(-c2c(F)cccc2Cl)noc1C1CC1 ; CO/C=C/c1c(-c2c(F)cccc2Cl)noc1C1CC1]

Follow the procedure of Synthesis of 13 to give 75. LCMS: [M+H]+=294.1; ¹H NMR (400 MHz, CDCl₃) δ ppm: 7.31-7.23 (m, 2H), 7.06-7.21 (m, 2H), 6.43 (d, J=13.2 Hz, 1H), 5.25 (d, J=13.2 Hz, 1H), 3.48 (s, 3H), 2.02-1.93 (m, 1H), 1.50-1.12 (m, 2H), 1.04-1.00 (m, 2H).

### Step 6: Synthesis of 76

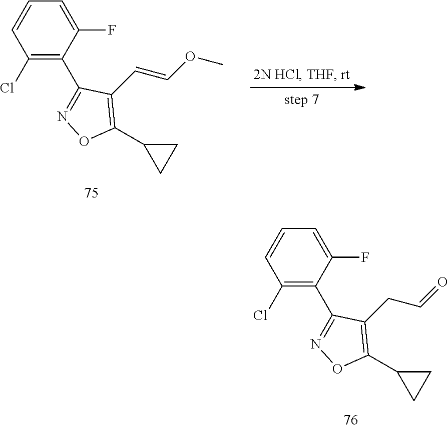 [SMILES: CO/C=C/c1c(-c2c(F)cccc2Cl)noc1C1CC1 ; O=CCc1c(-c2c(F)cccc2Cl)noc1C1CC1]

Follow the procedure of Synthesis of 14 to give 76. LCMS: [M+H]+=280.0; ¹H NMR (400 MHz, CDCl₃) δ ppm: 9.51 (s, 1H), 7.36-7.31 (m, 1H), 7.26-7.24 (m, 1H), 7.07-7.03 (m, 1H), 3.32 (s, 2H), 1.89-1.85 (m, 1H), 1.16-1.14 (m, 2H), 1.07-1.03 (m, 2H).

### Step 7: Synthesis of 77

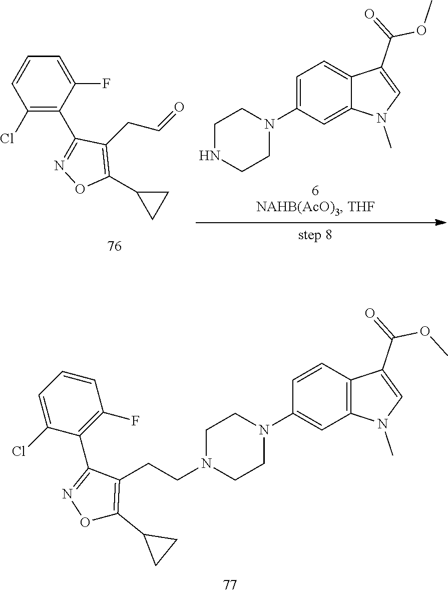

Follow the procedure of Synthesis of 15 to give 77. LCMS: [M+H]+=537.2.

### Step 8: Synthesis of I10

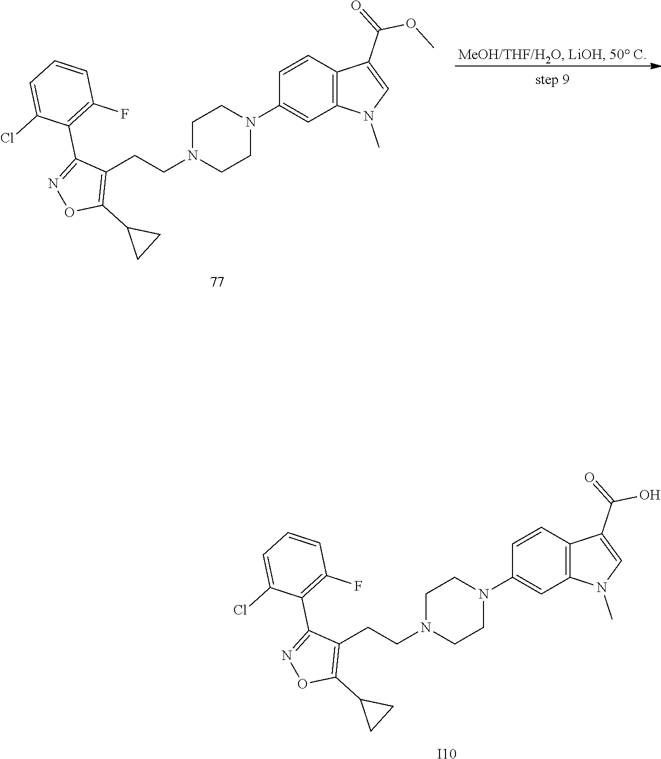

Follow the procedure of Synthesis of I1 to give I10. LCMS: [M+H]+=523.2; ¹H NMR (400 MHz, DMSO-d6) δ ppm: 11.77 (br, 1H), 7.83-7.77 (m, 2H), 7.68-7.64 (m, 1H), 7.56-7.53 (m, 1H), 7.47-7.42 (m, 1H), 6.92-6.87 (m, 2H), 3.76 (s, 3H), 3.04 (s, 4H), 2.52 (s, 2H), 2.40 (s, 4H), 2.33-2.28 (m, 3H), 1.12-1.05 (m, 4H).

### Example 11

### 6-(4-(2-(5-cyclopropyl-3-(2,6-difluorophenyl)isoxazol-4-yl)ethyl) piperazin-1-yl)-1-methyl-1H-indole-3-carboxylic Acid (I11)

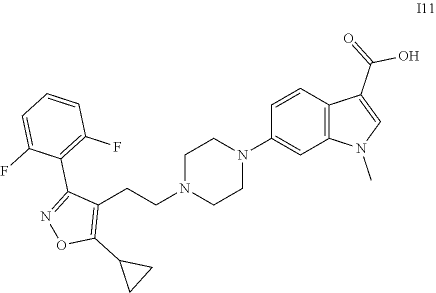 [SMILES: Cn1cc(C(=O)O)c2ccc(N3CCN(CCc4c(-c5c(F)cccc5F)noc4C4CC4)CC3)cc21]

### Step 1: Synthesis of 80

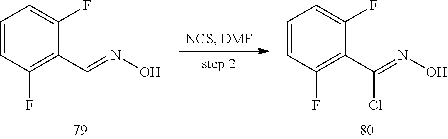

Follow the procedure of Synthesis of 9 to give 80 used directly in the next step. ¹H NMR (400 MHz, DMSO-d6) δ ppm: 12.88 (br, 1H), 7.65-7.61 (m, 1H), 7.27-7.23 (m, 2H).

### Step 2: Synthesis of 81

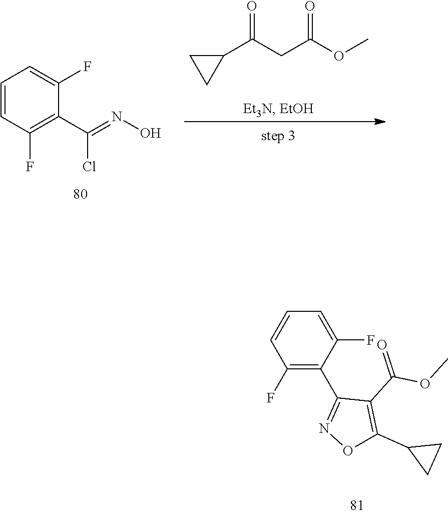

Follow the procedure of Synthesis of 10 to give 81. LCMS: [M+H]+=280.1; ¹H NMR (400 MHz, CDCl₃) δ ppm: 7.49-7.41 (m, 1H), 7.04-7.00 (m, 2H), 3.75 (s, 3H), 2.93-2.89 (m, 1H), 1.42-1.38 (m, 2H), 1.31-1.30 (m, 2H).

### Step 3: Synthesis of 82

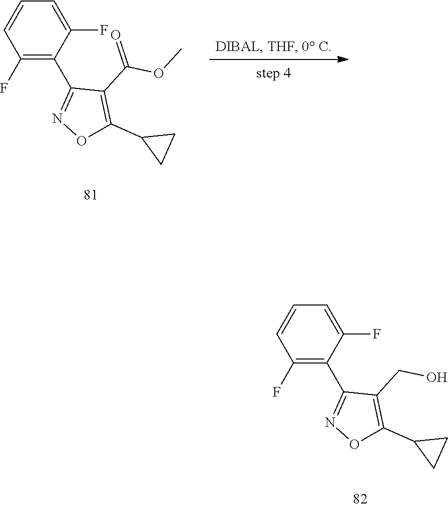

Follow the procedure of Synthesis of 11 to give 82. LCMS: [M+H]+=252.1; ¹H NMR (400 MHz, CDCl₃) δ ppm: 7.40-7.33 (m, 1H), 6.98-6.94 (m, 2H), 4.40 (s, 2H), 2.15-2.08 (m, 1H), 1.19-1.16 (m, 2H), 1.15-1.06 (m, 2H).

### Step 4: Synthesis of 83

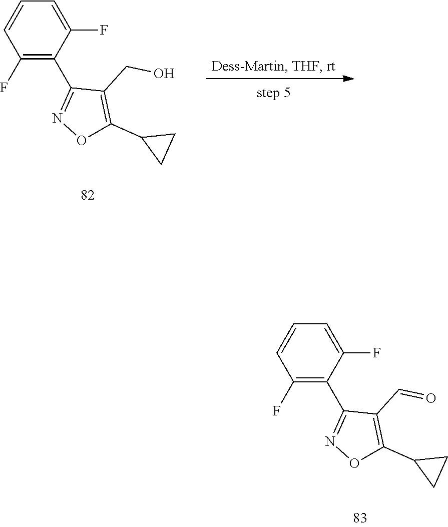 [SMILES: OCc1c(-c2c(F)cccc2F)noc1C1CC1 ; O=Cc1c(-c2c(F)cccc2F)noc1C1CC1]

Follow the procedure of Synthesis of 12 to give the 83. LCMS: [M+H]+=250.1; ¹H NMR (400 MHz, CDCl₃) δ ppm: 9.81 (s, 1H), 7.54-7.50 (m, 1H), 7.11-7.07 (m, 2H), 2.87-2.81 (m, 1H), 1.49-1.45 (m, 2H), 1.39-1.35 (m, 2H).

### Step 5: Synthesis of 84

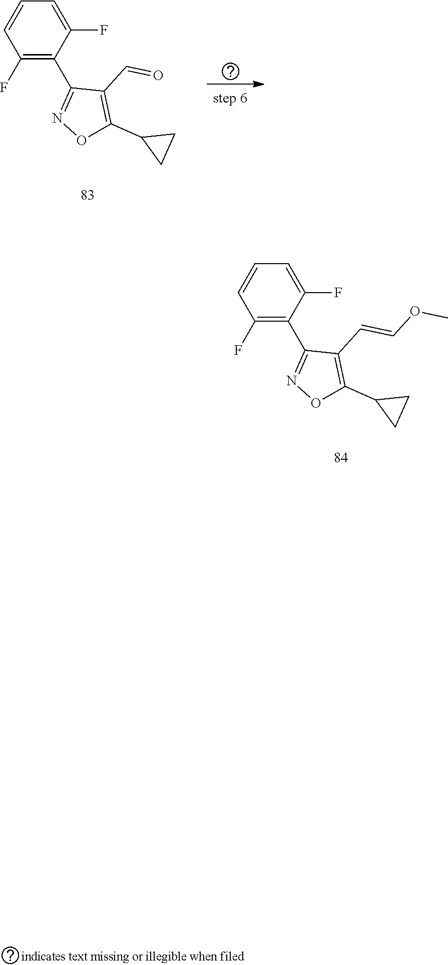 [SMILES: O=Cc1c(-c2c(F)cccc2F)noc1C1CC1 ; CO/C=C/c1c(-c2c(F)cccc2F)noc1C1CC1]

Follow the procedure of Synthesis of 13 to give 84. LCMS: [M+H]+=278.1; ¹H NMR (400 MHz, CDCl₃) δ ppm: 7.33-7.29 (m, 1H), 6.96-6.89 (m, 2H), 6.52 (d, J=12.8 Hz, 1H), 5.26 (d, J=12.8 Hz, 1H), 3.50 (s, 3H), 1.98-1.96 (m, 1H), 1.14-1.10 (m, 2H), 1.03-0.97 (m, 2H).

### Step 6: Synthesis of 85

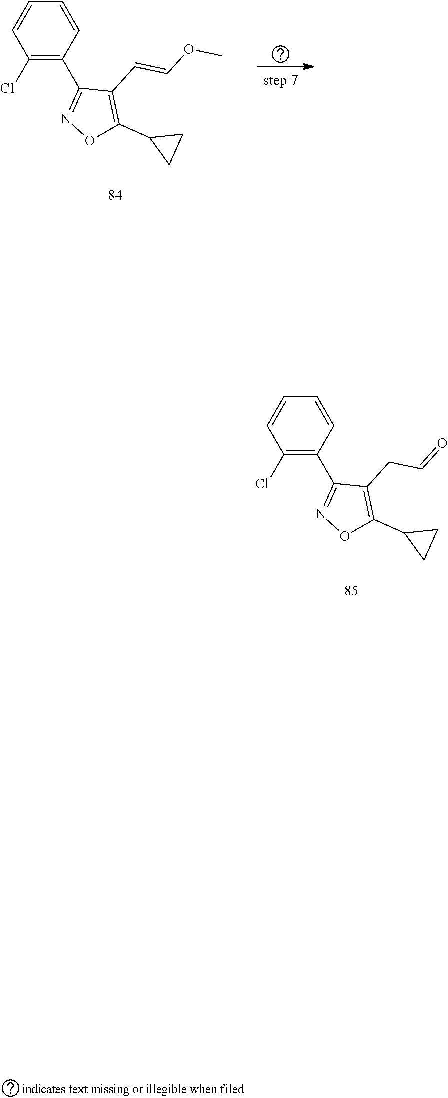 [SMILES: CO/C=C/c1c(-c2ccccc2Cl)noc1C1CC1 ; O=CCc1c(-c2ccccc2Cl)noc1C1CC1]

Follow the procedure of Synthesis of 14 to give 85. LCMS: [M+H]+=264.1; ¹H NMR (400 MHz, CDCl₃) δ ppm: 9.54 (s, 1H), 7.39-7.35 (m, 1H), 6.98-6.94 (m, 2H), 3.37 (s, 2H), 1.88-1.83 (m, 1H), 1.18-1.14 (m, 2H), 1.06-1.01 (m, 2H).

### Step 7: Synthesis of 86

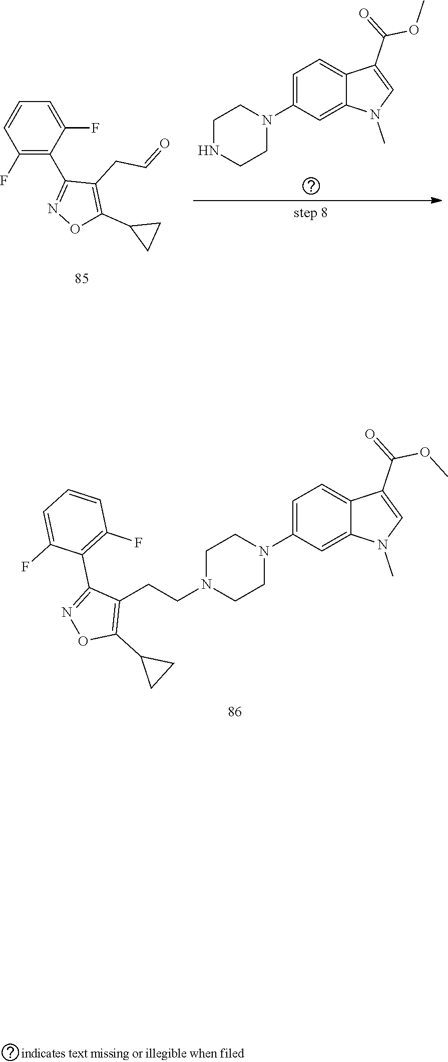 [SMILES: O=CCc1c(-c2c(F)cccc2F)noc1C1CC1 ; COC(=O)c1cn(C)c2cc(N3CCNCC3)ccc12 ; COC(=O)c1cn(C)c2cc(N3CCN(CCc4c(-c5c(F)cccc5F)noc4C4CC4)CC3)ccc12]

Follow the procedure of Synthesis of 15 to give 86. LCMS: [M+H]+=521.2;

### Step 8: Synthesis of I11

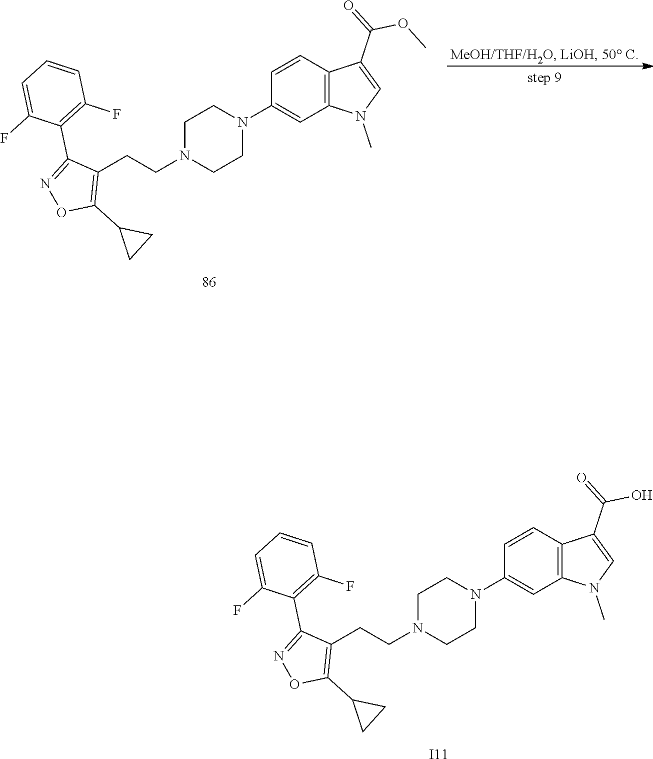

Follow the procedure of Synthesis of I1 to give I11. LCMS: [M+H]+=507.2; ¹H NMR (400 MHz, DMSO-d6) δ ppm: 11.78 (br, 1H), 7.83-7.77 (m, 2H), 7.70-7.65 (m, 1H), 7.35-7.30 (m, 2H), 6.92-6.87 (m, 2H), 3.76 (s, 3H), 3.02 (s, 4H), 2.55-2.50 (m, 2H), 2.40 (s, 4H), 2.35-2.25 (m, 3H), 1.15-1.02 (m, 4H).

### Example 12

### 6-(4-(2-(5-cyclopropyl-3-(2,6-dichlorophenyl)isoxazol-4-yl)ethyl)-2-methylpiperazin-1-yl)-1-methyl-1H-indole-3-carboxylic Acid (I12)

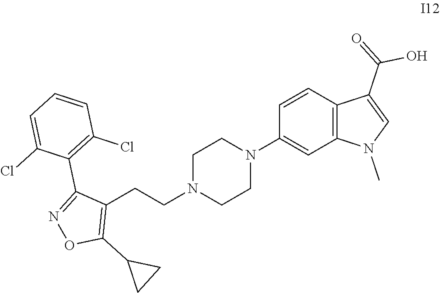 [SMILES: Cn1cc(C(=O)O)c2ccc(N3CCN(CCc4c(-c5c(Cl)cccc5Cl)noc4C4CC4)CC3)cc21]

### Step 1: Synthesis of 87

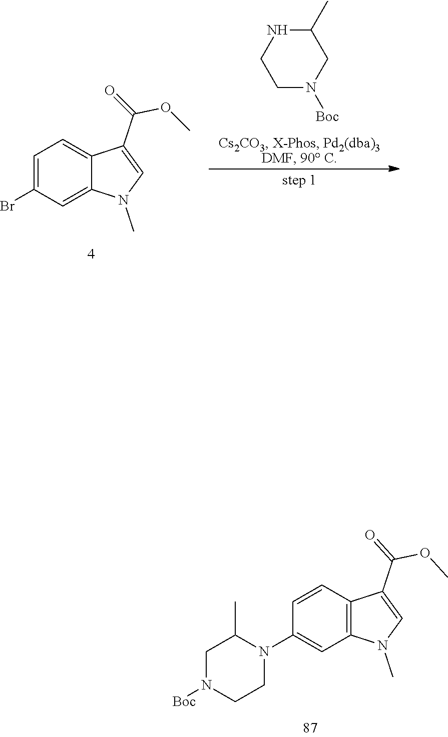

Under nitrogen, a mixture of compound 4 (1.5 g, 5.59 mmol), tert-butyl 3-methylpiperazine-1-carboxylate (1.34 g, 6.71 mmol), Cs₂CO₃ (5.46 g, 16 mmol), X-Phos (0.477 g, 1.1 mmol) and Pd₂(dba)₃ (256 mg, 0.28 mmol) were successively added to DMF (15 mL). Then the mixture was heated to 90° C. and stirred overnight. After completion of the reaction, the suspension was filtered; the filter cake was washed with EA. The filtrate was concentrated, dried and purified by silica column with PE/EA (10:1) to give the compound 87 (1.0 g, black oil, yield: 47%). LCMS: [M+H]+=388.3.

### Step 2: Synthesis of 88

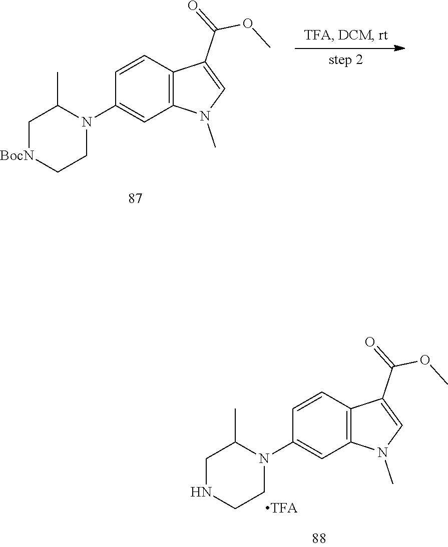 [SMILES: COC(=O)c1cn(C)c2cc(N3CCN(C(=O)OC(C)(C)C)CC3C)ccc12 ; "TFA, DCM, rt" ; "step 2" ; COC(=O)c1cn(C)c2cc(N3CCNCC3C)ccc12]

Follow the procedure of Synthesis of 6 to give 88. LCMS: [M+H]+=288.2. ¹H NMR (400 MHz, CDCl₃) δ 8.22 (d, J=8.6 Hz, 1H), 7.83 (s, 1H), 7.66 (s, 1H), 7.35 (d, J=8.5 Hz, 1H), 4.31 (s, 1H), 4.19-4.08 (m, 1H), 3.86 (s, 3H), 3.81 (s, 3H), 3.71 (dd, J=21.7, 8.1 Hz, 2H), 3.61 (d, J=12.0 Hz, 4H), 1.10 (d, J=6.4 Hz, 3H).

### Step 3: Synthesis of 89

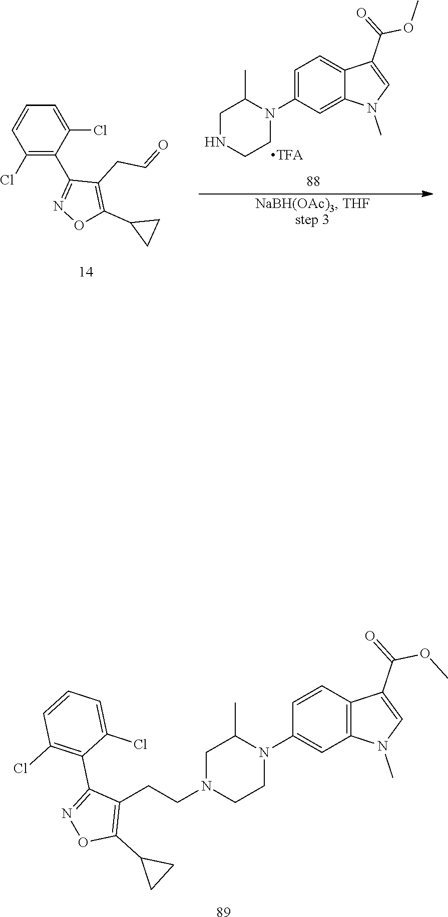 [SMILES: O=CCc1c(-c2c(Cl)cccc2Cl)noc1C1CC1 ; COC(=O)c1cn(C)c2cc(N3CCNCC3C)ccc12 ; "NaBH(OAc)3, THFstep 3" ; COC(=O)c1cn(C)c2cc(N3CCN(CCc4c(-c5c(Cl)cccc5Cl)noc4C4CC4)CC3C)ccc12 ; "89"]

Follow the procedure of Synthesis of 15 to give 89. ¹H NMR (400 MHz, CDCl₃) δ 7.95 (d, J=8.6 Hz, 1H), 7.62 (s, 1H), 7.36 (d, J=7.9 Hz, 1H), 7.32-7.25 (m, 1H), 6.96 (dd, J=8.7, 1.6 Hz, 1H), 6.82 (s, 1H), 3.82 (s, 3H), 3.69 (s, 3H), 3.50 (s, 1H), 3.03 (s, 2H), 2.56 (m, 8H), 1.16 (dt, J=6.2, 5.2 Hz, 3H), 1.04 (dt, J=7.2, 4.3 Hz, 2H), 0.85 (d, J=6.2 Hz, 3H). LCMS: [M+H]+=569.2.

### Step 4: Synthesis of I12

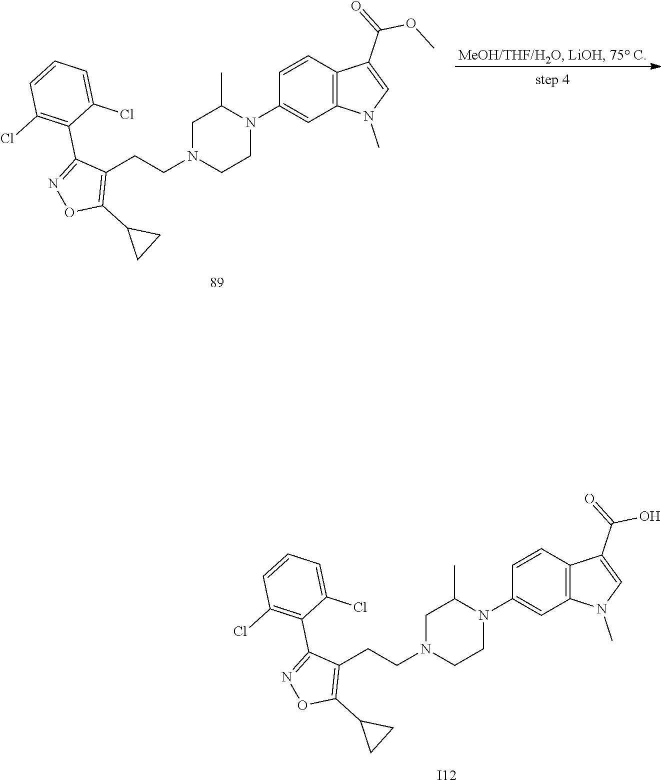

Follow the procedure of Synthesis of I1 to give 112. ¹H NMR (400 MHz, MeOD+D₂O): δ 7.98 (d, J=8.6 Hz, 1H), 7.85 (s, 1H), 7.62-7.49 (m, 3H), 7.13 (s, 1H), 7.06 (d, J=8.7 Hz, 1H), 3.82 (s, 3H), 3.49 (s, 1H), 3.09 (d, J=4.2 Hz, 2H), 2.72 (d, J=8.8 Hz, 1H), 2.67-2.54 (m, 4H), 2.48 (dd, J=10.3, 6.3 Hz, 2H), 2.35-2.19 (m, 2H), 1.22-1.11 (m, 4H), 0.89 (d, J=6.3 Hz, 3H); LCMS: [M+H]+=555.2.

### Example 13

### 2-(4-(4-(2-(5-cyclopropyl-3-(2,6-dichlorophenyl)isoxazol-4-yl)ethyl)-2-methylpiperazin-1-yl)phenyl)acetic Acid (I13)

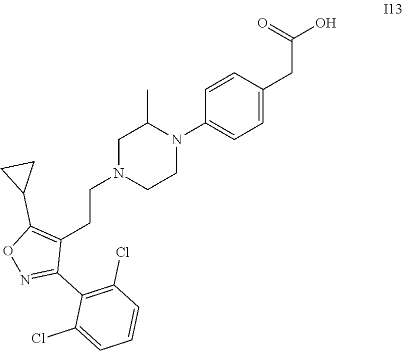 [SMILES: CC1CN(CCc2c(-c3c(Cl)cccc3Cl)noc2C2CC2)CCN1c1ccc(CC(=O)O)cc1]

### Step 1: Synthesis of 90

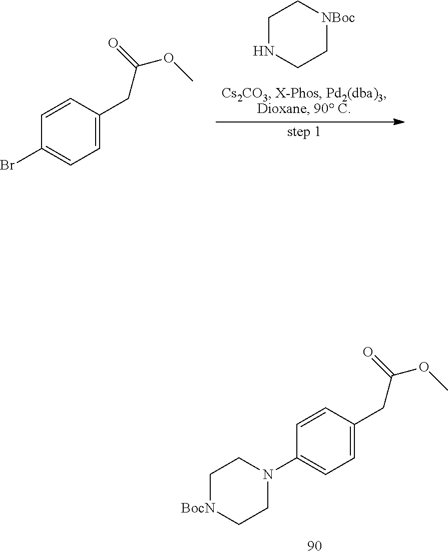

Follow the procedure of Synthesis of 87 to give 90. LCMS: [M+H]+=335.2.

### Step 2: Synthesis of 91

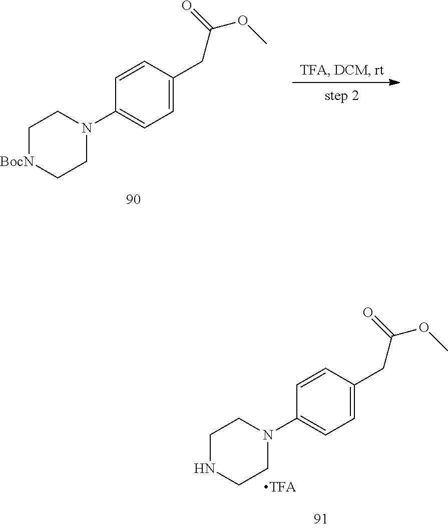 [SMILES: COC(=O)Cc1ccc(N2CCN(C(=O)OC(C)(C)C)CC2)cc1 ; "TFA, DCM, rt" ; "step 2" ; COC(=O)Cc1ccc(N2CCNCC2)cc1]

Follow the procedure of Synthesis of 6 to give 91. ¹H NMR (400 HMz, CDCl₃) δ 9.22 (s, 2H), 7.39 (d, J=8.6 Hz, 2H), 7.31 (d, J=8.6 Hz, 2H), 3.78-3.71 (m, 10H), 3.68 (s, 3H). LCMS: [M+H]+=235.1.

### Step 3: Synthesis of 92

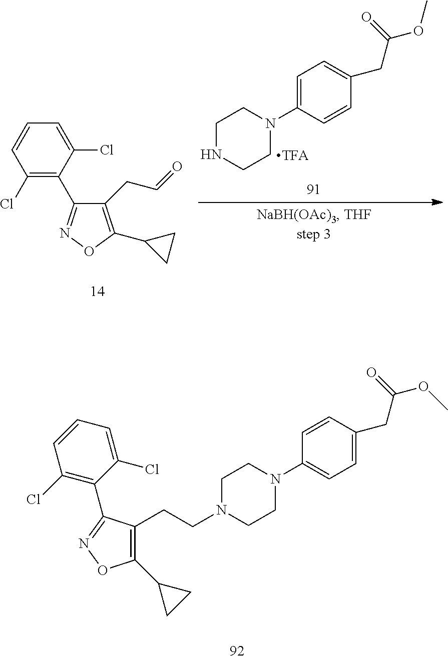

Follow the procedure of Synthesis of 15 to give 92. ¹H NMR (400 HMz, CDCl₃) δ 7.37-7.32 (m, 2H), 7.27 (dd, J=9.1, 6.9 Hz, 1H), 7.08 (d, J=8.6 Hz, 2H), 6.77 (d, J=8.6 Hz, 2H), 3.60 (s, 3H), 3.47 (s, 2H), 3.41 (s, 1H), 3.07 (s, 4H), 2.59-2.32 (m, 8H), 1.16-1.12 (m, 2H), 1.02 (dt, J=7.3, 4.3 Hz, 2H). LCMS: [M+H]+=516.2.

### Step 4: Synthesis of I13

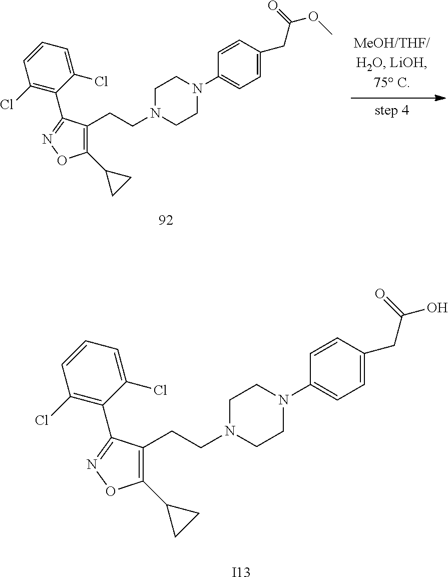 [SMILES: COC(=O)Cc1ccc(N2CCN(CCc3c(-c4c(Cl)cccc4Cl)noc3C3CC3)CC2)cc1 ; O=C(O)Cc1ccc(N2CCN(CCc3c(-c4c(Cl)cccc4Cl)noc3C3CC3)CC2)cc1 ; "I13"]

Follow the procedure of Synthesis of I1 to give I13. ¹H NMR (400 HMz, MeOD) δ 7.61-7.51 (m, 3H), 7.18 (d, J=8.6 Hz, 2H), 6.90 (d, J=8.6 Hz, 2H), 3.49 (s, 2H), 3.18-3.10 (m, 4H), 2.73-2.54 (m, 8H), 2.24 (m, 1H), 1.23-1.13 (m, 4H). LCMS: [M+H]+=502.1.

### Example 14

### 4-(4-(2-(5-cyclopropyl-3-(2,6-dichlorophenyl)isoxazol-4-yl)ethyl)piperazin-1-yl)benzoic Acid (I14)

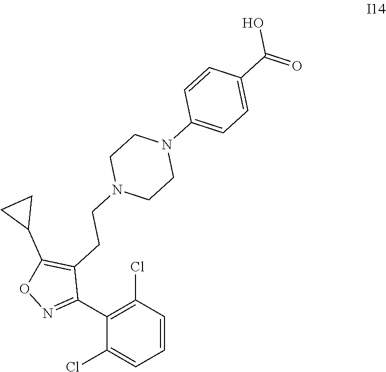 [SMILES: O=C(O)c1ccc(N2CCN(CCc3c(-c4c(Cl)cccc4Cl)noc3C3CC3)CC2)cc1]

### Step 1: Synthesis of 95

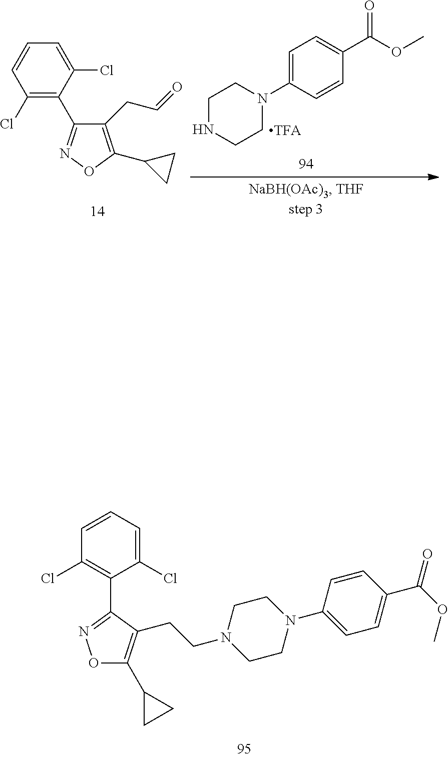

Follow the procedure of Synthesis of 15 to give 95. ¹H NMR (400 HMz, CDCl₃) δ 7.83 (d, J=9.0 Hz, 2H), 7.44-7.22 (m, 3H), 6.74 (t, J=5.9 Hz, 2H), 3.79 (s, 3H), 3.21 (s, 4H), 2.62-2.30 (m, 8H), 1.99 (d, J=3.4 Hz, 1H), 1.19-1.10 (m, 2H), 1.03 (ddd, J=11.3, 6.9, 4.4 Hz, 2H). LCMS: [M+H]+=502.1.

### Step 2: Synthesis of I14

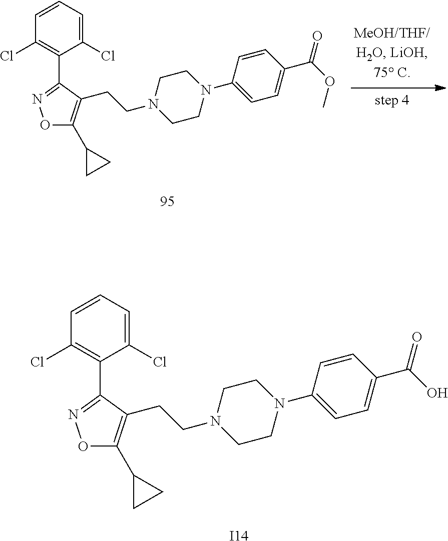

Follow the procedure of Synthesis of I1 to give I14. ¹H NMR (400 HMz, MeOD) δ 7.95 (d, J=8.9 Hz, 2H), 7.69-7.51 (m, 3H), 7.04 (d, J=9.0 Hz, 2H), 3.95-3.34 (m, 8H), 3.28-3.17 (m, 2H), 2.88 (dd, J=10.5, 6.8 Hz, 2H), 2.29 (td, J=8.0, 4.0 Hz, 1H), 1.27-1.18 (m, 4H).

### Example 15

### 6-(4-(2-(5-cyclopropyl-3-(2,6-dichlorophenyl)isoxazol-4-yl)ethyl)piperazin-1-yl)-1H-indole-3-carboxylic Acid (I15)

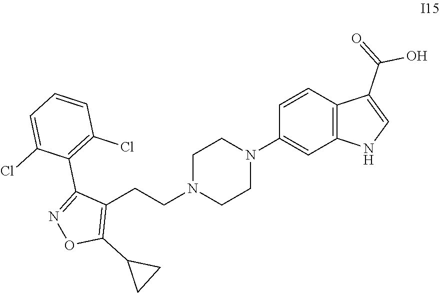 [SMILES: O=C(O)c1c[nH]c2cc(N3CCN(CCc4c(-c5c(Cl)cccc5Cl)noc4C4CC4)CC3)ccc12]

### Step 1: Synthesis of 97

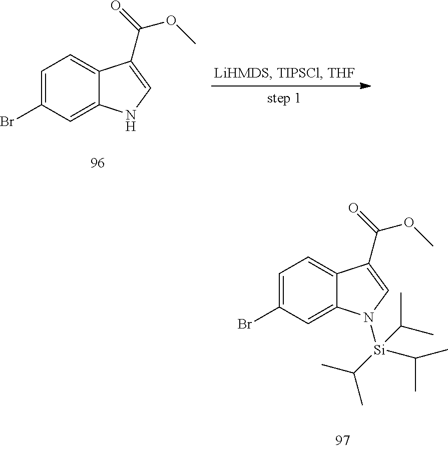 [SMILES: COC(=O)c1c[nH]c2cc(Br)ccc12 ; COC(=O)c1cn([Si](C(C)C)(C(C)C)C(C)C)c2cc(Br)ccc12]

Under nitrogen a solution of 96 (2.54 g, 10 mmol) in THF (25 mL) was cooled to 0° C., then 1.3 N LiHMDS (9.3 mL, 12 mmol) was added dropwise. The reaction mixture was stirred at 0° C. for 15 min. Then a solution of TIPSCl (2.3 g, 12 mmol) in THF (10 mL) was added dropwise to the reaction mixture. Then the reaction mixture was allowed to return to rt and stirred overnight. After the reaction completed, quenched with water, extracted with EA, and the combined organic layers were washed with brine, dried, concentrated and purified by silica gel column with heptanes/EA (50:1) to give 97. LCMS: [M+H]+=253.9, 255.9; ¹H NMR (400 HMz, CDCl₃) δ ppm: 8.06 (d, J=8.4 Hz, 1H), 7.92 (s, 1H), 7.63 (s, 1H), 7.37 (d, J=8.4 Hz, 1H), 3.92 (s, 3H), 1.72-1.67 (m, 3H), 1.17-1.14 (m, 18H).

### Step 2: Synthesis of 98

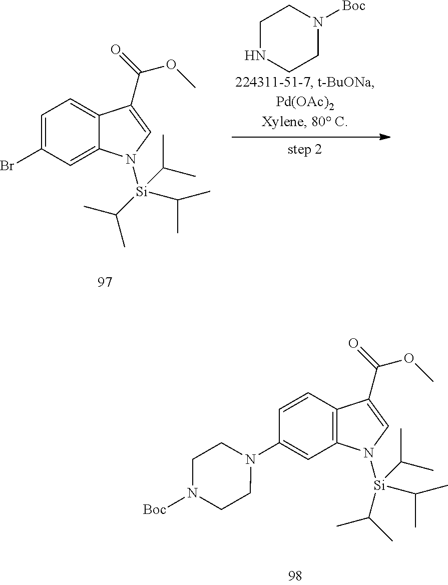 [SMILES: COC(=O)c1cn([Si](C(C)C)(C(C)C)C(C)C)c2cc(Br)ccc12 ; CC(C)(C)OC(=O)N1CCNCC1 ; COC(=O)c1cn([Si](C(C)C)(C(C)C)C(C)C)c2cc(N3CCN(C(=O)OC(C)(C)C)CC3)ccc12]

Under nitrogen, a mixture of compound 97 (4.1 g, 10 mmol), tert-butyl piperazine-1-carboxylate (2.24 g, 12 mmol), t-BuONa (1.44 g, 15 mmol), 2-(Di-tert-butylphosphino)biphenyl (CAS: 224311-51-7, 600 mg, 2 mmol), Pd(OAc)₂ (224 mg, 1 mmol) were successively added to xylene (40 mL). Then the mixture was heated to 120° C. and stirred for 4 h. After completion of the reaction, the suspension was filtered, the filter cake was washed with EA. The filtrate was concentrated and purified by silica column with heptanes/EA (50:1) to give 98). LCMS: [M+H]+=360.0; ¹H NMR (400 HMz, CDCl₃) δ ppm: 8.04 (d, J=8.7 Hz, 1H), 7.85 (s, 1H), 7.03-6.70 (m, 2H), 3.90 (s, 3H), 3.65-3.60 (m, 4H), 3.10-3.06 (s, 4H), 1.72-1.67 (m, 3H), 1.50 (s, 9H), 1.17-1.15 (m, 18H).

### Step 3: Synthesis of 99

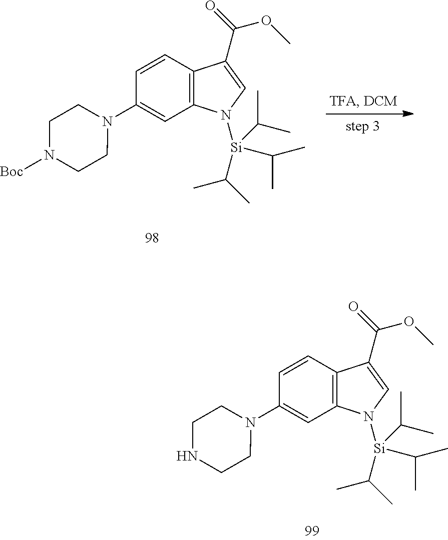 [SMILES: COC(=O)c1cn([Si](C(C)C)(C(C)C)C(C)C)c2cc(N3CCN(C(=O)OC(C)(C)C)CC3)ccc12 ; COC(=O)c1cn([Si](C(C)C)(C(C)C)C(C)C)c2cc(N3CCNCC3)ccc12]

Follow the procedure of Synthesis of 6 to give 99. LCMS: [M+H]+=416.3.

### Step 4: Synthesis of 100

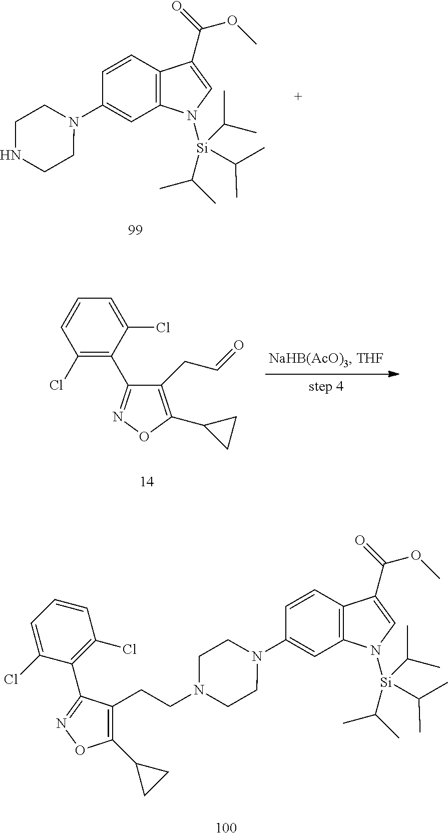 [SMILES: COC(=O)c1cn([Si](C(C)C)(C(C)C)C(C)C)c2cc(N3CCNCC3)ccc12 ; O=CCc1c(-c2c(Cl)cccc2Cl)noc1C1CC1 ; COC(=O)c1cn([Si](C(C)C)(C(C)C)C(C)C)c2cc(N3CCN(CCc4c(-c5c(Cl)cccc5Cl)noc4C4CC4)CC3)ccc12]

Follow the procedure of Synthesis of 15 to give 100. LCMS: [M+H]+=697.2; ¹H NMR (400 HMz, CDCl₃) δ ppm: 8.01 (m, 1H), 7.84 (s, 1H), 7.44-7.42 (m, 2H), 7.37-7.35 (m, 2H), 7.00-6.97 (m, 2H), 3.90 (s, 3H), 3.10 (s, 4H), 2.58-2.48 (m, 6H), 1.72-1.70 (m, 3H), 1.68-1.66 (m, 2H), 1.26-1.21 (m, 4H), 1.16-1.14 (m, 18H).

### Step 5: Synthesis of I15

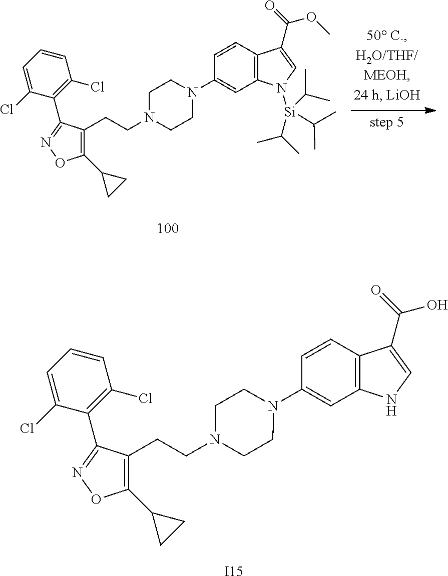

Follow the procedure of Synthesis of I1 to give 115. LCMS: [M+H]+=225.1; ¹H NMR (4DMSO-d6) δ ppm: 11.60 (br, 1H), 7.85-7.83 (m, 2H), 7.70-7.62 (m, 3H), 6.96-6.92 (m, 2H), 3.72-3.70 (m, 2H), 3.58-3.57 (m, 2H), 3.34-3.07 (m, 6H), 2.86 (s, 2H), 2.49 (br, 1H), 1.23-1.17 (m, 4H).

### Example 16

### 6-(4-(2-(5-cyclopropyl-3-(2,4-dichlorophenyl)isoxazol-4-yl)ethyl)piperazin-1-yl)-1-methyl-1H-indole-3-carboxylic Acid (I16)

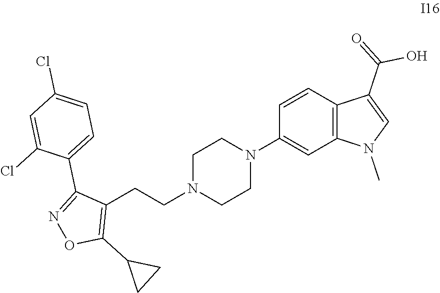 [SMILES: Cn1cc(C(=O)O)c2ccc(N3CCN(CCc4c(-c5ccc(Cl)cc5Cl)noc4C4CC4)CC3)cc21]

### Step 1: Synthesis of 102

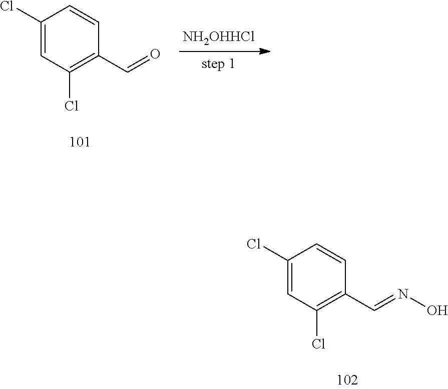

Follow the procedure of Synthesis of 8 to give 102 used directly in the next step. LCMS: [M+H]+=190; ¹H NMR (400 HMz, DMSO-d6) δ ppm: 11.84 (br, 1H), 8.30 (s, 1H), 7.81-7.79 (m, 1H), 7.63-7.62 (m, 1H), 7.44-7.41 (m, 1H).

### Step 2: Synthesis of 103

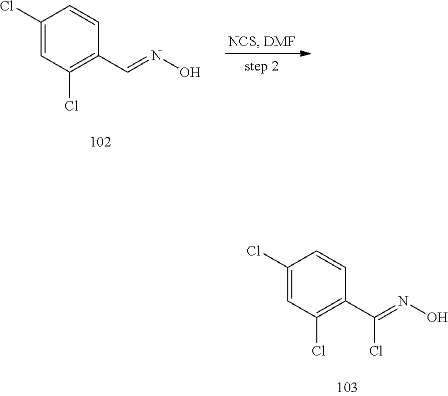

Follow the procedure of Synthesis of 9 to give 103, which was used directly in the next step. ¹H NMR (DMSO-d6) δ ppm: 12.67 (s, 1H), 7.79-7.78 (m, 1H), 7.62-7.60 (m, 1H), 7.56-7.53 (m, 1H).

### Step 3: Synthesis of 104

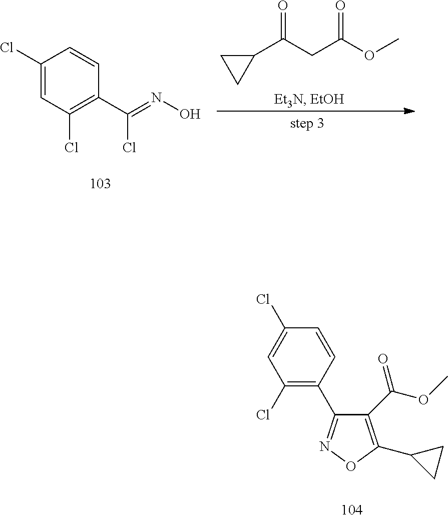

Follow the procedure of Synthesis of 10 to give 104. ¹H NMR (400 HMz, CDCl₃) δ ppm: 7.52 (s, 1H), 7.36-7.28 (m, 2H), 3.74 (s, 3H), 2.89-2.88 (m, 1H), 1.41-1.37 (m, 2H), 1.31-1.26 (m, 2H).

### Step 4: Synthesis of 105

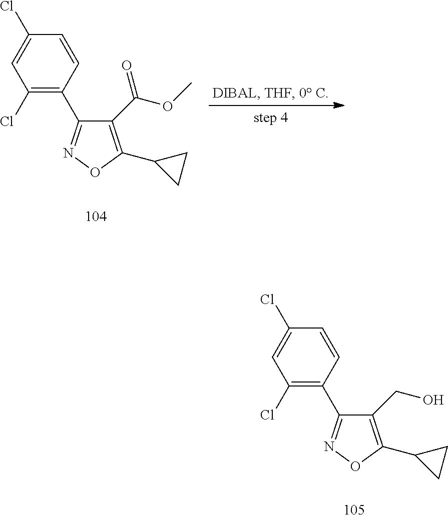

Follow the procedure of Synthesis of 11 to give 105. ¹H NMR (400 HMz, CDCl₃) δ ppm: 7.45 (s, 1H), 7.33-7.26 (m, 2H), 4.41 (s, 2H), 2.14-2.07 (m, 1H), 1.19-1.15 (m, 2H), 1.08-1.03 (m, 2H).

### Step 5: Synthesis of 106

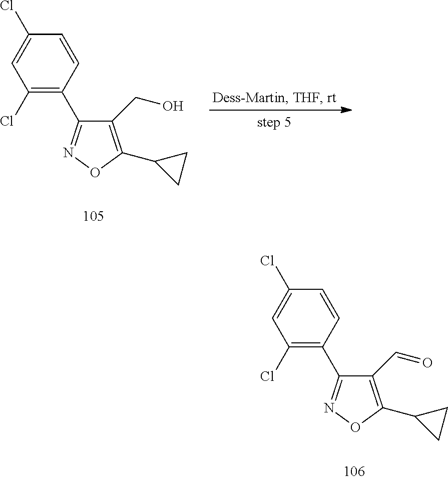 [SMILES: OCc1c(-c2ccc(Cl)cc2Cl)noc1C1CC1 ; "Dess-Martin, THF, rt" ; O=Cc1c(-c2ccc(Cl)cc2Cl)noc1C1CC1]

Follow the procedure of Synthesis of 12 to give 106. ¹H NMR (400 HMz, CDCl₃) δ ppm: 9.73 (s, 1H), 7.56 (s, 1H), 7.44-7.27 (m, 2H), 7.89-7.80 (m, 1H), 1.60 (s, 2H), 1.44-1.26 (m, 2H).

### Step 6: Synthesis of 107

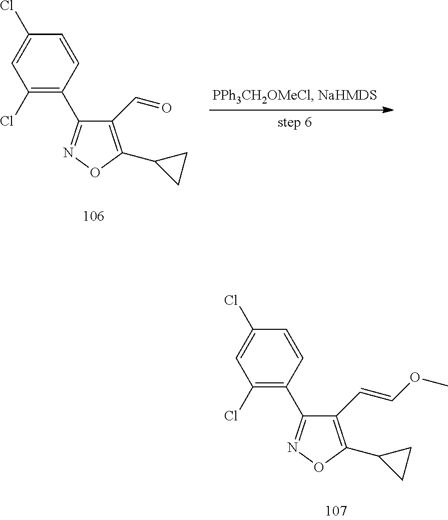

Follow the procedure of Synthesis of 13 to give 107. LCMS: [M+H]+=309.9.

### Step 7: Synthesis of 108

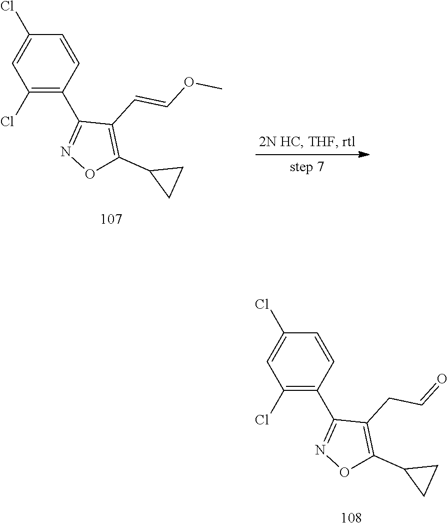 [SMILES: CO/C=C/c1c(-c2ccc(Cl)cc2Cl)noc1C1CC1 ; O=CCc1c(-c2ccc(Cl)cc2Cl)noc1C1CC1]

Follow the procedure of Synthesis of 14 to give 108. ¹H NMR (400 HMz, CDCl₃) δ ppm: 9.62 (s, 1H), 7.52 (s, 1H), 7.35 (s, 2H), 3.47 (s, 2H), 1.93-1.87 (m, 1H), 1.26-1.10 (m, 4H).

### Step 8: Synthesis of 109

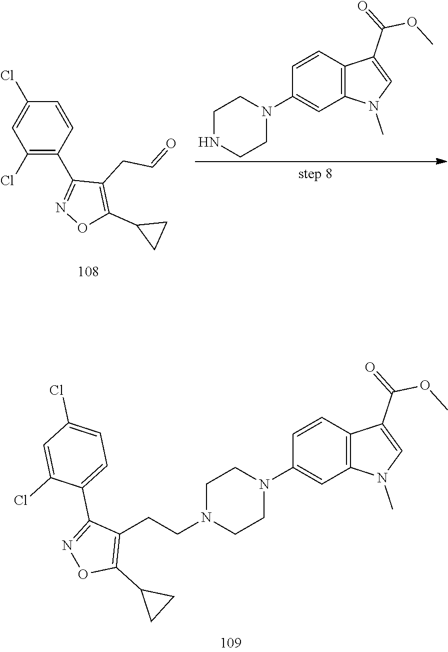

Follow the procedure of Synthesis of 15 to give 109. LCMS: [M+H]+=553.1; 1H NMR (400 HMz, DMSO-d6) δ ppm: 7.89 (s, 1H), 7.83 (s, 1H), 7.75 (d, J=8.7 Hz, 1H), 7.58-7.48 (m, 2H), 6.93-6.86 (m, 2H), 3.77 (s, 6H), 2.54 (s, 4H), 2.38 (s, 2H), 2.30 (s, 4H), 2.27-2.23 (m, 3H), 1.06-0.85 (m, 4H).

### Step 9: Synthesis of I16

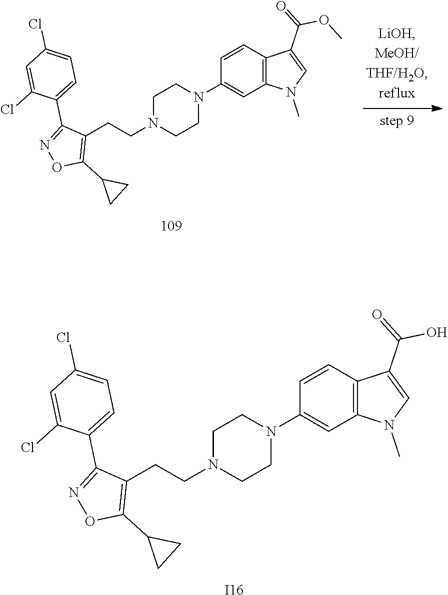

Follow the procedure of Synthesis of I1 to give I16. LCMS: [M+H]+=539.1; ¹H NMR (400 HMz, DMSO-d6) δ ppm: 11.80 (br, 1H), 7.86-7.78 (m, 3H), 7.60-7.52 (m, 2H), 6.92-6.87 (m, 2H), 3.76 (s, 3H), 3.03 (s, 4H), 2.58-2.55 (m, 2H), 2.40 (s, 4H), 2.32-2.25 (m, 3H), 1.11-1.09 (m, 4H).

### Example 17

### 6-(4-(2-(5-cyclopropyl-3-(2,6-dichlorophenyl)isoxazol-4-yl)ethyl)piperazin-1-yl)-1-methyl-1H-indazole-3-carboxylic Acid (I17)

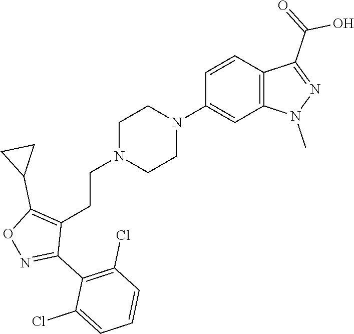 [SMILES: Cn1nc(C(=O)O)c2ccc(N3CCN(CCc4c(-c5c(Cl)cccc5Cl)noc4C4CC4)CC3)cc21]

### Step 1: Synthesis of 113

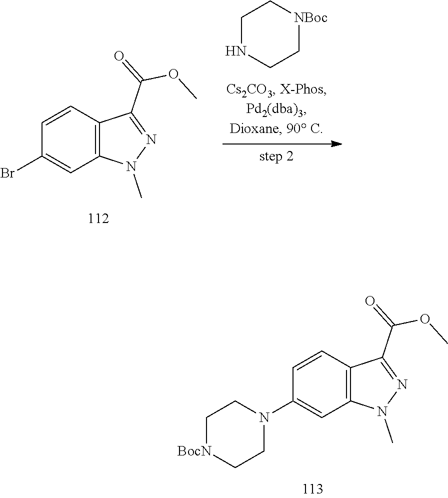 [SMILES: COC(=O)c1nn(C)c2cc(Br)ccc12 ; CC(C)(C)OC(=O)N1CCNCC1 ; COC(=O)c1nn(C)c2cc(N3CCN(C(=O)OC(C)(C)C)CC3)ccc12]

Follow the procedure of Synthesis of 87 to give 113. ¹H NMR (400 HMz, CDCl₃) δ 8.01 (d, J=9.0 Hz, 1H), 7.02 (dd, J=9.0, 1.6 Hz, 1H), 6.74 (s, 1H), 4.03 (d, J=5.0 Hz, 3H), 3.95 (s, 3H), 3.64-3.52 (m, 4H), 3.22-3.14 (m, 4H), 1.43 (s, 9H). LCMS: [M+H]+=375.2.

### Step 2: Synthesis of 114

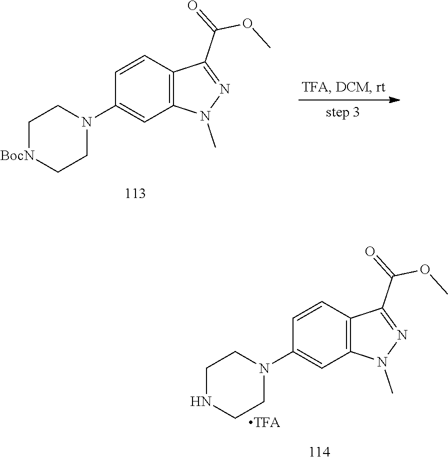 [SMILES: COC(=O)c1nn(C)c2cc(N3CCN(C(=O)OC(C)(C)C)CC3)ccc12 ; COC(=O)c1nn(C)c2cc(N3CCNCC3)ccc12]

Follow the procedure of Synthesis of 6 to give the 114. ¹H NMR (CDCl₃) δ 8.13 (d, J=8.9 Hz, 1H), 7.06 (dd, J=9.0, 1.7 Hz, 1H), 6.81 (s, 1H), 4.14 (s, 3H), 4.05 (s, 3H), 3.58 (s, 4H), 3.48 (s, 4H). LCMS: [M+H]+=275.1.

### Step 3: Synthesis of 115

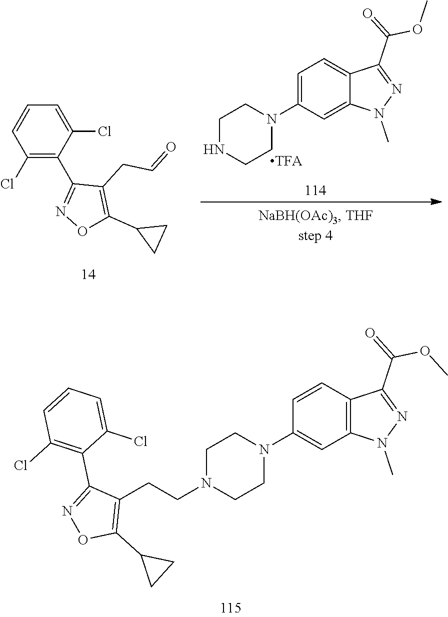 [SMILES: O=CCc1c(-c2c(Cl)cccc2Cl)noc1C1CC1 ; COC(=O)c1nn(C)c2cc(N3CCNCC3)ccc12 ; COC(=O)c1nn(C)c2cc(N3CCN(CCc4c(-c5c(Cl)cccc5Cl)noc4C4CC4)CC3)ccc12]

Follow the procedure of Synthesis of 15 to give 115. ¹H NMR (400 HMz, CDCl₃) δ 7.95 (d, J=9.0 Hz, 1H), 7.41-7.24 (m, 3H), 6.95 (dd, J=9.0, 1.8 Hz, 1H), 6.57 (s, 1H), 4.00 (s, 3H), 3.94 (s, 3H), 3.19 (s, 4H), 2.70-2.32 (m, 8H), 1.26-1.11 (m, 3H), 1.07-0.96 (m, 2H). LCMS: [M+H]+=556.2.

### Step 4: Synthesis of I17

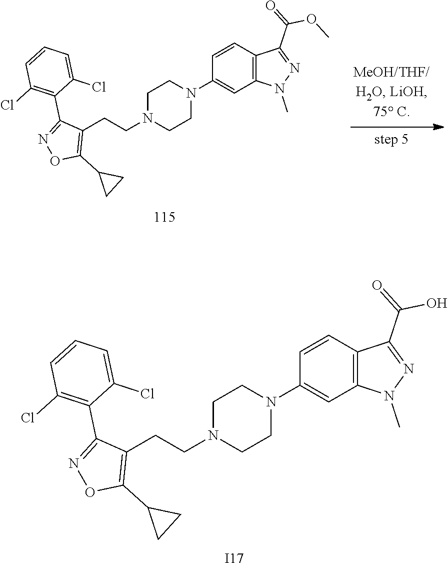

Follow the procedure of Synthesis of I1 to give I17. ¹H NMR (400 HMz, MeOD) δ 8.01 (d, J=9.0 Hz, 1H), 7.69-7.51 (m, 3H), 7.15 (dd, J=9.1, 1.8 Hz, 1H), 7.01 (s, 1H), 4.09 (s, 3H), 3.52 (s, 4H), 3.35 (s, 4H), 3.15 (dd, J=10.3, 6.8 Hz, 2H), 2.87 (dd, J=10.3, 6.8 Hz, 2H), 2.35-2.25 (m, 1H), 1.27-1.12 (m, 4H).

### Example 18

### 6-(4-(2-(5-cyclopropyl-3-(2,6-dichlorophenyl)isoxazol-4-yl)ethyl) piperazin-1-yl)-2-methyl-2H-indazole-3-carboxylic Acid (I18)

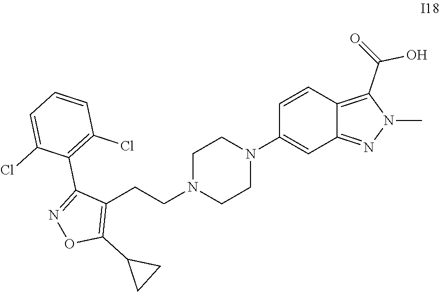 [SMILES: Cn1nc2cc(N3CCN(CCc4c(-c5c(Cl)cccc5Cl)noc4C4CC4)CC3)ccc2c1C(=O)O]

### Step 1: Synthesis of 116

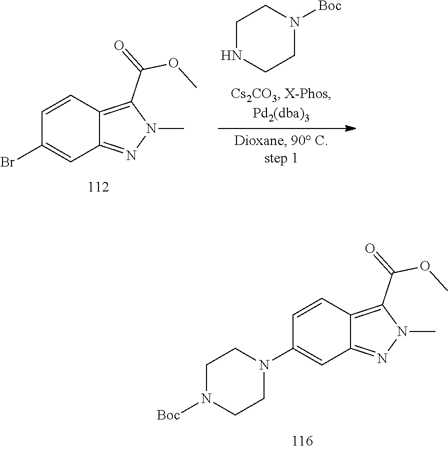 [SMILES: COC(=O)c1c2ccc(Br)cc2nn1C ; CC(C)(C)OC(=O)N1CCNCC1 ; COC(=O)c1c2ccc(N3CCN(C(=O)OC(C)(C)C)CC3)cc2nn1C]

Follow the procedure of Synthesis of 87 to give 116. ¹H NMR (400 HMz, CDCl₃) δ 7.83 (d, J=9.2 Hz, 1H), 7.14-6.97 (m, 2H), 4.39 (s, 3H), 3.95 (s, 3H), 3.65-3.53 (m, 4H), 3.12 (s, 3H), 1.42 (s, 9H). LCMS: [M+H]+=375.2.

### Step 2: Synthesis of 117

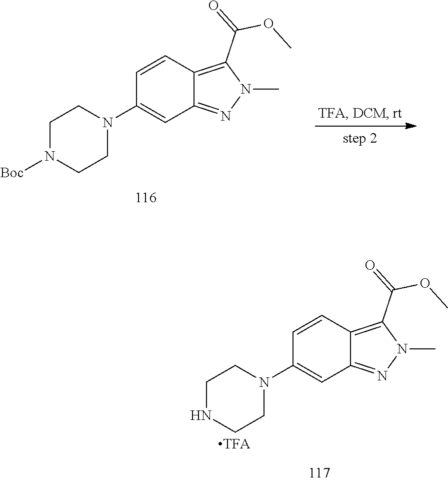 [SMILES: COC(=O)c1c2ccc(N3CCN(C(=O)OC(C)(C)C)CC3)cc2nn1C ; COC(=O)c1c2ccc(N3CCNCC3)cc2nn1C]

Follow the procedure of Synthesis of 6 to give 117. ¹H NMR (CDCl₃) δ 9.70 (s, 1H), 7.85 (d, J=9.1 Hz, 1H), 7.04-6.91 (m, 2H), 4.40 (s, 3H), 3.96 (s, 3H), 3.38 (d, J=26.4 Hz, 8H). LCMS: [M+H]+=275.1.

### Step 3: Synthesis of 118

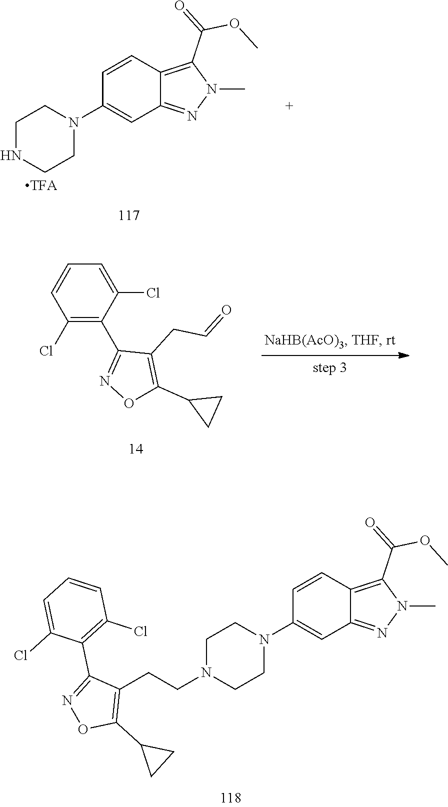 [SMILES: COC(=O)c1c2ccc(N3CCNCC3)cc2nn1C ; O=CCc1c(-c2c(Cl)cccc2Cl)noc1C1CC1 ; COC(=O)c1c2ccc(N3CCN(CCc4c(-c5c(Cl)cccc5Cl)noc4C4CC4)CC3)cc2nn1C]

Follow the procedure of Synthesis of 15 to give 118. ¹H NMR (400 HMz, CDCl₃) δ 7.78 (d, J=9.2 Hz, 1H), 7.40-7.34 (m, 2H), 7.28 (dd, J=9.0, 6.9 Hz, 1H), 6.99 (dd, J=9.2, 1.8 Hz, 1H), 6.91 (s, 1H), 4.37 (s, 3H), 3.95 (d, J=5.7 Hz, 3H), 3.15 (s, 4H), 2.51 (d, J=31.6 Hz, 8H), 1.22-1.11 (m, 3H), 1.08-0.98 (m, 2H). LCMS: [M+H]+=556.2.

### Step 4: Synthesis of I18

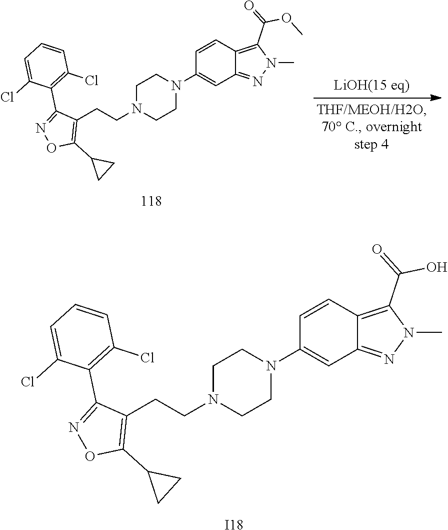

Follow the procedure of Synthesis of I1 to give I18. ¹H NMR (400 HMz, MeOD): δ 7.97 (d, J=9.2 Hz, 1H), 7.65-7.59 (m, 2H), 7.55 (dd, J=9.4, 6.6 Hz, 1H), 7.16 (dd, J=9.2, 1.8 Hz, 1H), 7.00 (s, 1H), 4.42 (s, 3H), 3.48-3.35 (m, 8H), 3.17 (dd, J=10.4, 6.7 Hz, 2H), 2.87 (dd, J=10.4, 6.7 Hz, 2H), 2.35-2.25 (m, 1H), 1.30-1.17 (m, 4H). LCMS: [M+H]+=539.8.

### Example 19

### 6-(4-(2-(5-cyclopropyl-3-(2-fluorophenyl)isoxazol-4-yl)ethyl)piperazin-1-yl)-1-methyl-1H-indole-3-carboxylic Acid (I19)

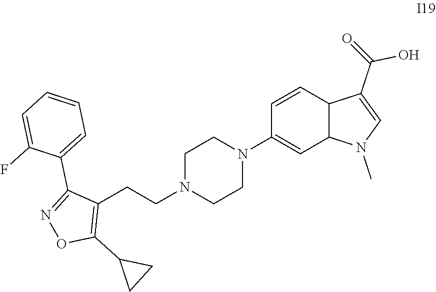 [SMILES: CN1C=C(C(=O)O)C2C=CC(N3CCN(CCc4c(-c5ccccc5F)noc4C4CC4)CC3)=CC21]

### Step 1: Synthesis of 121

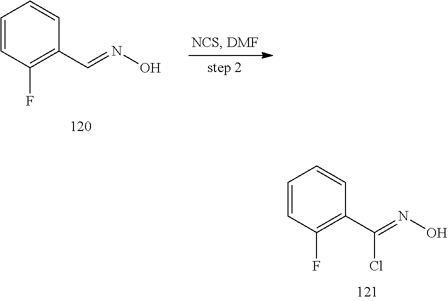 [SMILES: O/N=C/c1ccccc1F ; O/N=C(\Cl)c1ccccc1F]

Follow the procedure of Synthesis of 9 to give 121, used directly in the next step without further purification. ¹H NMR (400 HMz, DMSO-d6) δ ppm: 12.62 (br, 1H), 7.67-7.56 (m, 1H), 7.55-7.52 (m, 1H), 7.37-7.30 (m, 2H).

### Step 2: Synthesis of 122

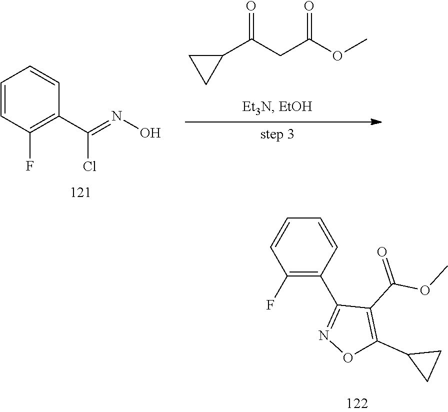

Follow the procedure of Synthesis of 10 to give 122. LCMS: [M+H]+=262.1; 1H NMR (400 HMz, DMSO-d6) δ ppm: 7.47-7.41 (m, 2H), 7.31-7.27 (m, 2H), 3.62 (s, 3H), 2.81-2.77 (m, 1H), 1.31-1.25 (m, 2H), 1.23-1.16 (m, 2H).

### Step 3: Synthesis of 123

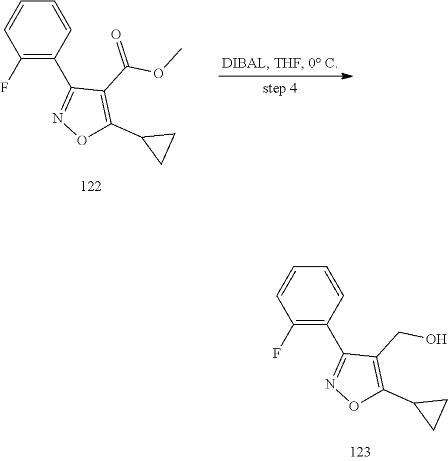 [SMILES: COC(=O)c1c(-c2ccccc2F)noc1C1CC1 ; OCc1c(-c2ccccc2F)noc1C1CC1]

Follow the procedure of Synthesis of 11 to give 123. LCMS: [M+H]+=234.1; ¹H NMR (400 HMz, CDCl₃) δ ppm: 7.47-7.40 (m, 2H), 7.31-7.27 (m, 2H), 4.38 (s, 2H), 2.10-2.06 (m, 1H), 1.14-1.10 (m, 2H), 1.03-0.98 (m, 2H).

### Step 4: Synthesis of 124

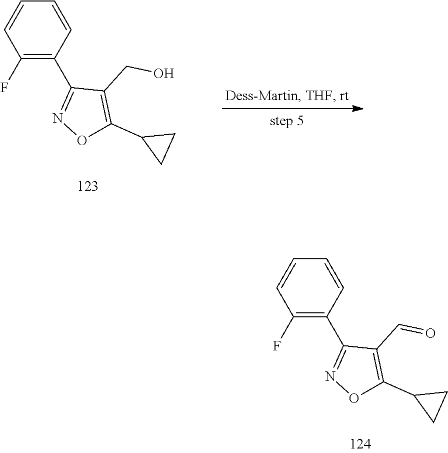 [SMILES: OCc1c(-c2ccccc2F)noc1C1CC1 ; O=Cc1c(-c2ccccc2F)noc1C1CC1]

Follow the procedure of Synthesis of 12 to give 124. LCMS: [M+H]+=232.1; ¹H NMR (400 HMz, CDCl₃) δ ppm: 9.74 (s, 1H), 7.53-7.45 (m, 2H), 7.24-7.14 (m, 2H), 2.84-2.80 (m, 1H), 1.35-1.33 (m, 2H), 1.27-1.22 (m, 2H).

### Step 5: Synthesis of 125

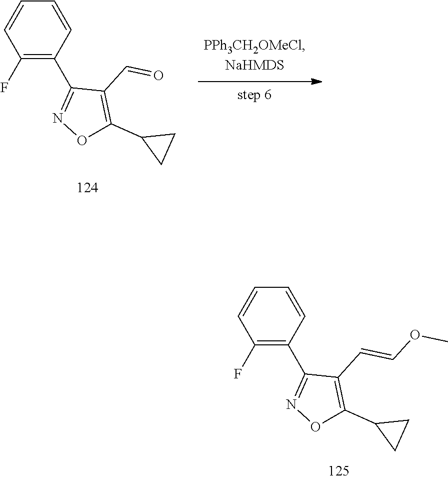 [SMILES: O=Cc1c(-c2ccccc2F)noc1C1CC1 ; CO/C=C/c1c(-c2ccccc2F)noc1C1CC1]

Follow the procedure of Synthesis of 13 to give 125. LCMS: [M+H]+=260.1; ¹H NMR (400 HMz, CDCl₃) δ ppm: 7.43-7.36 (m, 2H), 7.19-7.08 (m, 2H), 6.57 (d, J=12.8 Hz, 1H), 5.31 (d, J=12.8 Hz, 1H), 3.51 (s, 3H), 1.98-1.97 (m, 1H), 1.11-1.08 (m, 2H), 1.02-0.95 (m, 2H).

### Step 6: Synthesis of 126

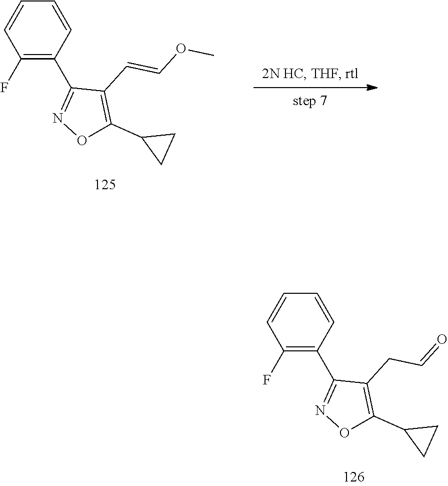 [SMILES: CO/C=C/c1c(-c2ccccc2F)noc1C1CC1 ; O=CCc1c(-c2ccccc2F)noc1C1CC1]

Follow the procedure of Synthesis of 14 to give 126. LCMS: [M+H]+=246.1.

### Step 7: Synthesis of 127

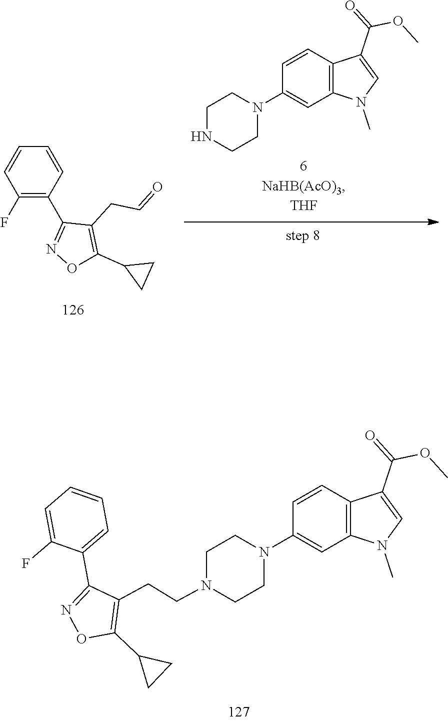 [SMILES: O=CCc1c(-c2ccccc2F)noc1C1CC1 ; COC(=O)c1cn(C)c2cc(N3CCNCC3)ccc12 ; COC(=O)c1cn(C)c2cc(N3CCN(CCc4c(-c5ccccc5F)noc4C4CC4)CC3)ccc12]

Follow the procedure of Synthesis of 15 to give 127. LCMS: [M+H]+=503.2. ¹H NMR (400 HMz, CDCl₃) δ ppm: 7.94-7.92 (m, 1H), 7.58 (s, 1H), 7.43-7.36 (m, 2H), 7.19-7.01 (m, 3H), 6.92-6.90 (m, 2H), 6.67 (s, 1H), 3.81 (s, 3H), 3.67 (s, 3H), 3.12 (s, 4H), 2.66-2.45 (m, 7H), 1.97 (s, 2H), 1.21-1.13 (m, 2H), 1.11-1.05 (m, 2H).

### Step 8: Synthesis of 119

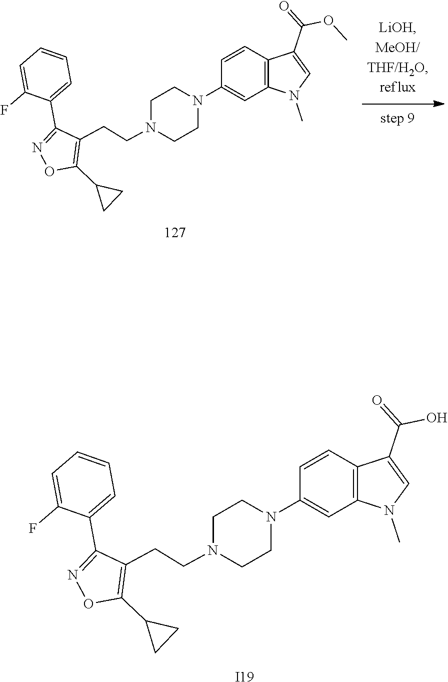

Follow the procedure of Synthesis of I1 to give I19. LCMS: [M+H]+=489.2; ¹H NMR (400 HMz, DMSO-d6) δ ppm: 11.78 (br, 1H), 7.83-7.78 (m, 2H), 7.61-7.42 (m, 2H), 7.39-7.34 (m, 2H), 6.92-6.87 (m, 2H), 3.76 (s, 3H), 3.03 (s, 4H), 2.64-2.60 (m, 2H), 2.22 (s, 4H), 2.33-2.27 (m, 2H), 2.27-2.24 (m, 1H), 1.11-1.02 (m, 4H).

### Example 20

### 6-(4-(2-(5-cyclopropyl-3-(2-(trifluoromethoxy)phenyl)isoxazol-4-yl)ethyl)piperazin-1-yl)-1-methyl-1H-indole-3-carboxylic Acid (120)

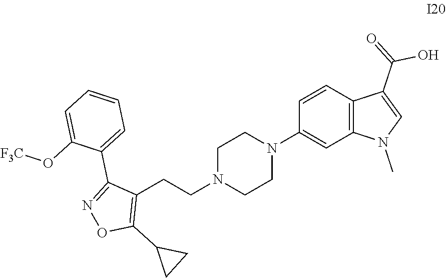 [SMILES: Cn1cc(C(=O)O)c2ccc(N3CCN(CCc4c(-c5ccccc5OC(F)(F)F)noc4C4CC4)CC3)cc21]

### Step 1: Synthesis of 136

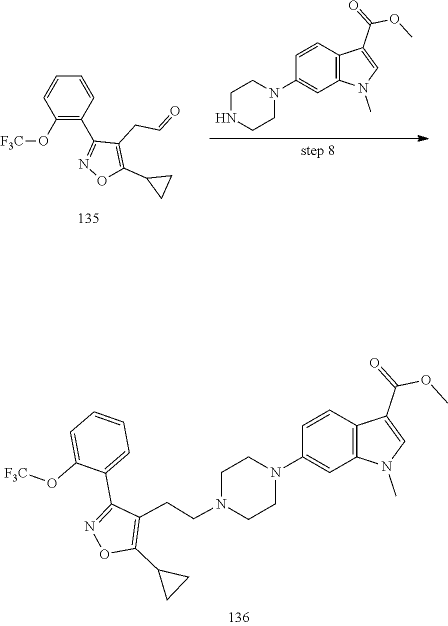 [SMILES: O=CCc1c(-c2ccccc2OC(F)(F)F)noc1C1CC1 ; COC(=O)c1cn(C)c2cc(N3CCNCC3)ccc12 ; COC(=O)c1cn(C)c2cc(N3CCN(CCc4c(-c5ccccc5OC(F)(F)F)noc4C4CC4)CC3)ccc12]

Follow the procedure of Synthesis of 15 to give 136. LCMS: [M+H]+=569.2; ¹H NMR (400 HMz, DMSO-d6) δ ppm: 7.89 (s, 1H), 7.75 (d, J=8.7 Hz, 1H), 7.66-7.64 (m, 1H), 7.56-7.53 (m, 2H), 6.92-6.86 (m, 2H), 5.72 (s, 1H), 3.74 (s, 6H), 3.02 (s, 4H), 2.55-2.53 (m, 2H), 2.48 (s, 4H), 2.30-2.23 (m, 3H), 1.20-1.12 (m, 2H), 1.09-0.99 (m, 2H).

### Step 2: Synthesis of I20

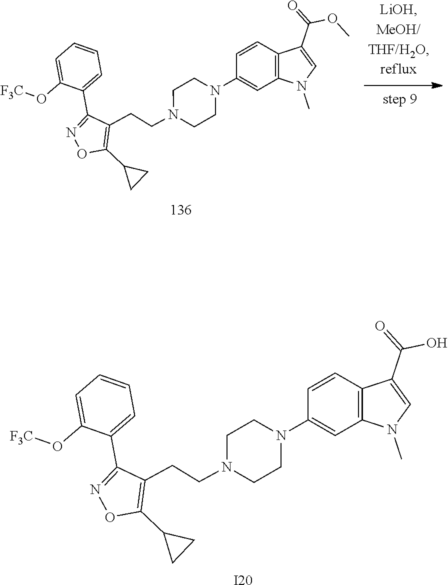

Follow the procedure of Synthesis of I1 to give 120. LCMS: [M+H]+=555.2; ¹H NMR (400 HMz, DMSO-d6) δ ppm: 11.82 (br, 1H), 7.83-7.77 (m, 2H), 7.71-7.67 (m, 1H), 7.61-7.54 (m, 3H), 6.92-6.87 (m, 2H), 3.76 (s, 3H), 3.04 (s, 4H), 2.58-2.51 (m, 2H), 2.41 (s, 4H), 2.32-2.26 (m, 3H), 1.12-1.04 (m, 4H).

### Example 21

### 6-(4-(2-(3-(2-chloro-6-methoxyphenyl)-5-cyclopropylisoxazol-4-yl)ethyl)piperazin-1-yl)-1-methyl-1H-indole-3-carboxylic Acid (I21)

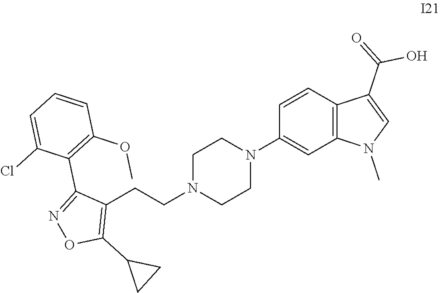 [SMILES: COc1cccc(Cl)c1-c1noc(C2CC2)c1CCN1CCN(c2ccc3c(C(=O)O)cn(C)c3c2)CC1]

### Step 1: Synthesis of 138

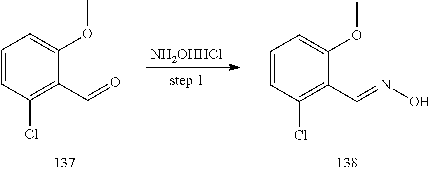

Follow the procedure of Synthesis of 8 to give 138, which was used directly in the next step. LCMS: [M+H]+=186.1.

### Step 2: Synthesis of 139

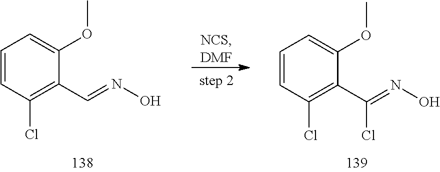

Follow the procedure of Synthesis of 9 to give 139 used directly in the next step.

### Step 3: Synthesis of 140

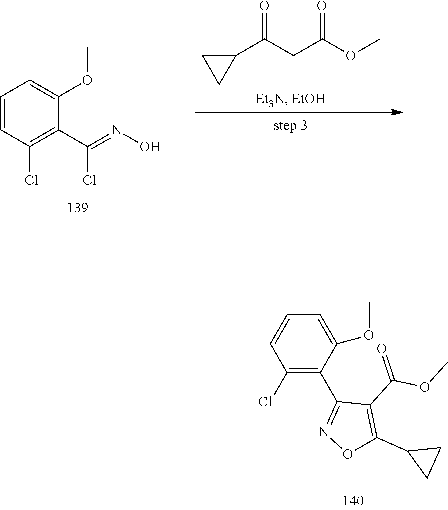

Follow the procedure of Synthesis of 10 to give 140. LCMS: [M+H]+=308.0.

### Step 4: Synthesis of 141

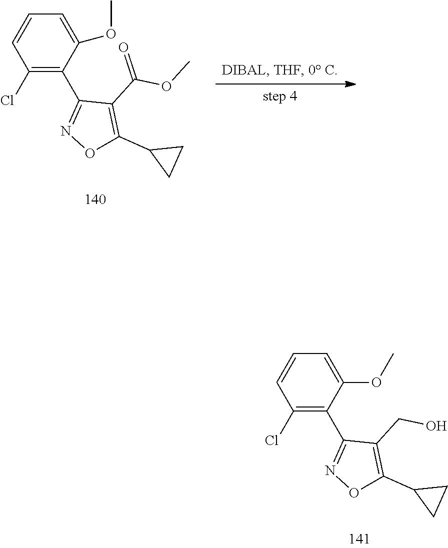 [SMILES: COC(=O)c1c(-c2c(Cl)cccc2OC)noc1C1CC1 ; COc1cccc(Cl)c1-c1noc(C2CC2)c1CO]

Follow the procedure of Synthesis of 11 to give 141. LCMS: [M+H]+=280.1.

### Step 5: Synthesis of 142

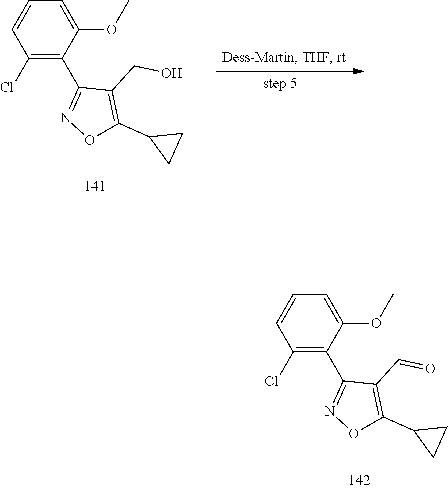 [SMILES: COc1cccc(Cl)c1-c1noc(C2CC2)c1CO ; COc1cccc(Cl)c1-c1noc(C2CC2)c1C=O]

Follow the procedure of Synthesis of 12 to give 142. LCMS: [M+H]+=278.0.

### Step 6: Synthesis of 143

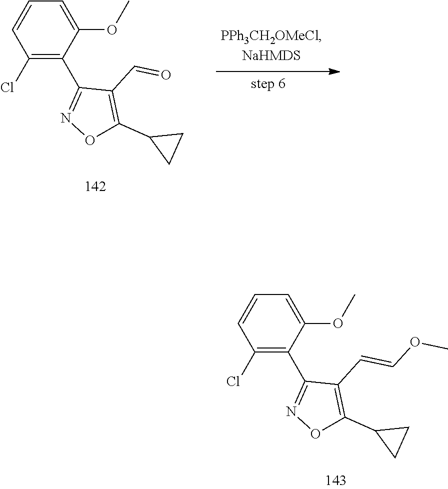 [SMILES: COc1cccc(Cl)c1-c1noc(C2CC2)c1C=O ; CO/C=C/c1c(-c2c(Cl)cccc2OC)noc1C1CC1]

Follow the procedure of Synthesis of 13 to give 143. LCMS: [M+H]+=306.1.

### Step 7: Synthesis of 144

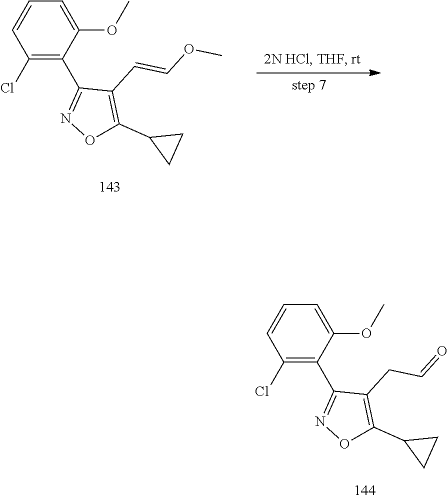 [SMILES: CO/C=C/c1c(-c2c(Cl)cccc2OC)noc1C1CC1 ; COc1cccc(Cl)c1-c1noc(C2CC2)c1CC=O]

Follow the procedure of Synthesis of 14 to give 144. LCMS: [M+H]+=292.0.

### Step 8: Synthesis of 145

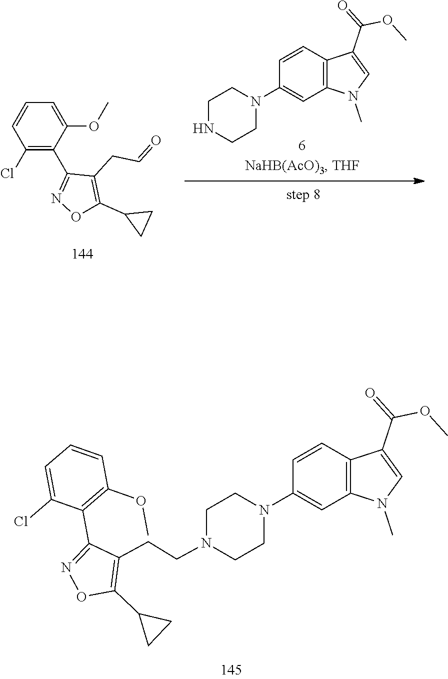 [SMILES: COc1cccc(Cl)c1-c1noc(C2CC2)c1CC=O ; COC(=O)c1cn(C)c2cc(N3CCNCC3)ccc12 ; COC(=O)c1cn(C)c2cc(N3CCN(CCc4c(-c5c(Cl)cccc5OC)noc4C4CC4)CC3)ccc12]

Follow the procedure of Synthesis of 15 to give 145. LCMS: [M+H]+=549.2.

### Step 9: Synthesis of I21

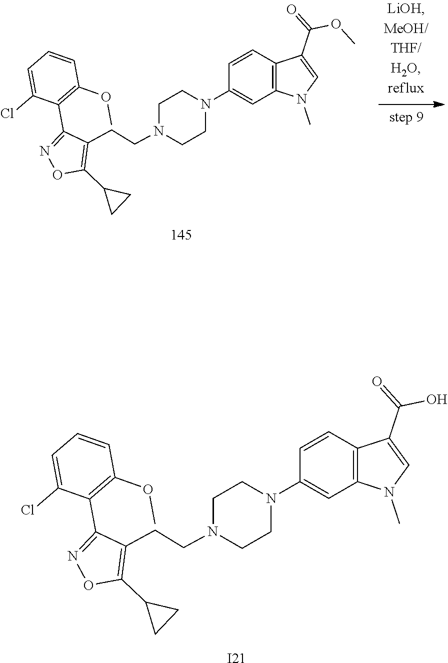

Follow the procedure of Synthesis of I1 to give I21. LCMS: [M+H]+=535.2; ¹H NMR (400 HMz, DMSO-d6) δ ppm: 10.07 (br, 1H), 7.89-7.84 (m, 2H), 7.58-7.53 (m, 1H), 7.24-7.20 (m, 2H), 7.47-7.42 (m, 1H), 7.01-6.98 (m, 2H), 3.85-3.79 (m, 8H), 3.58-3.56 (m, 2H), 3.18-3.13 (m, 4H), 3.02-2.96 (m, 2H), 2.79-2.67 (m, 2H), 2.36-2.32 (m, 1H), 1.16-1.07 (m, 4H).

### Example 22

### 6-(4-(2-(3-(2-chloro-6-methylphenyl)-5-cyclopropylisoxazol-4-yl)ethyl)piperazin-1-yl)-1-methyl-1H-indole-3-carboxylic Acid (I22)

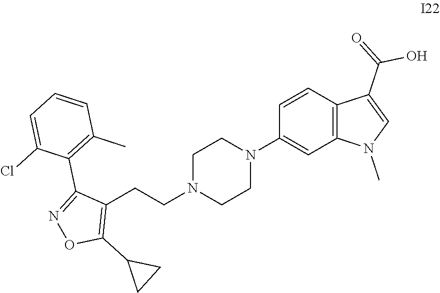 [SMILES: Cc1cccc(Cl)c1-c1noc(C2CC2)c1CCN1CCN(c2ccc3c(C(=O)O)cn(C)c3c2)CC1]

### Step 2: Synthesis of 150

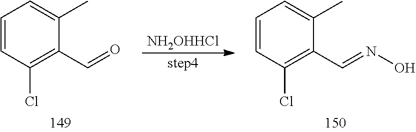

Follow the procedure of Synthesis 8 to give 150, and which was used directly in the next step. LCMS: [M⁺H]+=170;¹H NMR (400 HMz, CDCl₃) δ ppm:8.46 (s,1H), 7.20-7.06 (m, 3H), 2.40 (s,3H).

### Step 2: Synthesis of 151

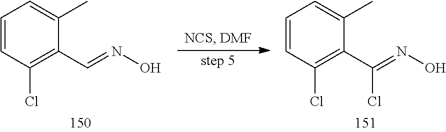

Follow the procedure of Synthesis of 9 to give 151, and which was used directly in the next step.

### Step 3: Synthesis of 152

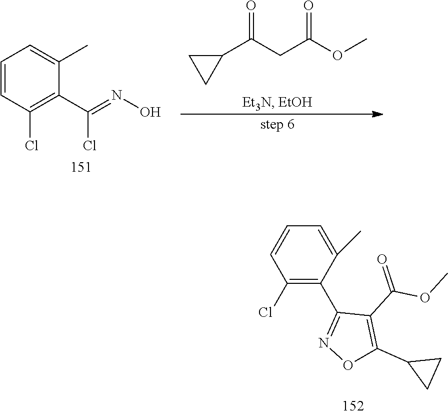

Follow the procedure of Synthesis of 10 to give 152. LCMS: [M+H]+=292.07; ¹H NMR (400 HMz, CDCl₃) δ ppm: 7.33-7.19 (m, 3H), 3.66 (s, 3H), 2.98-2.89 (m, 1H), 2.09 (s, 3H), 1.47-1.20 (m, 4H).

### Step 4: Synthesis of 153

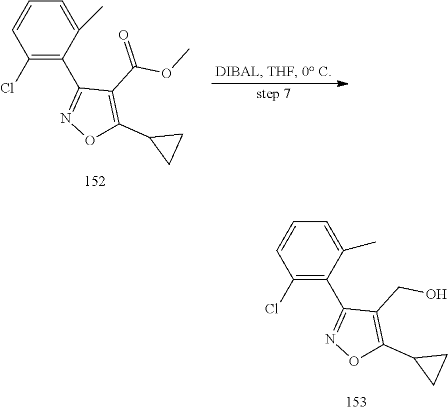 [SMILES: COC(=O)c1c(-c2c(C)cccc2Cl)noc1C1CC1 ; "DIBAL, THF, 0° C." ; "step 7" ; Cc1cccc(Cl)c1-c1noc(C2CC2)c1CO]

Follow the procedure of Synthesis of 11 to give 153. LCMS: [M+H]+=264; ¹H NMR (400 HMz, CDCl₃) δ ppm: 7.26-7.19 (m, 2H), 7.14-7.12 (m, 1H), 4.30 (s, 2H), 2.11 (s, 3H), 1.48 (s, 1H), 1.21-1.17 (m, 2H), 1.09-1.05 (m, 2H).

### Step 5: Synthesis of 154

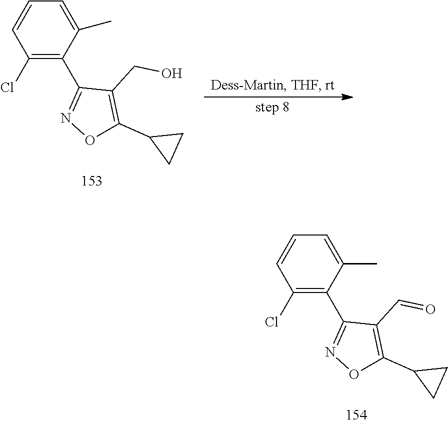 [SMILES: Cc1cccc(Cl)c1-c1noc(C2CC2)c1CO ; "Dess-Martin, THF, rt" ; "step 8" ; Cc1cccc(Cl)c1-c1noc(C2CC2)c1C=O]

Follow the procedure of Synthesis of 12 to give 154. LCMS: [M+H]+=262; ¹H NMR (400 HMz, CDCl₃) δ ppm: 9.50 (s, 1H), 7.28-7.15 (m, 3H), 2.82-2.78 (m, 1H), 2.15 (s, 3H), 1.40-1.38 (m, 2H), 1.29-1.27 (m, 2H).

### Step 6: Synthesis of 155

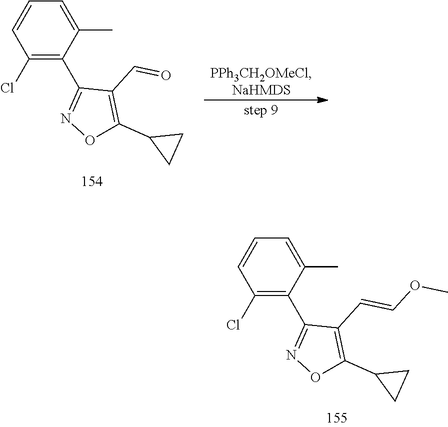

Follow the procedure of Synthesis of 13 to give 155. LCMS: [M+H]+=290.

### Step 7: Synthesis of 156

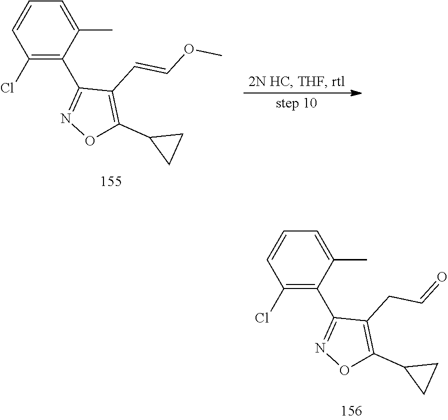 [SMILES: CO/C=C/c1c(-c2c(C)cccc2Cl)noc1C1CC1 ; "2N HC, THF, rtl" ; "step 10" ; Cc1cccc(Cl)c1-c1noc(C2CC2)c1CC=O]

Follow the procedure of Synthesis of 14 to give 156. LCMS: [M+H]+=276.1.

### Step 8: Synthesis of 157

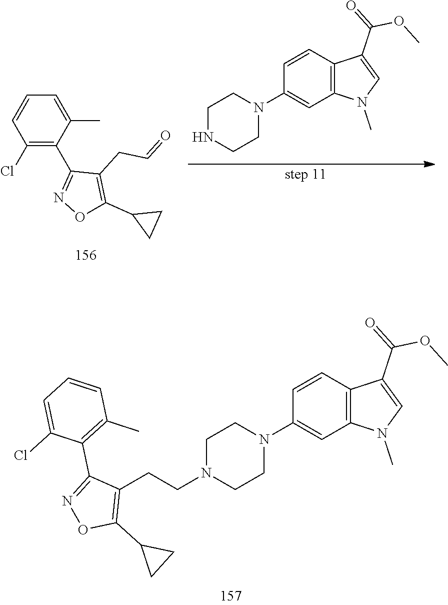

Follow the procedure of Synthesis of 15 to give 157. LCMS: [M+H]+=533.2.

### Step 9: Synthesis of 122

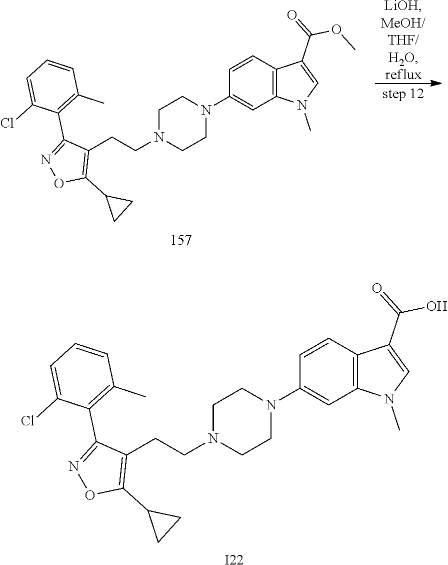

Follow the procedure of Synthesis of I1 to give I22. LCMS: [M+H]+=519.2; ¹H NMR (400 HMz, DMSO-d6) δ ppm: 11.78 (br, 1H), 7.80-7.77 (m, 2H), 7.46-7.44 (m, 2H), 7.37-7.36 (m, 1H), 6.91-6.87 (m, 2H), 3.76 (s, 3H), 3.05-3.04 (m, 4H), 2.50-2.25 (m, 9H), 2.13 (s, 3H), 1.23-1.02 (m, 4H).

### Example 23

### 6-(4-(1-(5-cyclopropyl-3-(2,6-dichlorophenyl)isoxazol-4-yl)propan-2-yl)piperazin-1-yl)-1-methyl-1H-indole-3-carboxylic Acid (I23)

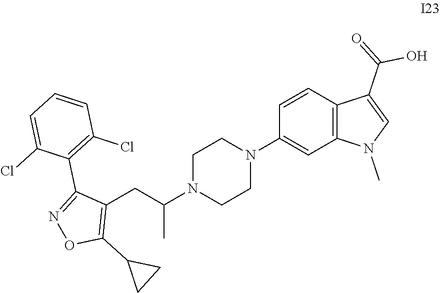 [SMILES: CC(Cc1c(-c2c(Cl)cccc2Cl)noc1C1CC1)N1CCN(c2ccc3c(C(=O)O)cn(C)c3c2)CC1]

### Step 1: Synthesis of 158

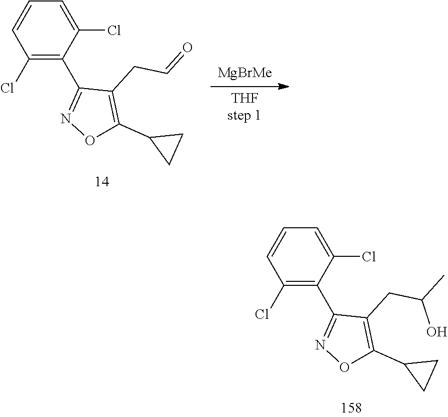 [SMILES: O=CCc1c(-c2c(Cl)cccc2Cl)noc1C1CC1 ; "THFstep 1" ; CC(O)Cc1c(-c2c(Cl)cccc2Cl)noc1C1CC1 ; "MgBrMe"]

To a solution of compound 14 (500 mg, 1.69 mmol) in THF (10 mL) was cooled in an ice water bath was added methylmagnesium bromide (3.0 M in diethyl ether, 1.13 mL, 3.39 mmol) was added via syringe over 5 min at 0° C. under nitrogen. The reaction was stirred at 0° C. for 40 min. After LCMS indicated the reaction was completed, the reaction was treated with saturated ammonium chloride (10 mL) at 0° C. Then, the reaction was diluted with water (25 mL) and the layers were separated. The aqueous phase was extracted with EtOAc, and the organic phases were combined, dried over Na₂SO₄, filtered and concentrated to give 158. LCMS: [M+H]+=313.8.

### Step 2: Synthesis of 159

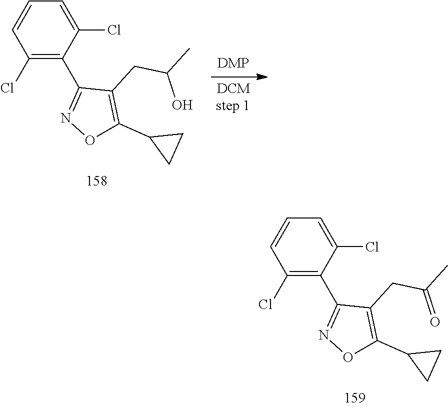

To a solution of compound 158 (1.3 g, 4.16 mmol) in DCM (20 mL) at 0° C., the Dess-martin (2.65 g, 6.24 mol) was added portionwise. Then the reaction mixture was allowed to return to room temperature and stirred for 3 h. After LCMS indicated the reaction was completed, a saturated aqueous solution of NaHCO₃ (30 mL) and Na₂S₂O₃ (30 mL) was added to the reaction and stirred for 10 min. Then the solution extracted with DCM and the combined organic layers were washed with brine, dried, concentrated and purified by silica gel column chromatography (PE/EA=5:1) to give 159 (491 mg, white solid, yield 38%). ¹H NMR (400 HMz, CDCl₃) δ 7.37-7.34 (m, 2H), 7.28 (dd, J=9.1, 6.9 Hz, 1H), 3.28 (s, 2H), 2.00 (s, 3H), 1.93-1.87 (m, 1H) 1.25-1.12 (m, 2H), 1.04 (m, 2H). LCMS: [M+H]+=311.7.

### Step 3: Synthesis of 160

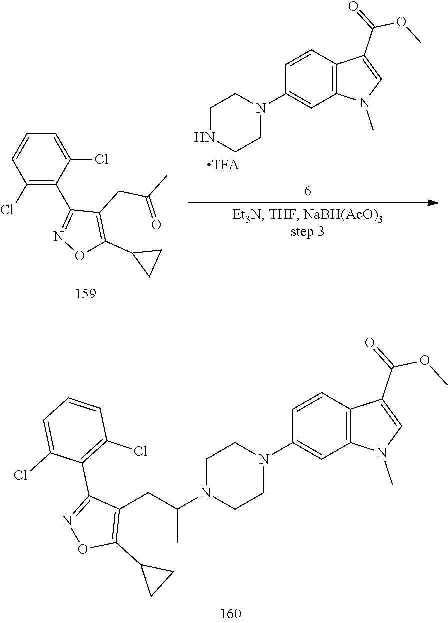 [SMILES: CC(=O)Cc1c(-c2c(Cl)cccc2Cl)noc1C1CC1 ; COC(=O)c1cn(C)c2cc(N3CCNCC3)ccc12 ; "Et3N, THF, NaBH(AcO)3step 3" ; COC(=O)c1cn(C)c2cc(N3CCN(C(C)Cc4c(-c5c(Cl)cccc5Cl)noc4C4CC4)CC3)ccc12]

Follow the procedure of Synthesis of 15 to give 160. LCMS: [M+H]+=554.9

### Step 4: Synthesis of I23

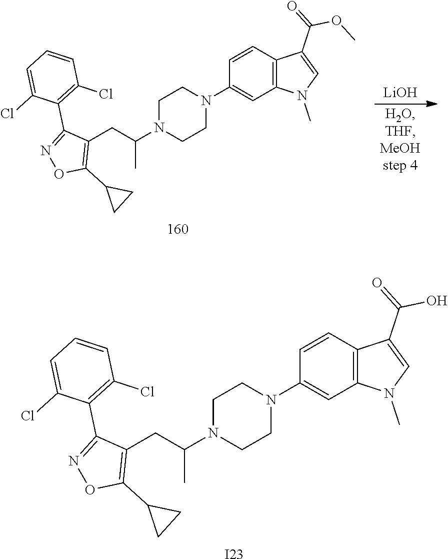 [SMILES: COC(=O)c1cn(C)c2cc(N3CCN(C(C)Cc4c(-c5c(Cl)cccc5Cl)noc4C4CC4)CC3)ccc12 ; "LiOH" ; "H2O,THF,MeOHstep 4" ; CC(Cc1c(-c2c(Cl)cccc2Cl)noc1C1CC1)N1CCN(c2ccc3c(C(=O)O)cn(C)c3c2)CC1 ; "I23"]

Follow the procedure of Synthesis of I1 to give I23. ¹H NMR (400 HMz, MeOD) δ 7.96 (d, J=9.3 Hz, 1H), 7.82 (s, 1H), 7.66-7.58 (m, 2H), 7.57 (d, J=7.7 Hz, 1H), 7.01 (d, J=5.9 Hz, 2H), 3.82 (s, 3H), 3.42-3.39 (m, 4H), 3.30-3.21 (m, 4H), 3.04 (d, J=11.9 Hz, 2H), 2.79 (d, J=12.0 Hz, 1H), 2.29 (s, 1H), 1.36 (d, J=6.6 Hz, 3H), 1.22 (dd, J=10.4, 4.6 Hz, 4H). [M+H]+=552.9.

### Example 24

### 6-(4-(2-(5-cyclopropyl-3-(2,6-dichlorophenyl)isoxazol-4-yl)propyl)piperazin-1-yl)-1-methyl-1H-indole-3-carboxylic Acid (I24)

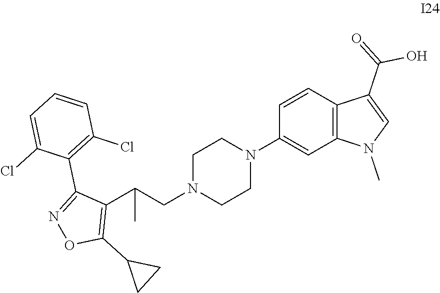 [SMILES: CC(CN1CCN(c2ccc3c(C(=O)O)cn(C)c3c2)CC1)c1c(-c2c(Cl)cccc2Cl)noc1C1CC1]

### Step 1: Synthesis of 161

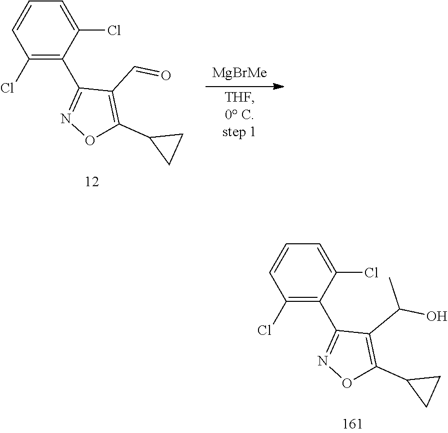 [SMILES: O=Cc1c(-c2c(Cl)cccc2Cl)noc1C1CC1 ; "THF,0° C.step 1" ; CC(O)c1c(-c2c(Cl)cccc2Cl)noc1C1CC1 ; "MgBrMe"]

Follow the procedure of Synthesis of 158 to give 161. LCMS: [M+H]+=300.0

### Step 2: Synthesis of 162

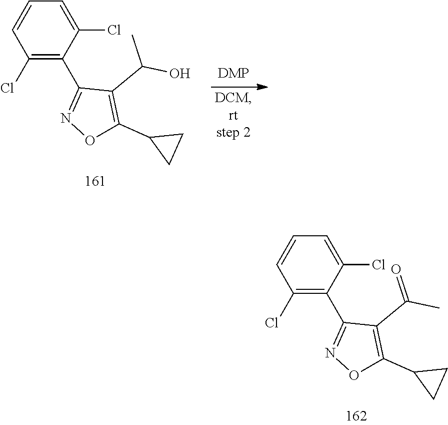

Follow the procedure of Synthesis of 159 to give 162. ¹H NMR (400 HMz, CDCl₃) δ 7.49-7.30 (m, 3H), 3.07-2.95 (m, 1H), 2.03 (s, 3H), 1.47-1.37 (m, 2H), 1.34-1.23 (m, 2H). LCMS: [M+H]+=298.0

### Step 3: Synthesis of 163

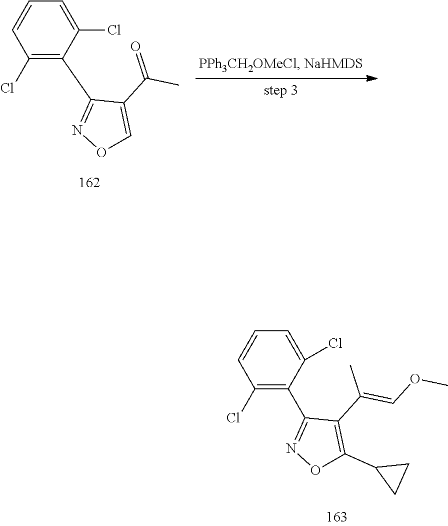 [SMILES: CC(=O)c1conc1-c1c(Cl)cccc1Cl ; CO/C=C(\C)c1c(-c2c(Cl)cccc2Cl)noc1C1CC1]

Follow the procedure of Synthesis of 13 to give 163. ¹H NMR (400 HMz, CDCl₃) δ 7.33-7.28 (m, 3H), 7.26-7.20 (m, 1H), 5.82 (d, J=1.1 Hz, 1H), 3.24 (s, 3H), 1.99-1.90 (m, 1H), 1.61 (s, 3H), 1.16-1.12 (m, 2H), 0.98 (dt, J=7.3, 4.3 Hz, 2H). LCMS: [M+H]+=326.0.

### Step 4: Synthesis of 164

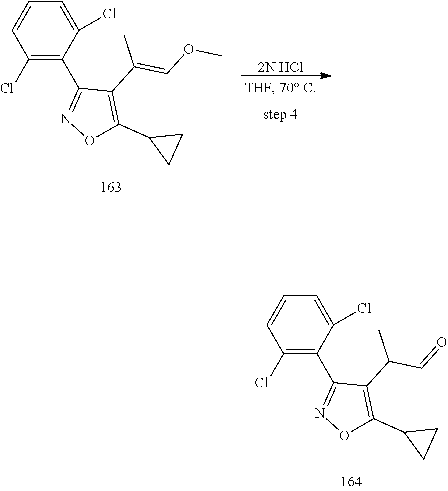

Follow the procedure of Synthesis of 14 to give 164. LCMS: [M+H]+=312.0.

### Step 5: Synthesis of 165

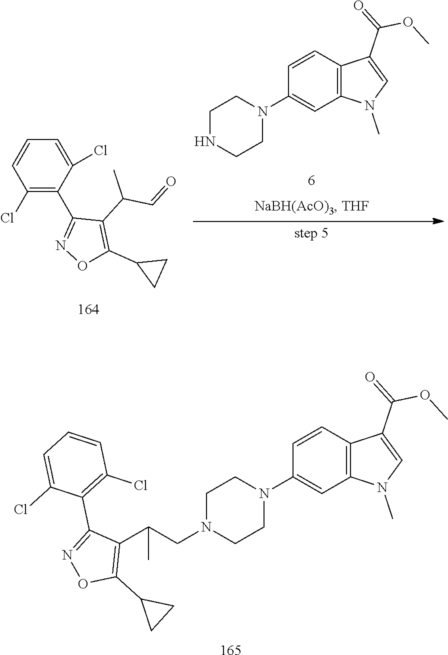 [SMILES: CC(C=O)c1c(-c2c(Cl)cccc2Cl)noc1C1CC1 ; COC(=O)c1cn(C)c2cc(N3CCNCC3)ccc12 ; COC(=O)c1cn(C)c2cc(N3CCN(CC(C)c4c(-c5c(Cl)cccc5Cl)noc4C4CC4)CC3)ccc12 ; "165"]

Follow the procedure of Synthesis of 15 to give 165. LCMS: [M+H]+=569.2

### Step 6: Synthesis of I24

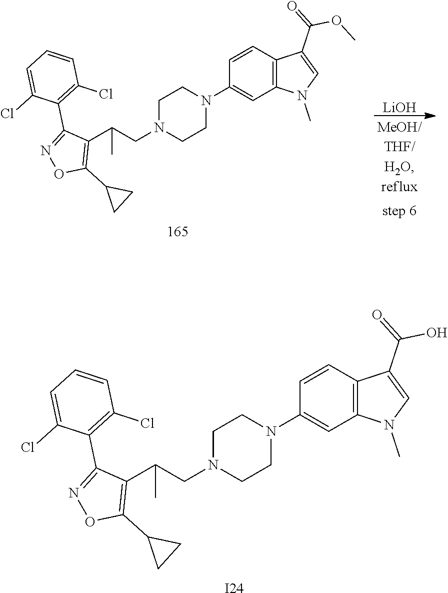 [SMILES: COC(=O)c1cn(C)c2cc(N3CCN(CC(C)c4c(-c5c(Cl)cccc5Cl)noc4C4CC4)CC3)ccc12 ; "LiOH" ; "165" ; CC(CN1CCN(c2ccc3c(C(=O)O)cn(C)c3c2)CC1)c1c(-c2c(Cl)cccc2Cl)noc1C1CC1 ; "I24"]

Follow the procedure of Synthesis of I1 to give compound I24 (11.7 mg, off-white solid, yield 21%). 1H NMR (400 MHz, MeOD) δ 7.85 (d, J=8.6 Hz, 1H), 7.74-7.69 (m, 1H), 7.54-7.42 (m, 3H), 6.91 (dd, J=10.7, 1.8 Hz, 2H), 3.72 (s, 3H), 3.31-3.20 (m, 4H), 3.13-2.96 (m, 5H), 2.95-2.86 (m, 1H), 2.24-2.15 (m, 1H), 1.27 (d, J=6.9 Hz, 3H), 1.11 (dd, J=10.2, 7.6 Hz, 4H). [M+H]+=552.8.

### Example 25

### 6-(4-(2-(5-cyclopropyl-3-(2,6-dichlorophenyl)isoxazol-4-yl)ethyl)-2,2-dimethylpiperazin-1-yl)-1-methyl-1H-indole-3-carboxylic Acid (I25)

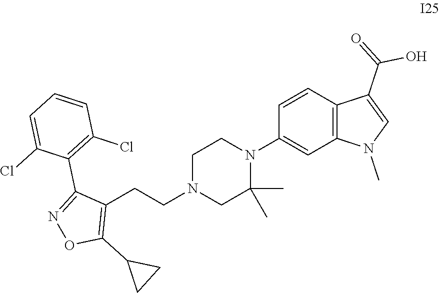 [SMILES: Cn1cc(C(=O)O)c2ccc(N3CCN(CCc4c(-c5c(Cl)cccc5Cl)noc4C4CC4)CC3(C)C)cc21]

### Step 1: Synthesis of 166

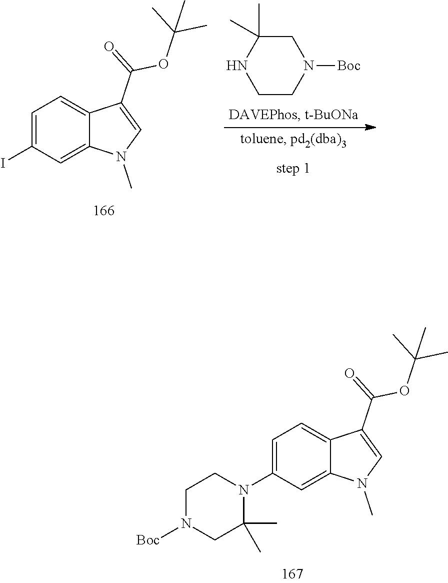

Under nitrogen, a mixture of tert-butyl 6-iodo-1-methyl-1H-indole-3-carboxylate (166) (0.75 g, 2.12 mmol), tert-butyl 3,3-dimethylpiperazine-1-carboxylate (0.5 g, 2.33 mmol), t-BuONa (0.51 g, 5.3 mmol), DAVE Phos (37.5 mg, 0.1 mmol) and Pd2(dba)3 (98 mg, 0.1 mmol) were successively added to toluene (8 mL). Then the mixture was heated to 100° C. and stirred overnight. After completion of the reaction, the suspension is filtered, the filter cake was washed with EA. The filtrate is concentrated, dried and purified by silica column with PE/EA (5:1) to give 167. ¹H NMR (400 HMz, CDCl₃) δ 7.92 (d, J=8.4 Hz, 1H), 7.62 (s, 1H), 7.00 (d, J=12.1 Hz, 2H), 3.71 (s, 3H), 3.53 (s, 2H), 3.29 (s, 2H), 3.08 (s, 2H), 1.53 (d, J=16.5 Hz, 9H), 1.43 (s, 9H), 0.97 (s, 6H). LCMS: [M+H]+=443.9.

### Step 2: Synthesis of 168

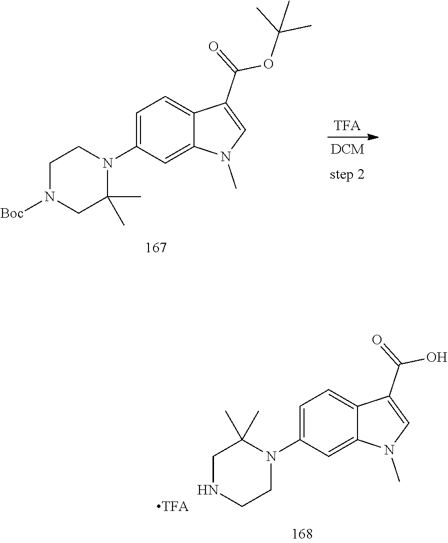 [SMILES: Cn1cc(C(=O)OC(C)(C)C)c2ccc(N3CCN(C(=O)OC(C)(C)C)CC3(C)C)cc21 ; "·TFA" ; Cn1cc(C(=O)O)c2ccc(N3CCNCC3(C)C)cc21]

Follow the procedure of Synthesis of 6 to give 168 used directly for next step. LCMS: [M+H]+=343.9.

### Step 3: Synthesis of I25

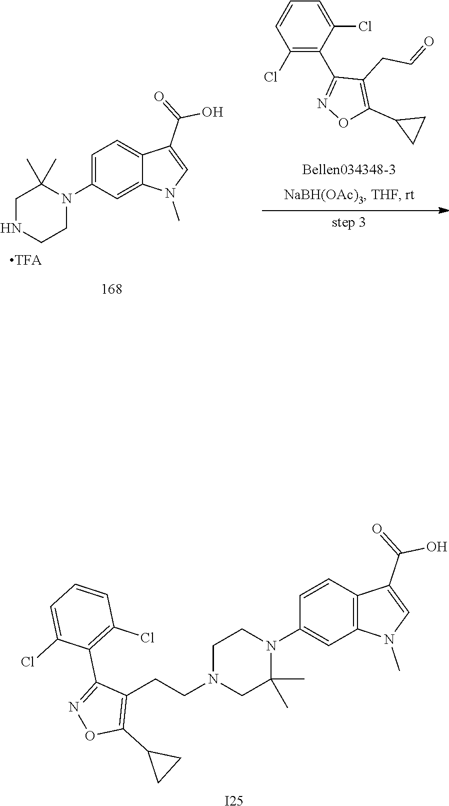 [SMILES: Cn1cc(C(=O)O)c2ccc(N3CCNCC3(C)C)cc21 ; O=CCc1c(-c2c(Cl)cccc2Cl)noc1C1CC1 ; "·TFA" ; Cn1cc(C(=O)O)c2ccc(N3CCN(CCc4c(-c5c(Cl)cccc5Cl)noc4C4CC4)CC3(C)C)cc21 ; "I25"]

Follow the procedure of Synthesis of 15 to give I25. ¹H NMR (400 HMz, DMSO-d6) δ 9.78 (s, 1H), 8.02 (s, 1H), 7.89 (d, J=8.2 Hz, 1H), 7.77-7.67 (m, 2H), 7.67-7.62 (m, 1H), 7.21 (s, 1H), 7.01 (s, 1H), 3.82 (s, 3H), 3.69-3.36 (m, 1H), 3.51-3.48 (m, 2H), 3.10-3.05 (m, 2H), 2.99-2.94 (m, 2H), 2.81-2.78 (m, 2H), 2.39 (t, J=19.6 Hz, 2H), 1.31-1.08 (m, 8H), 1.03 (s, 2H). LCMS: [M+H]+=567.2.

### Example 26

### 6-(4-(2-(5-cyclopropyl-3-(2-(trifluoromethyl)phenyl)isoxazol-4-yl)ethyl)piperazin-1-yl)-1-methyl-1H-indole-3-carboxylic Acid (I26)

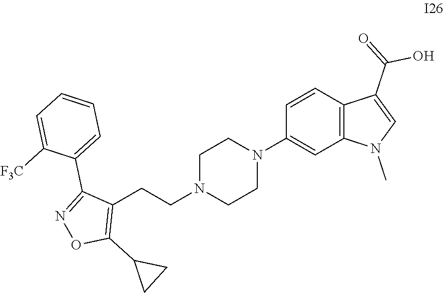 [SMILES: Cn1cc(C(=O)O)c2ccc(N3CCN(CCc4c(-c5ccccc5C(F)(F)F)noc4C4CC4)CC3)cc21]

### Step 1: Synthesis of 171

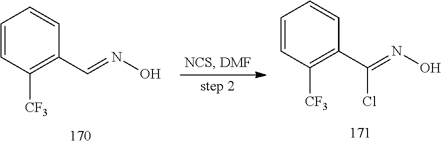 [SMILES: O/N=C/c1ccccc1C(F)(F)F ; O/N=C(\Cl)c1ccccc1C(F)(F)F]

Follow the procedure of Synthesis of 9 to give 171, which was used directly in the next step.

### Step 2: Synthesis of 172

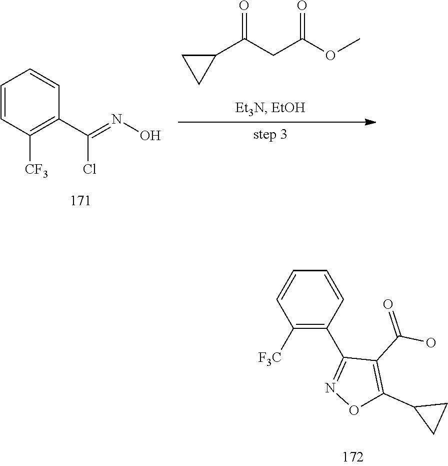

Follow the procedure of Synthesis of 10 to give 172. ¹H NMR (400 HMz, CDCl₃) δ ppm: 7.81-7.78 (m, 1H), 7.64-7.62 (m, 2H), 7.43-7.40 (m, 1H), 3.65 (s, 3H), 2.93-2.86 (m, 1H), 1.43-1.38 (m, 2H), 1.32-1.27 (m, 2H).

### Step 3: Synthesis of 173

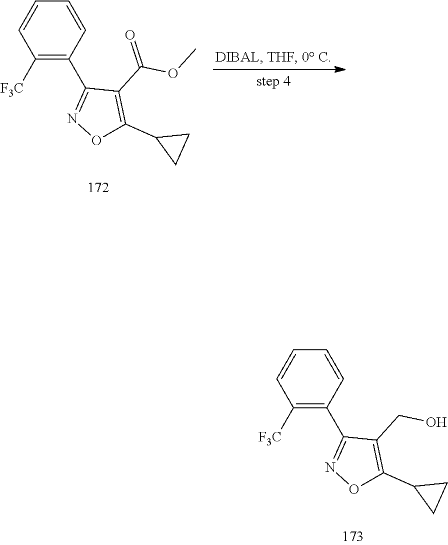 [SMILES: COC(=O)c1c(-c2ccccc2C(F)(F)F)noc1C1CC1 ; OCc1c(-c2ccccc2C(F)(F)F)noc1C1CC1]

Follow the procedure of Synthesis of 11 to give 173. LCMS: [M+H]+=284.1; ¹H NMR (CDCl₃) δ ppm: 7.74-7.72 (m, 1H), 7.57-7.50 (m, 2H), 7.39-7.37 (m, 1H), 4.31 (s, 2H), 2.11-2.04 (m, 1H), 1.51 (s, 1H), 1.19-1.15 (m, 2H), 1.07-1.04 (m, 2H).

### Step 4: Synthesis of 174

Follow the procedure of Synthesis of 12 to give 174. LCMS: [M+H]+=282.1; ¹H NMR (400 HMz, CDCl₃) δ ppm: 9.53 (s, 1H), 7.78-7.76 (m, 1H), 7.61-7.58 (m, 2H), 7.41-7.39 (m, 1H), 2.79-2.74 (m, 1H), 1.39-1.36 (m, 2H), 1.30-1.26 (m, 2H).

### Step 5: Synthesis of 175

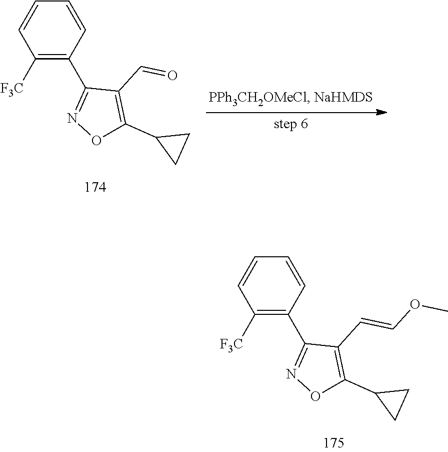 [SMILES: O=Cc1c(-c2ccccc2C(F)(F)F)noc1C1CC1 ; CO/C=C/c1c(-c2ccccc2C(F)(F)F)noc1C1CC1]

Follow the procedure of Synthesis of 13 to give 175. LCMS: [M+H]+=310.1; ¹H NMR (400 HMz, CDCl₃) δ ppm: 7.81-7.76 (m, 1H), 7.63-7.56 (m, 2H), 7.41-7.39 (m, 1H), 6.42 (d, J=13.2 Hz, 1H), 5.27 (d, J=13.2 Hz, 1H), 3.51 (s, 3H), 2.07-2.03 (m, 1H), 1.21-1.17 (m, 2H), 1.11-1.10 (m, 2H).

### Step 6: Synthesis of 176

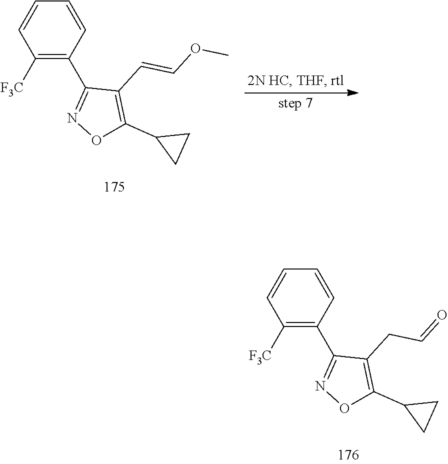 [SMILES: CO/C=C/c1c(-c2ccccc2C(F)(F)F)noc1C1CC1 ; O=CCc1c(-c2ccccc2C(F)(F)F)noc1C1CC1]

Follow the procedure of Synthesis of 14 to give 176. ¹H NMR (400 HMz, CDCl₃) δ ppm: 9.49 (s, 1H), 7.73-7.71 (m, 1H), 7.56-7.52 (m, 2H), 7.30-7.28 (m, 1H), 3.30 (s, 2H), 1.85-1.78 (m, 1H), 1.19-1.05 (m, 4H).

### Step 7: Synthesis of 177

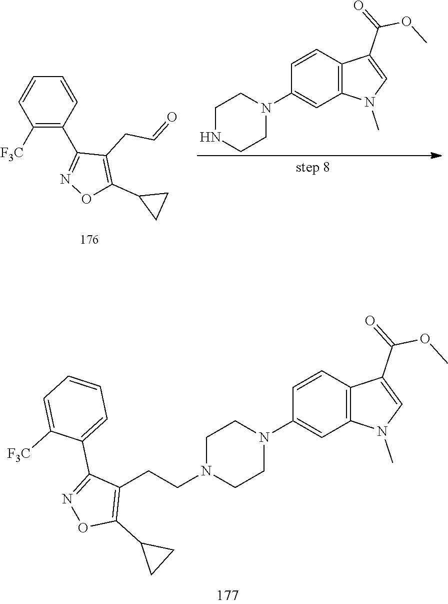

Follow the procedure of Synthesis of 15 to give 177. LCMS: [M+H]+=553.2.

### Step 8: Synthesis of 126

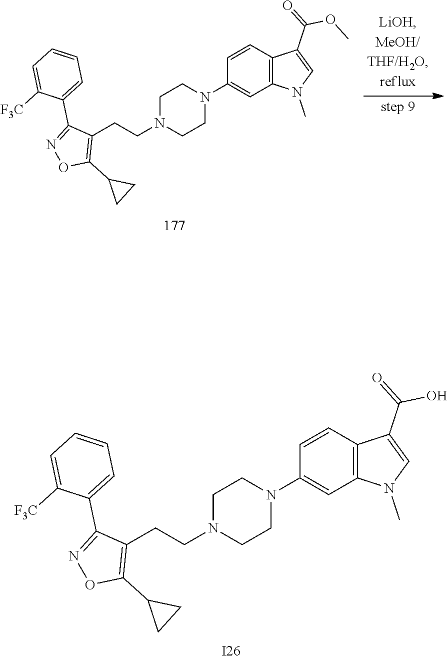 [SMILES: COC(=O)c1cn(C)c2cc(N3CCN(CCc4c(-c5ccccc5C(F)(F)F)noc4C4CC4)CC3)ccc12 ; Cn1cc(C(=O)O)c2ccc(N3CCN(CCc4c(-c5ccccc5C(F)(F)F)noc4C4CC4)CC3)cc21 ; "I26"]

Follow the procedure of Synthesis of I1 to give I26. LCMS: [M+H]+=539.2; ¹H NMR (400 HMz, DMSO-d6) δ ppm: 11.83 (br, 1H), 7.96-7.95 (m, 1H), 7.88-7.79 (m, 4H), 7.64-7.62 (m, 1H), 7.00-6.96 (m, 2H), 3.79 (s, 5H), 3.55-3.53 (m, 2H), 3.33 (s, 6H), 3.13-3.11 (m, 2H), 2.41 (s, 1H), 1.17-1.09 (m, 4H).

### Example 27

### 6-(4-(2-(3-(2,6-dichlorophenyl)-5-methylisoxazol-4-yl)ethyl)piperazin-1-yl)-1-methyl-1H-indole-3-carboxylic Acid (I127)

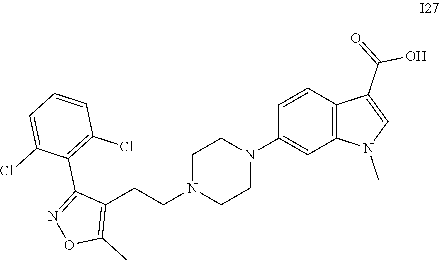 [SMILES: Cc1onc(-c2c(Cl)cccc2Cl)c1CCN1CCN(c2ccc3c(C(=O)O)cn(C)c3c2)CC1]

### Step 3: Synthesis of 181

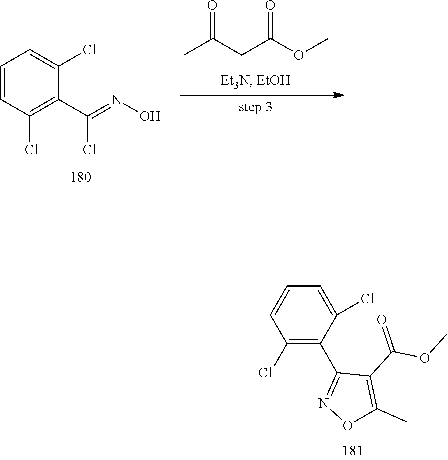

Follow the procedure of Synthesis of 10 to give 181. LCMS: [M+H]+=286.0; ¹H NMR (400 HMz, CDCl₃) δ ppm: 7.35-7.34 (m, 1H), 7.29-7.25 (m, 2H), 3.62 (s, 3H), 2.72 (s, 3H).

### Step 4: Synthesis of 182

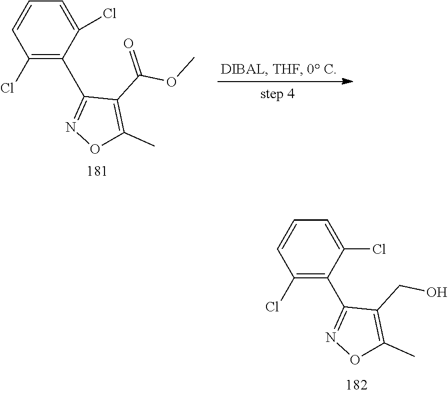 [SMILES: COC(=O)c1c(-c2c(Cl)cccc2Cl)noc1C ; Cc1onc(-c2c(Cl)cccc2Cl)c1CO]

Follow the procedure of Synthesis of 11 to give 182. LCMS: [M+H]+=258.0; ¹H NMR (400 HMz, CDCl₃) δ ppm: 7.37-7.27 (m, 3H), 4.28 (s, 2H), 2.48 (s, 3H), 1.44 (s, 1H).

### Step 5: Synthesis of 183

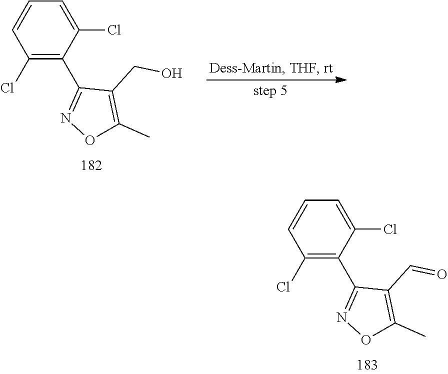 [SMILES: Cc1onc(-c2c(Cl)cccc2Cl)c1CO ; Cc1onc(-c2c(Cl)cccc2Cl)c1C=O]

Follow the procedure of Synthesis of 12 to give 183. LCMS: [M+H]+=256.0; ¹H NMR (400 HMz, CDCl₃) δ ppm: 9.58 (s, 3H), 7.38-7.35 (m, 3H), 2.76 (s, 2H).

### Step 6: Synthesis of 184

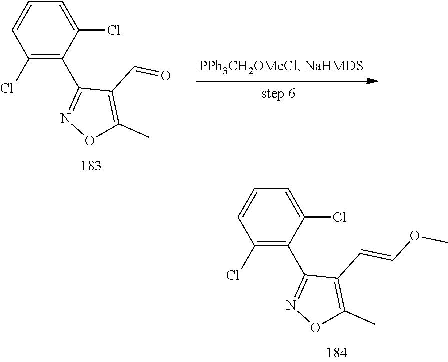 [SMILES: Cc1onc(-c2c(Cl)cccc2Cl)c1C=O ; CO/C=C/c1c(-c2c(Cl)cccc2Cl)noc1C]

Follow the procedure of Synthesis of 13 to give 184. LCMS: [M+H]+=284.0; ¹H NMR (400 HMz, CDCl₃) δ ppm: 7.35-7.22 (m, 3H), 6.25 (d, J=12.8 Hz, 1H), 5.16 (d, J=12.8 Hz, 1H), 3.46 (s, 3H), 2.42 (s, 3H).

### Step 7: Synthesis of 185

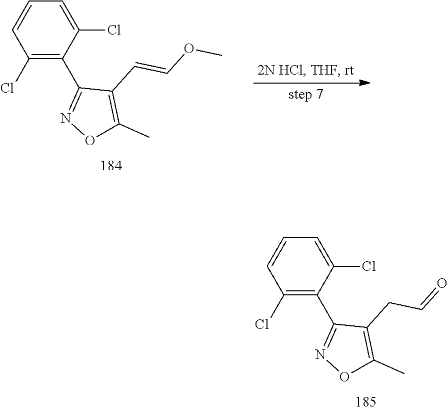 [SMILES: CO/C=C/c1c(-c2c(Cl)cccc2Cl)noc1C ; Cc1onc(-c2c(Cl)cccc2Cl)c1CC=O]

Follow the procedure of Synthesis of 14 to give 185. LCMS: [M+H]+=270.0; ¹H NMR (400 HMz, CDCl₃) δ ppm: 9.47 (s, 1H), 7.37-7.29 (m, 3H), 3.22 (s, 2H), 2.41 (s, 3H).

### Step 8: Synthesis of 186

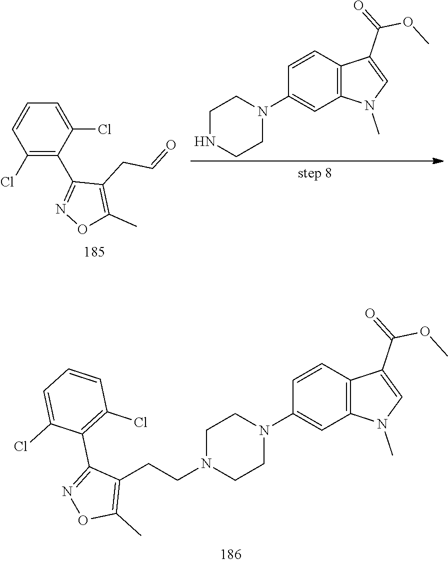

Follow the procedure of Synthesis of 15 to give 186. LCMS: [M+H]+=527.1; ¹H NMR (400 HMz, CDCl₃) δ ppm: 8.06 (d, J=8.4 Hz, 1H), 7.72 (s, 1H), 7.51-7.44 (m, 3H), 6.96-6.95 (m, 1H), 6.84 (s, 1H), 3.91 (s, 3H), 3.80 (s, 3H), 3.56-3.54 (m, 4H), 2.95-2.91 (m, 4H), 2.63 (s, 3H), 1.30-1.28 (m, 4H).

### Step 9: Synthesis of I27

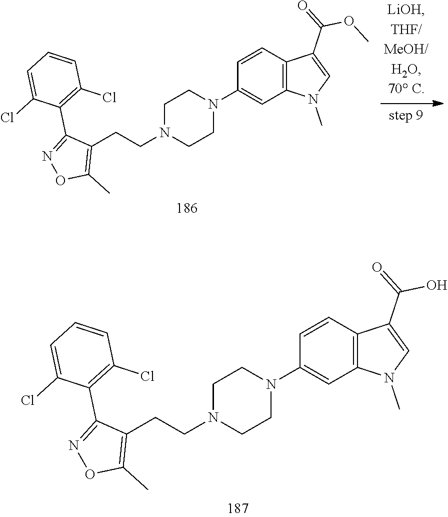 [SMILES: COC(=O)c1cn(C)c2cc(N3CCN(CCc4c(-c5c(Cl)cccc5Cl)noc4C)CC3)ccc12 ; Cc1onc(-c2c(Cl)cccc2Cl)c1CCN1CCN(c2ccc3c(C(=O)O)cn(C)c3c2)CC1]

Follow the procedure of Synthesis of I1 to give 127. LCMS: [M+H]+=513.1; ¹H NMR (400 HMz, DMSO-d6) δ ppm: 11.71 (br, 1H), 7.82-7.77 (m, 2H), 7.69-7.67 (m, 2H), 7.62-7.58 (m, 1H), 6.92-6.87 (m, 2H), 3.76 (s, 3H), 3.06-3.05 (m, 4H), 2.49 (s, 3H), 2.41-2.39 (m, 6H), 2.29-2.25 (m, 2H).

### Example 28

### 6-((1R,4R)-5-(2-(5-cyclopropyl-3-(2,6-dichlorophenyl)isoxazol-4-yl)ethyl)-2,5-diazabicyclo[2.2.1]heptan-2-yl)-1-methyl-1H-indole-3-carboxylic Acid (I28)

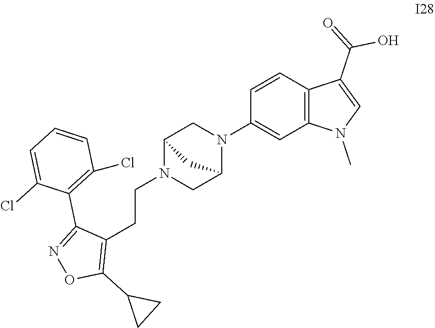 [SMILES: Cn1cc(C(=O)O)c2ccc(N3C[C@H]4C[C@@H]3CN4CCc3c(-c4c(Cl)cccc4Cl)noc3C3CC3)cc21]

### Step 1: Synthesis of 188

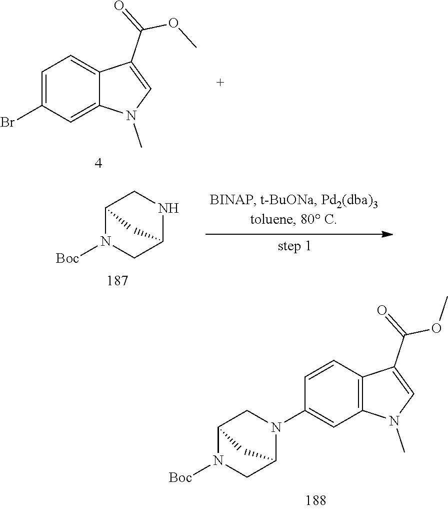

Follow the procedure of Synthesis of 5 to give 188. LCMS: [M+H]+=385.2; ¹H NMR (400 HMz, CDCl₃) δ ppm: 7.92-7.89 (m, 1H), 7.52 (s, 1H), 6.60-6.58 (m, 1H), 6.30 (s, 1H), 4.58-4.38 (m, 2H), 3.81 (s, 3H), 3.66 (s, 3H), 3.62-3.20 (m, 4H), 1.87 (s, 1H), 1.38-1.31 (m, 9H), 0.81-0.78 (m, 1H).

### Step 2: Synthesis of 189

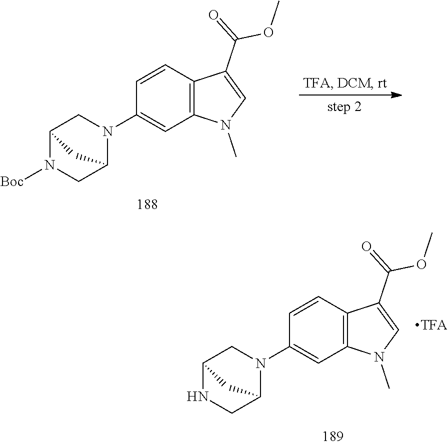 [SMILES: COC(=O)c1cn(C)c2cc(N3C[C@H]4C[C@@H]3CN4C(=O)OC(C)(C)C)ccc12 ; COC(=O)c1cn(C)c2cc(N3C[C@H]4C[C@@H]3CN4)ccc12]

Follow the procedure of Synthesis of 6 to give 189. LCMS: [M+H]+=286.1.

### Step 3: Synthesis of 190

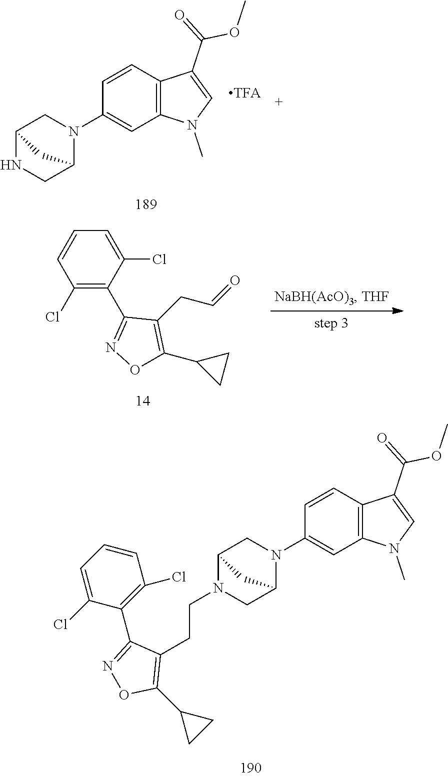 [SMILES: COC(=O)c1cn(C)c2cc(N3C[C@H]4C[C@@H]3CN4)ccc12 ; O=CCc1c(-c2c(Cl)cccc2Cl)noc1C1CC1 ; COC(=O)c1cn(C)c2cc(N3C[C@H]4C[C@@H]3CN4CCc3c(-c4c(Cl)cccc4Cl)noc3C3CC3)ccc12]

Follow the procedure of Synthesis of 15 to give 190. LCMS: [M+H]+=565.1. ¹H NMR (400 HMz, CDCl₃) δ ppm: 7.94-7.92 (m, 1H), 7.61 (s, 1H), 7.19-7.16 (m, 2H), 6.84 (s, 1H), 6.44-6.42 (m, 1H), 6.21 (s, 1H), 4.25 (s, 1H), 3.85 (s, 3H), 3.71 (s, 3H), 3.48-3.46 (m, 1H), 3.27-3.24 (m, 2H), 2.79-2.64 (m, 2H), 2.50-2.32 (m, 2H), 2.10 (s, 3H), 2.08-1.92 (m, 2H), 1.19-1.10 (m, 2H), 0.90-0.85 (m, 2H).

### Step 4: Synthesis of I28

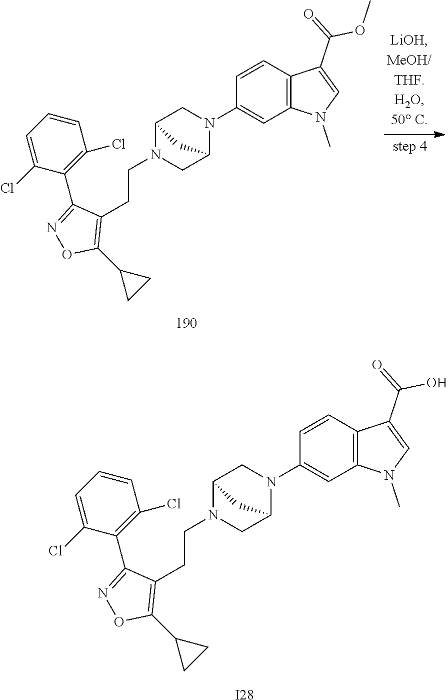 [SMILES: COC(=O)c1cn(C)c2cc(N3C[C@H]4C[C@@H]3CN4CCc3c(-c4c(Cl)cccc4Cl)noc3C3CC3)ccc12 ; "I28" ; Cn1cc(C(=O)O)c2ccc(N3C[C@H]4C[C@@H]3CN4CCc3c(-c4c(Cl)cccc4Cl)noc3C3CC3)cc21]

Follow the procedure of Synthesis of I1 to give 128. LCMS: [M+H]+=551.1; ¹H NMR (400 HMz, DMSO-d6) δ ppm: 11.76 (br, 1H), 7.76-7.74 (m, 2H), 7.54 (s, 1H), 7.24-7.20 (m, 1H), 7.04 (s, 1H), 6.51 (d, J=1.6 Hz, 1H), 6.49-6.43 (m, 1H), 4.23 (s, 1H), 3.74 (s, 3H), 3.40-3.27 (m, 2H), 3.07-3.05 (m, 1H), 2.68-2.66 (m, 1H), 2.50-2.09 (m, 6H), 1.73 (s, 2H), 1.04-0.98 (m, 4H).

### Example 29

### 6-(5-(2-(5-cyclopropyl-3-(2,6-dichlorophenyl)isoxazol-4-yl)ethyl)-2,5-diazabicyclo[2.2.1]heptan-2-yl)-1-methyl-1H-indole-3-carboxylic Acid (I29)

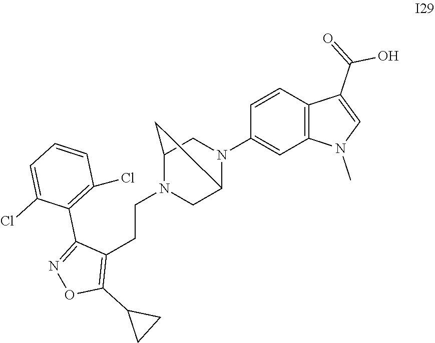 [SMILES: Cn1cc(C(=O)O)c2ccc(N3CC4CC3CN4CCc3c(-c4c(Cl)cccc4Cl)noc3C3CC3)cc21]

### Step 1: Synthesis of 192

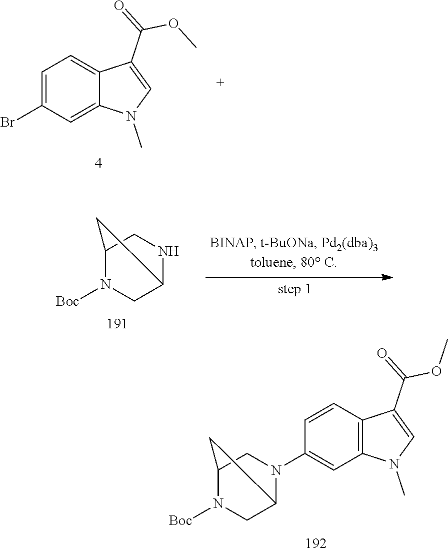 [SMILES: COC(=O)c1cn(C)c2cc(Br)ccc12 ; CC(C)(C)OC(=O)N1CC2CC1CN2 ; COC(=O)c1cn(C)c2cc(N3CC4CC3CN4C(=O)OC(C)(C)C)ccc12]

Follow the procedure of Synthesis of 5 to give 192. LCMS: [M+H]+=385.2; ¹H NMR (400 HMz, CDCl₃) δ ppm: 7.92-7.89 (m, 1H), 7.52 (s, 1H), 6.60-6.58 (m, 1H), 6.30 (s, 1H), 4.58-4.38 (m, 2H), 3.81 (s, 3H), 3.66 (s, 3H), 3.62-3.20 (m, 4H), 1.87 (s, 1H), 1.38-1.31 (m, 9H), 0.81-0.78 (m, 1H).

### Step 2: Synthesis of 193

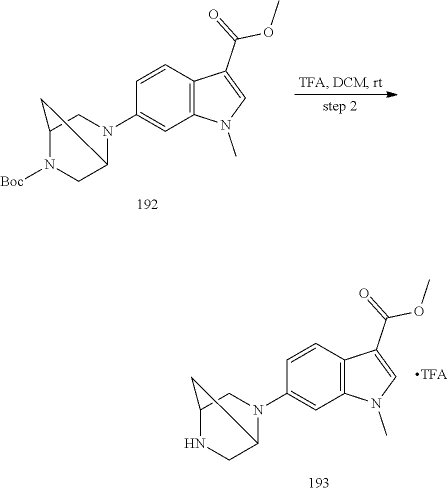 [SMILES: COC(=O)c1cn(C)c2cc(N3CC4CC3CN4C(=O)OC(C)(C)C)ccc12 ; COC(=O)c1cn(C)c2cc(N3CC4CC3CN4)ccc12]

Follow the procedure of Synthesis of 6 to give 193. LCMS: [M+H]+=286.1.

### Step 3: Synthesis of 194

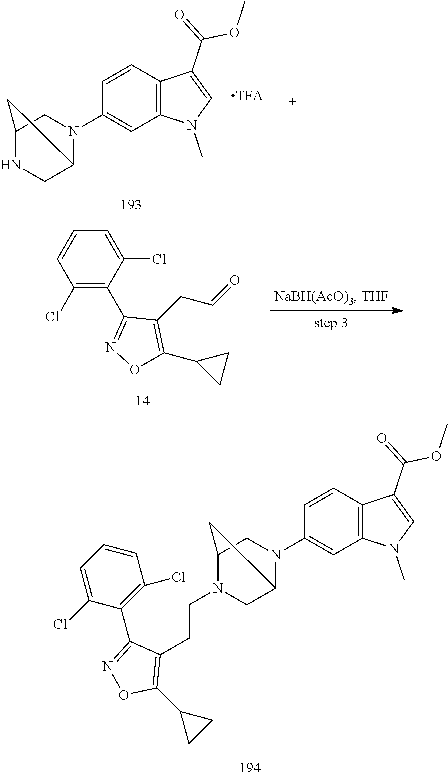 [SMILES: COC(=O)c1cn(C)c2cc(N3CC4CC3CN4)ccc12 ; O=CCc1c(-c2c(Cl)cccc2Cl)noc1C1CC1 ; COC(=O)c1cn(C)c2cc(N3CC4CC3CN4CCc3c(-c4c(Cl)cccc4Cl)noc3C3CC3)ccc12]

Follow the procedure of Synthesis of 15 to give 194. LCMS: [M+H]+=565.1.

### Step 4: Synthesis of I29

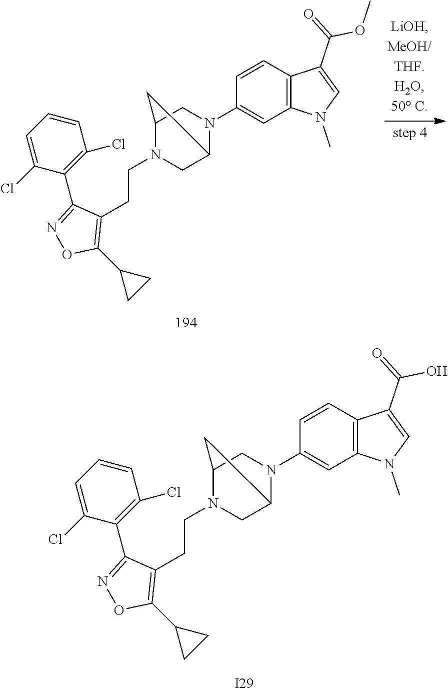 [SMILES: COC(=O)c1cn(C)c2cc(N3CC4CC3CN4CCc3c(-c4c(Cl)cccc4Cl)noc3C3CC3)ccc12 ; "I29" ; Cn1cc(C(=O)O)c2ccc(N3CC4CC3CN4CCc3c(-c4c(Cl)cccc4Cl)noc3C3CC3)cc21]

Follow the procedure of Synthesis of I1 to give I29. LCMS: [M+H]+=551.1; ¹H NMR (400 HMz, DMSO-d6) δ ppm: 11.76 (br, 1H), 7.80 (s, 2H), 7.61-7.54 (m, 1H), 7.22 (s, 1H), 7.04 (s, 1H), 6.67-6.44 (m, 1H), 4.68-4.23 (m, 1H), 3.74 (s, 3H), 3.40 (s, 1H), 3.07 (s, 1H), 2.67 (s, 1H), 2.40-2.08 (m, 5H), 1.74 (s, 1H), 1.05-0.99 (m, 4H).

### Example 30

### 6-(3-(2-(5-cyclopropyl-3-(2,6-dichlorophenyl)isoxazol-4-yl)ethyl)-3,8-diazabicyclo[3.2.1]octan-8-yl)-1-methyl-1H-indole-3-carboxylic Acid (I30)

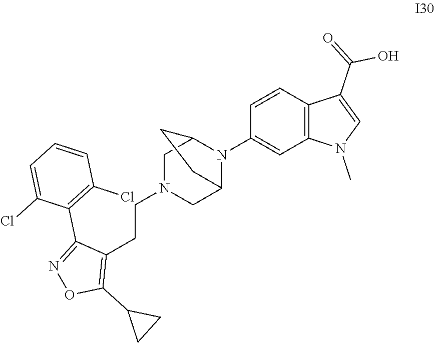 [SMILES: Cn1cc(C(=O)O)c2ccc(N3C4CCC3CN(CCc3c(-c5c(Cl)cccc5Cl)noc3C3CC3)C4)cc21]

### Step 1: Synthesis of 196

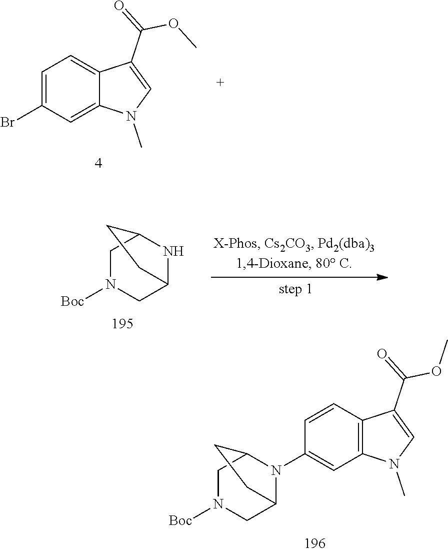 [SMILES: COC(=O)c1cn(C)c2cc(Br)ccc12 ; CC(C)(C)OC(=O)N1CC2CCC(C1)N2 ; COC(=O)c1cn(C)c2cc(N3C4CCC3CN(C(=O)OC(C)(C)C)C4)ccc12]

Under nitrogen, a mixture of compound 4 (224 mg, 0.83 mmol), compound 195 (212 mg, 1 mmol), Cs₂CO₃ (524 mg, 1.66 mmol), X-Phos (40 mg, 0.08 mmol), Pd2(dba)3 (40 mg, 0.04 mmol) were successively added to 1,4-Dioxane (5 mL). Then the mixture was heated to 80° C. and stirred overnight. After completion of the reaction, the suspension was filtered, the filter cake was washed with EA. The filtrate was concentrated, dried and purified by silica column with heptanes/EA (3:1) to give 196. LCMS: [M+H]+=400.0; ¹H NMR (400 HMz, CDCl₃) δ ppm: 7.93-7.91 (m, 1H), 7.55 (s, 1H), 6.82-6.79 (m, 1H), 6.57 (s, 1H), 4.21-4.16 (m, 2H), 3.81 (s, 3H), 3.72-3.67 (m, 4H), 3.57-3.54 (m, 1H), 3.31-3.22 (m, 2H), 1.99-1.98 (m, 2H), 1.81-1.78 (m, 2H), 1.38 (s, 9H).

### Step 2: Synthesis of 197

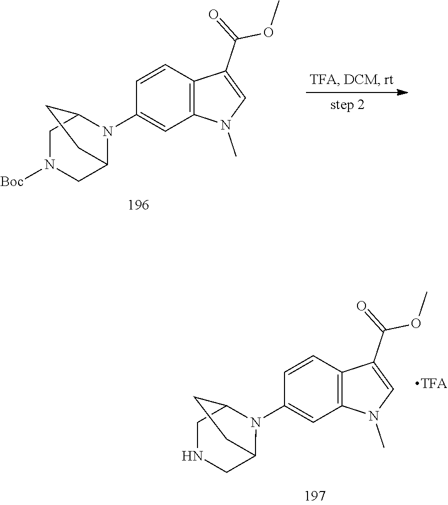 [SMILES: COC(=O)c1cn(C)c2cc(N3C4CCC3CN(C(=O)OC(C)(C)C)C4)ccc12 ; COC(=O)c1cn(C)c2cc(N3C4CCC3CNC4)ccc12]

Follow the procedure of Synthesis of 6 to give 197. LCMS: [M+H]+=300.1.

### Step 3: Synthesis of 198

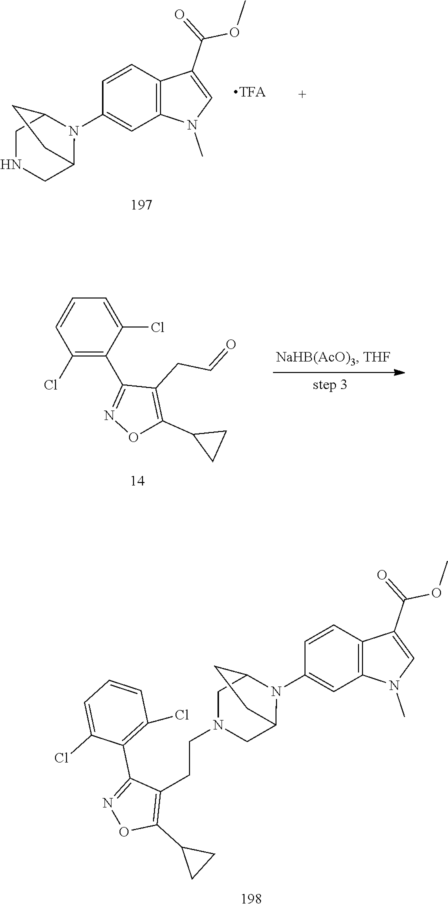 [SMILES: COC(=O)c1cn(C)c2cc(N3C4CCC3CNC4)ccc12 ; O=CCc1c(-c2c(Cl)cccc2Cl)noc1C1CC1 ; COC(=O)c1cn(C)c2cc(N3C4CCC3CN(CCc3c(-c5c(Cl)cccc5Cl)noc3C3CC3)C4)ccc12]

Follow the procedure of Synthesis of 15 to give 198. LCMS: [M+H]+=578.9; ¹H NMR (400 HMz, CDCl₃) δ ppm: 7.88-7.86 (m, 1H), 7.52 (s, 1H), 7.34-7.25 (m, 3H), 6.77-6.74 (m, 1H), 6.51 (s, 1H), 4.10 (s, 2H), 3.80 (s, 3H), 3.65 (s, 3H), 3.42 (s, 1H), 2.48-2.33 (m, 4H), 2.21-2.17 (m, 2H), 1.97-1.94 (m, 2H), 1.85 (s, 4H), 1.14-1.11 (m, 2H), 1.02-0.98 (m, 2H).

### Step 4: Synthesis of I30

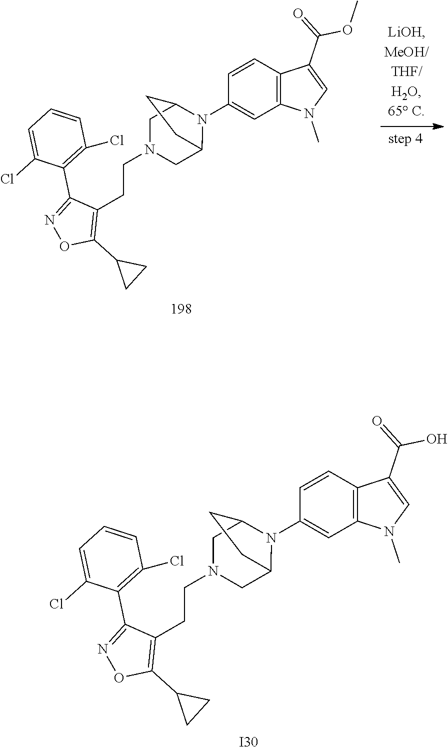 [SMILES: COC(=O)c1cn(C)c2cc(N3C4CCC3CN(CCc3c(-c5c(Cl)cccc5Cl)noc3C3CC3)C4)ccc12 ; "I30" ; Cn1cc(C(=O)O)c2ccc(N3C4CCC3CN(CCc3c(-c5c(Cl)cccc5Cl)noc3C3CC3)C4)cc21]

Follow the procedure of Synthesis of I1 to give I30. LCMS: [M+H]+=564.9; ¹H NMR (400 HMz, DMSO-d6) δ ppm: 9.55 (br, 1H), 7.85-7.74 (m, 2H), 7.67-7.57 (m, 3H), 6.96-6.88 (m, 2H), 4.51 (s, 1H), 3.76 (s, 3H), 3.43-3.41 (m, 2H), 3.17-3.01 (m, 4H), 2.70 (s, 2H), 2.33-2.30 (m, 1H), 2.03-1.95 (m, 4H), 1.12-1.06 (m, 4H).

### Example 31

### 2-(3-(2-(5-cyclopropyl-3-(2,6-dichlorophenyl)isoxazol-4-yl)ethyl)-3,8-diazabicyclo[3.2.1]octan-8-yl)-4-fluorobenzo[d]thiazole-6-carboxylic Acid (I31)

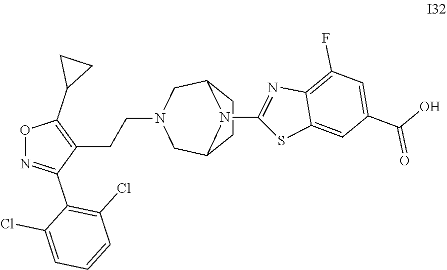 [SMILES: O=C(O)c1cc(F)c2nc(N3C4CCC3CN(CCc3c(-c5c(Cl)cccc5Cl)noc3C3CC3)C4)sc2c1]

### Step 2 Synthesis of 202

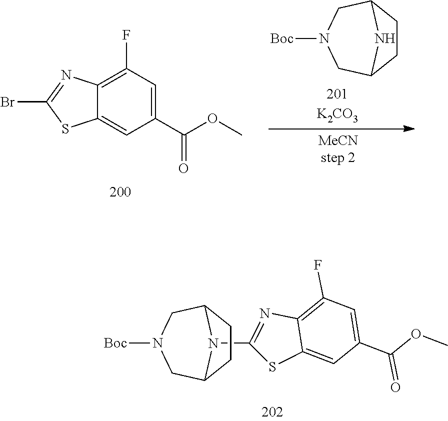 [SMILES: COC(=O)c1cc(F)c2nc(Br)sc2c1 ; CC(C)(C)OC(=O)N1CC2CCC(C1)N2 ; COC(=O)c1cc(F)c2nc(N3C4CCC3CN(C(=O)OC(C)(C)C)C4)sc2c1]

Follow the procedure of Synthesis of 29 to give 202. ¹H NMR (400 HMz, MeOD) δ 8.18 (s, 1H), 7.68 (d, J=11.5 Hz, 1H), 4.52 (s, 2H), 3.98-3.92 (m, 2H), 3.91 (s, 3H), 3.26-3.19 (m, 2H), 2.21-2.08 (m, 2H), 1.88 (d, J=7.6 Hz, 2H), 1.47 (s, 9H). LCMS: [M+H]+=422.0.

### Step 3 Synthesis of 203

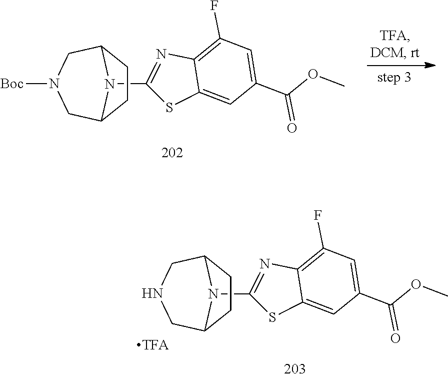 [SMILES: COC(=O)c1cc(F)c2nc(N3C4CCC3CN(C(=O)OC(C)(C)C)C4)sc2c1 ; COC(=O)c1cc(F)c2nc(N3C4CCC3CNC4)sc2c1]

Follow the procedure of Synthesis of 6 to give product compound 203 (670 mg, crude), which was used directly for the next step without further purification. LCMS: [M+H]+=322.1

### Step 4 Synthesis of 204

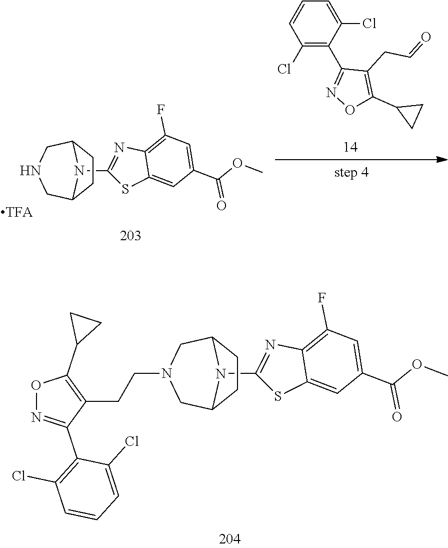 [SMILES: COC(=O)c1cc(F)c2nc(N3C4CCC3CNC4)sc2c1 ; O=CCc1c(-c2c(Cl)cccc2Cl)noc1C1CC1 ; COC(=O)c1cc(F)c2nc(N3C4CCC3CN(CCc3c(-c5c(Cl)cccc5Cl)noc3C3CC3)C4)sc2c1]

Follow the procedure of Synthesis of 15 to give 204 (302 mg, crude) used directly for the next step without further purification. LCMS: [M+H]+=602.8.

### Step 5 Synthesis of I31

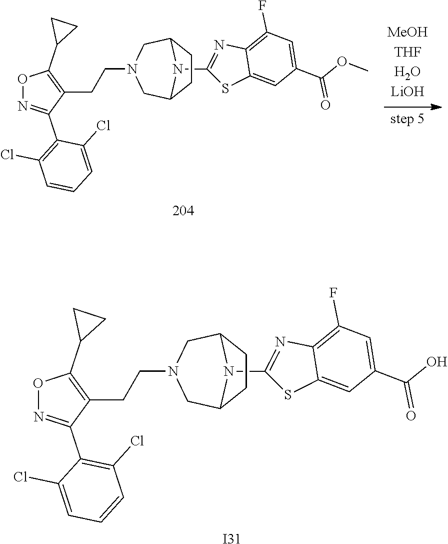

Follow the procedure of Synthesis of I1 to give 131. ¹H NMR (400 HMz, DMSO-d6) δ 13.07 (s, 0.7H), 9.77 (s, 0.5H), 8.31 (s, 1H), 7.82-7.46 (m, 4H), 4.57 (s, 3H), 3.75-3.46 (m, 1H), 3.10 (s, 1H), 2.91-2.66 (m, 3H), 2.31 (s, 2H), 2.08-1.91 (m, 5H), 1.17-0.94 (m, 4H). LCMS: [M+H]+=415.19.

### Example 32

### 2-(3-(2-(5-cyclopropyl-3-(2-(trifluoromethoxy)phenyl)isoxazol-4-yl)ethyl)-3,8-diazabicyclo[3.2.1]octan-8-yl)-4-fluorobenzo[d]thiazole-6-carboxylic Acid (I32)

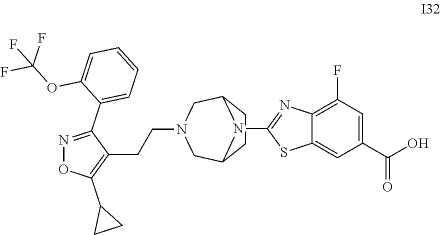 [SMILES: O=C(O)c1cc(F)c2nc(N3C4CCC3CN(CCc3c(-c5ccccc5OC(F)(F)F)noc3C3CC3)C4)sc2c1]

### Step 1 Synthesis of 204

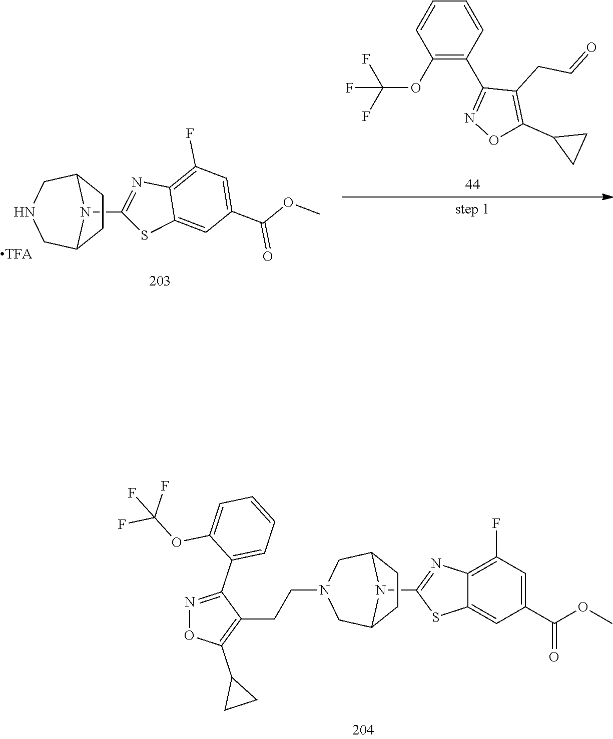 [SMILES: COC(=O)c1cc(F)c2nc(N3C4CCC3CNC4)sc2c1 ; O=CCc1c(-c2ccccc2OC(F)(F)F)noc1C1CC1 ; COC(=O)c1cc(F)c2nc(N3C4CCC3CN(CCc3c(-c5ccccc5OC(F)(F)F)noc3C3CC3)C4)sc2c1]

Follow the procedure of Synthesis of 15 to give 204 (315 mg, crude), used directly for next step. LCMS: [M+H]+=617.2.

### Step 2 Synthesis of 132

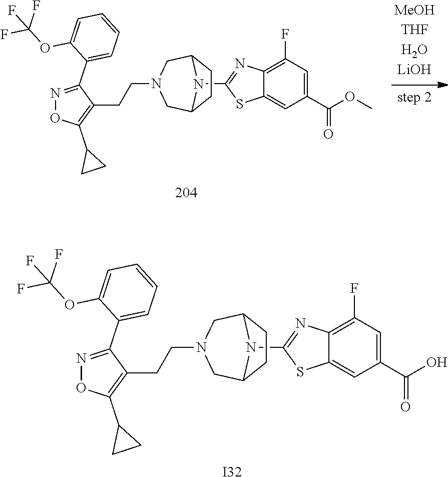 [SMILES: COC(=O)c1cc(F)c2nc(N3C4CCC3CN(CCc3c(-c5ccccc5OC(F)(F)F)noc3C3CC3)C4)sc2c1 ; "I32" ; O=C(O)c1cc(F)c2nc(N3C4CCC3CN(CCc3c(-c5ccccc5OC(F)(F)F)noc3C3CC3)C4)sc2c1]

Follow the procedure of Synthesis of I1 to give I32. ¹H NMR (400 HMz, DMSO-d6) δ 13.06 (s, 0.7H), 9.51 (s, 1H), 8.31 (s, 1H), 7.72-7.56 (m, 5H), 4.78-4.16 (m, 3H), 3.60 (s, 1H), 3.13 (s, 1H), 2.81-2.62 (m, 2H), 2.33-2.18 (m, 3H), 2.07-1.78 (m, 4H), 1.24 (s, 1H), 1.15-0.92 (m, 4H). LCMS: [M+H]+=603.1.

### Example 33

### 2-(3-(2-(5-cyclopropyl-3-(2,6-dichlorophenyl)isoxazol-4-yl)ethyl)-3,8-diazabicyclo[3.2.1]octan-8-yl)benzo[d]thiazole-6-carboxylic Acid (I33)

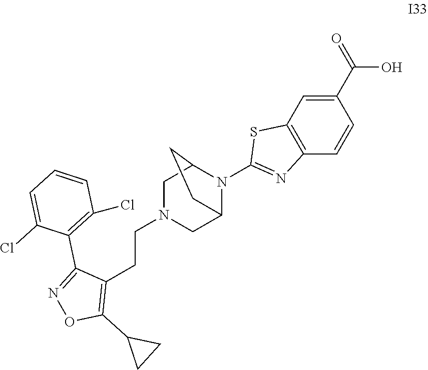 [SMILES: O=C(O)c1ccc2nc(N3C4CCC3CN(CCc3c(-c5c(Cl)cccc5Cl)noc3C3CC3)C4)sc2c1]

### Step 1: Synthesis of 206

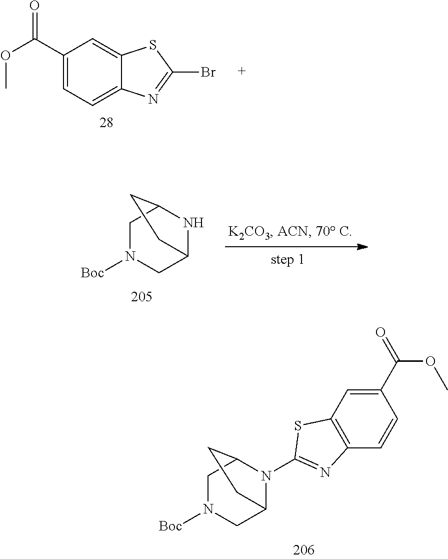 [SMILES: COC(=O)c1ccc2nc(Br)sc2c1 ; CC(C)(C)OC(=O)N1CC2CCC(C1)N2 ; COC(=O)c1ccc2nc(N3C4CCC3CN(C(=O)OC(C)(C)C)C4)sc2c1]

Follow the procedure of Synthesis of 29 to give 206. LCMS: [M+H]+=404.0; ¹H NMR (400 HMz, CDCl₃) δ ppm: 8.32 (s, 1H), 8.03-7.99 (m, 1H), 7.56-7.53 (m, 1H), 4.43 (s, 2H), 4.01-3.93 (m, 4H), 3.85-3.81 (m, 1H), 3.78-3.24 (m, 2H), 2.11 (s, 2H), 1.94-1.91 (m, 2H), 1.47 (s, 9H).

### Step 2: Synthesis of 207

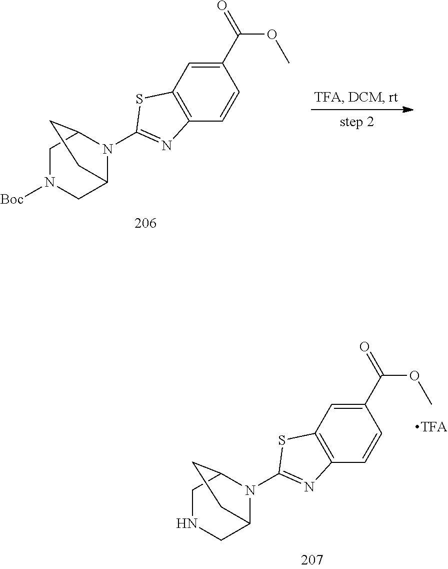 [SMILES: COC(=O)c1ccc2nc(N3C4CCC3CN(C(=O)OC(C)(C)C)C4)sc2c1 ; COC(=O)c1ccc2nc(N3C4CCC3CNC4)sc2c1]

Follow the procedure of Synthesis of 6 to give 207. LCMS: [M+H]+=304.0;

### Step 3: Synthesis of 208

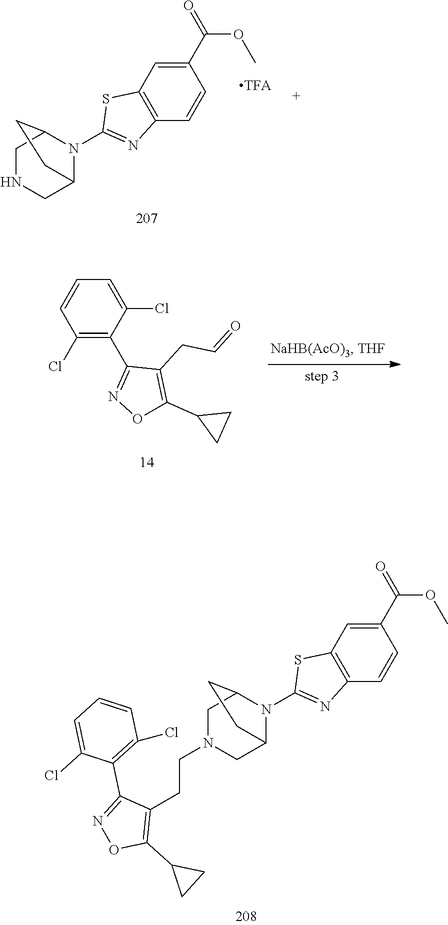 [SMILES: COC(=O)c1ccc2nc(N3C4CCC3CNC4)sc2c1 ; O=CCc1c(-c2c(Cl)cccc2Cl)noc1C1CC1 ; COC(=O)c1ccc2nc(N3C4CCC3CN(CCc3c(-c5c(Cl)cccc5Cl)noc3C3CC3)C4)sc2c1]

Follow the procedure of Synthesis of 15 to give 208. LCMS: [M+H]+=582.8; ¹H NMR (400 HMz, CDCl₃) δ ppm: 8.21 (s, 1H), 7.92-7.89 (m, 1H), 7.44-7.25 (m, 4H), 4.25 (s, 2H), 3.84 (s, 3H), 2.61-2.58 (m, 2H), 2.38-2.36 (m, 4H), 2.29-2.26 (m, 2H), 1.94-1.90 (m, 5H), 1.19-0.99 (m, 4H).

### Step 4: Synthesis of I33

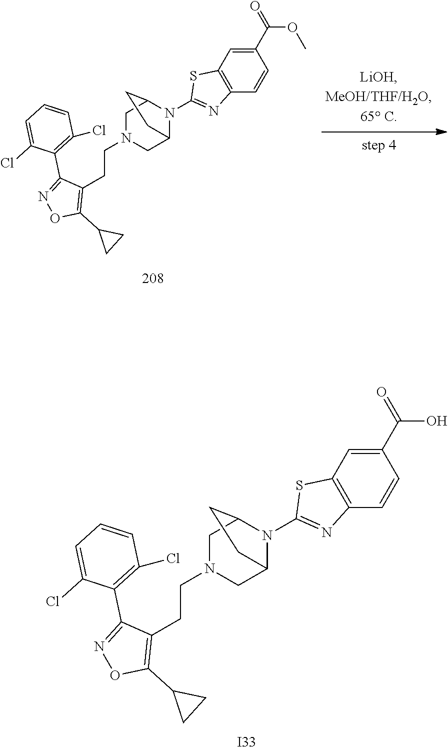 [SMILES: COC(=O)c1ccc2nc(N3C4CCC3CN(CCc3c(-c5c(Cl)cccc5Cl)noc3C3CC3)C4)sc2c1 ; "I33" ; O=C(O)c1ccc2nc(N3C4CCC3CN(CCc3c(-c5c(Cl)cccc5Cl)noc3C3CC3)C4)sc2c1]

Follow the procedure of Synthesis of I1 to give I33. LCMS: [M+H]+=568.8; ¹H NMR (400 HMz, DMSO-d6) δ ppm: 12.80 (br, 7.92-7.89 (m, 1H), 7.44-7.7.52 (m, 4H), 4.57 (s, 2H), 3.57-3.12 (m, 6H), 2.73-2.67 (m, 2H), 2.33-2.31 (m 2H), 2.07-2.01 (m, 3H), 1.13-1.07 (m, 4H).

### Example 34

### 2-(3-(2-(5-cyclopropyl-3-(2-(trifluoromethoxy)phenyl)isoxazol-4-yl)ethyl)-3,8-diazabicyclo[3.2.1]octan-8-yl)benzo[d]thiazole-6-carboxylic Acid (I34)

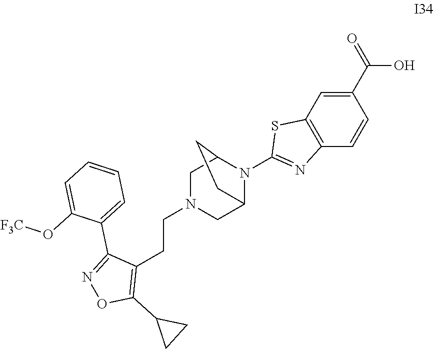 [SMILES: O=C(O)c1ccc2nc(N3C4CCC3CN(CCc3c(-c5ccccc5OC(F)(F)F)noc3C3CC3)C4)sc2c1]

### Step 3: Synthesis of 209

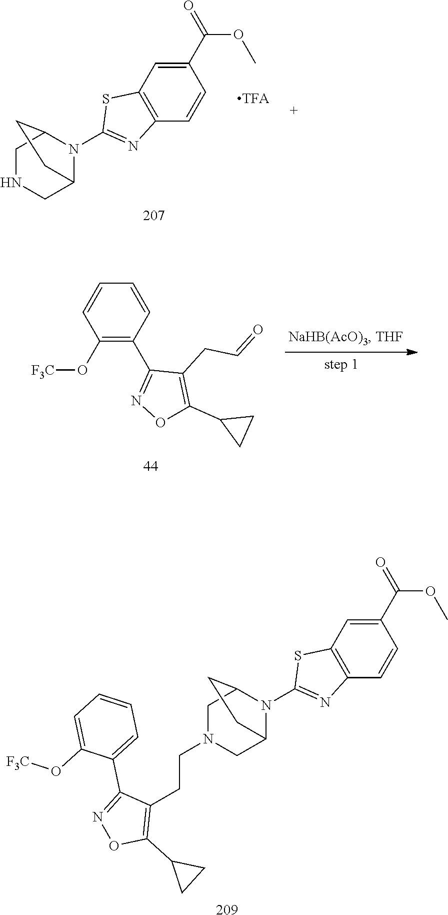

Follow the procedure of Synthesis of 15 to give 209. LCMS: [M+H]+=598.9; ¹H NMR (400 HMz, CDCl₃) δ ppm: 8.20 (s, 1H), 7.91-7.89 (m, 1H), 7.44-7.38 (m, 3H), 7.31-7.30 (m, 2H), 4.22 (s, 2H), 3.83 (s, 3H), 2.50-2.47 (m, 4H), 2.38-2.36 (m, 4H), 2.36-2.25 (m, 4H), 1.90-1.80 (m, 5H), 1.11-0.98 (m, 4H).

### Step 4: Synthesis of I34

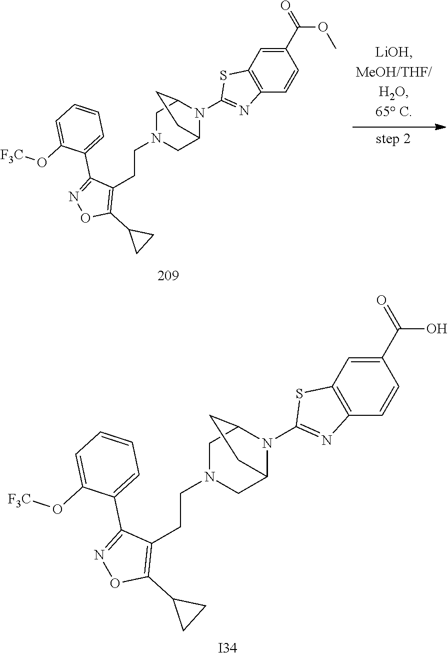

Follow the procedure of Synthesis of I1 to give I34. LCMS: [M+H]+=585.2; ¹H NMR (400 HMz, DMSO-d6) δ ppm: 12.75 (br, 1H), 8.45 (s, 1H), 7.90 (s, 1H), 7.73-7.70 (m, 1H), 7.69-7.54 (m, 4H), 4.61 (s, 2H), 3.57-3.11 (m, 5H), 2.78 (s, 2H), 2.33-2.31 (m, 2H), 2.29-2.02 (m, 4H), 1.12-1.05 (m, 4H).

### Example 35

### (R)-6-(4-(2-(5-cyclopropyl-3-(2,6-dichlorophenyl)isoxazol-4-yl)ethyl)-3-methylpiperazin-1-yl)-1-methyl-1H-indole-3-carboxylic Acid (I35)

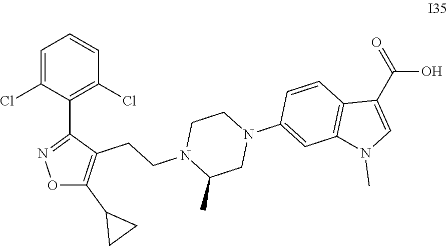 [SMILES: C[C@@H]1CN(c2ccc3c(C(=O)O)cn(C)c3c2)CCN1CCc1c(-c2c(Cl)cccc2Cl)noc1C1CC1]

LCMS: [M+H]+=553.17

### Example 36

### 6-(1-(2-(5-cyclopropyl-3-(2,6-dichlorophenyl)isoxazol-4-yl)ethyl)piperidin-4-yl)-1-methyl-1H-indole-3-carboxylic Acid (I36)

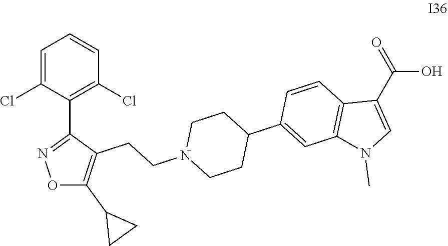 [SMILES: Cn1cc(C(=O)O)c2ccc(C3CCN(CCc4c(-c5c(Cl)cccc5Cl)noc4C4CC4)CC3)cc21]

### Step 2: Synthesis of 212

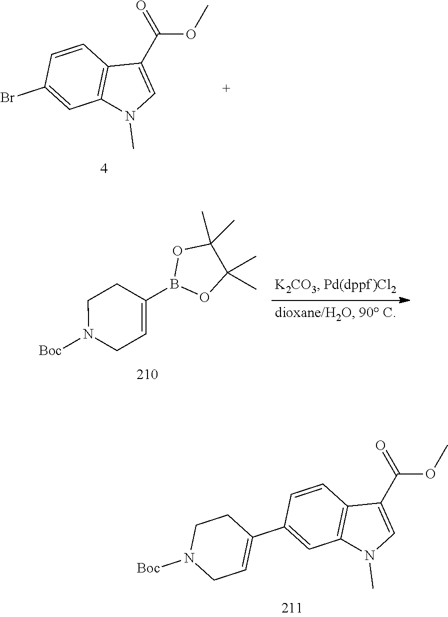 [SMILES: COC(=O)c1cn(C)c2cc(Br)ccc12 ; CC(C)(C)OC(=O)N1CC=C(B2OC(C)(C)C(C)(C)O2)CC1 ; COC(=O)c1cn(C)c2cc(C3=CCN(C(=O)OC(C)(C)C)CC3)ccc12]

Under nitrogen, a mixture of compound 4 (1.0 g, 3.7 mmol), tert-butyl-4-(4,4,5,5-tetramethyl-1,3,2-dioxaborolan-2-yl)-3,6-dihydropyridine-1(2H)-carboxylate (1.27 g, 4.1 mmol), K₂CO₃ (1.03 g, 7.4 mmol) and Pd(dppf)Cl₂ (273 mg, 0.37 mmol) were successively added to dioxane/H₂O (15/1.5 mL). Then the mixture was heated to 90° C. and stirred overnight. After completion of the reaction, the suspension was filtered, the filter cake was washed with EA. The filtrate was concentrated, dried and purified by silica gel column with PE/EA (2:1) to give 211. ¹H NMR (400 HMz, CDCl₃) δ 8.02 (d, J=8.4 Hz, 1H), 7.68 (s, 1H), 7.33 - 7.20 (m, 2H), 6.01 (s, 1H), 4.04 (s, 2H), 3.83 (s, 3H), 3.75 (s, 3H), 3.60 (t, J=5.4 Hz, 2H), 2.55 (s, 2H), 1.43 (s, 9H). LCMS: [M-56=1]+=315.1.

### Step 2: Synthesis of 212

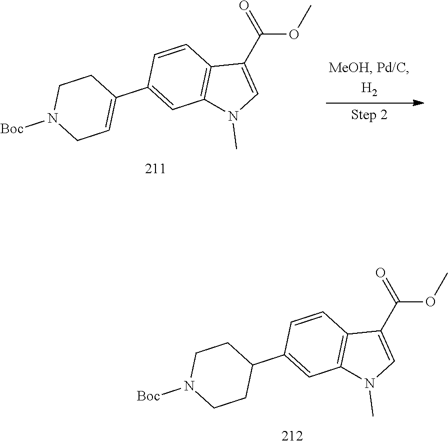 [SMILES: COC(=O)c1cn(C)c2cc(C3=CCN(C(=O)OC(C)(C)C)CC3)ccc12 ; COC(=O)c1cn(C)c2cc(C3CCN(C(=O)OC(C)(C)C)CC3)ccc12]

To a solution of compound 211 (1.1 g, 2.97 mmol) in 15 mL of MeOH, Pd/C (0.5 g) was added and the mixture was stirred at rt for 2 h under hydrogen atmosphere. After HPLC shows no starting material, the solid was removed by filtration and the filterate was concentrated to give 212 as black oil. LCMS: [M+Na]+=395.2.

### Step 3: Synthesis of 213

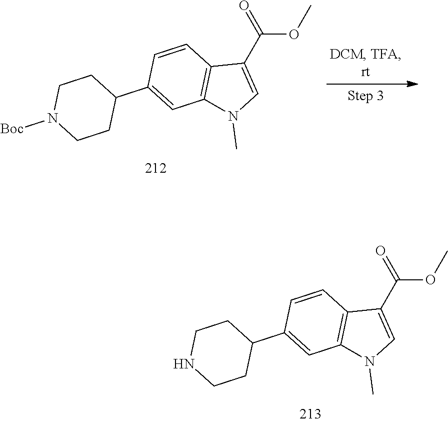 [SMILES: COC(=O)c1cn(C)c2cc(C3CCN(C(=O)OC(C)(C)C)CC3)ccc12 ; COC(=O)c1cn(C)c2cc(C3CCNCC3)ccc12]

Follow the procedure of Synthesis of 6 to give 213. LCMS: [M+H]+=273.1.

### Step 4: Synthesis of 214

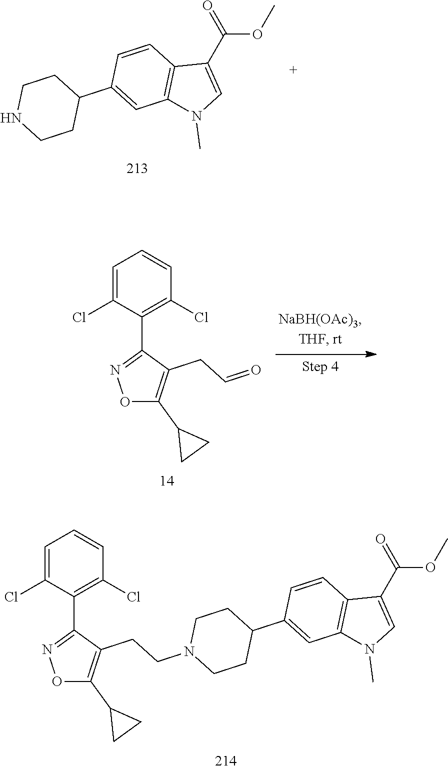 [SMILES: COC(=O)c1cn(C)c2cc(C3CCNCC3)ccc12 ; O=CCc1c(-c2c(Cl)cccc2Cl)noc1C1CC1 ; COC(=O)c1cn(C)c2cc(C3CCN(CCc4c(-c5c(Cl)cccc5Cl)noc4C4CC4)CC3)ccc12]

Follow the procedure of Synthesis of 15 to give 214 used directly for next step without further purification. ¹H NMR (400 HMz, CDCl₃) δ 7.99 (d, J=8.2 Hz, 1H), 7.66 (s, 1H), 7.42-7.26 (m, 3H), 7.16-7.00 (m, 2H), 3.82 (s, 3H), 3.72 (s, 3H), 3.06-2.91 (m, 3H), 2.54 (dt, J=8.3, 5.6 Hz, 3H), 2.49-2.39 (m, 2H), 2.01 (m, 3H), 1.88-1.71 (m, 4H), 1.17-1.15 (m, 2H), 1.05-1.03 (m, 2H). LCMS: [M+H]+=554.2.

### Step 5: Synthesis of I36

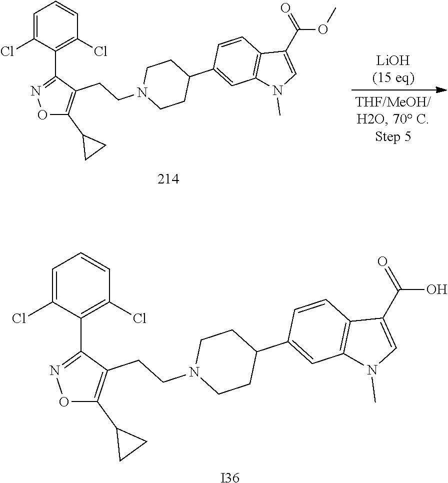 [SMILES: COC(=O)c1cn(C)c2cc(C3CCN(CCc4c(-c5c(Cl)cccc5Cl)noc4C4CC4)CC3)ccc12 ; "I36" ; Cn1cc(C(=O)O)c2ccc(C3CCN(CCc4c(-c5c(Cl)cccc5Cl)noc4C4CC4)CC3)cc21]

Follow the procedure of Synthesis of I1 to give 136. ¹H NMR (400 HMz, DMSO-d6) δ 10.38 (s, 1H), 8.00 (s, 1H), 7.95 (d, J=8.3 Hz, 1H), 7.78-7.61 (m, 3H), 7.31 (s, 1H), 7.08 (d, J=8.3 Hz, 1H), 3.83 (s, 3H), 3.56 (d, J=10.7 Hz, 3H), 3.08 (s, 4H), 2.91 (s, 1H), 2.84 (s, 2H), 2.01 (s, 4H), 1.18 (d, J=7.9 Hz, 2H), 1.13 (s, 2H). LCMS: [M+H]+=538.1.

### Example 37

### (S)-6-(4-(2-(5-cyclopropyl-3-(2,6-dichlorophenyl)isoxazol-4-yl)ethyl)-3-methylpiperazin-1-yl)-1-methyl-1H-indole-3-carboxylic Acid (I37)

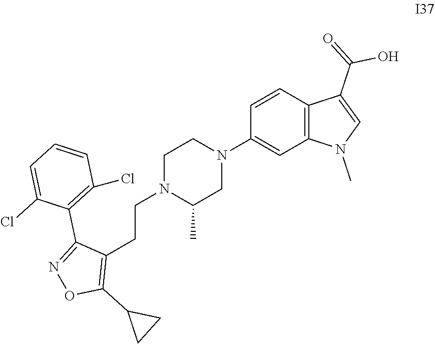 [SMILES: C[C@H]1CN(c2ccc3c(C(=O)O)cn(C)c3c2)CCN1CCc1c(-c2c(Cl)cccc2Cl)noc1C1CC1]

### Step 1: Synthesis of 216

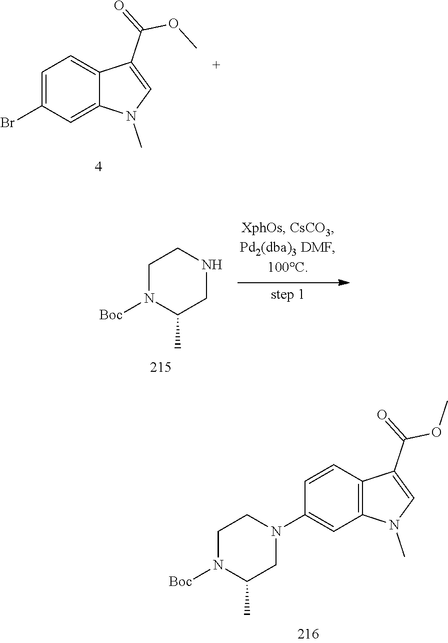 [SMILES: COC(=O)c1cn(C)c2cc(Br)ccc12 ; C[C@H]1CNCCN1C(=O)OC(C)(C)C ; COC(=O)c1cn(C)c2cc(N3CCN(C(=O)OC(C)(C)C)[C@@H](C)C3)ccc12]

Follow the procedure of Synthesis of 87 to give 216. LCMS: [M+H]+=388.2; ¹H NMR (400 HMz, CDCl₃) δ ppm: 7.95-7.93 (m, 1H), 7.57 (s, 1H), 6.91-6.88 (m, 1H), 6.66 (s, 1H), 4.30 (s, 1H), 3.92-3.89 (m, 1H), 3.80 (s, 3H), 3.68 (s, 3H), 3.45-3.33 (m, 3H), 2.88-2.85 (m, 1H), 2.73-2.66 (m, 1H), 1.42 (s, 9H), 1.29-1.27 (m, 3H).

### Step 2: Synthesis of 217

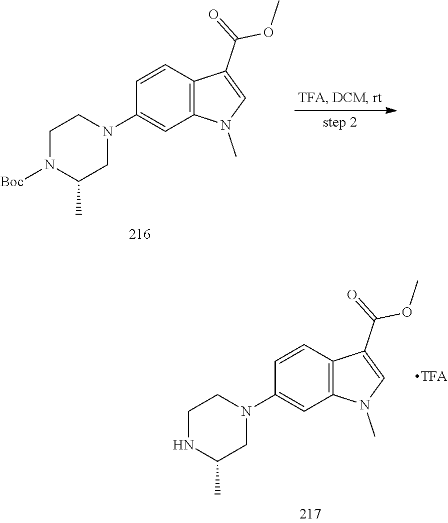 [SMILES: COC(=O)c1cn(C)c2cc(N3CCN(C(=O)OC(C)(C)C)[C@@H](C)C3)ccc12 ; COC(=O)c1cn(C)c2cc(N3CCN[C@@H](C)C3)ccc12]

Follow the procedure of Synthesis of 6 to give 217. LCMS: [M+H]+=288.2.

### Step 3: Synthesis of 218

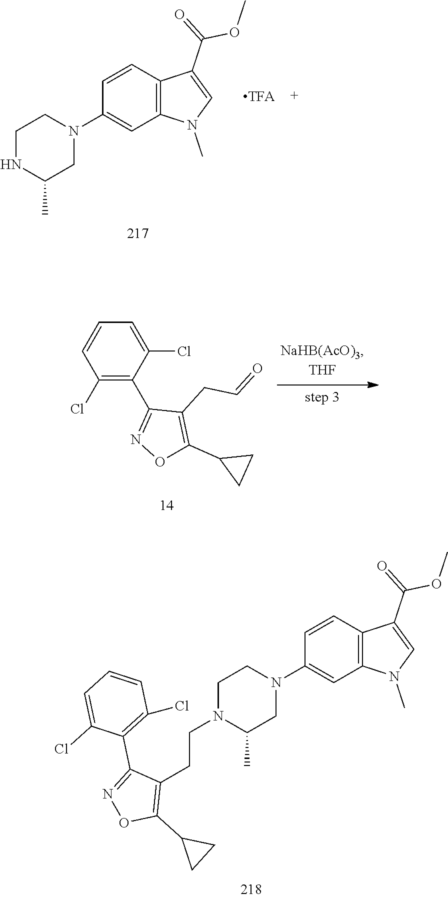 [SMILES: COC(=O)c1cn(C)c2cc(N3CCN[C@@H](C)C3)ccc12 ; O=CCc1c(-c2c(Cl)cccc2Cl)noc1C1CC1 ; COC(=O)c1cn(C)c2cc(N3CCN(CCc4c(-c5c(Cl)cccc5Cl)noc4C4CC4)[C@@H](C)C3)ccc12]

Follow the procedure of Synthesis of 15 to give 218. LCMS: [M+H]+=567.2.

### Step 4: Synthesis of 137

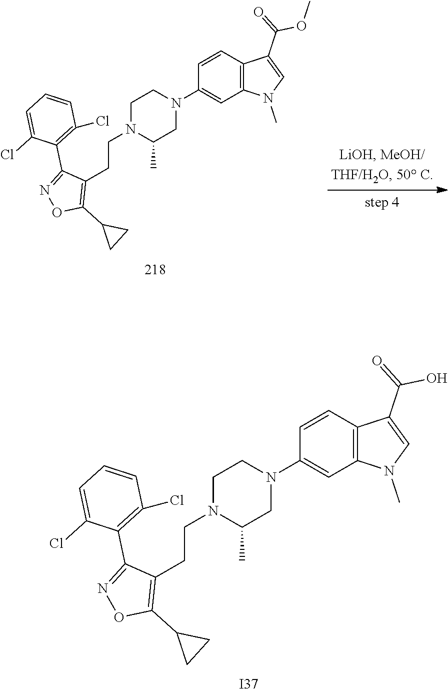 [SMILES: COC(=O)c1cn(C)c2cc(N3CCN(CCc4c(-c5c(Cl)cccc5Cl)noc4C4CC4)[C@@H](C)C3)ccc12 ; "I37" ; C[C@H]1CN(c2ccc3c(C(=O)O)cn(C)c3c2)CCN1CCc1c(-c2c(Cl)cccc2Cl)noc1C1CC1]

Follow the procedure of Synthesis of I1 to give 137. LCMS: [M+H]+=553.2; ¹H NMR (400 HMz, DMSO-d6) δ ppm: 9.75 (br, 1H), 7.89-7.84 (m, 2H), 7.74-7.63 (m, 3H), 7.01-6.99 (m, 2H), 3.86 (s, 2H), 3.78 (s, 3H), 3.73-3.70 (m, 1H), 3.48 (s, 1H), 3.31 (s, 1H), 3.20-2.93 (m, 4H), 2.78-2.62 (m, 2H), 2.50 (s, 1H), 1.23-1.19 (m, 7H).

### Example 38

### 6-(4-(2-(5-cyclopropyl-3-(2,6-dichlorophenyl)isoxazol-4-yl)ethyl)-3,3-dimethylpiperazin-1-yl)-1-methyl-1H-indole-3-carboxylic Acid (I38)

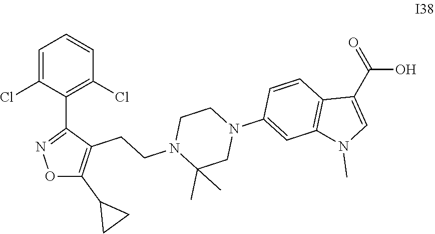 [SMILES: Cn1cc(C(=O)O)c2ccc(N3CCN(CCc4c(-c5c(Cl)cccc5Cl)noc4C4CC4)C(C)(C)C3)cc21]

LCMS: [M+H]+=567.51.

### Example 39

### (S)-6-(4-(2-(5-cyclopropyl-3-(2,6-dichlorophenyl)isoxazol-4-yl)ethyl)-2-methylpiperazin-1-yl)-1-methyl-1H-indole-3-carboxylic Acid (I39)

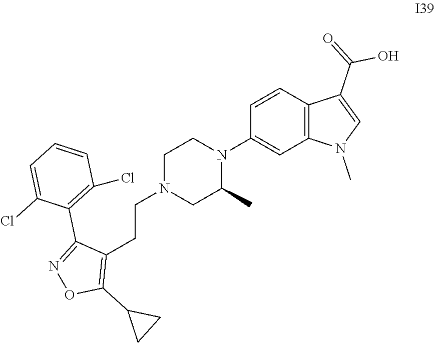 [SMILES: C[C@H]1CN(CCc2c(-c3c(Cl)cccc3Cl)noc2C2CC2)CCN1c1ccc2c(C(=O)O)cn(C)c2c1]

### Step 1: Synthesis of 220

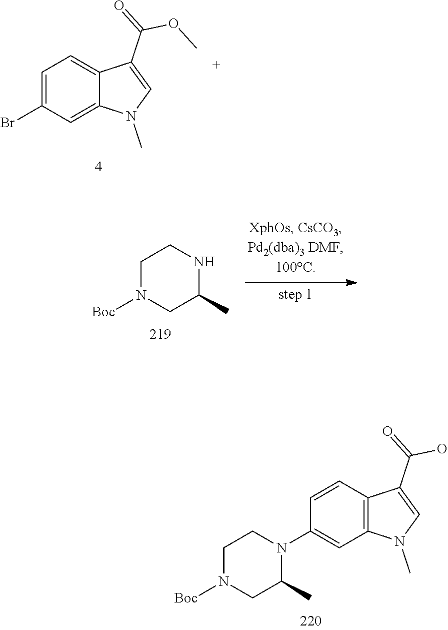 [SMILES: COC(=O)c1cn(C)c2cc(Br)ccc12 ; C[C@H]1CN(C(=O)OC(C)(C)C)CCN1 ; C[C@H]1CN(C(=O)OC(C)(C)C)CCN1c1ccc2c(C(=O)O)cn(C)c2c1]

Follow the procedure of Synthesis of 87 to give 220. LCMS: [M+H]+=388.2; ¹H NMR (400 HMz, CDCl₃) δ ppm: 7.97-7.94 (m, 1H), 7.61 (s, 1H), 6.97-6.94 (m, 1H), 6.75 (s, 1H), 3.84 (s, 3H), 3.82 (s, 3H), 3.79-3.71 (m, 2H), 3.70-3.68 (m, 2H), 3.50-3.49 (m, 1H), 3.07-3.03 (m, 2H), 1.42 (s, 9H), 0.88-0.87 (m, 3H).

### Step 2: Synthesis of 221

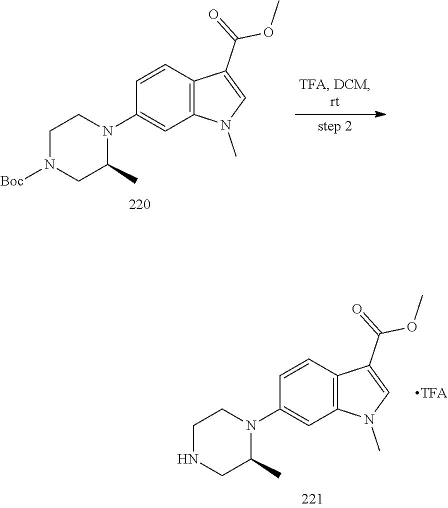 [SMILES: COC(=O)c1cn(C)c2cc(N3CCN(C(=O)OC(C)(C)C)C[C@@H]3C)ccc12 ; COC(=O)c1cn(C)c2cc(N3CCNC[C@@H]3C)ccc12]

Follow the procedure of Synthesis of 6 to give 221. LCMS: [M+H]+=288.

### Step 3: Synthesis of 222

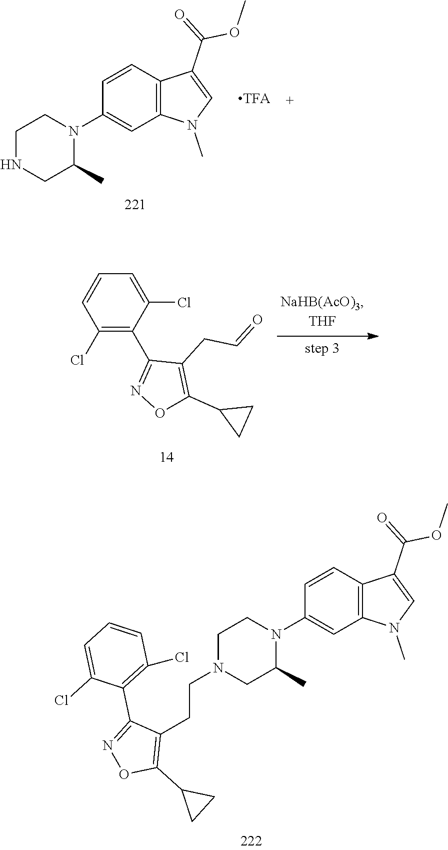 [SMILES: COC(=O)c1cn(C)c2cc(N3CCNC[C@@H]3C)ccc12 ; O=CCc1c(-c2c(Cl)cccc2Cl)noc1C1CC1 ; COC(=O)c1cn(C)c2cc(N3CCN(CCc4c(-c5c(Cl)cccc5Cl)noc4C4CC4)C[C@@H]3C)ccc12]

Follow the procedure of Synthesis of 15 to give 222. LCMS: [M+H]+=567.2;

### Step 4: Synthesis of I39

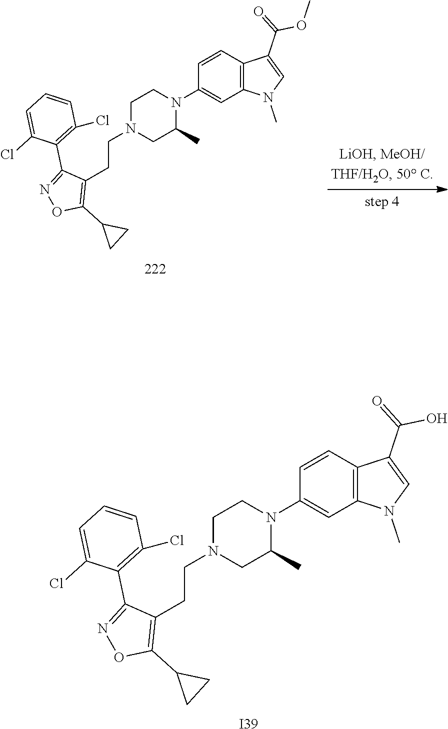 [SMILES: COC(=O)c1cn(C)c2cc(N3CCN(CCc4c(-c5c(Cl)cccc5Cl)noc4C4CC4)C[C@@H]3C)ccc12 ; "I39" ; C[C@H]1CN(CCc2c(-c3c(Cl)cccc3Cl)noc2C2CC2)CCN1c1ccc2c(C(=O)O)cn(C)c2c1]

Follow the procedure of Synthesis of I1 to give 139. LCMS: [M+H]+=553.2; ¹H NMR (400 HMz, DMSO-d6) δ ppm: 9.56 (br, 1H), 8.02-7.87 (m, 2H), 7.72-7.62 (m, 3H), 7.25-6.95 (m, 2H), 3.81 (s, 3H), 3.78 (s, 2H), 3.55-3.18 (m, 6H), 2.79 (s, 3H), 2.37 (s, 1H), 1.22-1.10 (m, 4H), 1.06-0.86 (m, 3H).

### Example 40

### (R)-6-(4-(2-(5-cyclopropyl-3-(2,6-dichlorophenyl)isoxazol-4-yl)ethyl)-2-methylpiperazin-1-yl)-1-methyl-1H-indole-3-carboxylic Acid (I40)

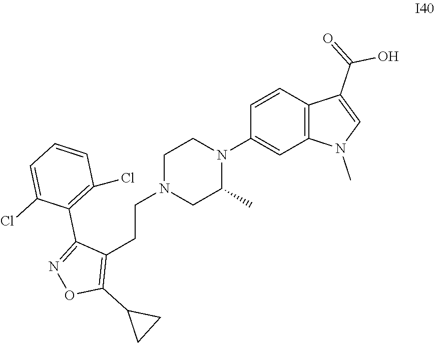 [SMILES: C[C@@H]1CN(CCc2c(-c3c(Cl)cccc3Cl)noc2C2CC2)CCN1c1ccc2c(C(=O)O)cn(C)c2c1]

### Step 1: Synthesis of 224

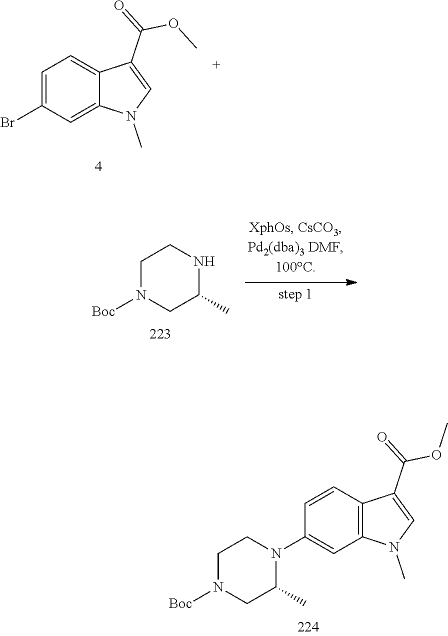 [SMILES: COC(=O)c1cn(C)c2cc(Br)ccc12 ; C[C@@H]1CN(C(=O)OC(C)(C)C)CCN1 ; COC(=O)c1cn(C)c2cc(N3CCN(C(=O)OC(C)(C)C)C[C@H]3C)ccc12]

Follow the procedure of Synthesis of 87 to give 224. LCMS: [M+H]+=388.2; ¹H NMR (400 HMz, CDCl₃) δ ppm: 7.97-7.94 (m, 1H), 7.61 (s, 1H), 6.97-6.94 (m, 1H), 6.75 (s, 1H), 3.84 (s, 3H), 3.82 (s, 3H), 3.79-3.71 (m, 2H), 3.70-3.68 (m, 2H), 3.50-3.49 (m, 1H), 3.07-3.03 (m, 2H), 1.42 (s, 9H), 0.88-0.87 (m, 3H).

### Step 2: Synthesis of 225

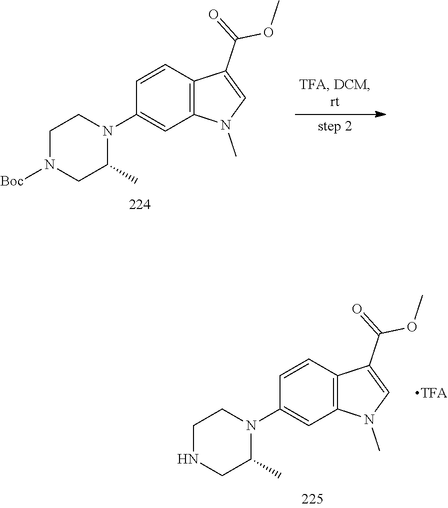 [SMILES: COC(=O)c1cn(C)c2cc(N3CCN(C(=O)OC(C)(C)C)C[C@H]3C)ccc12 ; COC(=O)c1cn(C)c2cc(N3CCNC[C@H]3C)ccc12]

Follow the procedure of Synthesis of 6 to give 225. LCMS: [M+H]+=288.2.

### Step 3: Synthesis of 226

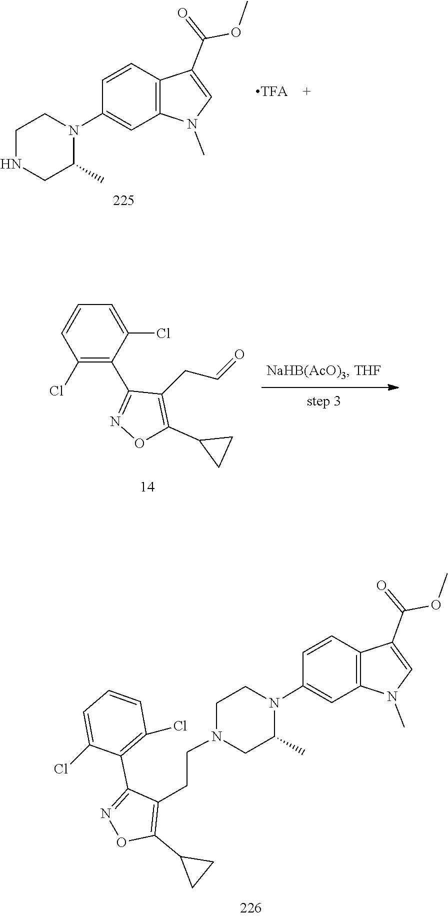 [SMILES: COC(=O)c1cn(C)c2cc(N3CCNC[C@H]3C)ccc12 ; O=CCc1c(-c2c(Cl)cccc2Cl)noc1C1CC1 ; COC(=O)c1cn(C)c2cc(N3CCN(CCc4c(-c5c(Cl)cccc5Cl)noc4C4CC4)C[C@H]3C)ccc12]

Follow the procedure of Synthesis of 15 to give 226. LCMS: [M+H]+=565.1; ¹H NMR (400 HMz, CDCl₃) δ ppm: 7.96-7.94 (m, 1H), 7.61 (s, 1H), 7.37-7.28 (m, 3H), 6.98-6.95 (m, 1H), 6.80 (s, 1H), 3.81 (s, 3H), 3.69 (s, 3H), 3.48 (s, 1H), 3.02 (s, 2H), 2.52-2.34 (m, 8H), 2.00 (s, 1H), 1.16-1.01 (m, 4H), 0.86-0.79 (m, 3H).

### Step 4: Synthesis of 140

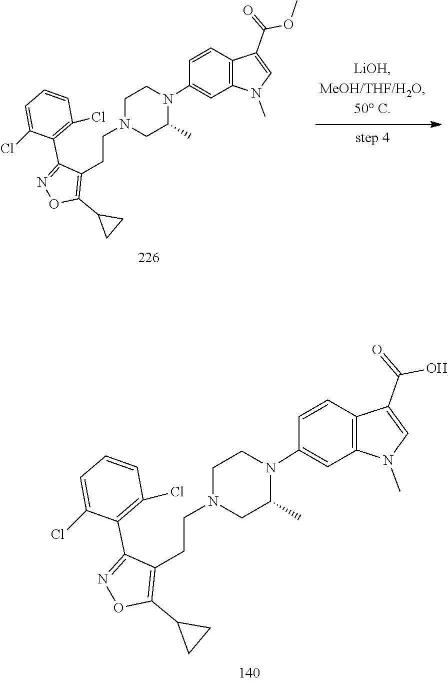 [SMILES: COC(=O)c1cn(C)c2cc(N3CCN(CCc4c(-c5c(Cl)cccc5Cl)noc4C4CC4)C[C@H]3C)ccc12 ; C[C@@H]1CN(CCc2c(-c3c(Cl)cccc3Cl)noc2C2CC2)CCN1c1ccc2c(C(=O)O)cn(C)c2c1]

Follow the procedure of Synthesis of I1 to give I40. LCMS: [M+H]+=553.2; tH NMR (400 HMz, DMSO-d6) δ ppm: 9.56 (br, 1H), 8.02-7.87 (m, 2H), 7.72-7.62 (m, 3H), 7.25-6.95 (m, 2H), 3.81 (s, 3H), 3.78 (s, 2H), 3.55-3.18 (m, 6H), 2.79 (s, 3H), 2.37 (s, 1H), 1.22-1.10 (m, 4H), 1.06-0.86 (m, 3H).

### Example 41

### 6-(8-(2-(5-cyclopropyl-3-(2,6-dichlorophenyl)isoxazol-4-yl)ethyl)-3,8-diazabicyclo[3.2.1]octan-3-yl)-1-methyl-1H-indole-3-carboxylic Acid (I41)

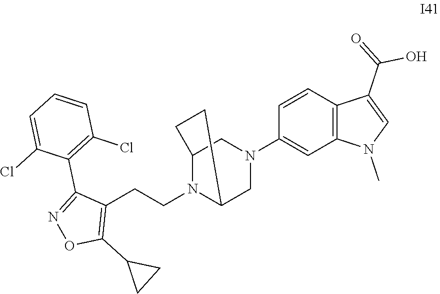 [SMILES: Cn1cc(C(=O)O)c2ccc(N3CC4CCC(C3)N4CCc3c(-c4c(Cl)cccc4Cl)noc3C3CC3)cc21]

LCMS: [M+H]+=565.5.

### Example 42

### 6-(4-(2-(5-cyclopropyl-3-(2,6-dichlorophenyl)isoxazol-4-yl)ethyl)piperazin-1-yl)-5-fluoro-1-methyl-1H-indole-3-carboxylic Acid (I42)

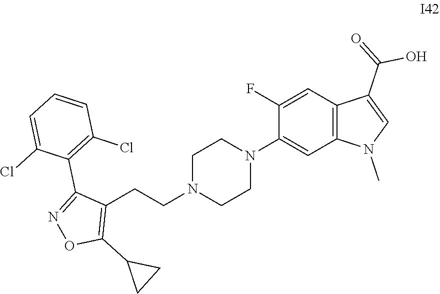 [SMILES: Cn1cc(C(=O)O)c2cc(F)c(N3CCN(CCc4c(-c5c(Cl)cccc5Cl)noc4C4CC4)CC3)cc21]

### Step 1: Synthesis of 229

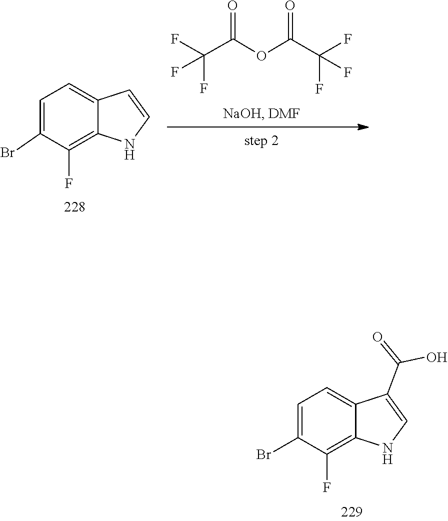 [SMILES: Fc1c(Br)ccc2cc[nH]c12 ; O=C(OC(=O)C(F)(F)F)C(F)(F)F ; O=C(O)c1c[nH]c2c(F)c(Br)ccc12]

To a solution of 228 (1.15 g, 5.4 mmol) in DMF (15 mL) was added TFAA (2.2 mL). After 2 h the reaction mixture was poured into 10% sodium bicarbonate solution (40 mL) and the precipitate was filtered, and washed with water (60 mL). The solid was dissolved in EtOAc (50 mL) and dried over Na₂SO₄, filtered and concentrated in vacuo to afford the mid product. The mid product in 5 N NaOH (35 mL) is heated at 140° C. for 1 h. The reaction mixture is allowed to cool, diluted with water (50 mL) and extracted with ether (50 mL). The aqueous layer is brought to pH=1 using concentrated HCl and extracted with EtOAc. The organic layers are washed with brine, dried and concentrated in vacuo to provide compound 229 (1.0 g, white solid crude). LCMS: [M+H]+=255.3, 257.3.

### Step 2: Synthesis of 230

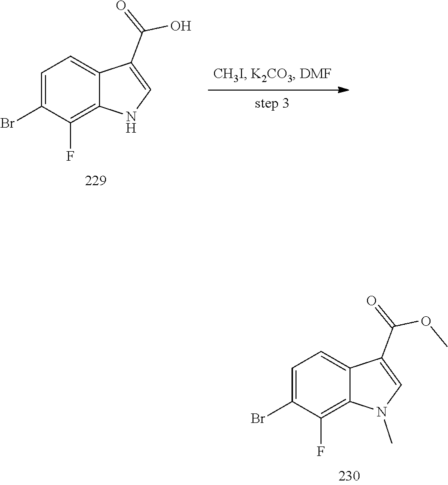 [SMILES: O=C(O)c1c[nH]c2c(F)c(Br)ccc12 ; COC(=O)c1cn(C)c2c(F)c(Br)ccc12]

To a solution of compound 229 (1.0 g, 3.88 mmol) in DMF (15 mL), K₂CO₃ (1.6 g, 11.6 mmol) was added at rt. Then the MeI (1.4 g, 9.69 mmol) was added dropwise to the mixture. The reaction mixture was stirred overnight at room temperature. After completion of the reaction, the reaction mixture was concentrated, then diluted with water, extracted with EA, and the combined organic layers were washed with brine (50 mL), dried, concentrated and purified by silica column with heptanes/EA (8:1) to give compound 230 (1.0 g, white solid, yield 89%). LCMS: [M+H]+=286.0, 288.0; ¹H NMR (300 MHz, CDCl₃) δ ppm: 7.82-7.80 (m, 1H), 7.69 (s, 1H), 7.35-7.31 (m, 1H), 4.04 (s, 3H), 3.91 (s, 3H).

### Step 3: Synthesis of 231

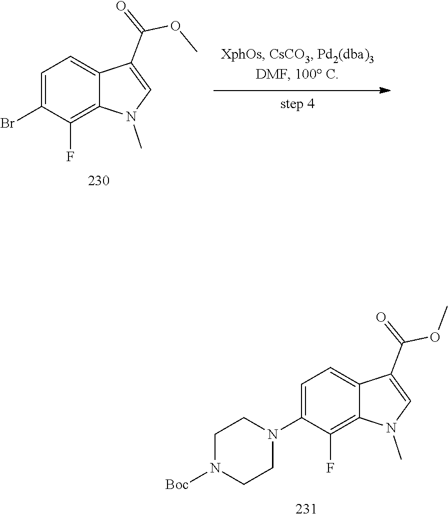 [SMILES: COC(=O)c1cn(C)c2c(F)c(Br)ccc12 ; COC(=O)c1cn(C)c2c(F)c(N3CCN(C(=O)OC(C)(C)C)CC3)ccc12]

Follow the procedure of Synthesis of 87 to give 231 (480 mg, white solid, yield 61%). LCMS: [M+H]+=292.2; ¹H NMR (300 MHz, CDCl₃) δ ppm: 7.83-7.80 (m, 1H), 7.65 (s, 1H), 6.99-6.94 (m, 1H), 4.01 (s, 3H), 3.90 (s, 3H), 3.65 (s, 4H), 3.08 (s, 4H), 1.51 (s, 9H).

### Step 4: Synthesis of 232

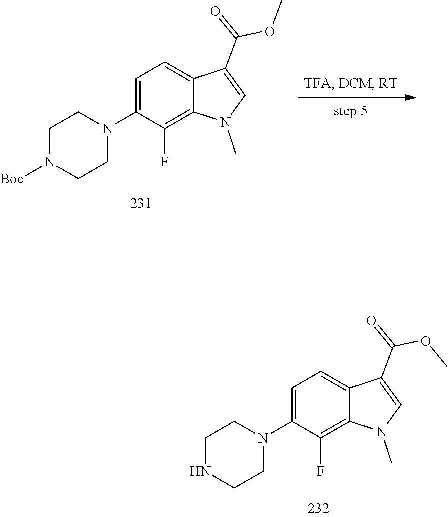 [SMILES: COC(=O)c1cn(C)c2c(F)c(N3CCN(C(=O)OC(C)(C)C)CC3)ccc12 ; COC(=O)c1cn(C)c2c(F)c(N3CCNCC3)ccc12]

Follow the procedure of Synthesis of 6 to give 232. LCMS: [M+H]+=292.1.

### Step 5: Synthesis of 233

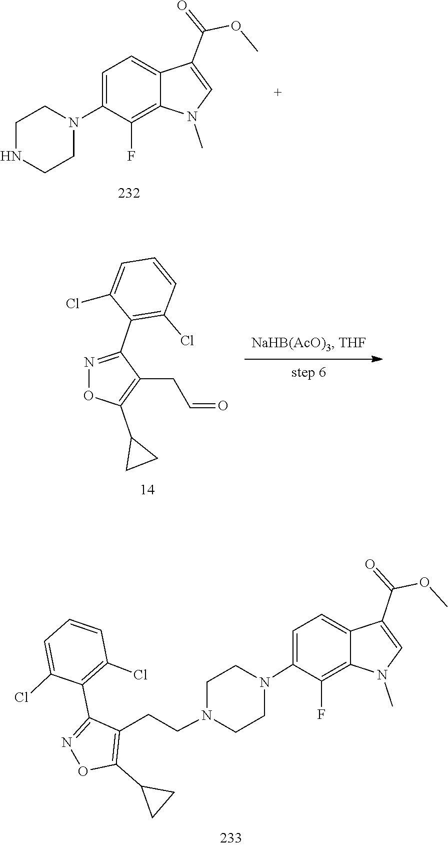 [SMILES: COC(=O)c1cn(C)c2c(F)c(N3CCNCC3)ccc12 ; O=CCc1c(-c2c(Cl)cccc2Cl)noc1C1CC1 ; COC(=O)c1cn(C)c2c(F)c(N3CCN(CCc4c(-c5c(Cl)cccc5Cl)noc4C4CC4)CC3)ccc12]

Follow the procedure of Synthesis of 15 to give 233. ¹H NMR (400 MHz, CDCl₃) δ ppm: 7.74-7.71 (m, 1H), 7.56 (s, 1H), 7.39-7.28 (m, 3H), 6.87-6.83 (m, 1H), 3.91 (s, 3H), 3.81 (s, 3H), 3.16 (s, 4H), 2.76-2.65 (m, 6H), 2.06-1.99 (m, 1H), 1.98 (s, 2H), 1.19-1.05 (m, 4H).

### Step 6: Synthesis of I42

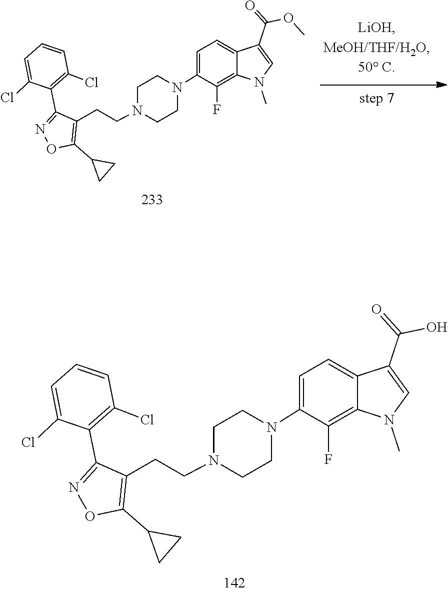 [SMILES: COC(=O)c1cn(C)c2c(F)c(N3CCN(CCc4c(-c5c(Cl)cccc5Cl)noc4C4CC4)CC3)ccc12 ; Cn1cc(C(=O)O)c2ccc(N3CCN(CCc4c(-c5c(Cl)cccc5Cl)noc4C4CC4)CC3)c(F)c21]

Follow the procedure of Synthesis of I1 to give I42. LCMS: [M+H]+=557.1; ¹H NMR (400 MHz, DMSO-d6) δ ppm: 9.75 (br, 1H), 7.99 (s, 1H), 7.73-7.62 (m, 4H), 7.02-6.98 (m, 1H), 3.97 (s, 3H), 3.63-3.60 (m, 4H), 3.45-3.21 (m, 4H), 3.08-3.05 (m, 2H), 2.78-2.74 (m, 2H), 2.40-2.36 (m, 1H), 1.20-1.13 (m, 4H).

### Example 43 and 44

### 6-((2S,6R)-4-(2-(5-cyclopropyl-3-(2,6-dichlorophenyl)isoxazol-4-yl)ethyl)-2,6-dimethylpiperazin-1-yl)-1-methyl-1H-indole-3-carboxylic Acid (I43) and 6-((2R,6R)-4-(2-(5-cyclopropyl-3-(2,6-dichlorophenyl)isoxazol-4-yl)ethyl)-2,6-dimethylpiperazin-1-yl)-1-methyl-1H-indole-3-carboxylic Acid (I44)

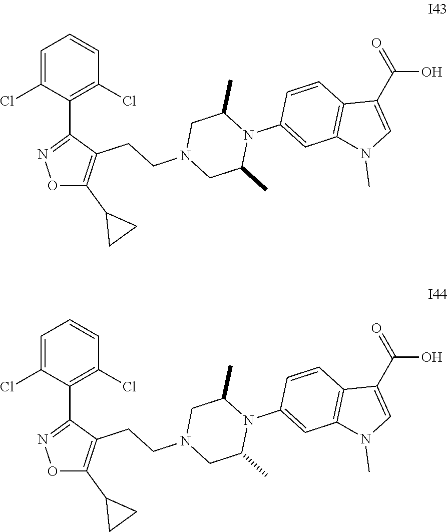 [SMILES: C[C@@H]1CN(CCc2c(-c3c(Cl)cccc3Cl)noc2C2CC2)C[C@H](C)N1c1ccc2c(C(=O)O)cn(C)c2c1 ; C[C@@H]1CN(CCc2c(-c3c(Cl)cccc3Cl)noc2C2CC2)C[C@@H](C)N1c1ccc2c(C(=O)O)cn(C)c2c1]

### Step 1: Synthesis of 235

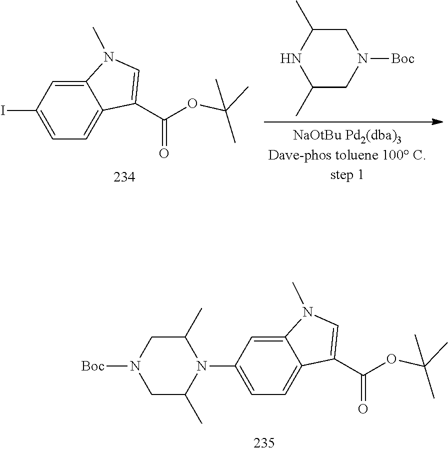 [SMILES: Cn1cc(C(=O)OC(C)(C)C)c2ccc(I)cc21 ; CC1CN(C(=O)OC(C)(C)C)CC(C)N1 ; CC1CN(C(=O)OC(C)(C)C)CC(C)N1c1ccc2c(C(=O)OC(C)(C)C)cn(C)c2c1]

Under nitrogen, a mixture of compound 234 (1 g, 4.16 mmol), tert-butyl 3,5-dimethylpiperazine-1-carboxylate (1.83 g, 5.13 mmol), NaO^{t}Bu (1.12 g, 11.65 mmol), Dave-Phos (32.9 mg, 0.09 mmol) and Pd₂(dba)₃ (43 mg, 0.047 mmol) were successively added to toluene (20 mL). Then the mixture was heated to 100° C. and stirred for 24 h. After completion of the reaction, added water, the organic phase was dried and concentrated, and purified by silica column with PE/EA (7:3) to give the desired product compound 235 (0.35 g, yellow solid, yield 28%). ¹H NMR (400 MHz, CDCl₃) δ 8.58 (s, 1H), 8.25 (d, J=8.6 Hz, 1H), 7.77 (s, 1H), 6.99 (d, J=7.2 Hz, 1H), 4.31-4.21 (m, 1H), 4.06-4.02 (m, 2H), 3.98-3.92 (m, 1H), 3.84 (s, 3H), 3.53 (s, 2H), 1.56 (s, 9H), 1.50 (s, 6H), 1.44 (s, 9H). LCMS: [M+1]+=444.2.

### Synthesis of 236

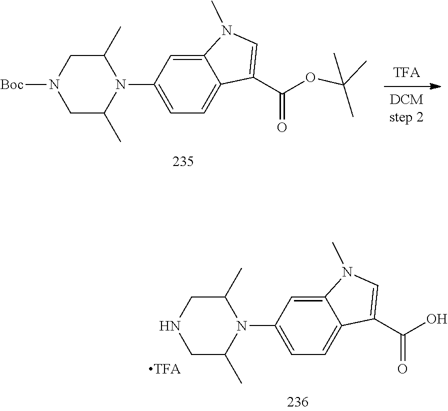 [SMILES: CC1CN(C(=O)OC(C)(C)C)CC(C)N1c1ccc2c(C(=O)OC(C)(C)C)cn(C)c2c1 ; CC1CNCC(C)N1c1ccc2c(C(=O)O)cn(C)c2c1]

Follow the procedure of Synthesis of 6 to give 236 (320 mg, crude). LCMS: [M+H]+=288.2.

### Step 4: Synthesis of I43 and I44

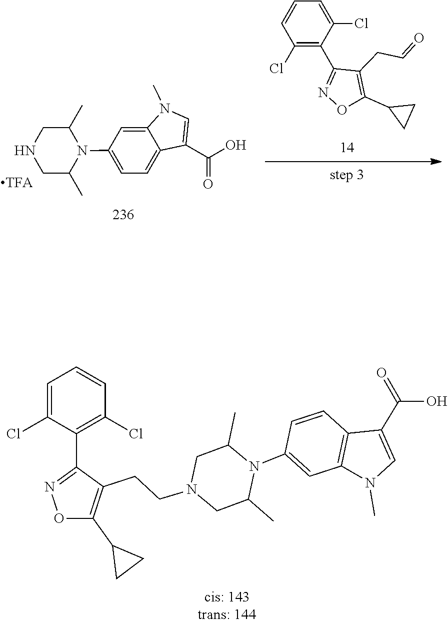 [SMILES: CC1CNCC(C)N1c1ccc2c(C(=O)O)cn(C)c2c1 ; O=CCc1c(-c2c(Cl)cccc2Cl)noc1C1CC1 ; CC1CN(CCc2c(-c3c(Cl)cccc3Cl)noc2C2CC2)CC(C)N1c1ccc2c(C(=O)O)cn(C)c2c1]

Follow the procedure of Synthesis of 15 to give 143 (49.2 mg, white solid, yield 2.66%) and I44. I43: ¹H NMR (400 MHz, DMSO-d6) δ 10.38 (s, 1H), 8.00 (s, 1H), 7.95 (d, J=8.3 Hz, 1H), 7.78-7.61 (m, 3H), 7.31 (s, 1H), 7.08 (d, J=8.3 Hz, 1H), 3.83 (s, 3H), 3.56 (d, J=10.7 Hz, 3H), 3.08 (s, 4H), 2.91 (s, 4H), 2.84 (s, 1H), 2.01 (s, 2H), 1.18 (d, J=7.9 Hz, 2H), 1.13 (s, 6H). 144: ¹H NMR (DMSO-d6) δ 9.38 (s, 1H), 7.98 (s, 1H), 7.89 (d, J=8.3 Hz, 1H), 7.73-7.71 (m, 2H), 7.66 (d, J=6.8 Hz, 1H), 7.16 (s, 1H), 6.96 (d, J=7.3 Hz, 1H), 3.81 (s, 3H), 3.74 (s, 2H), 3.57 (m, 4H), 3.19 (s, 2H), 2.81 (s, 1H), 1.19 (s, 2H), 1.13 (s, 2H), 0.96 (s, 6H). LCMS: [M+H]+=567.2.

### Example 45

### 6-(4-(2-(5-cyclopropyl-3-(2,6-dichlorophenyl)isoxazol-4-yl)ethyl)piperazin-1-yl)-5-fluoro-1-methyl-1H-indole-3-carboxylic Acid (I45)

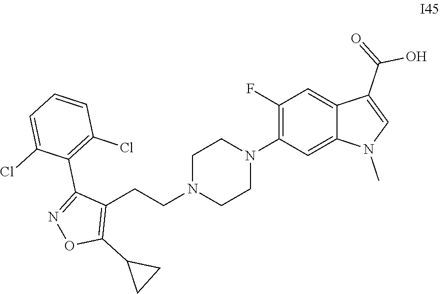 [SMILES: Cn1cc(C(=O)O)c2cc(F)c(N3CCN(CCc4c(-c5c(Cl)cccc5Cl)noc4C4CC4)CC3)cc21]

### Step 1: Synthesis of 239

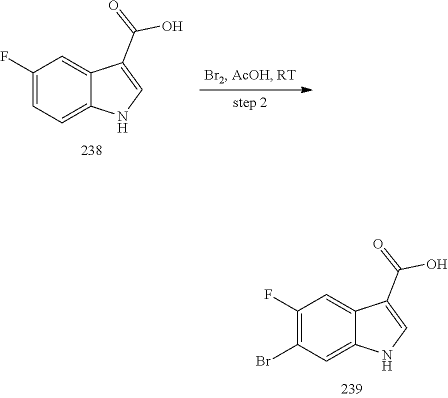 [SMILES: O=C(O)c1c[nH]c2ccc(F)cc12 ; O=C(O)c1c[nH]c2cc(Br)c(F)cc12]

Follow the procedure of Synthesis of 2 to give 239 as a solid (4.1 g) used directly in the next step. LCMS: [M+H]+=258.0, 260.0.

### Step 2: Synthesis of 240

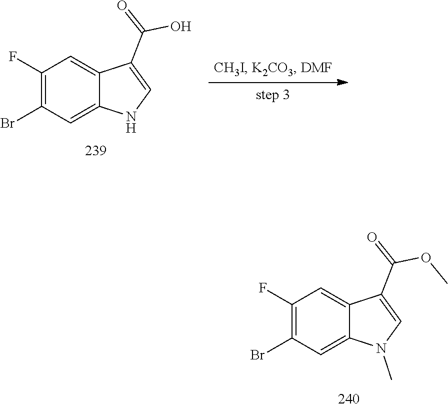 [SMILES: O=C(O)c1c[nH]c2cc(Br)c(F)cc12 ; COC(=O)c1cn(C)c2cc(Br)c(F)cc12]

Follow the procedure of Synthesis of 230 to give 240. LCMS: [M+H]+=286.0, 288.0; ¹H NMR (300 MHz, CDCl₃) δ ppm: 7.91-7.88 (m, 1H), 7.79 (s, 1H), 7.54-7.52 (m, 1H), 3.91 (s, 3H), 3.82 (s, 3H).

### Step 3: Synthesis of 241

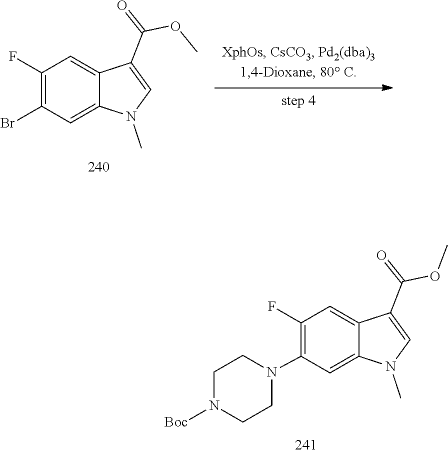 [SMILES: COC(=O)c1cn(C)c2cc(Br)c(F)cc12 ; COC(=O)c1cn(C)c2cc(N3CCN(C(=O)OC(C)(C)C)CC3)c(F)cc12]

Follow the procedure of Synthesis of 196 to give 241. LCMS: [M+H]+=392.2; ¹H NMR (400 HMz, CDCl₃) δ ppm: 7.83-7.79 (m, 1H), 7.72 (s, 1H), 6.85-6.83 (m, 1H), 3.90 (s, 3H), 3.81 (s, 3H), 3.65 (s, 4H), 3.07 (s, 4H), 1.51 (s, 9H).

### Step 4: Synthesis of 242

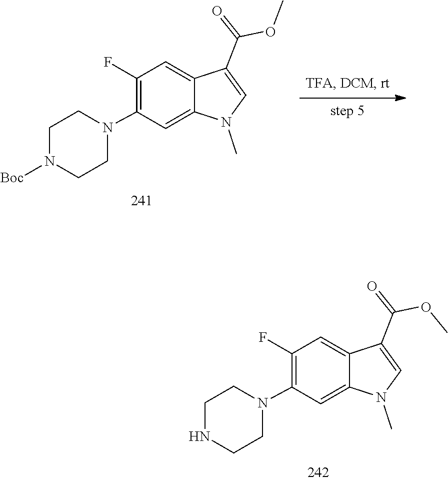 [SMILES: COC(=O)c1cn(C)c2cc(N3CCN(C(=O)OC(C)(C)C)CC3)c(F)cc12 ; COC(=O)c1cn(C)c2cc(N3CCNCC3)c(F)cc12]

Follow the procedure of Synthesis of 6 to give 242.

### Step 5: Synthesis of 243

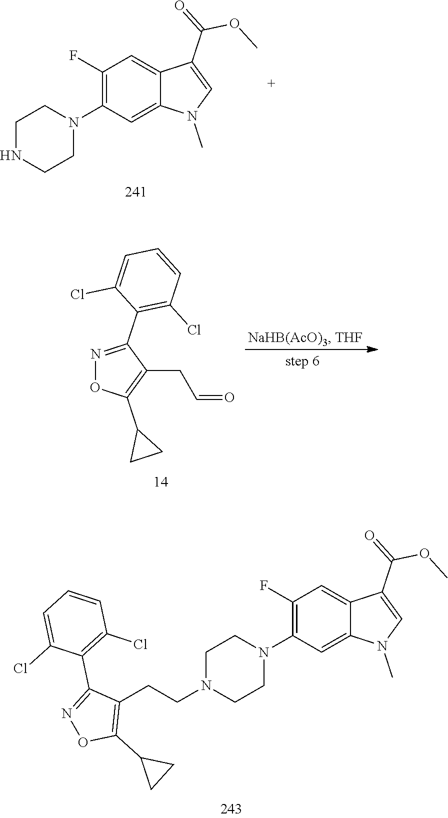 [SMILES: COC(=O)c1cn(C)c2cc(N3CCNCC3)c(F)cc12 ; O=CCc1c(-c2c(Cl)cccc2Cl)noc1C1CC1 ; COC(=O)c1cn(C)c2cc(N3CCN(CCc4c(-c5c(Cl)cccc5Cl)noc4C4CC4)CC3)c(F)cc12]

Follow the procedure of Synthesis of 15 to give compound 243. LCMS: [M+H]+=571.2;

### Step 6: Synthesis of I45

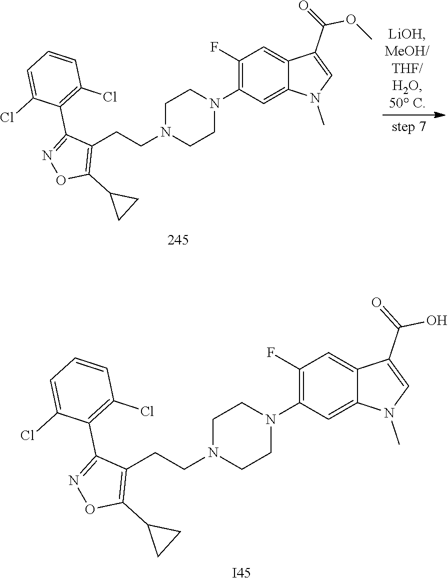 [SMILES: COC(=O)c1cn(C)c2cc(N3CCN(CCc4c(-c5c(Cl)cccc5Cl)noc4C4CC4)CC3)c(F)cc12 ; "I45" ; Cn1cc(C(=O)O)c2cc(F)c(N3CCN(CCc4c(-c5c(Cl)cccc5Cl)noc4C4CC4)CC3)cc21]

Follow the procedure of Synthesis of I1 to give I45. LCMS: [M+H]+=557.1; ¹H NMR (400 MHz, DMSO) δ ppm: 9.85 (br, 1H), 7.99 (s, 1H), 7.72-7.62 (m, 4H), 7.20-7.18 (m, 1H), 3.83 (s, 3H), 3.62-3.50 (m, 4H), 3.28-3.20 (m, 4H), 3.01 (s, 2H), 2.79-2.75 (m, 2H), 2.40-2.36 (m, 1H), 1.20-1.13 (m, 4H).

### Example 46

### 6-(4-(2-(5-cyclopropyl-3-(2,6-dichlorophenyl)isoxazol-4-yl)ethyl)piperazin-1-yl)-4-fluoro-1-methyl-1H-indole-3-carboxylic Acid (I46)

### Step 1: Synthesis of I46-4

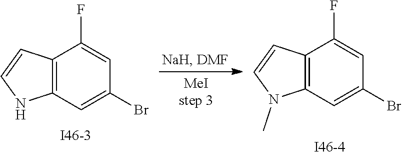

To a solution of compound I46-3 (50 mg, 0.224 mmol) in DMF (2 mL) was added NaH (18 mg, 0.448 mmol) port wise at 0° C. and stirred at rt for 1 h. MeI (63 mg, 0.448 mmol) was added to. The resulting mixture was stirred at rt for 3 h. The reaction was diluted with H₂O (50 mL) and extracted with EA. The combined organic layers were washed with brine, dried over Na₂SO₄, filtered and concentrated. The residue was purified by flash chromatography on silica gel (PE/EA=6/1) to afford compound I46-4 (45 mg, 85% yield) as a white solid.

### Step 2: Synthesis of I46-5

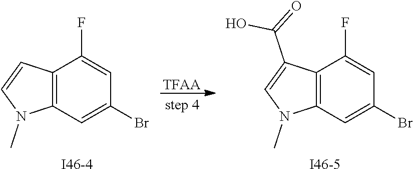

Follow the procedure of Synthesis of 229 to give I46-5 as a white solid. LCMS: [M−H]+=270.

### Step 3: Synthesis of I46-6

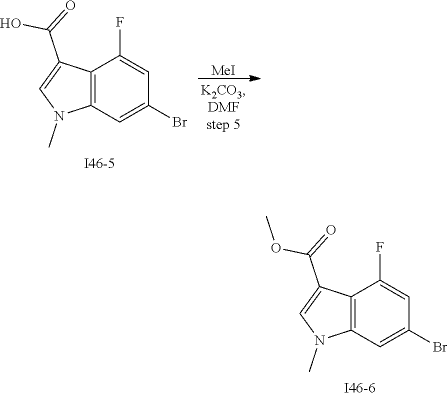 [SMILES: "MeI" ; "I46-5" ; Cn1cc(C(=O)O)c2c(F)cc(Br)cc21 ; "I46-6" ; COC(=O)c1cn(C)c2cc(Br)cc(F)c12]

To a solution of compound I46-5 (300 mg, 1.1 mmol) in DMF (2 mL) was added Cs₂CO₃ (1.06 g, 3.3 mmol) and stirred at rt for 0.5 hour. MeI (468 mg, 3.3 mmol) was added to. The resulting mixture was stirred at rt for 3 h. The reaction was diluted with H₂O (60 mL) and extracted with EA. The combined organic layers were washed with brine, dried over Na₂SO₄, filtered and concentrated. The residue was purified by flash chromatography on silica gel (PE/EA=5/1) to afford compound I46-6 (200 mg, 64% yield) as a white solid.

### Step 4: Synthesis of I46-7

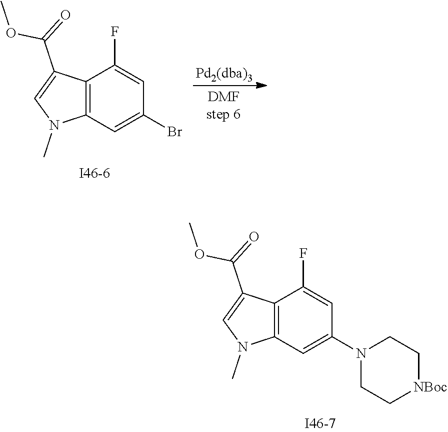 [SMILES: "I46-6" ; COC(=O)c1cn(C)c2cc(Br)cc(F)c12 ; COC(=O)c1cn(C)c2cc(N3CCN(C(=O)OC(C)(C)C)CC3)cc(F)c12]

To a solution of compound I46-6 (100 mg, 0.35 mmol) in DMF (10 mL) was added tert-butyl piperazine-1-carboxylate (195 mg, 1.05 mmol), Cs₂CO₃ (340 mg, 1.05 mmol), Pd₂(dba)₃ (50 mg) and t-Buxphos (50 mg). The reaction mixture was heated to 120° C. under N₂ for 3 hours. The reaction was cooled to rt, diluted with H₂O (100 mL) and extracted with EA. The combined organic layers were washed with brine (100 mL×2), dried over Na₂SO₄, filtered and concentrated. The residue was purified by flash chromatography on silica gel (PE/EA=1/1) to afford compound I46-7 as a brown oil. LCMS: [M+H]+=392.0.

### Step 5: Synthesis of I46-8

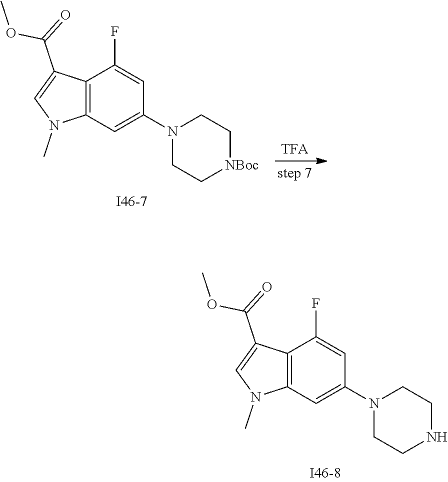 [SMILES: COC(=O)c1cn(C)c2cc(N3CCN(C(=O)OC(C)(C)C)CC3)cc(F)c12 ; COC(=O)c1cn(C)c2cc(N3CCNCC3)cc(F)c12]

Follow the procedure of Synthesis of 6 to give I46-8 as a dark solid.

### Step 6: Synthesis of I46-9

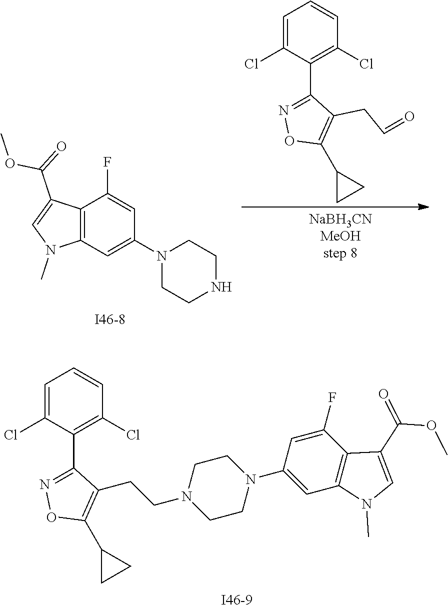 [SMILES: COC(=O)c1cn(C)c2cc(N3CCNCC3)cc(F)c12 ; O=CCc1c(-c2c(Cl)cccc2Cl)noc1C1CC1 ; "I46-8" ; COC(=O)c1cn(C)c2cc(N3CCN(CCc4c(-c5c(Cl)cccc5Cl)noc4C4CC4)CC3)cc(F)c12 ; "I46-9"]

To a solution of I46-8 (30 mg, 1.034 mmol) in MeOH (1 mL) was added compound 8 (46 mg, 1.55 mmol) and CH₃COOH (13 mg, 0.207 mmol). The reaction mixture was stirred at rt for 3 h. NaBH₃CN (13 mg, 0.207 mmol) was added and the resulting mixture was stirred at rt for 2 h. The reaction was diluted with H₂O (100 mL) and extracted with EA. The combined organic layers were washed with brine, dried over Na₂SO₄, filtered and concentrated. The residue was purified by flash chromatography on silica gel (PE/EA=1/2) to afford compound I46-9 (40 mg, 59% yield) as a white solid.

### Step 7: Synthesis of I46

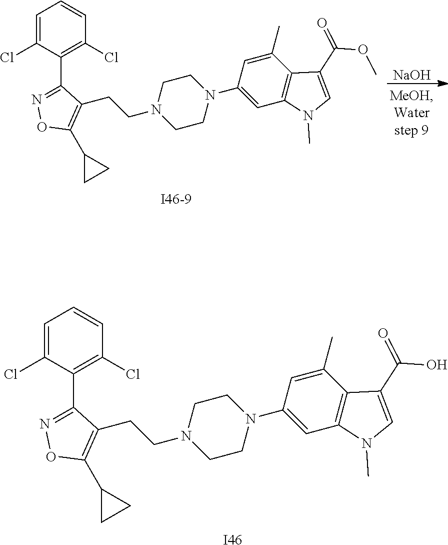

To a solution of compound I46-9 (40 mg, 0.070 mmol) in MeOH (1 mL) and water (1 mL) was added NaOH (28 mg, 0.70 mmol). The reaction mixture was stirred at rt for 3 h. The reaction was diluted with H₂O (100 mL), adjusted pH to 7 with 5% HCl and extracted with EA. The combined organic layers were washed with brine (50 mL), dried over Na₂SO₄, filtered and concentrated, purified by pre-HPLC to afford 146 as a white solid. LC-MS: [M+H]+=557.1. ¹H NMR (400 MHz, DMSO-d6) δ ppm: 7.68 (s, 1H), 7.48-7.39 (m, 3H), 6.66-6.51 (m, 2H), 3.65 (s, 3H), 3.06 (s, 4H), 2.65-2.45 (m, 6H), 2.38-2.34 (m, 2H), 2.14-2.09 (m, 1H), 1.07-1.03 (m, 4H).

### Example 47

### 6-(1-(2-(5-cyclopropyl-3-(2,6-dichlorophenyl)isoxazol-4-yl)ethyl)-4-hydroxypiperidin-4-yl)-1-methyl-1H-indole-3-carboxylic Acid (I47)

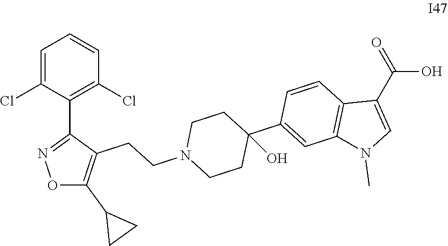 [SMILES: Cn1cc(C(=O)O)c2ccc(C3(O)CCN(CCc4c(-c5c(Cl)cccc5Cl)noc4C4CC4)CC3)cc21]

### Example 48

### 6-(5-(2-(5-cyclopropyl-3-(2,6-dichlorophenyl)isoxazol-4-yl)ethyl)-2,5-diazabicyclo[2.2.1]heptan-2-yl)-1-methyl-1H-indole-3-carboxylic Acid (I48)

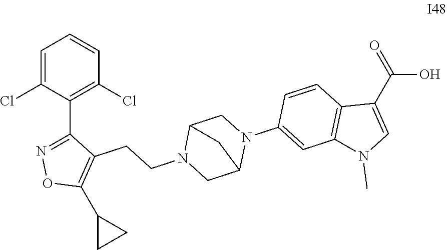 [SMILES: Cn1cc(C(=O)O)c2ccc(N3CC4CC3CN4CCc3c(-c4c(Cl)cccc4Cl)noc3C3CC3)cc21]

### Step 1: Synthesis of I48-3

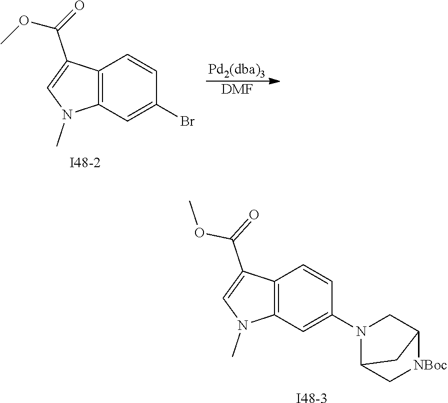

Follow the procedure of Synthesis of I46-7 to give I48-3 as a yellow solid.

MS Calcd.: 385; MS Found: 386[M+H]⁺.

### Step 3: Synthesis of I48-4

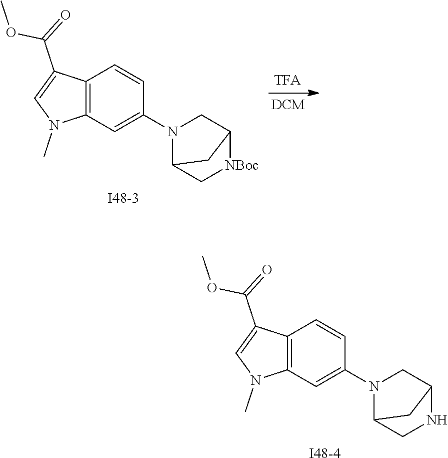 [SMILES: "I48-3" ; COC(=O)c1cn(C)c2cc(N3CC4CC3CN4C(=O)OC(C)(C)C)ccc12 ; "I48-4" ; COC(=O)c1cn(C)c2cc(N3CC4CC3CN4)ccc12]

Follow the procedure of Synthesis of 6 to give I48-4 as a dark oil.

### Step 4: Synthesis of I48-5

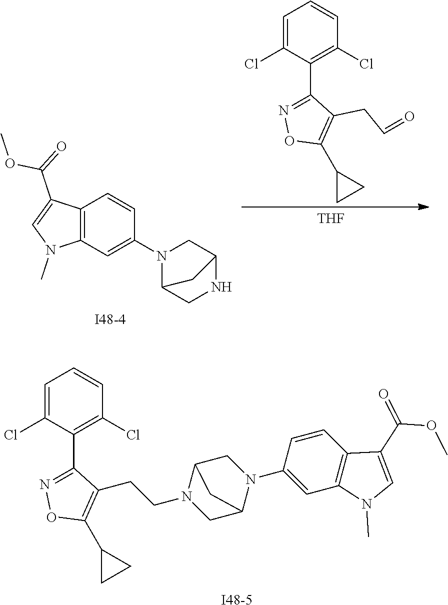 [SMILES: "I48-4" ; "I48-5" ; COC(=O)c1cn(C)c2cc(N3CC4CC3CN4)ccc12 ; COC(=O)c1cn(C)c2cc(N3CC4CC3CN4CCc3c(-c4c(Cl)cccc4Cl)noc3C3CC3)ccc12 ; O=CCc1c(-c2c(Cl)cccc2Cl)noc1C1CC1 ; "THF"]

Follow the procedure of Synthesis of I46-9 to give I48-5 as a yellow solid.

### Step 5: Synthesis of I48

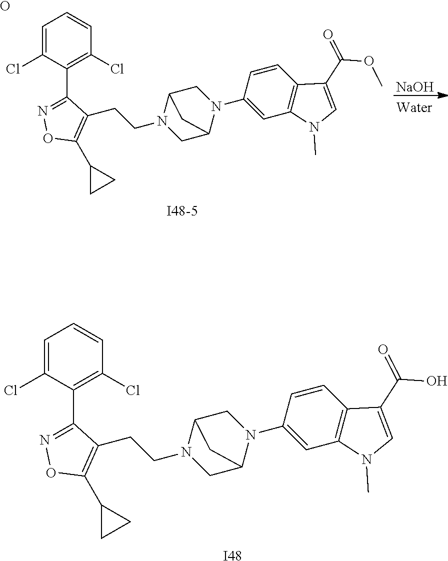

Follow the procedure of Synthesis of I-46 to give 148 as a white solid.

¹H NMR (400 MHz, DMSO-d6) δ: 11.67 (s, 1H), 7.78-7.74 (m, 2H), 7.56 (d, J=8.0 Hz, 1H), 7.22 (t, J=7.6 Hz, 1H), 7.05 (d, J=7.6 Hz, 1H), 6.51 (d, J=7.6 Hz, 1H), 6.44 (s, 1H), 4.24 (s, 1H), 3.74 (s, 3H), 3.40 (s, 1H), 3.31 (s, 2H), 3.07 (s, 1H), 2.67 (s, 1H), 2.43-2.38 (m, 2H), 2.28-2.18 (m, 2H), 2.11-2.07 (m, 1H), 1.74 (s, 2H), 1.05-0.99 (m, 4H). MS Calcd.: 551; MS Found: 552 [M+H]+.

### Example 49

### 6-(6-(2-(5-cyclopropyl-3-(2,6-dichlorophenyl)isoxazol-4-yl)ethyl)-3,6-diazabicyclo[3.1.1]heptan-3-yl)-1-methyl-1H-indole-3-carboxylic Acid (I49)

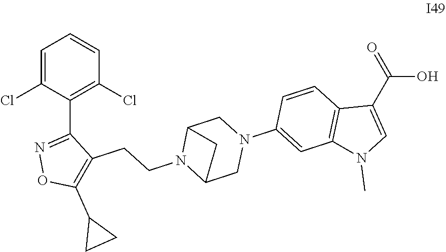 [SMILES: Cn1cc(C(=O)O)c2ccc(N3CC4CC(C3)N4CCc3c(-c4c(Cl)cccc4Cl)noc3C3CC3)cc21]

### Step 1: Synthesis of I49-2

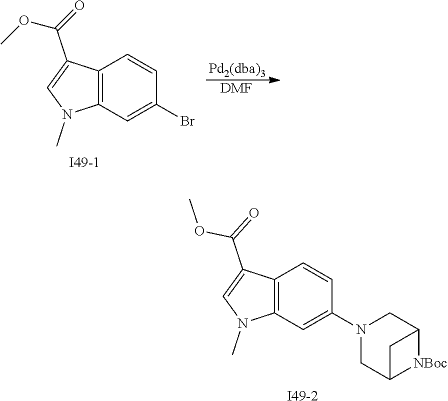

Follow the procedure of Synthesis of I46-7 to give I49-2 as a yellow solid. MS Calcd.: 385; MS Found: 386[M+H]⁺.

### Step 2: Synthesis of I49-3

Follow the procedure of Synthesis of 6 to give I49-3 as a dark oil.

MS Calcd.: 285; MS Found: 286[M+H]⁺.

### Step 3: Synthesis of I49-4

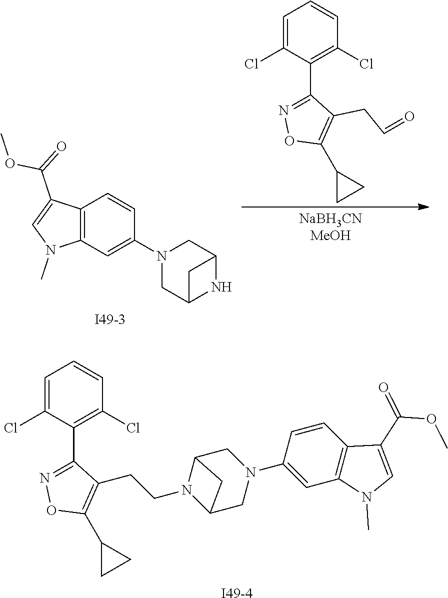 [SMILES: "I49-3" ; COC(=O)c1cn(C)c2cc(N3CC4CC(C3)N4)ccc12 ; O=CCc1c(-c2c(Cl)cccc2Cl)noc1C1CC1 ; "I49-4" ; COC(=O)c1cn(C)c2cc(N3CC4CC(C3)N4CCc3c(-c4c(Cl)cccc4Cl)noc3C3CC3)ccc12]

Follow the procedure of Synthesis of I46-9 to give I49-4 as a yellow solid.

### Step 4: Synthesis of I49

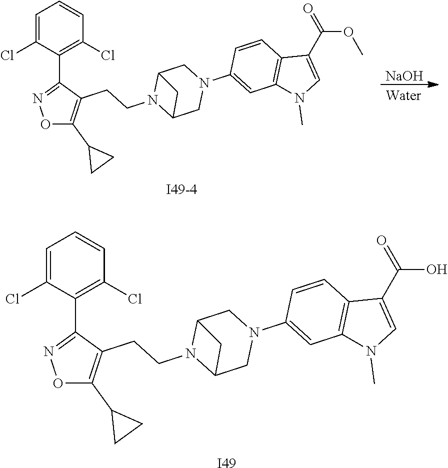

Follow the procedure of Synthesis of I46 to give I49 as a white solid.

¹H NMR (DMSO-d6) δ: 7.85 (d, J=9.2 Hz, 1H), 7.79 (s, 1H), 7.39-7.36 (m, 2H), 7.34-7.31 (m, 1H), 6.68-6.66 (m, 1H), 6.54-6.52 (m, 1H), 3.77 (s, 3H), 3.70 (s, 1H), 2.67 (s, 1H), 2.45-2.28 (m, 4H), 2.26-2.18 (m, 4H), 1.51 (s, 1H), 1.24 (s, 1H), 1.04-0.94 (m, 5H). MS Calcd.: 551; MS Found: 552 [M+H]⁺.

### Example 50

### 6-(3-(2-(5-cyclopropyl-3-(2,6-dichlorophenyl)isoxazol-4-yl)ethyl)-3,6-diazabicyclo[3.1.1]heptan-6-yl)-1-methyl-1H-indole-3-carboxylic Acid (I50)

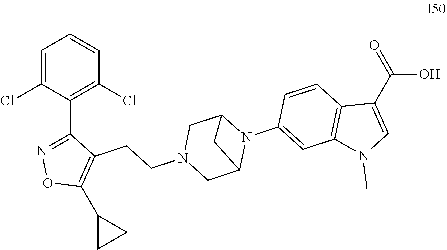 [SMILES: Cn1cc(C(=O)O)c2ccc(N3C4CC3CN(CCc3c(-c5c(Cl)cccc5Cl)noc3C3CC3)C4)cc21]

### Example 51

### 6-(4-(2-(5-cyclopropyl-3-(2,6-dichlorophenyl)isoxazol-4-yl)ethyl)-2-methylpiperazin-1-yl)quinoline-2-carboxylic Acid (I51)

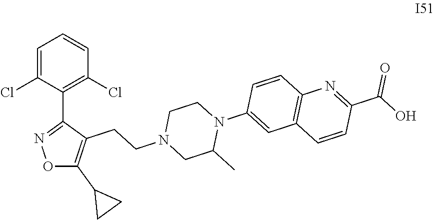 [SMILES: CC1CN(CCc2c(-c3c(Cl)cccc3Cl)noc2C2CC2)CCN1c1ccc2nc(C(=O)O)ccc2c1]

### Step 1: Synthesis of I51-15

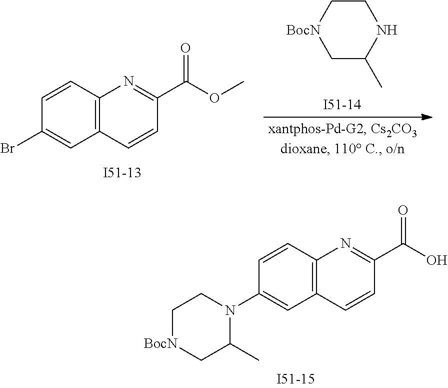 [SMILES: COC(=O)c1ccc2cc(Br)ccc2n1 ; CC1CN(C(=O)OC(C)(C)C)CCN1 ; CC1CN(C(=O)OC(C)(C)C)CCN1c1ccc2nc(C(=O)O)ccc2c1]

To a solution of compound I51-13 (same as Compound 49, 760.0 mg, 2.86 mmol) in dioxane (20 mL) was added compound I51-14 (1.14 g, 5.71 mmol), Cs₂CO₃ (2.79 g, 8.57 mmol) and xantphos-Pd-G2 (254.0 mg, 0.29 mmol). The reaction mixture was stirred at 110° C. overnight. The mixture was completed detected by LCMS. The reaction mixture was diluted with H₂O (30 mL) and extracted with EA. The combined organic layers were washed with brine, dried over Na₂SO₄, filtered and concentrated to remove the solution. The residue was purified by chromatography on silica gel (DCM:MeOH=50:1) to give compound I51-15 (420.0 mg, 39.6% yield) as a yellow solid. MS Calcd.: 371.4; MS Found: 372.5 [M+H]+.

### Step 2: Synthesis of I51-16

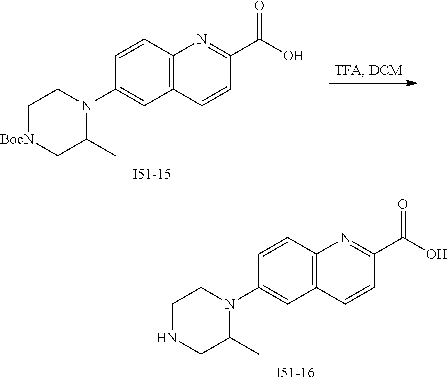 [SMILES: CC1CN(C(=O)OC(C)(C)C)CCN1c1ccc2nc(C(=O)O)ccc2c1 ; CC1CNCCN1c1ccc2nc(C(=O)O)ccc2c1]

Follow the procedure of Synthesis of 6 to give compound I51-16 as a yellow solid. The crude was used into the following reaction without the further purification.

### Step 3: Synthesis of I51

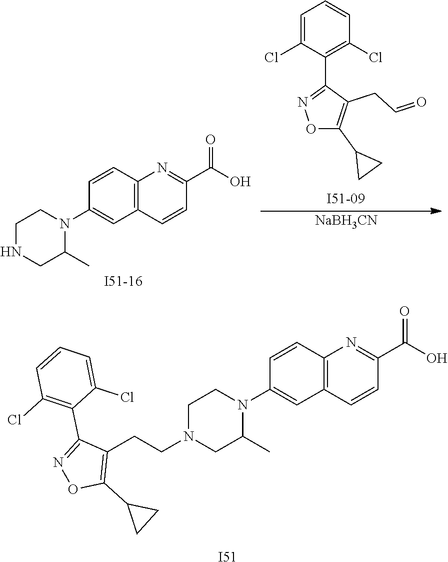 [SMILES: CC1CNCCN1c1ccc2nc(C(=O)O)ccc2c1 ; O=CCc1c(-c2c(Cl)cccc2Cl)noc1C1CC1 ; "NaBH3CN" ; "I51" ; CC1CN(CCc2c(-c3c(Cl)cccc3Cl)noc2C2CC2)CCN1c1ccc2nc(C(=O)O)ccc2c1]

Follow the procedure of Synthesis of I46-9 to give compound I51 as a yellow solid. ¹H NMR (400 MHz, CD₃OD) δ: 8.37 (d, J=6.4 Hz, 1H), 8.16 (d, J=8.8 Hz, 2H), 7.73 (dd, J=2.4, 9.6 Hz, 1H), 7.64-7.55 (m, 3H), 7.38 (brs, 1H), 4.49 (brs, 1H), 3.66-3.54 (m, 2H), 3.51-3.46 (m, 3H), 3.32-3.24 (overlap, 3H), 2.98-2.88 (m, 2H), 2.33-2.26 (m, 1H), 1.28-1.22 (m, 7H). MS Found: 552.1 [M+H]⁺.

### Example 52 and 53

### 6-(4-(2-(5-cyclopropyl-3-(2,6-dichlorophenyl)isoxazol-4-yl)ethyl)-3-methylpiperazin-1-yl)quinoline-2-carboxylic Acid (I52) and methyl 6-(4-(2-(5-cyclopropyl-3-(2,6-dichlorophenyl)isoxazol-4-yl)ethyl)-3-methylpiperazin-1-yl)quinoline-2-carboxylate (I53)

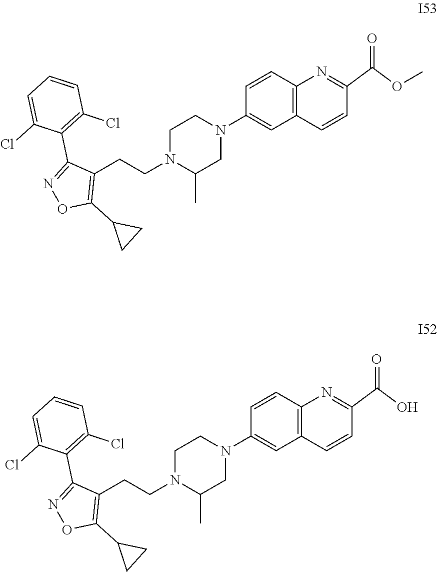 [SMILES: COC(=O)c1ccc2cc(N3CCN(CCc4c(-c5c(Cl)cccc5Cl)noc4C4CC4)C(C)C3)ccc2n1 ; CC1CN(c2ccc3nc(C(=O)O)ccc3c2)CCN1CCc1c(-c2c(Cl)cccc2Cl)noc1C1CC1]

### Step 1: Synthesis of I52-02

Follow the procedure of Synthesis of I51-15 to give I52-02 as a yellow solid. MS Calcd.: 385.5; MS Found: 386.5 [M+H]+.

### Step 2: Synthesis of I52-03

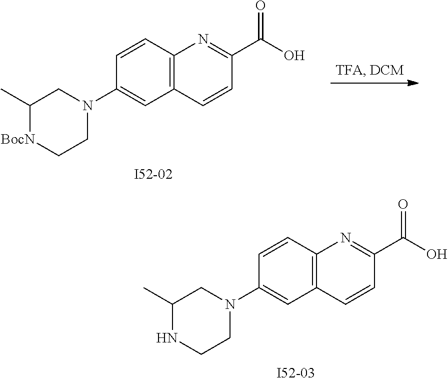 [SMILES: CC1CN(c2ccc3nc(C(=O)O)ccc3c2)CCN1C(=O)OC(C)(C)C ; CC1CN(c2ccc3nc(C(=O)O)ccc3c2)CCN1]

Follow the procedure of Synthesis of 6 to give I52-03 as a yellow solid. The crude was used into the following reaction without the further purification.

### Step 3: Synthesis of I53

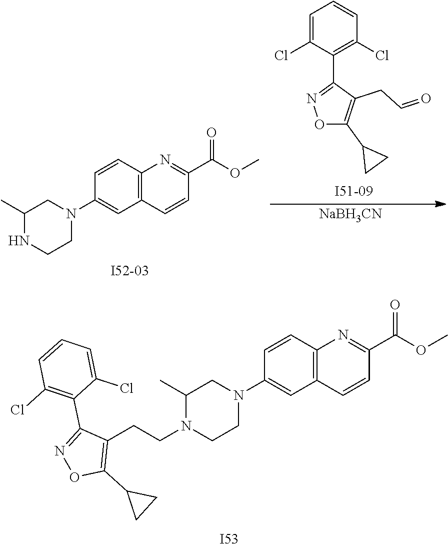

Follow the procedure of Synthesis of I46-9 to give I53 as a yellow solid. ¹H NMR (400 MHz, CD₃OD) δ: 8.22 (d, J=8.4 Hz, 1H), 8.03 (t, J=8.8 Hz, 2H), 7.64 (dd, J=2.4, 9.6 Hz, 1H), 7.59-7.57 (m, 2H), 7.52 (dd, J=6.8, 9.2 Hz, 1H), 7.14 (d, J=2.4 Hz, 1H), 4.02 (s, 3H), 3.68 (t, J=9.2 Hz, 2H), 3.31-3.04 (m, 2H), 3.01-2.98 (m, 1H), 2.97-2.44 (m, 6H), 2.25-2.19 (m, 1H), 1.28-1.18 (m, 4H), 0.86 (d, J=6.4 Hz, 3H). MS Calcd.: 565.5; MS Found: 566.1 [M+H]⁺.

### Step 4: Synthesis of I52

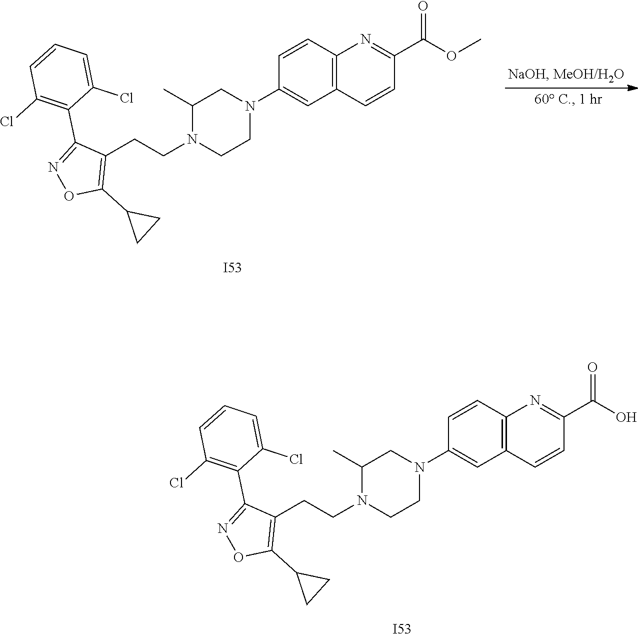 [SMILES: "I53" ; COC(=O)c1ccc2cc(N3CCN(CCc4c(-c5c(Cl)cccc5Cl)noc4C4CC4)C(C)C3)ccc2n1 ; "I53" ; CC1CN(c2ccc3nc(C(=O)O)ccc3c2)CCN1CCc1c(-c2c(Cl)cccc2Cl)noc1C1CC1]

Follow the procedure of Synthesis of I46 to give I52 (52.0 mg, 53.6% yield) as an orange solid. ¹H NMR (CD₃OD) δ: 8.34 (d, J=8.4 Hz, 1H), 8.15-8.11 (m, 2H), 7.72 (dd, J=2.8, 9.6 Hz, 1H), 7.65-7.62 (m, 2H), 7.59-7.55 (m, 1H), 7.33 (d, J=2.8 Hz, 1H), 3.97-3.91 (m, 2H), 3.72-3.62 (m, 2H), 3.48-3.37 (m, 3H), 3.26-3.15 (m, 2H), 3.03-2.98 (m, 1H), 2.80-2.75 (m, 1H), 1.27 (d, J=6.8 Hz, 3H), 1.26-1.17 (m, 4H). MS Calcd.: 551.5; MS Found: 552.2 [M+H]+.

### Example 54

### 6-(4-(2-(5-cyclopropyl-3-(2,6-dichlorophenyl)isoxazol-4-yl)ethyl)-7-methyl-1,4-diazepan-1-yl)-1-methyl-1H-indole-3-carboxylic Acid (I54)

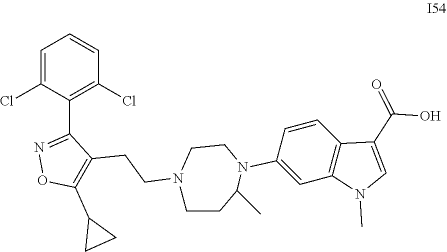 [SMILES: CC1CCN(CCc2c(-c3c(Cl)cccc3Cl)noc2C2CC2)CCN1c1ccc2c(C(=O)O)cn(C)c2c1]

### Example 55 and Example 56

### 6-(4-(2-(5-cyclopropyl-3-(2,6-dichlorophenyl)isoxazol-4-yl)ethyl)-5-methyl-1,4-diazepan-1-yl)-1-methyl-1H-indole-3-carboxylic Acid (I55) and methyl 6-(4-(2-(5-cyclopropyl-3-(2,6-dichlorophenyl)isoxazol-4-yl)ethyl)-5-methyl-1,4-diazepan-1-yl)-1-methyl-1H-indole-3-carboxylate (I56)

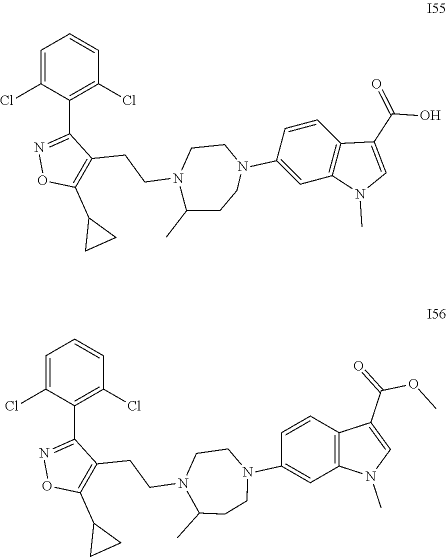 [SMILES: CC1CCN(c2ccc3c(C(=O)O)cn(C)c3c2)CCN1CCc1c(-c2c(Cl)cccc2Cl)noc1C1CC1 ; COC(=O)c1cn(C)c2cc(N3CCC(C)N(CCc4c(-c5c(Cl)cccc5Cl)noc4C4CC4)CC3)ccc12]

### Step 1: Synthesis of I55-3

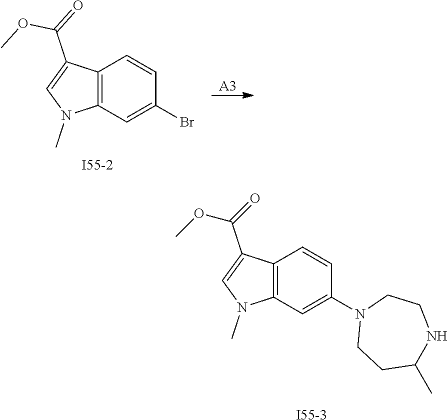 [SMILES: "I55-2" ; "I55-3" ; COC(=O)c1cn(C)c2cc(Br)ccc12 ; COC(=O)c1cn(C)c2cc(N3CCNC(C)CC3)ccc12 ; "A3"]

To a solution of compound I55-2 (1.0 g, 3.73 mmol) in DMF (30 mL) was added compound 5-Methyl-[1,4]diazepane A3 (639 mg, 5.59 mmol), Cs₂CO₃ (3.30 g, 10.11 mmol), t-Buxphos (716 mg, 1.69 mmol) and Pd₂(dba)₃ (1.55 g, 1.69 mmol). The reaction mixture was stirred at 110° C. overnight. The reaction mixture was diluted with H₂O (50 mL) and extracted with EA. The combined organic layers were washed with brine, dried over Na₂SO₄, filtered and concentrated to give I55-3 as a yellow solid. MS Calcd.: 301; MS Found: 302 [M+H]+.

### Step 2: Synthesis of I55-4

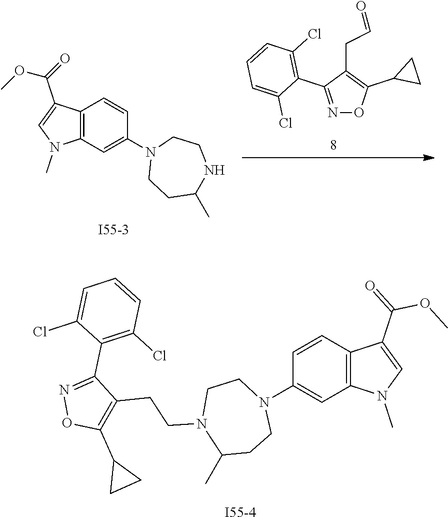

Follow the procedure of Synthesis of I46-9 to give 156 as a yellow solid. MS Calcd.: 581; MS Found: 582 [M+H]⁺.

### Step 3: Synthesis of I55

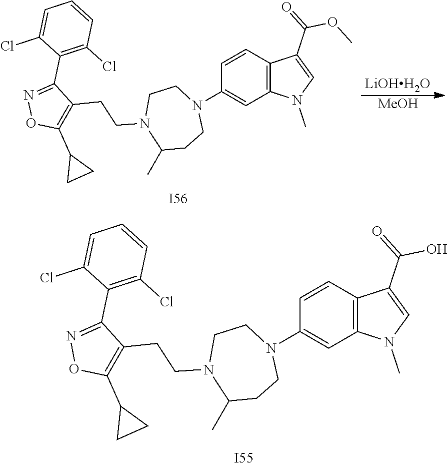

Follow the procedure of Synthesis of 146 to give compound I55 as a white solid. MS Calcd.: 567; MS Found: 568 [M+H]⁺. ¹H NMR (400 MHz, CD₃OD) δ: 7.89 (d, J=8.8 Hz, 1H), 7.71 (s, 1H), 7.52-7.40 (m, 3H), 6.80-6.76 (m, 1H), 6.61 (s, 1H), 3.79 (s, 3H), 3.51 (t, J=5.2 Hz, 2H), 3.28-2.94 (m, 4H), 2.64-2.62 (m, 4H), 2.43-2.39 (m, 1H), 2.08-1.98 (m, 2H), 1.81-1.75 (m, 1H), 1.08-1.02 (m, 3H), 0.98-0.93 (m, 4H).

### Example 57

### 6-(1-(2-(5-cyclopropyl-3-(2,6-dichlorophenyl)isoxazol-4-yl)ethyl)-2-methylpiperidin-4-yl)-1-methyl-1H-indole-3-carboxylic Acid (I57)

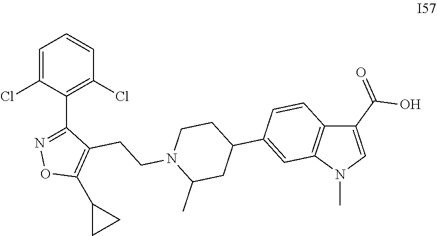 [SMILES: CC1CC(c2ccc3c(C(=O)O)cn(C)c3c2)CCN1CCc1c(-c2c(Cl)cccc2Cl)noc1C1CC1]

### Step 1: Synthesis of I57-2

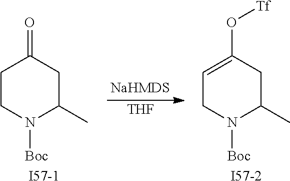

To a solution of compound I57-1 (1 g, 4.7 mmol) in THF (10 mL) was added NaHMDS (3.5 mL, 7.0 mmol) at −70° C. and the mixture was stirred at −70° C. for 1 h. Then 1,1,1-trifluoro-N-phenyl-N-((trifluoromethyl)sulfonyl)methanesulfonamide (2.51 g, 7.0 mmol) in THF (5 ml) was added at −70° C., and the mixture was stirred at room temperature overnight. The reaction mixture was diluted with H₂O (50 mL) and extracted with EA (50 mL×2). The combined organic layers were washed with brine (50 mL), dried over Na₂SO₄, filtered and concentrated. The residue was purified by flash chromatography on silica gel (PE/EA=10/1) to afford compound I57-2 (0.4 g, 24.8% yield) as a yellow oil. MS Calcd.: 345; MS Found: 346[M+H]⁺.

### Step 2: Synthesis of I57-3

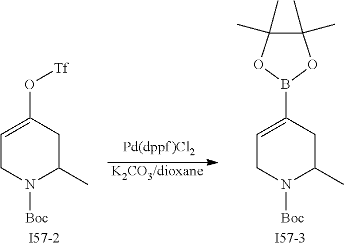

To a solution of compound I57-2 (710 mg, 2.06 mmol) in 1,4-dioxane (10 mL) was added Pin₂B₂ (789.5 mg, 3.09 mmol), K₂CO₃ (854.5 mg, 6.19 mmol) and Pd(dppf)Cl₂ (150.9 mg, 0.21 mmol). The reaction mixture was heated to 100° C. and stirred under N₂ overnight. The reaction was cooled to room temperature, diluted with H₂O (50 mL) and extracted with EA. The combined organic layers were washed with brine, dried over Na₂SO₄, filtered and concentrated. The residue was purified by flash chromatography on silica gel (PE/EA=10/1) to afford compound I57-3 as a yellow oil. MS Calcd.: 323; MS Found: 324[M+H]⁺.

### Step 3: Synthesis of I57-4

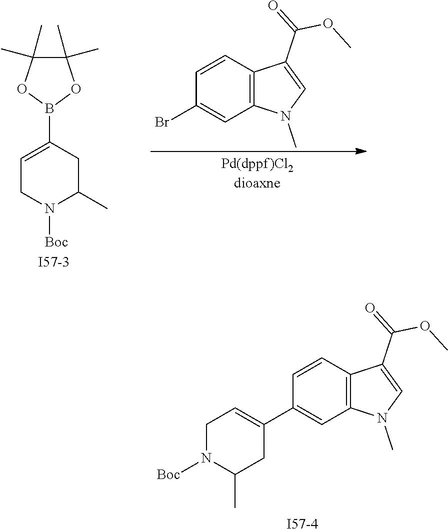

To a solution of compound I57-3 (500 mg, 1.3 mmol) in 1,4-dioxane (50 mL) and H₂O (1 mL) was added methyl 6-bromo-1-methyl-1H-indole-3-carboxylate (523.4 mg, 1.95 mmol), K₂CO₃ (539.1 mg, 3.91 mmol) and Pd(dppf)Cl₂ (95.2 mg, 0.13 mmol). The reaction mixture was heated to 100° C. under N₂ overnight. The reaction was cooled to rt, diluted with H₂O (50 mL) and extracted with EA. The combined organic layers were washed with brine, dried over Na₂SO₄, filtered and concentrated. The residue was purified by flash chromatography on silica gel (PE/EA=10/1) to afford compound I57-4 (100 mg, 16.8% yield) as a yellow solid. MS Calcd.: 384; MS Found: 385[M+H]⁺.

### Step 4: Synthesis of I57-5

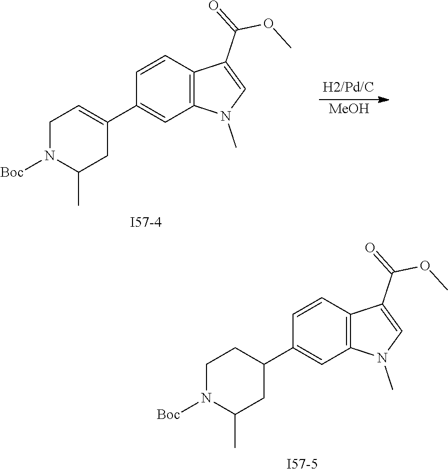 [SMILES: "MeOH" ; "I57-4" ; "I57-5" ; COC(=O)c1cn(C)c2cc(C3=CCN(C(=O)OC(C)(C)C)C(C)C3)ccc12 ; COC(=O)c1cn(C)c2cc(C3CCN(C(=O)OC(C)(C)C)C(C)C3)ccc12]

To a solution of compound I57-5 (70 mg, 0.18 mmol) in MeOH (5 mL) was added Pd/C (20 mg). The reaction mixture was stirred at room temperature under H₂ atmosphere for 1 h. The reaction mixture was filtered and concentrated to give compound I57-6 (50 mg, 71.1% yield) as yellow oil. MS Calcd.: 386; MS Found: 387[M+H]⁺.

### Step 5: Synthesis of I57-6

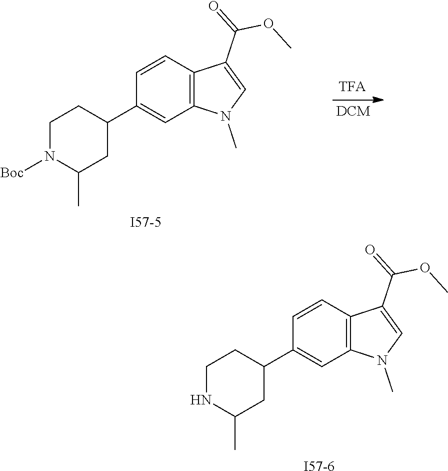 [SMILES: "I57-5" ; "I57-6" ; COC(=O)c1cn(C)c2cc(C3CCN(C(=O)OC(C)(C)C)C(C)C3)ccc12 ; COC(=O)c1cn(C)c2cc(C3CCNC(C)C3)ccc12]

Follow the procedure of Synthesis of 6 to give I57-6 as a yellow oil. MS Calcd.: 286; MS Found: 287[M+H]⁺.

### Step 6: Synthesis of I57-7

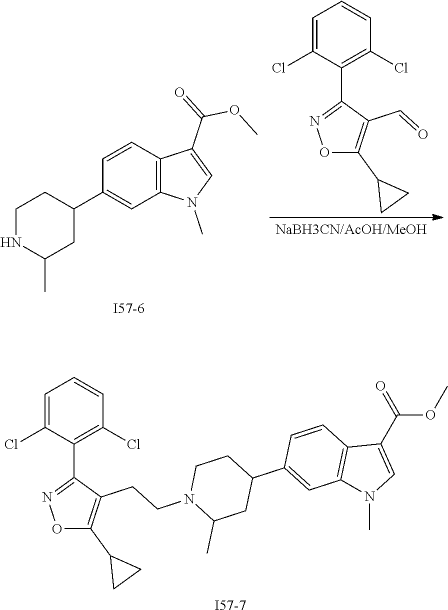 [SMILES: "I57-6" ; COC(=O)c1cn(C)c2cc(C3CCNC(C)C3)ccc12 ; O=Cc1c(-c2c(Cl)cccc2Cl)noc1C1CC1 ; "I57-7" ; COC(=O)c1cn(C)c2cc(C3CCN(CCc4c(-c5c(Cl)cccc5Cl)noc4C4CC4)C(C)C3)ccc12]

Follow the procedure of Synthesis of I46-9 to give I57-7 as a yellow solid. MS Calcd.: 565; MS Found: 566[M+H]⁺.

### Step 7: Synthesis of I57

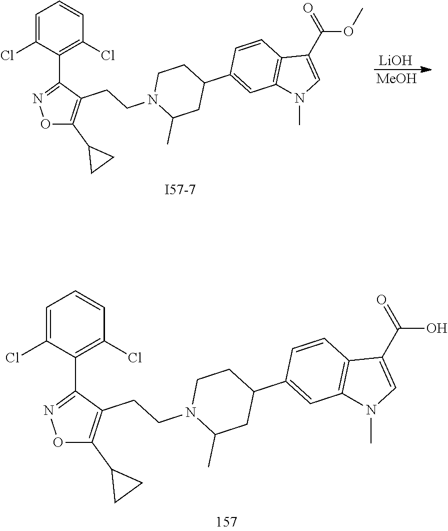

Follow the procedure of Synthesis of I46-9 to give 157 (12 mg, 41.0% yield) as a white solid. MS Calcd.: 551; MS Found: 552[M+H]⁺. ¹H NMR (400 MHz, DMSO-d6) δ:11.83 (s, 1H), 7.98 (s, 1H), 7.92 (d, J=3.4 Hz, 1H), 7.70 (d, J=3.8 Hz, 2H), 7.63 (d, J=3.4 Hz, 1H), 7.30 (d, J=2.8 Hz, 1H), 7.09-7.05 (t, J=7.4 Hz, 1H), 3.82 (d, J=1 Hz, 3H), 3.24 (s, 3H), 3.13-2.84 (m, 2H), 2.72-2.62 (m, 1H), 2.37-2.26 (m, 2H), 2.10-1.58 (m, 4H), 1.57-1.30 (m, 1H), 1.24 (s, 1H), 1.20-1.01 (m, 5H), 0.90-0.75 (m, 1H).

### Example 58 and Example 59

### 6-(4-(((5-cyclopropyl-3-(2,6-dichlorophenyl)isoxazol-4-yl)methyl)(methyl)amino)piperidin-1-yl)-1-methyl-1H-indole-3-carboxylic Acid (I58) and methyl 6-(4-(((5-cyclopropyl-3-(2,6-dichlorophenyl)isoxazol-4-yl)methyl)(methyl)amino)piperidin-1-yl)-1-methyl-1H-indole-3-carboxylate (I59)

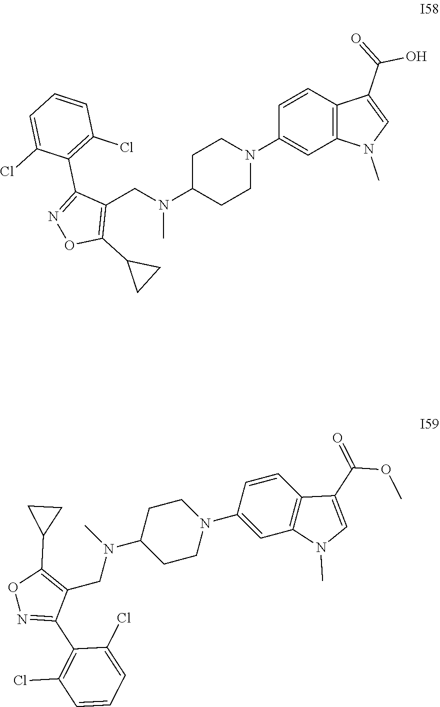 [SMILES: CN(Cc1c(-c2c(Cl)cccc2Cl)noc1C1CC1)C1CCN(c2ccc3c(C(=O)O)cn(C)c3c2)CC1 ; COC(=O)c1cn(C)c2cc(N3CCC(N(C)Cc4c(-c5c(Cl)cccc5Cl)noc4C4CC4)CC3)ccc12]

### Step 1: Synthesis of I58-3

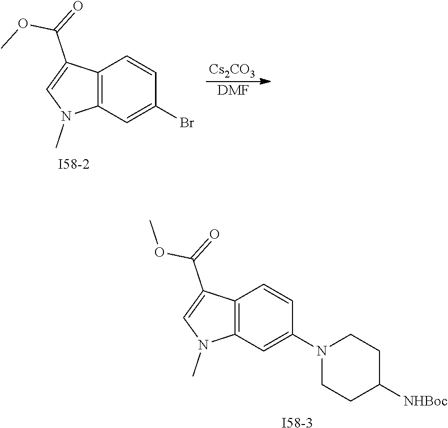

To a solution of compound I58-2 (500 mg, 1.87 mmol) in DMF (20 mL) was added tert-butyl 2,5-diazabicyclo[2.2.1]heptane-2-carboxylate (743 mg, 3.74 mmol), Cs₂CO₃ (1.2 g, 3.74 mmol), Pd₂(dba)₃ (250 mg) and t-Buxphos (250 mg). The reaction mixture was heated to 110° C. under N₂ for 3 h. The reaction was cooled to rt, diluted with H₂O (20 mL) and extracted with EA. The combined organic layers were washed with brine, dried over Na₂SO₄, and concentrated. The residue was purified by flash chromatography on silica gel (EA/PE=5%-25%) to afford compound I58-3 (550 mg, 76% yield) as a yellow solid. MS Calcd.: 387; MS Found: 388[M+H]⁺.

### Step 2: Synthesis of I58-4

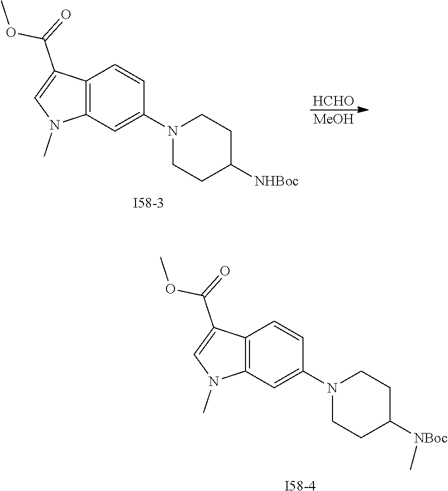 [SMILES: "MeOH" ; "I58-3" ; COC(=O)c1cn(C)c2cc(N3CCC(NC(=O)OC(C)(C)C)CC3)ccc12 ; "HCHO" ; "I58-4" ; COC(=O)c1cn(C)c2cc(N3CCC(N(C)C(=O)OC(C)(C)C)CC3)ccc12]

To a solution of compound I58-3 (100 mg, 0.25 mmol) in MeOH (10 mL) was added HCHO (15.5 mg, 0.50 mmol), AcOH (two drops). The reaction mixture was stirred at rt for 2 h. NaBH₃CN (31 mg, 0.50 mmol) was added and the resulting mixture was stirred at rt for 2 h. Then diluted with H₂O (20 mL) and extracted with EA. The combined organic layers were washed with brine (50 mL), dried over Na₂SO₄, and concentrated. The residue was purified by flash chromatography on silica gel (EA/PE=5%-25%) to afford compound I58-4 as a yellow solid. MS Calcd.: 401; MS Found: 402[M+H]⁺.

### Step 3: Synthesis of I58-5

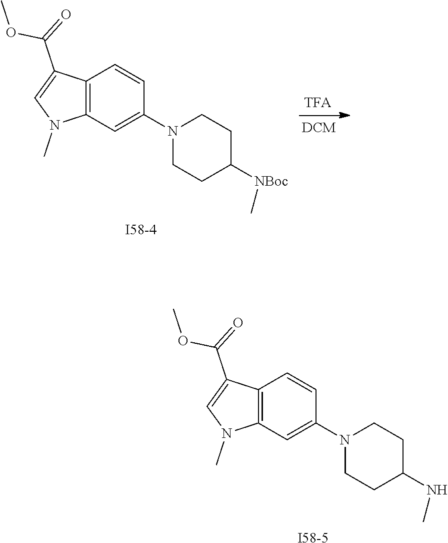 [SMILES: "I58-4" ; COC(=O)c1cn(C)c2cc(N3CCC(N(C)C(=O)OC(C)(C)C)CC3)ccc12 ; "I58-5" ; CNC1CCN(c2ccc3c(C(=O)OC)cn(C)c3c2)CC1]

Follow the procedure of Synthesis of 6 to give I58-5 as a dark oil. MS Calcd.: 301; MS Found: 302[M+H]⁺

### Step 4: Synthesis of I59

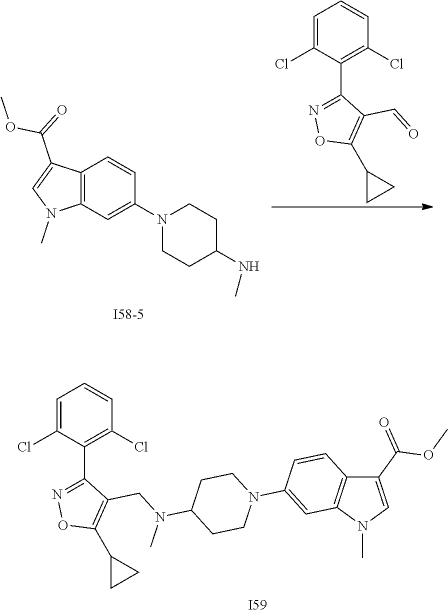 [SMILES: "I58-5" ; CNC1CCN(c2ccc3c(C(=O)OC)cn(C)c3c2)CC1 ; O=Cc1c(-c2c(Cl)cccc2Cl)noc1C1CC1 ; "I59" ; COC(=O)c1cn(C)c2cc(N3CCC(N(C)Cc4c(-c5c(Cl)cccc5Cl)noc4C4CC4)CC3)ccc12]

Follow the procedure of Synthesis of I46-9 to give 159 as a yellow solid. MS Calcd.: 566; MS Found: 567[M+H]+. ¹H NMR (400 MHz, DMSO-d6) δ: 7.96 (s, 1H), 7.81 (d, J=9.2 Hz, 1H), 7.71-7.69 (m, 3H), 6.97 (d, J=8.8 Hz, 2H), 4.50 (brs, 1H), 4.08 (brs, 2H), 3.82 (s, 3H), 3.79 (s, 3H), 3.25 (brs, 2H), 2.67 (brs, 3H), 2.55 (brs, 3H), 1.87 (brs, 1H), 1.74-1.54 (m, 3H), 1.24 (brs, 4H).

### Step 5: Synthesis of I58

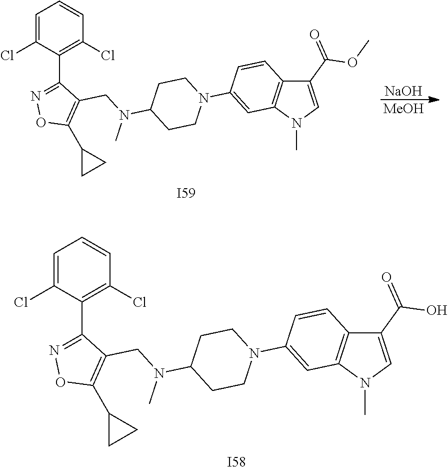

Follow the procedure of Synthesis of 146 to give 158 (31.5 mg, 37% yield) as a white solid. MS Calcd.: 552; MS Found: 553[M+H]^{+ 1}H NMR (400 MHz, DMSO-d6) δ: 7.79 (d, J=5.6 Hz, 2H), 7.61-7.59 (m, 3H), 7.52-7.48 (m, 1H), 6.89-6.86 (m, 2H), 5.32 (t, J=4.8 Hz, 1H), 3.75 (s, 3H), 3.58 (d, J=10.8 Hz, 2H), 2.33-2.26 (m, 3H), 2.08 (s, 3H), 2.03-1.97 (m, 2H), 1.50-1.23 (m, 3H), 1.15-1.04 (m, 4H), 0.87-0.79 (m, 2H).

### Example 60

### 2-(6-((5-cyclopropyl-3-(2-(trifluoromethoxy)phenyl)isoxazol-4-yl)methoxy)-3-azabicyclo[3.1.1]heptan-3-yl)-4-fluorobenzo[d]thiazole-6-carboxylic Acid (I60)

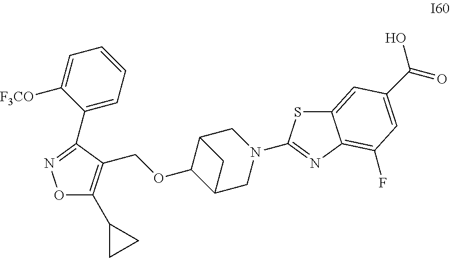 [SMILES: O=C(O)c1cc(F)c2nc(N3CC4CC(C3)C4OCc3c(-c4ccccc4OC(F)(F)F)noc3C3CC3)sc2c1]

### Step 1: Synthesis of I60-6

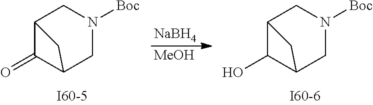 [SMILES: "MeOH" ; CC(C)(C)OC(=O)N1CC2CC(C1)C2=O ; "NaBH4" ; CC(C)(C)OC(=O)N1CC2CC(C1)C2O]

To a solution of compound I60-5 (0.9 g, 4.3 mmol) in MeOH (20 mL) was added NaBH₄ (324.1 mg, 8.53 mmol) port wise at 0° C. The reaction mixture was stirred at 0° C. for 3 h. The reaction was diluted with H₂O (50 mL) and extracted with EA. The combined organic layers were washed with brine, dried over Na₂SO₄, filtered and concentrated to give compound I60-6 as a yellow oil. MS Calcd.: 213; MS Found: 214[M+H]⁺.

### Step 2: Synthesis of I60-7

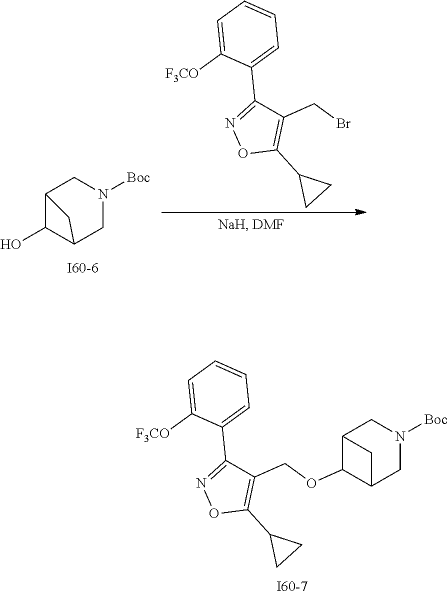 [SMILES: CC(C)(C)OC(=O)N1CC2CC(C1)C2O ; FC(F)(F)Oc1ccccc1-c1noc(C2CC2)c1CBr ; CC(C)(C)OC(=O)N1CC2CC(C1)C2OCc1c(-c2ccccc2OC(F)(F)F)noc1C1CC1]

To a solution of compound I60-6 (510 mg, 2.39 mmol) in DMF (10 mL) was added NaH (143.7 mg, 3.59 mmol) port wise at 0° C. and stirred at 0° C. for 1 h. Then 4-(bromomethyl)-5-cyclopropyl-3-(2-(trifluoromethoxy)phenyl)isoxazole (624.9 mg, 2.39 mmol) was added. The resulting mixture was stirred at 50° C. for 1 h. Then the reaction was diluted with H₂O (50 mL) and extracted with EA. The combined organic layers were washed with brine, dried over Na₂SO₄, filtered and concentrated. The residue was purified by flash chromatography on silica gel (EA/PE=10/1) to afford compound I60-7 as a yellow oil. MS Calcd.: 494; MS Found: 495[M+H]⁺.

### Step 3: Synthesis of I60-8

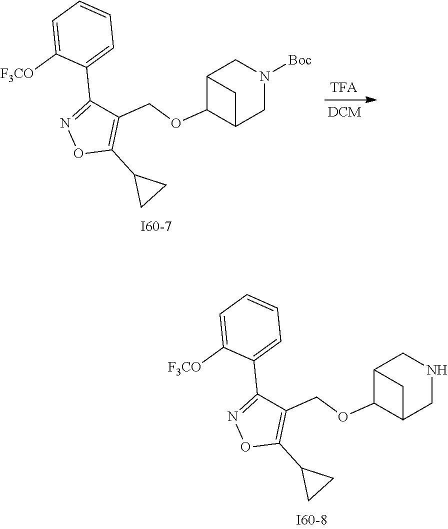 [SMILES: CC(C)(C)OC(=O)N1CC2CC(C1)C2OCc1c(-c2ccccc2OC(F)(F)F)noc1C1CC1 ; FC(F)(F)Oc1ccccc1-c1noc(C2CC2)c1COC1C2CNCC1C2]

Follow the procedure of Synthesis of 6 to give I60-8 as a yellow oil. MS Calcd.: 394; MS Found: 395[M+H]⁺.

### Step 4: Synthesis of I60-9

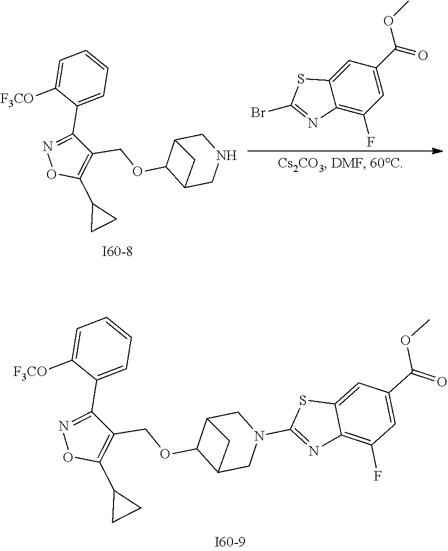 [SMILES: FC(F)(F)Oc1ccccc1-c1noc(C2CC2)c1COC1C2CNCC1C2 ; COC(=O)c1cc(F)c2nc(Br)sc2c1 ; COC(=O)c1cc(F)c2nc(N3CC4CC(C3)C4OCc3c(-c4ccccc4OC(F)(F)F)noc3C3CC3)sc2c1]

Follow the procedure of Synthesis of 29 to give I60-9 as colorless oil. MS Calcd.: 603; MS Found: 604[M+H]⁺.

### Step 4: Synthesis of I60

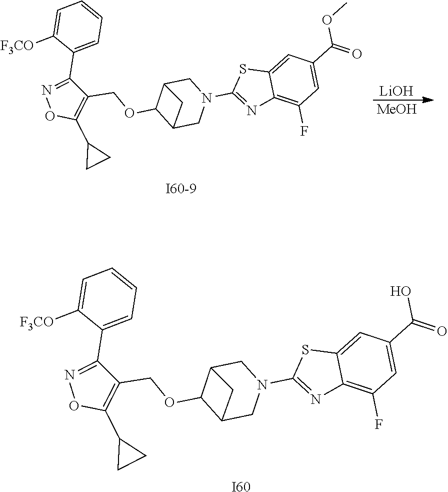 [SMILES: "MeOH" ; COC(=O)c1cc(F)c2nc(N3CC4CC(C3)C4OCc3c(-c4ccccc4OC(F)(F)F)noc3C3CC3)sc2c1 ; "LiOH" ; "I60" ; O=C(O)c1cc(F)c2nc(N3CC4CC(C3)C4OCc3c(-c4ccccc4OC(F)(F)F)noc3C3CC3)sc2c1]

Follow the procedure of Synthesis of 146 to give 160 (12 mg, 30.7% yield) as a white solid. ¹H NMR (400 MHz, DMSO-d6) δ: 12.81 (s, 1H), 8.28 (s, 1H), 7.64-7.36 (m, 5H), 4.37 (d, J=8.4 Hz, 2H), 3.88-3.85 (m, 2H), 3.60-3.47 (m, 2H), 2.67-2.64 (m, 2H), 2.33-2.25 (m, 1H), 1.68-1.66 (m, 1H), 1.42 (d, J=5.2 Hz, 1H), 1.01-0.94 (m, 4H). MS Calcd.: 589; MS Found: 590 [M+H]+.

### Example 61

### 6-(6-((5-cyclopropyl-3-(2,6-dichlorophenyl)isoxazol-4-yl)methoxy)-3-azabicyclo[3.1.1]heptan-3-yl)-1-methyl-1H-indole-3-carboxylic Acid (I61)

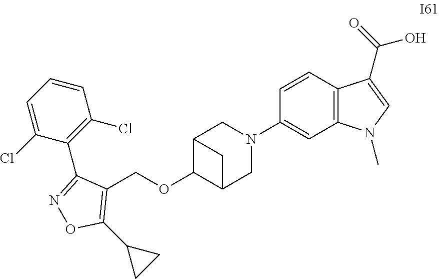 [SMILES: Cn1cc(C(=O)O)c2ccc(N3CC4CC(C3)C4OCc3c(-c4c(Cl)cccc4Cl)noc3C3CC3)cc21]

### Step 1: Synthesis of I61-3

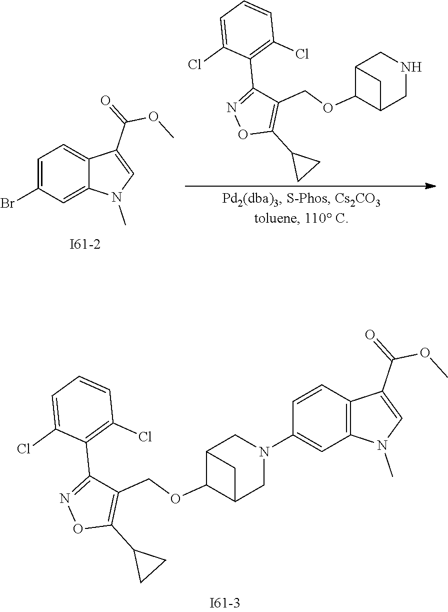 [SMILES: COC(=O)c1cn(C)c2cc(Br)ccc12 ; Clc1cccc(Cl)c1-c1noc(C2CC2)c1COC1C2CNCC1C2 ; COC(=O)c1cn(C)c2cc(N3CC4CC(C3)C4OCc3c(-c4c(Cl)cccc4Cl)noc3C3CC3)ccc12]

To a solution of compound I61-2 (572 mg, 2.13 mmol) in DMF (10 mL) was added 4-((3-azabicyclo[3.1.1]heptan-6-yloxy)methyl)-5-cyclopropyl-3-(2,6-dichlorophenyl)isoxazole (270 mg, 0.71 mmol), Cs₂CO₃ (698 mg, 2.13 mmol), Pd₂(dba)₃ (140 mg) and S-Phos (140 mg). The reaction mixture was heated to 110° C. under N₂ for 23 h. The reaction was cooled to rt, diluted with H₂O and extracted with EA. The combined organic layers were washed with brine, dried over Na₂SO₄, filtered and concentrated. The residue was purified by flash chromatography on silica gel (EA/PE=3%-25%) to afford compound I61-3 (100 mg, 26% yield) as a yellow solid. MS Calcd.: 565; MS Found: 566[M+H]⁺.

### Step 2: Synthesis of I61

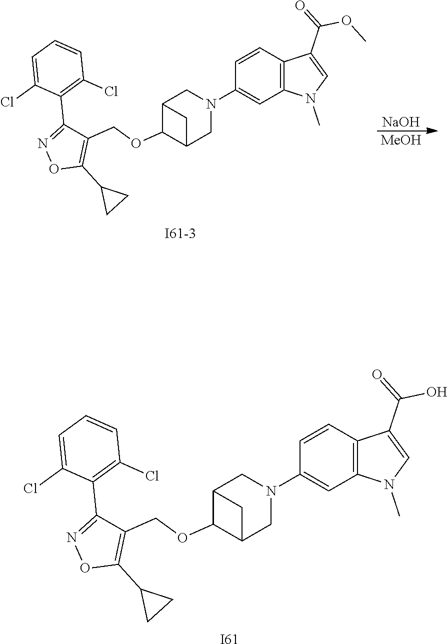 [SMILES: "MeOH" ; COC(=O)c1cn(C)c2cc(N3CC4CC(C3)C4OCc3c(-c4c(Cl)cccc4Cl)noc3C3CC3)ccc12 ; "NaOH" ; "I61" ; Cn1cc(C(=O)O)c2ccc(N3CC4CC(C3)C4OCc3c(-c4c(Cl)cccc4Cl)noc3C3CC3)cc21]

Follow the procedure of Synthesis of I46 to give I61 as a white solid. ¹H NMR (400 MHz, DMSO-d6) δ: 7.80-7.76 (m, 2H), 7.43-7.41 (m, 2H), 7.37-7.34 (m, 1H), 6.65-6.62 (m, 1H), 6.47 (d, J=1.6 Hz, 2H), 4.25 (s, 2H), 3.75-3.72 (m, 1H), 3.75 (s, 3H), 3.48 (d, J=9.6 Hz, 2H), 3.19 (d, J=9.6 Hz, 2H), 2.66-2.64 (m, 2H), 2.24-2.20 (m, 1H), 1.56-1.54 (m, 1H), 1.43 (d, J=9.6 Hz, 1H), 0.98-0.94 (m, 2H), 0.82-0.78 (m, 2H). MS Calcd.: 551; MS Found: 552 [M+H]⁺.

### Example 62

### 6-(6-((5-cyclopropyl-3-(2-(trifluoromethoxy)phenyl)isoxazol-4-yl)methoxy)-3-azabicyclo[3.1.1]heptan-3-yl)-1-methyl-1H-indole-3-carboxylic Acid (I62)

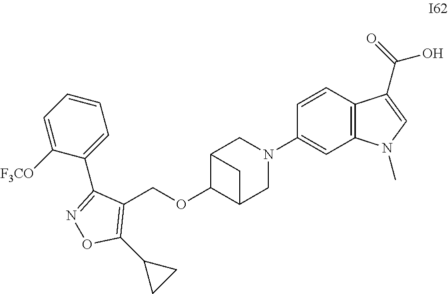 [SMILES: Cn1cc(C(=O)O)c2ccc(N3CC4CC(C3)C4OCc3c(-c4ccccc4OC(F)(F)F)noc3C3CC3)cc21]

### Step 1: Synthesis of I62-1

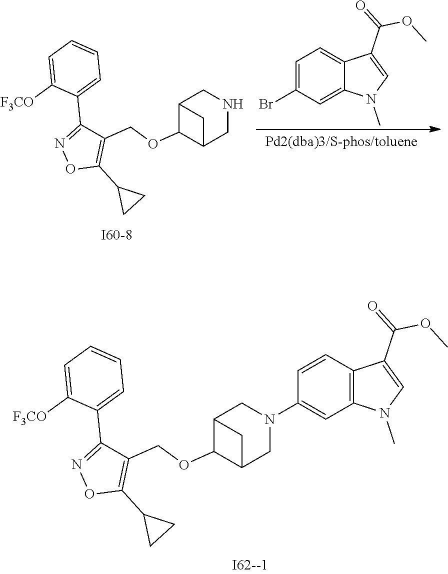 [SMILES: FC(F)(F)Oc1ccccc1-c1noc(C2CC2)c1COC1C2CNCC1C2 ; COC(=O)c1cn(C)c2cc(Br)ccc12 ; COC(=O)c1cn(C)c2cc(N3CC4CC(C3)C4OCc3c(-c4ccccc4OC(F)(F)F)noc3C3CC3)ccc12]

Follow the procedure of Synthesis of I61-3 to give I62-1 as a yellow solid. MS Calcd.: 581; MS Found: 582[M+H]⁺.

### Step 2: Synthesis of I62

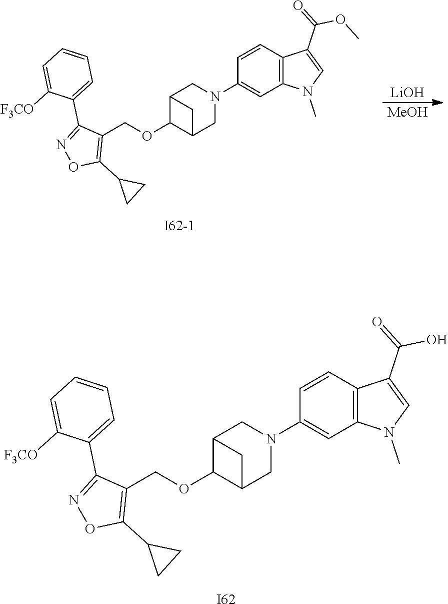 [SMILES: "MeOH" ; "I62-1" ; COC(=O)c1cn(C)c2cc(N3CC4CC(C3)C4OCc3c(-c4ccccc4OC(F)(F)F)noc3C3CC3)ccc12 ; "LiOH" ; "I62" ; Cn1cc(C(=O)O)c2ccc(N3CC4CC(C3)C4OCc3c(-c4ccccc4OC(F)(F)F)noc3C3CC3)cc21]

Follow the procedure of Synthesis of I46 to give I62 as a yellow solid.

¹H NMR (400 MHz, DMSO-d6) δ: 11.68 (s, 1H), 7.79 (d, J=8.4 Hz, 1H), 7.75 (s, 1H), 7.59-7.55 (m, 1H), 7.52-7.45 (m, 2H), 7.35-7.31 (m, 1H), 6.66-6.64 (m, 1H), 6.49 (d, J=1.6 Hz, 1H), 4.33 (s, 2H), 3.78-7.76 (m, 1H), 3.74 (s, 3H), 3.49 (d, J=9.6 Hz, 2H), 3.24 (d, J=9.6 Hz, 2H), 2.67 (t, J=5.2 Hz, 2H), 2.20-2.18 (m, 1H), 1.57-1.48 (m, 1H), 1.43 (d, J=9.2 Hz, 1H), 0.91-0.88 (m, 2H), 0.73-0.70 (m, 2H).

MS Calcd.: 567; MS Found: 568 [M+H]⁺.

### Example 63

### 6-(6-((5-cyclopropyl-3-(2,6-dichlorophenyl)isoxazol-4-yl)methoxy)-3-azabicyclo[3.1.1]heptan-3-yl)-4-fluoro-1-methyl-1H-indole-3-carboxylic Acid (I63)

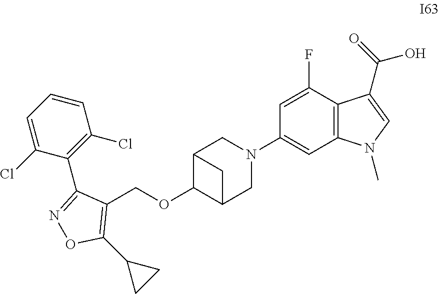 [SMILES: Cn1cc(C(=O)O)c2c(F)cc(N3CC4CC(C3)C4OCc3c(-c4c(Cl)cccc4Cl)noc3C3CC3)cc21]

### Step 1: Synthesis of I63-8

Follow the procedure of Synthesis of I61-3 to give I63-8 as a yellow oil. MS Calcd.: 583; MS Found: 584[M+H]⁺.

### Step 2: Synthesis of I63

Follow the modified procedure of Synthesis of I46 to give I63 as a white solid. ¹H NMR (DMSO-d6) δ: 11.66 (s, 1H), 7.81 (s, 1H), 7.42-7.34 (m, 3H), 6.34-6.30 (m, 1H), 6.27 (d, J=1.6 Hz, 1H), 4.25 (s, 2H), 3.75-3.72 (m, 1H), 3.73 (s, 3H), 3.44 (d, J=9.6 Hz, 2H), 3.15 (d, J=9.6 Hz, 2H), 2.65-2.62 (m, 2H), 2.26-2.22 (m, 1H), 1.57-1.54 (m, 1H), 1.40 (d, J=9.6 Hz, 1H), 1.01-0.97 (m, 2H), 0.88-0.84 (m, 2H). MS Calcd.: 570; MS Found: 571 [M+H]⁺.

### Example 64

### 6-(6-((5-cyclopropyl-3-(2-(trifluoromethoxy)phenyl)isoxazol-4-yl)methoxy)-3-azabicyclo[3.1.1]heptan-3-yl)-4-fluoro-1-methyl-1H-indole-3-carboxylic Acid (I64)

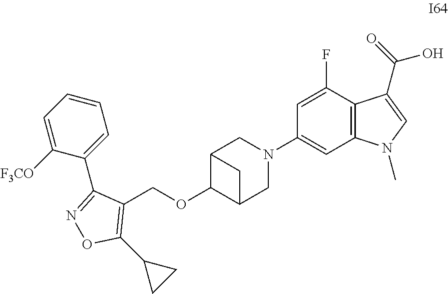 [SMILES: Cn1cc(C(=O)O)c2c(F)cc(N3CC4CC(C3)C4OCc3c(-c4ccccc4OC(F)(F)F)noc3C3CC3)cc21]

### Step 1: Synthesis of I64-2

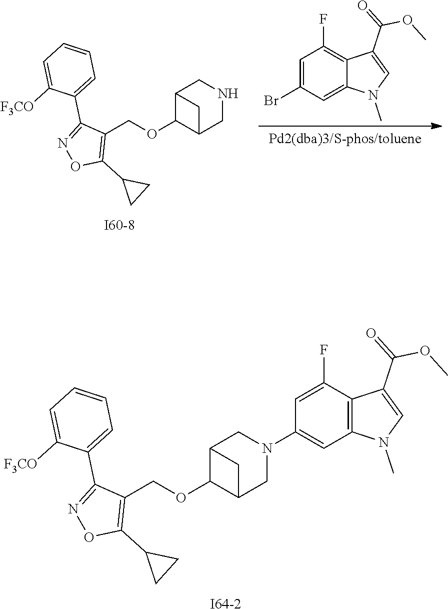

Follow the procedure of Synthesis of I61-3 to give I64-2 as a yellow solid. MS Calcd.: 599; MS Found: 600[M+H]⁺.

### Step 2: Synthesis of I64

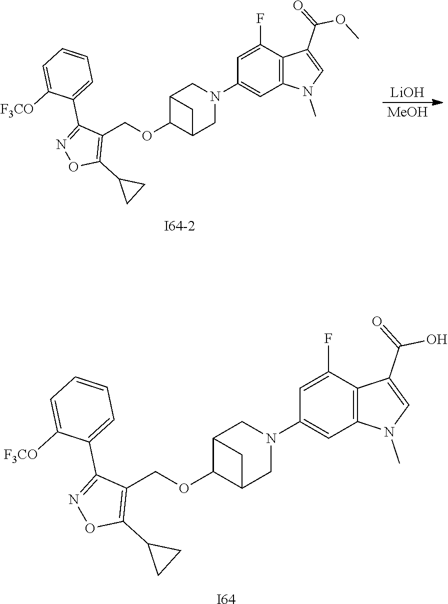 [SMILES: "MeOH" ; "I64-2" ; COC(=O)c1cn(C)c2cc(N3CC4CC(C3)C4OCc3c(-c4ccccc4OC(F)(F)F)noc3C3CC3)cc(F)c12 ; "LiOH" ; "I64" ; Cn1cc(C(=O)O)c2c(F)cc(N3CC4CC(C3)C4OCc3c(-c4ccccc4OC(F)(F)F)noc3C3CC3)cc21]

Follow the procedure of Synthesis of 146 to give 164 as a yellow solid. ¹H NMR (400 MHz, DMSO-d6) δ: 7.64 (s, 1H), 7.60-7.45 (m, 3H), 7.37-7.33 (m, 1H), 6.29 (d, J=6.8 Hz, 1H), 6.27 (s, 1H), 4.34 (s, 2H), 3.79-3.76 (t, J=5.6 Hz, 1H), 3.70 (s, 3H), 3.44 (d, J=9.6 Hz, 2H), 3.21-3.16 (m, 2H), 2.67-2.65 (t, J=4.6 Hz, 2H), 2.22-2.09 (m, 1H), 1.58-1.56 (m, 1H), 1.40 (d, J=9.6 Hz, 1H), 0.94-0.91 (m, 2H), 0.77-0.74 (m, 2H). MS Calcd.: 585; MS Found: 586 [M+H]+.

### Example 65

### 2-(6-((5-cyclopropyl-3-(2,6-dichlorophenyl)isoxazol-4-yl)methoxy)-3-azabicyclo[3.1.1]heptan-3-yl)benzo[d]thiazole-6-carboxylic Acid (I65)

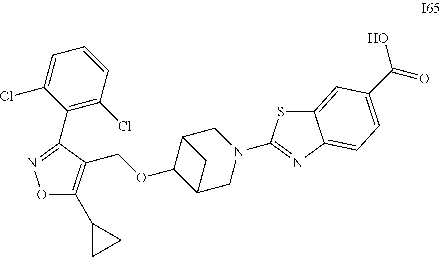 [SMILES: O=C(O)c1ccc2nc(N3CC4CC(C3)C4OCc3c(-c4c(Cl)cccc4Cl)noc3C3CC3)sc2c1]

### Step 1: Synthesis of I65-2

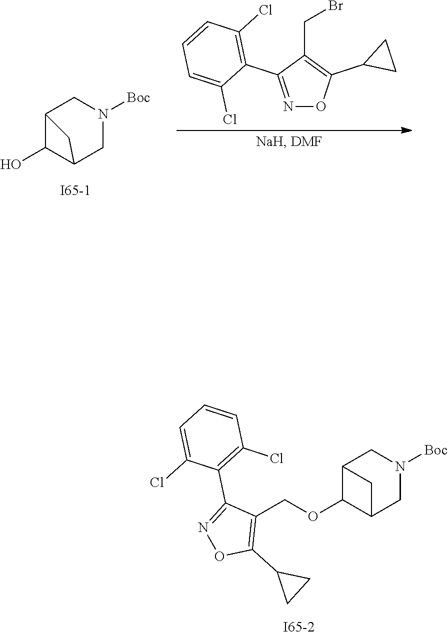 [SMILES: "I65-1" ; CC(C)(C)OC(=O)N1CC2CC(C1)C2O ; Clc1cccc(Cl)c1-c1noc(C2CC2)c1CBr ; "I65-2" ; CC(C)(C)OC(=O)N1CC2CC(C1)C2OCc1c(-c2c(Cl)cccc2Cl)noc1C1CC1]

Follow the procedure of Synthesis of I60-7 to give I65-2 as a yellow solid. MS Calcd.: 478; MS Found: 479[M+H]⁺.

### Step 2: Synthesis of I65-3

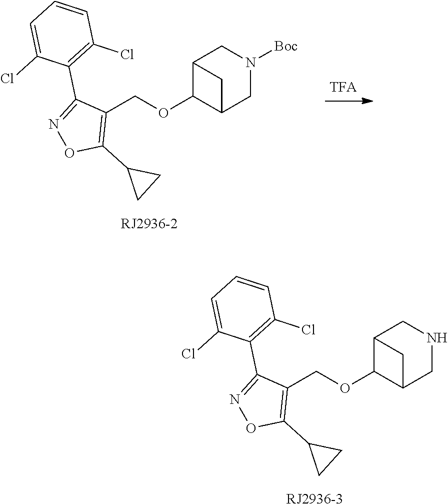

Follow the procedure of Synthesis of 6 to give I65-3 as a dark oil. MS Calcd.: 385; MS Found: 386[M+H]⁺.

### Step 3: Synthesis of I65-4

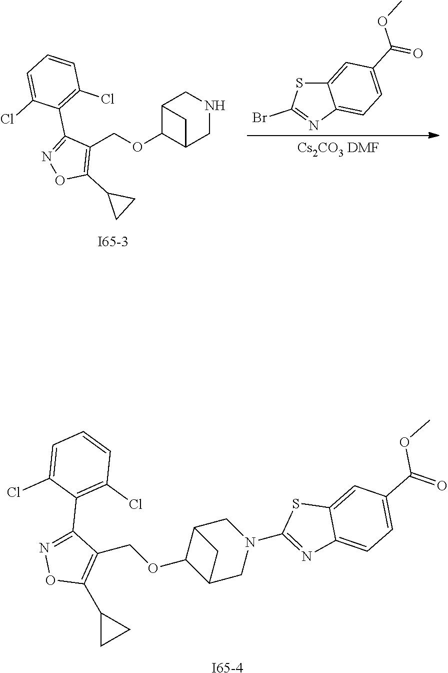

Follow the modified procedure of Synthesis of 29 to give compound I65-4 as a yellow oil. MS Calcd.: 569; MS Found: 570[M+H]⁺.

### Step 4: Synthesis of I65

Follow the procedure of Synthesis of 146 to give 165 as a white solid. ¹H NMR (400 MHz, DMSO-d6) δ: 8.38 (s, 1H), 7.90 (d, J=8.0 Hz, 1H), 7.05 (d, J=8.4 Hz, 1H), 7.40 (s, 2H), 7.32 (t, J=8.0 Hz, 1H), 4.30 (s, 2H), 3.82 (t, J=5.6 Hz, 1H), 3.33-3.17 (m, 4H), 2.64 (s, 2H), 2.32-2.28 (m, 1H), 1.64-1.62 (m, 1H), 1.40 (d, J=10.4 Hz, 1H), 1.04-0.98 (m, 4H). MS Calcd.: 555; MS Found: 556 [M+H]⁺.

### Example 66

### 2-(6-((5-cyclopropyl-3-(2-(trifluoromethoxy)phenyl)isoxazol-4-yl)methoxy)-3-azabicyclo[3.1.1]heptan-3-yl)benzo[d]thiazole-6-carboxylic Acid (I66)

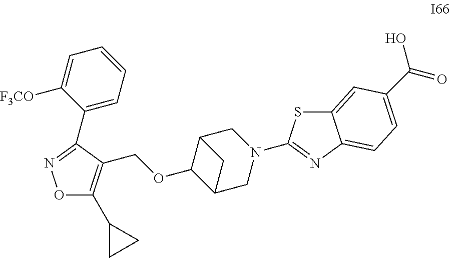 [SMILES: O=C(O)c1ccc2nc(N3CC4CC(C3)C4OCc3c(-c4ccccc4OC(F)(F)F)noc3C3CC3)sc2c1]

### Step 1: Synthesis of I66-1

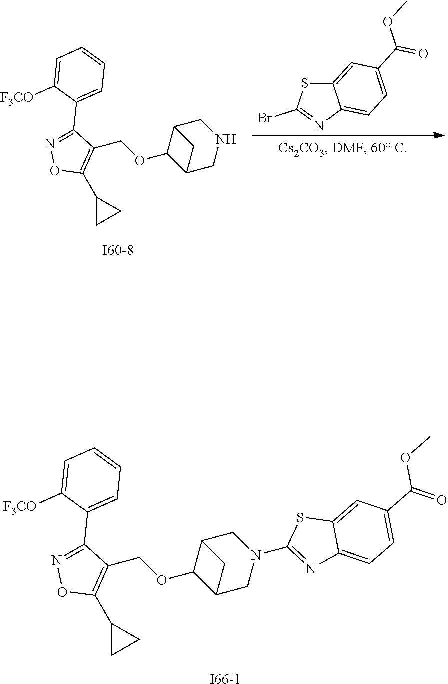

Follow the modified procedure of Synthesis of 29 to give I66-1 as a colorless oil. MS Calcd.: 585; MS Found: 586[M+H]⁺.

### Step 2: Synthesis of I66

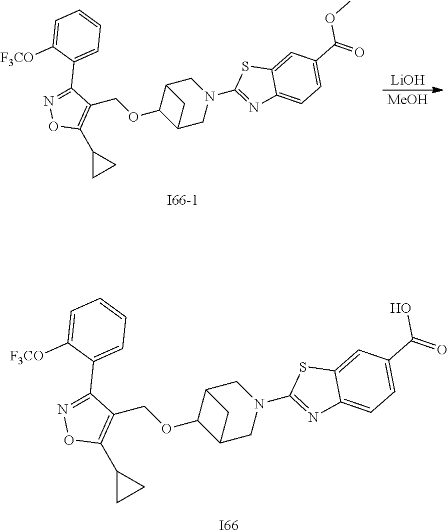 [SMILES: "MeOH" ; "I66-1" ; COC(=O)c1ccc2nc(N3CC4CC(C3)C4OCc3c(-c4ccccc4OC(F)(F)F)noc3C3CC3)sc2c1 ; "LiOH" ; "I66" ; O=C(O)c1ccc2nc(N3CC4CC(C3)C4OCc3c(-c4ccccc4OC(F)(F)F)noc3C3CC3)sc2c1]

Follow the modified procedure of Synthesis of 146 to give 166 as a white solid. ¹H NMR (400 MHz, DMSO-d6) δ: 8.37 (s, 1H), 7.87 (d, J=8.4 Hz, 1H), 7.52-7.44 (m, 4H), 7.36 (t, J=7.8 Hz, 1H). 4.38 (s, 2H), 3.85 (t, J=5.6 Hz, 1H), 3.76-3.42 (m, 2H), 2.64 (s, 2H), 2.39-2.25 (m, 2H), 1.65-1.62 (m, 1H), 1.25-1.21 (m, 1H), 1.40 (d, J=10.4 Hz, 1H), 1.04-0.90 (m, 4H). MS Calcd.: 571; MS Found: 572 [M+H]⁺.

### Example 67

### 2-(6-((5-cyclopropyl-3-(2,6-dichlorophenyl)isoxazol-4-yl)methoxy)-3-azabicyclo[3.1.1]heptan-3-yl)benzo[d]thiazole-6-carboxylic Acid (I67)

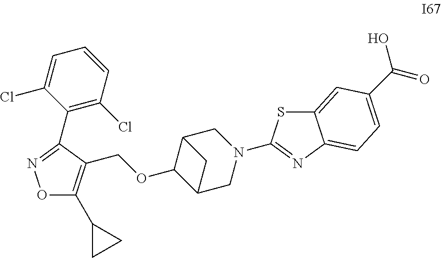 [SMILES: O=C(O)c1ccc2nc(N3CC4CC(C3)C4OCc3c(-c4c(Cl)cccc4Cl)noc3C3CC3)sc2c1]

### Step 1: Synthesis of I67-2

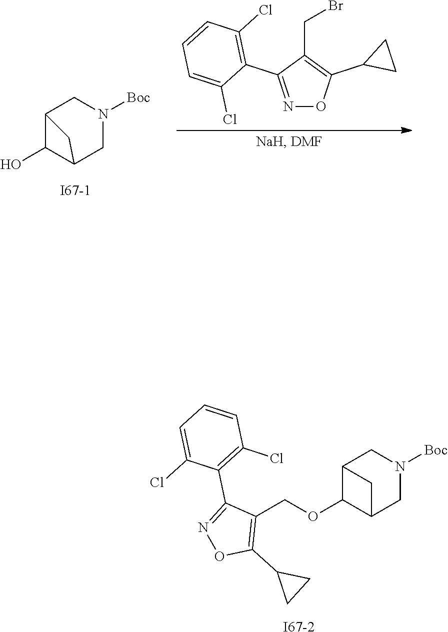 [SMILES: "I67-1" ; CC(C)(C)OC(=O)N1CC2CC(C1)C2O ; Clc1cccc(Cl)c1-c1noc(C2CC2)c1CBr ; "I67-2" ; CC(C)(C)OC(=O)N1CC2CC(C1)C2OCc1c(-c2c(Cl)cccc2Cl)noc1C1CC1]

Follow the procedure of Synthesis of I60-7 to give compound I67-2 as a yellow solid. MS Calcd.: 478; MS Found: 479[M+H]⁺.

### Step 2: Synthesis of I67-3

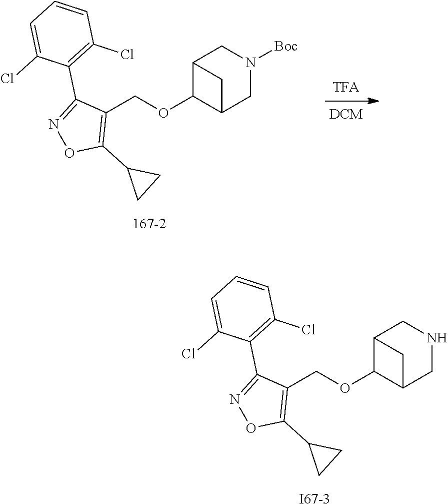

Follow the procedure of Synthesis of 6 to give compound I67-3 as a dark oil. MS Calcd.: 385; MS Found: 386[M+H]⁺.

### Step 3: Synthesis of I67-4

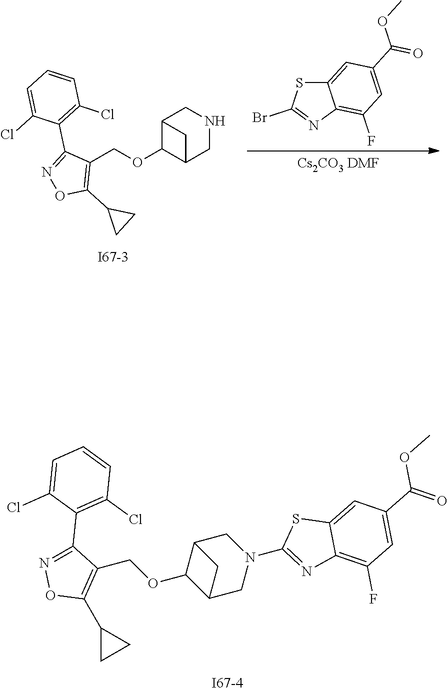

Follow the modified procedure of Synthesis of 29 to give I67-4 as a yellow oil. MS Calcd.: 587; MS Found: 588[M+H]⁺.

### Step 5: Synthesis of I67

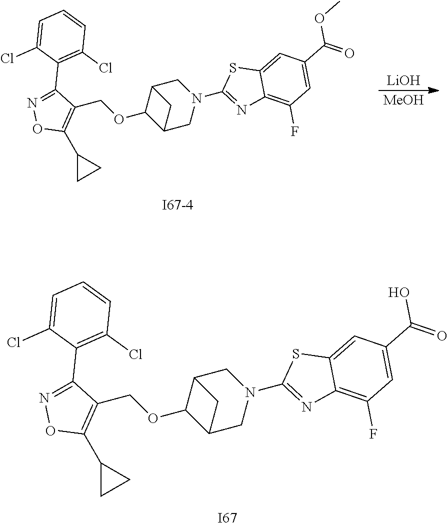 [SMILES: "MeOH" ; "I67-4" ; COC(=O)c1cc(F)c2nc(N3CC4CC(C3)C4OCc3c(-c4c(Cl)cccc4Cl)noc3C3CC3)sc2c1 ; "LiOH" ; "I67" ; O=C(O)c1cc(F)c2nc(N3CC4CC(C3)C4OCc3c(-c4c(Cl)cccc4Cl)noc3C3CC3)sc2c1]

Follow the procedure of Synthesis of 146 to give 167 as a white solid. ¹H NMR (400 MHz, DMSO-d6) δ: 8.21 (d, J=1.6 Hz, 1H), 7.62-7.59 (m, 1H), 7.41 (s, 2H), 7.32 (t, J=8.0 Hz, 1H), 4.30 (s, 2H), 3.82 (t, J=6.0 Hz, 1H), 3.34-3.24 (m, 4H), 2.64-2.62 (m, 2H), 2.34-2.27 (m, 1H), 1.66-1.60 (m, 1H), 1.40 (d, J=10.0 Hz, 1H), 1.05-0.99 (m, 4H). MS Calcd.: 574; MS Found: 575 [M+H]+.

Assay Description

The biological properties of the new compounds are investigated based on the following in vitro assay methods.

FXR Binding Assay

| Materials and Reagents | Vendor | Cat# |
|---|---|---|
| Fluorescein-SRC2-2 coactivator peptide | Invitrogen | PV4586 |
| FXR LBD, GST | Invitrogen | PV4834 |
| LanthaScreen ™ Tb-anti-GST antibody | Invitrogen | PV3550 |
| Coregulator Buffer G | Invitrogen | PV4553 |
| DTT | Invitrogen | P2325 |
| DMSO | Sigma | D8418 |

The interaction between the present compounds and FXR are evaluated by Time-Resolved Fluorescence Resonance Energy Transfer (TR-FRET) competition assay. FRET describes a radiation-free energy transfer between two chromophores: a donor fluorophore in its excited state can transfer energy to an acceptor fluorophore in close proximity (typically <10 nm). In contrast to standard FRET, TR-FRET unites time-resolved fluorescence (TRF) and the FRET principle, TRF use a long-lifetime lanthanide chelate as the donor species, while lanthanide chelates are unique in that their excited-state lifetime can be on the order of a millisecond or longer. Suitable neighbors for FRET are known in the art and can be obtained.

The assay was performed in following steps: first, prepared a) 1 mM compounds (100×) in DMSO; b) reaction buffer containing 1× buffer G with 10 mM DTT; c) 4×FXR LBD (20 nM) in 1× chilled buffer G. Second, performed 3-fold serial dilution of 1 mM test compounds using DMSO from 1 mM, 10 doses in a 96-well plate (249944, Nunc). Then, used complete buffer G to dilute each 100× compounds serial dilution to 2×. Third, added 10 μl 2× compound serial dilution into 384-well plate (3677, Corning). Then, added 5 μl FXR LBD into the assay plate. Prepared a solution containing 2 μM fluorescein-SRC2-2 (4×) and 20 nM Tb anti-GST antibody (4×) in buffer G at room temperature and started the reaction by adding 5 μl the above solution into each well of the assay plate. Centrifuged the assay plates at 1000 g for 1 min and then incubated at room temperature for lhr protected from light. Finally, the plate was read at wavelengths of 520 nm and 495 nm on Envision 2104 plate reader (PerkinElmer).

A ratio (Ratio_{520 nm/495 nm}−Ratio_{background}) was calculated for each well. The activity ratio was calculated as follow:

$$\% Activity = {⌊ \frac{{Ratio}_{cmpd} - {\overline{Ratio}}_{Vehicle}}{{\overline{Ratio}}_{positive} - {\overline{Ratio}}_{Vehicle}} ⌋}^{*} 100$$

R̅a̅t̅i̅o̅ₚₒₛᵢₜᵢᵥₑ: The average ratio for the positive control across the plate.

R̅a̅t̅i̅o̅_{vehicle}: The average ratio for negative controls across the plate.

EC₅₀ was calculated by fitting % Activity values and log of compound concentrations to nonlinear regression (dose response-variable slope) with GraphPad 5.0.

$$Y = Bottom + ( Top - Bottom ) / ( 1 + 1 {0}^{⩓} ( {( Log {EC}_{50} - X )}^{*} Hillslope ) ) .$$

X: log of compound concentration.

Y: % Activity.

Z factor>0.5; S/B>3.

The following results were obtained

| Cpd | EC₅₀ | Pct Effect |
|---|---|---|
| No. | (μM) | (Emax %) |
| GW4064 | 0.108 | 100.50 |
| I1 | 0.058 | 160.60 |
| I2 | 5.106 | 75.14 |
| I3 | 0.091 | 192.30 |
| I4 | 0.244 | 89.83 |
| I5 | 0.297 | 116.30 |
| I6 | 0.118 | 141.60 |
| I7 | 0.025 | 98.10 |
| I8 | 0.094 | 201.80 |
| I9 | 1.220 | 206.00 |
| I10 | 0.728 | 172.70 |
| I11 | 1.637 | NA |
| I12 | 0.133 | 146.80 |
| I13 | 1.530 | 34.92 |
| I14 | 0.655 | 142.10 |
| I15 | 0.419 | 167.50 |
| I16 | >10 | NA |
| I17 | 0.046 | 131.10 |
| I18 | 0.046 | 141.50 |
| I19 | 4.357 | 104.10 |
| I20 | 0.450 | 193.00 |
| I21 | 0.890 | 85.08 |
| I22 | 0.162 | 162.90 |
| I23 | 1.366 | 35.67 |
| I24 | 0.422 | 89.82 |
| I25 | 0.083 | 151.20 |
| I26 | 0.882 | 214.40 |
| I27 | 0.454 | 91.11 |
| I28 | 1.342 | 131.70 |
| I29 | 3.815 | 68.08 |
| I30 | 0.060 | 111.00 |
| I31 | 0.041 | 45.15 |
| I32 | 0.129 | 47.71 |
| I33 | 0.097 | 43.29 |
| I34 | 0.236 | 41.51 |
| I35 | 0.097 | 156.40 |
| I36 | 0.094 | 161.40 |
| I37 | 0.101 | 231.70 |
| I38 | 0.048 | 133.30 |
| I39 | 0.150 | 128.70 |
| I40 | 0.077 | 131.30 |
| I41 | 0.200 | 158.50 |
| I42 | 0.099 | 196.20 |
| I43 | 1.399 | 109.70 |
| I44 | 0.401 | 73.50 |
| I45 | 0.072 | 161.70 |
| I46 | 0.149 | 168.00 |
| I47 | NA | NA |
| I48 | 1.721 | 124.90 |
| I49 | >10 | NA |
| I50 | NA | NA |
| I51 | 0.074 | 96.54 |
| I52 | 0.209 | 121.20 |
| I53 | 0.436 | 68.39 |
| I54 | NA | NA |
| I55 | 2.771 | 70.93 |
| I56 | >10 | NA |
| I57 | 0.384 | 180.50 |
| I58 | 0.497 | 83.44 |
| I59 | >10 | NA |
| I60 | 0.732 | 35.30 |
| I61 | 0.616 | 67.68 |
| I62 | NA | NA |
| I63 | 0.102 | 106.00 |
| I64 | NA | NA |
| I65 | >10 | NA |
| I66 | >10 | NA |
| I67 | >10 | NA |

NA—Not applicable.

FXR Transactivation Assay

| Materials and Reagents | Vendor | Cat# |
|---|---|---|
| HEK293T | ATCC | CRL-3216 |
| pGL4.35 [luc2P/9XGAL4 UAS/Hygro] | Promega | E1370 |
| pBIND-FXR Vector | Pharmaron | |
| DMEM, high glucose | Gibico | 10569010 |
| fetal bovine serum | Gibico | 16000-044 |
| Penicillin-Streptomycin (10000 U/ml,100 ml) | Gibico | 15140-122 |
| DMEM, High Glucose, HEPES, no Phenol Red | Gibico | 21063-029 |
| PBS (pH 7.4, 1×, sterile) | Gibico | 10010 |
| Opti-MEM ® I Reduced Serum Medium | Gibico | 11058-021 |
| Steady-Glo ™ Luciferase Assay System | Promega | E2520 |
| TransIT-293 Transfection Reagent | MIRUS BIO | MIR2700 |

The present compounds were tested to assess their ability to stimulate FXR transactivation activity. The hFXR-LBD, coding sequence was inserted into the pBIND expression vector (Promega, E1581) to express FXR-GAL4 binding domain chimeric receptors. This expression vector and reporter vector (pGL4.35 which carry a stably integrated GAL4 promoter driven luciferase reporter gene) were co-transfected into HEK293T host cells. Upon agonist binding to the corresponding FXR-GAL4 chimeric receptor, the chimeric receptor binds to the GAL4 binding sites and stimulates the reporter gene.

The assay is performed in following steps: first, prepared a) 1000× positive control (5 mM, GW4064) and 1000× vehicle control (100% DMSO); b) 3-fold serial diluted of reference compound using DMSO from 5 mM for 10 dose; c) 3-fold serial diluted of test compounds using DMSO from 10 mM for 10 dose. All the working stock were shake for 5 min on plate shaker (QILINBEIER). Second, all cells were cultured as ATCC recommended. HEK293T cells were assayed in exponential growth phase. Removed culture medium from the flask and rinsed cells with PBS. Added TrypLE solution to the flask and make cells detach. Next, cells were washed once with complete growth medium. Then, cells were pelleted and washed twice with PBS to remove phenol red and resuspend them in medium to a proper concentration. Only cells with viability greater than 90% were used for assays. Seeded 6×10⁶ HEK293T cells into a 100 mm dish and incubated at 37° C. under 5% CO₂ atmosphere for 16 h. Third, Trans-IT reagent and Opti-MEM mix by inversion and incubate 5 min at room temperature. The cell transfection was performed by mixing DNA and the reagent mixture, as well as 6 μg GAL4-FXR plasmid or 2 μg pGL4.35 luciferase plasmid, respectively. The reagent mixture was added to a 100 mm dish and incubated for 4-7 hrs at 37° C. under 5% CO₂ atmosphere. Fourth, transferred 75 nl compound dilutions into 384-well assay plates (PerkinElmer) and seeded HEK293T cells at 17,000 cells/well using phenol red-free DMEM containing 5% charcoal/dextran-treated FBS. Then, cells were incubated for 16-20 h at 37° C. under 5% CO₂ atmosphere. Finally, added 25 μl Steady-Glo™ Luciferase Assay Reagent into each well of 384-well assay plate, and then shake plate for 5 min protected from light on a plate shaker. Then, the luminescence value was read on Evision 2104 plate reader (PerkinElmer).

The activity ratio was calculated as follow:

$$\% Activity = {⌊ \frac{{Ratio}_{cmpd} - {\overline{Ratio}}_{Vehicle}}{{\overline{Ratio}}_{positive} - {\overline{Ratio}}_{Vehicle}} ⌋}^{*} 100$$

RLU: Resulting Luminescence

R̅L̅U̅ₚₒₛᵢₜᵢᵥₑ: The average RLU for the positive controls across the plate.

R̅L̅U̅_{vehicle}: The average RLU for negative controls across the plate.

EC₅₀ was calculated by fitting % Activity values and log of compound concentrations to nonlinear regression (dose response-variable slope) with GraphPad 5.0.

$$Y = Bottom + ( Top - Bottom ) / ( 1 + 1 {0}^{⩓} ( {( Log {EC}_{50} - X )}^{*} Hillslope ) ) .$$

X: log of compound concentration.

Y: % Activity.

Z factor>0.5; S/B>3.

The following results were obtained

| Cpd No. | EC₅₀ (μM) |
|---|---|
| GW4064 | 0.278 |
| I1 | 0.101 |
| I2 | 11.11 |
| I3 | 0.239 |
| I4 | 1.502 |
| I5 | 2.378 |
| I6 | 0.459 |
| I7 | 0.743 |
| I8 | 0.138 |
| I9 | NA |
| I10 | 0.524 |
| I11 | NA |
| I12 | 0.129 |
| I13 | NA |
| I14 | 0.912 |
| I15 | 0.667 |
| I16 | NA |
| I17 | 1.232 |
| I18 | 0.436 |
| I19 | NA |
| I20 | 0.906 |
| I21 | 2.859 |
| I22 | 0.634 |
| I23 | NA |
| I24 | 1.856 |
| I25 | 0.288 |
| I26 | 1.210 |
| I27 | 1.796 |
| I28 | NA |
| I29 | NA |
| I30 | 1.200 |
| I31 | NA |
| I32 | NA |
| I33 | NA |
| I34 | NA |
| I35 | 0.494 |
| I36 | 0.577 |
| I37 | 0.574 |
| I38 | 0.377 |
| I39 | NA |
| I40 | 0.343 |
| I41 | NA |
| I42 | 0.551 |
| I43 | NA |
| I44 | NA |
| I45 | 0.224 |
| I46 | NA |
| I47 | NA |
| I48 | NA |
| I49 | NA |
| I50 | NA |
| I51 | NA |
| I52 | NA |
| I53 | NA |
| I54 | NA |
| I55 | NA |
| I56 | NA |
| I57 | NA |
| I58 | 2.274 |
| I59 | NA |
| I60 | NA |
| I61 | NA |
| I62 | NA |
| I63 | NA |
| I64 | NA |
| I65 | NA |
| I66 | NA |
| I67 | NA |

NA—not analyzed.

On the basis of their biological properties the compounds of formula (I) according to the disclosure, some of the compounds exhibit good properties as agonists of FXR and are suitable for treating FXR-mediated conditions such as cholestasis, intrahepatic cholestatis, estrogen-induced cholestasis, drug-induced cholestasis, cholestasis of pregnancy, parenteral nutrition-associated cholestasis, PBC, PSC, PFIC, NAFLD, NASH, drug-induced bile duct injury, gallstones, liver cirrhosis, alcohol-induced cirrhosis, cystic fibrosis, bile duct obstruction, cholelithiasis, liver fibrosis, dyslipidemia, atherosclerosis, diabetes, diabetic nephropathy, colitis, newborn jaundice, prevention of kernicterus, venocclusive disease, portal hypertension, metabolic syndrome, hypercholesterolemia, intestinal bacterial overgrowth, erectile dysfunction, progressive fibrosis of the liver caused by any of the diseases above or by infectious hepatitis, or other FXR-mediated conditions leading to extrahepatic cholestasis etc. The compounds of the disclosure are also useful for lowering total cholesterol, lowering LDL cholesterol, lowering VLDL cholesterol, raising HDL levels, and/or lowering triglyceride levels.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A compound having Formula I 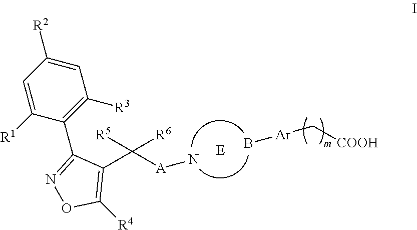
or a stereoisomer, enantiomer or a pharmaceutically acceptable salt thereof;
R¹, R² and R³ are independently selected from H, C₁₋₆alkyl, haloC₁₋₆alkyl, C₁₋₆ alkoxy, haloC₁₋₆alkoxy, or cyclopropyl;
R⁴ is selected from C₁₋₃alkyl, haloC₁₋₃alkyl or cyclopropyl optionally substituted with C₁₋₃ alkyl or haloC₁₋₃alkyl;
R⁵ and R⁶ are independently selected from H, C₁₋₃alkyl or haloC₁₋₃alkyl;
A is selected from C═O or CR⁷R⁸;
R⁷ and R⁸ are independently selected from H, C₁₋₃alkyl or C₁₋₃alkoxy;
B is CH or N;
ring E is a substituted or unsubstituted 6-8 membered heteroring or bridged-heteroring;
Ar is phenylene, C₅₋₇ cycloalkylene or 5-14 membered monocyclic or bicyclic heteroaryl containing 1-2 heteroatoms selected from N, O and S; each of which is optionally substituted with R¹⁰ and R¹¹,
R¹⁰ and R¹¹ are independently selected from H, halogen, C₁₋₆ alkyl, haloC₁₋₆ alkyl, C₁₋₆ alkoxy, haloC₁₋₆ alkoxy, or cyclopropyl;
m is 0 or 1.

2. The compound of claim 1, wherein R¹, R² and R³ are independently selected from H, Cl, F, CH₃, OCF₃, CF₃ and OMe;
R4 is C1-3 alkyl or cyclopropyl; preferably, R4 is methyl or i-Pr:
R5, R6, R7 and R8 are independently selected from hydrogen or Me.

3. (canceled)

4. (canceled)

5. (canceled)

6. (canceled)

7. The compound of claim 1, wherein Ar is phenylene, pyridylene, pyrimidinylene, pyrazinylene, pyridazinylene, thiazolylene, benzothiazolyl, benzo[d]isothiazolyl, imidazo[1,2-a]pyridinyl, quinolinyl, 1H-indolyl, pyrrolo[1,2-b]pyridazinyl, benzofuranyl, benzo[b]thiophenyl, 1H-indazolyl, benzo[d]isoxazolyl, quinazolinyl, 1H-pyrrolo[3,2-c]pyridinyl, pyrazolo[1,5-a]pyrimidinyl, imidazo[1,2-b]pyridazinyl, pyrazolo[1,5-a]pyridinyl; each of which is optionally substituted with R¹⁰ and R¹¹ selected from H, halogen, C₁₋₆ alkyl, haloC₁₋₆ alkyl, C₁₋₆ alkoxy, haloC₁₋₆ alkoxy, or cyclopropyl, preferably, Ar is selected from phenylene, benzothiazolyl, quinolinyl, 1H-indolyl, 1H-indazolyl, each of which is optionally substituted with 0-2 groups of Me or F.

8. (canceled)

9. The compound of claim 7, wherein Ar is phenylene or selected from the following structure:

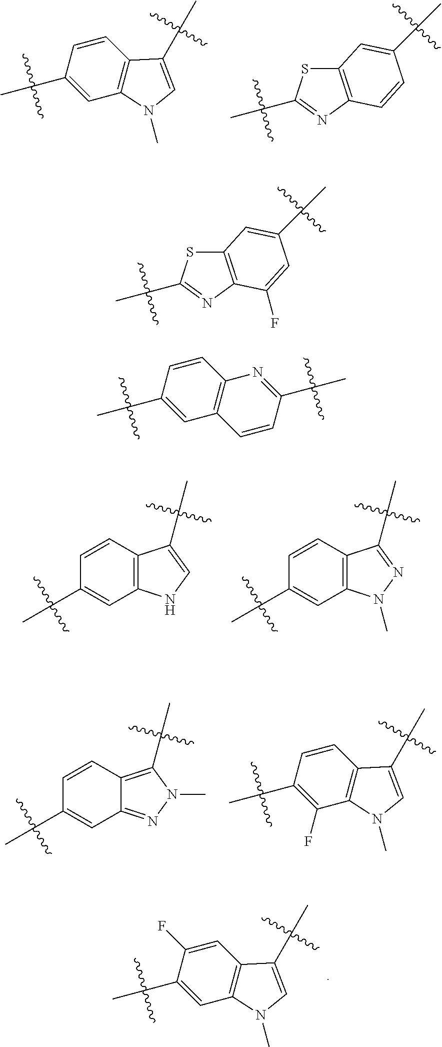

10. The compound of claim 1, wherein ring E is selected from the following structure, which is optionally substituted with 0-2 groups of Me: 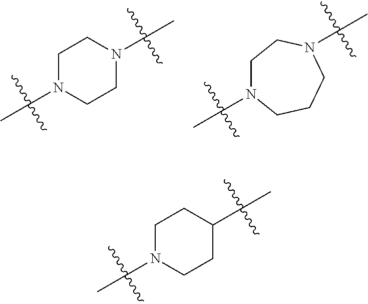 [SMILES: *N1CCN(*)CC1 ; *N1CCCN(*)CC1 ; *C1CCN(*)CC1]
preferably, ring E is selected from the following structure:

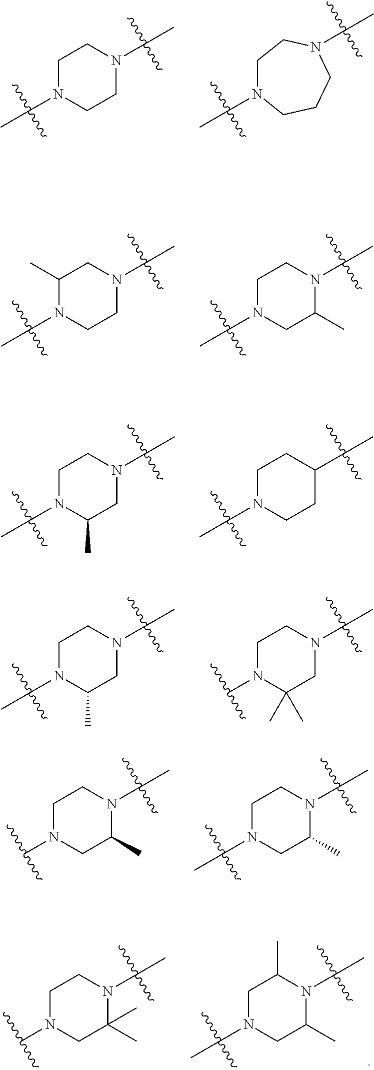

11. (canceled)

12. The compound of claim 1, wherein ring E is selected from the following structure

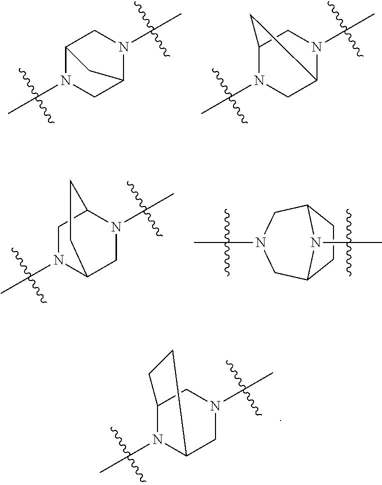 [SMILES: *N1CC2CC1CN2* ; *N1CC2CC1CN2* ; *N1CC2CCC1CN2* ; *N1CC2CCC(C1)N2* ; *N1CC2CCC(C1)N2*]

13. The compound of claim 1, wherein said compound is selected from the group consisting of: 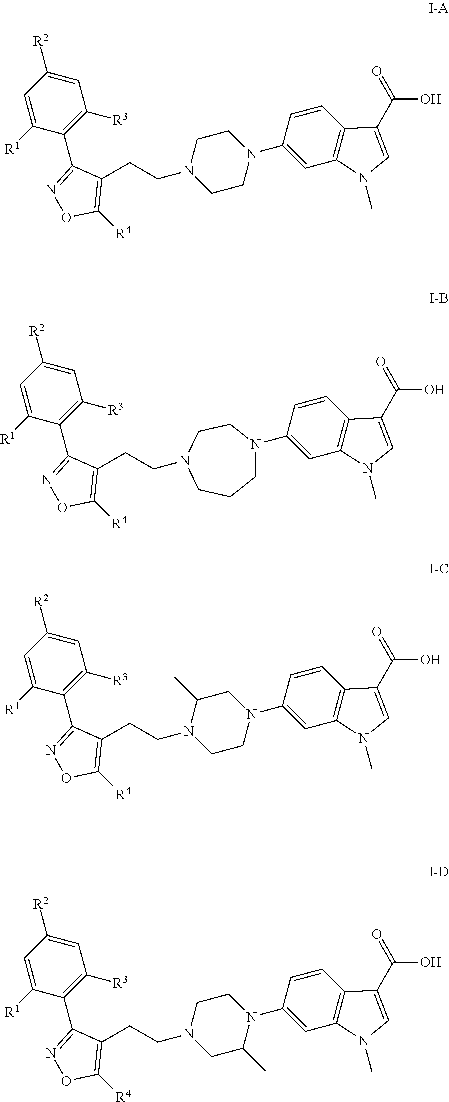 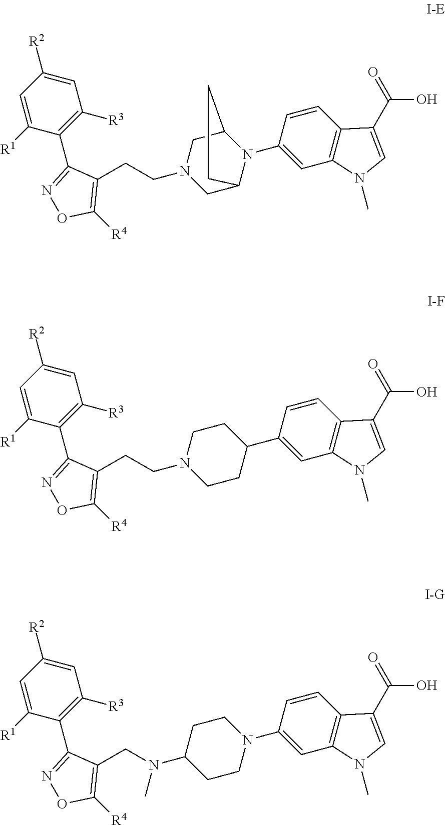
Wherein R¹, R² and R³ are independently selected from H, Cl, F, CH₃, OCF₃, CF₃ and OMe, R⁴ is cyclopropyl or i-Pr,
or a stereoisomer, enantiomer or a pharmaceutically acceptable salt thereof.

14. The compound of claim 1, wherein said compound is selected from the following structure: 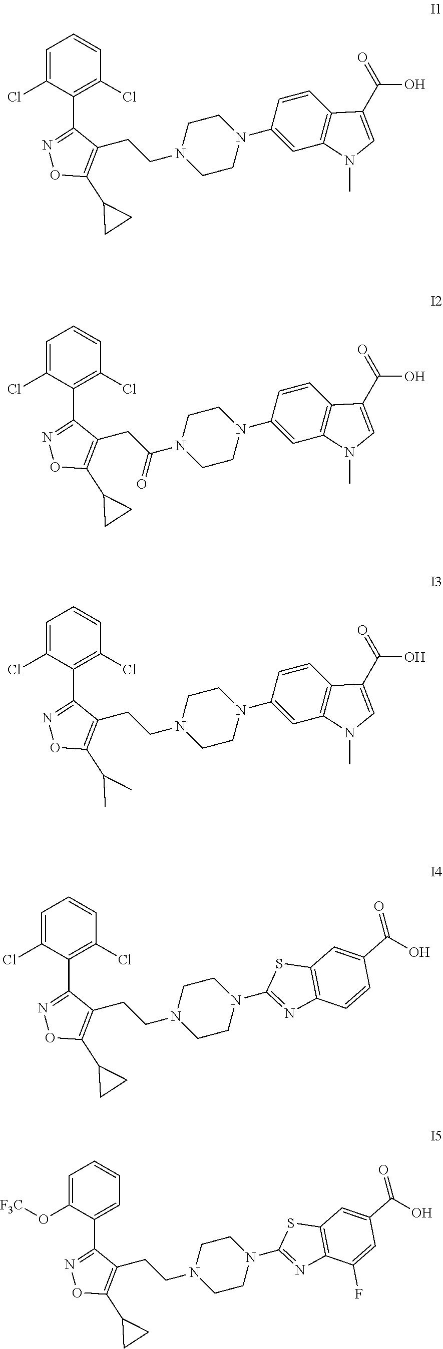 [SMILES: Cn1cc(C(=O)O)c2ccc(N3CCN(CCc4c(-c5c(Cl)cccc5Cl)noc4C4CC4)CC3)cc21 ; Cn1cc(C(=O)O)c2ccc(N3CCN(C(=O)Cc4c(-c5c(Cl)cccc5Cl)noc4C4CC4)CC3)cc21 ; CC(C)c1onc(-c2c(Cl)cccc2Cl)c1CCN1CCN(c2ccc3c(C(=O)O)cn(C)c3c2)CC1 ; O=C(O)c1ccc2nc(N3CCN(CCc4c(-c5c(Cl)cccc5Cl)noc4C4CC4)CC3)sc2c1 ; O=C(O)c1cc(F)c2nc(N3CCN(CCc4c(-c5ccccc5OC(F)(F)F)noc4C4CC4)CC3)sc2c1] 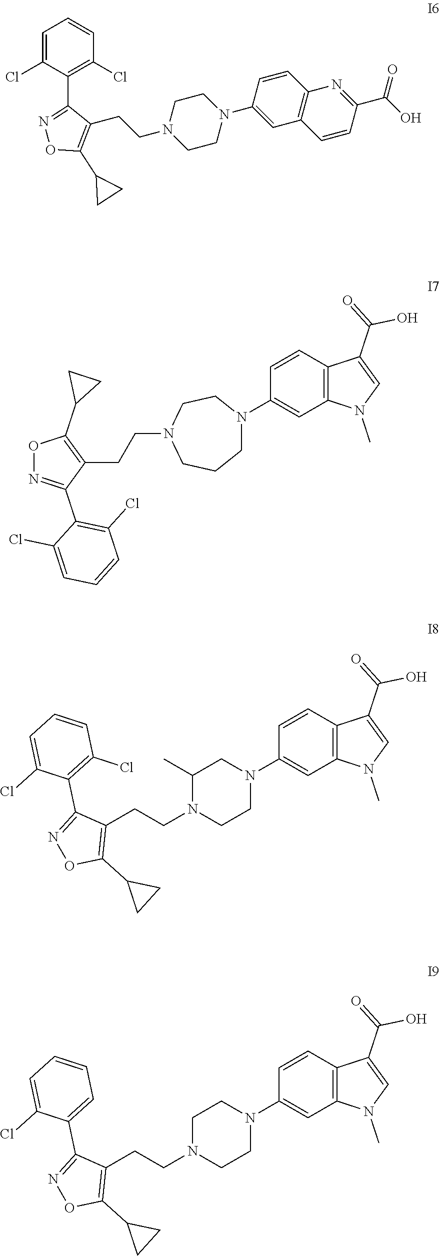 [SMILES: O=C(O)c1ccc2cc(N3CCN(CCc4c(-c5c(Cl)cccc5Cl)noc4C4CC4)CC3)ccc2n1 ; Cn1cc(C(=O)O)c2ccc(N3CCCN(CCc4c(-c5c(Cl)cccc5Cl)noc4C4CC4)CC3)cc21 ; CC1CN(c2ccc3c(C(=O)O)cn(C)c3c2)CCN1CCc1c(-c2c(Cl)cccc2Cl)noc1C1CC1 ; Cn1cc(C(=O)O)c2ccc(N3CCN(CCc4c(-c5ccccc5Cl)noc4C4CC4)CC3)cc21] 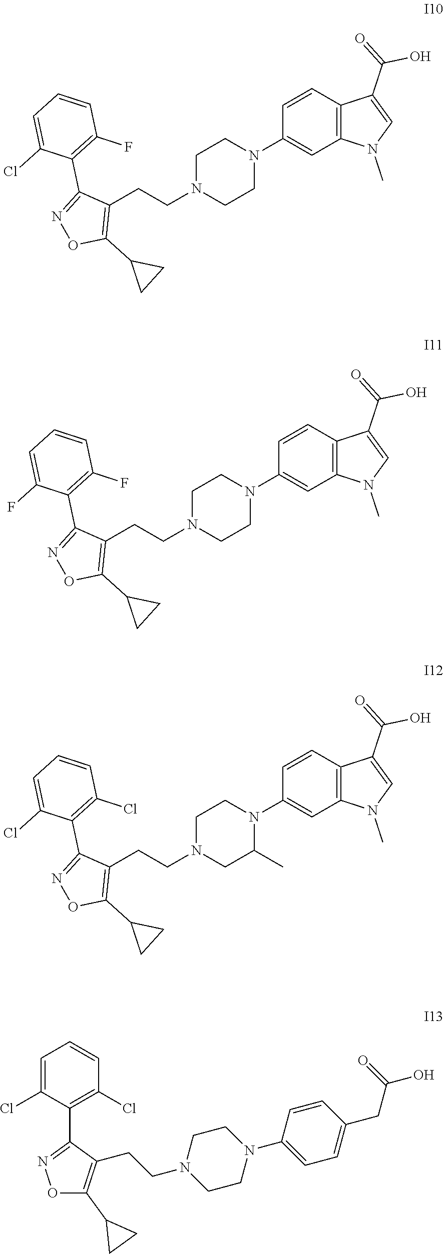 [SMILES: Cn1cc(C(=O)O)c2ccc(N3CCN(CCc4c(-c5c(F)cccc5Cl)noc4C4CC4)CC3)cc21 ; Cn1cc(C(=O)O)c2ccc(N3CCN(CCc4c(-c5c(F)cccc5F)noc4C4CC4)CC3)cc21 ; CC1CN(CCc2c(-c3c(Cl)cccc3Cl)noc2C2CC2)CCN1c1ccc2c(C(=O)O)cn(C)c2c1 ; O=C(O)Cc1ccc(N2CCN(CCc3c(-c4c(Cl)cccc4Cl)noc3C3CC3)CC2)cc1] 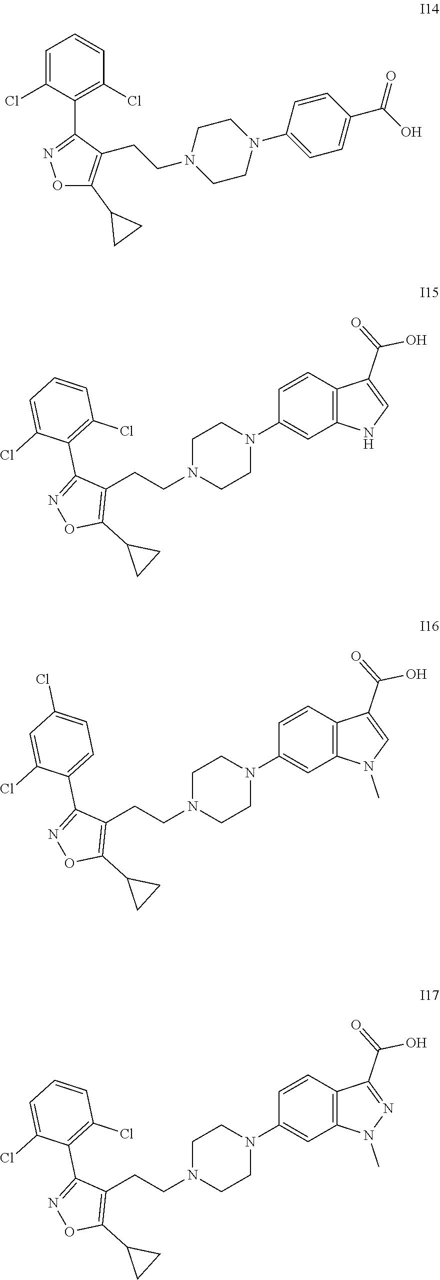 [SMILES: O=C(O)c1ccc(N2CCN(CCc3c(-c4c(Cl)cccc4Cl)noc3C3CC3)CC2)cc1 ; O=C(O)c1c[nH]c2cc(N3CCN(CCc4c(-c5c(Cl)cccc5Cl)noc4C4CC4)CC3)ccc12 ; Cn1cc(C(=O)O)c2ccc(N3CCN(CCc4c(-c5ccc(Cl)cc5Cl)noc4C4CC4)CC3)cc21 ; Cn1nc(C(=O)O)c2ccc(N3CCN(CCc4c(-c5c(Cl)cccc5Cl)noc4C4CC4)CC3)cc21] 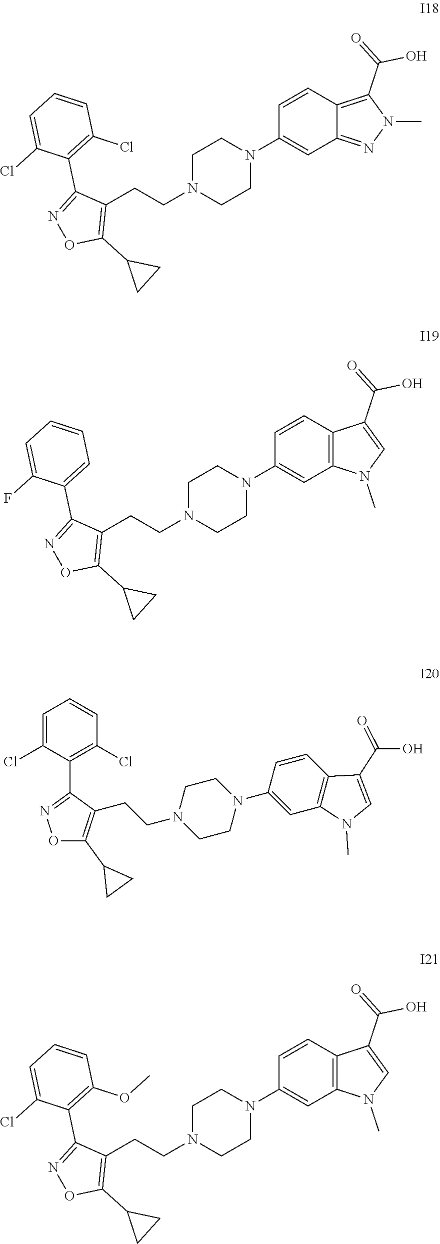 [SMILES: Cn1nc2cc(N3CCN(CCc4c(-c5c(Cl)cccc5Cl)noc4C4CC4)CC3)ccc2c1C(=O)O ; Cn1cc(C(=O)O)c2ccc(N3CCN(CCc4c(-c5ccccc5F)noc4C4CC4)CC3)cc21 ; Cn1cc(C(=O)O)c2ccc(N3CCN(CCc4c(-c5c(Cl)cccc5Cl)noc4C4CC4)CC3)cc21 ; COc1cccc(Cl)c1-c1noc(C2CC2)c1CCN1CCN(c2ccc3c(C(=O)O)cn(C)c3c2)CC1] 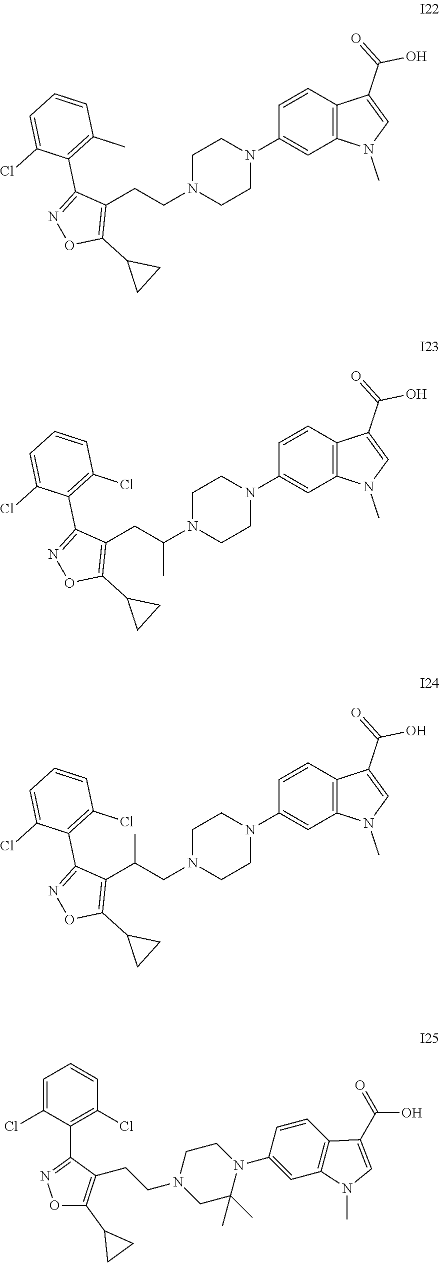 [SMILES: Cc1cccc(Cl)c1-c1noc(C2CC2)c1CCN1CCN(c2ccc3c(C(=O)O)cn(C)c3c2)CC1 ; CC(Cc1c(-c2c(Cl)cccc2Cl)noc1C1CC1)N1CCN(c2ccc3c(C(=O)O)cn(C)c3c2)CC1 ; CC(CN1CCN(c2ccc3c(C(=O)O)cn(C)c3c2)CC1)c1c(-c2c(Cl)cccc2Cl)noc1C1CC1 ; Cn1cc(C(=O)O)c2ccc(N3CCN(CCc4c(-c5c(Cl)cccc5Cl)noc4C4CC4)CC3(C)C)cc21] 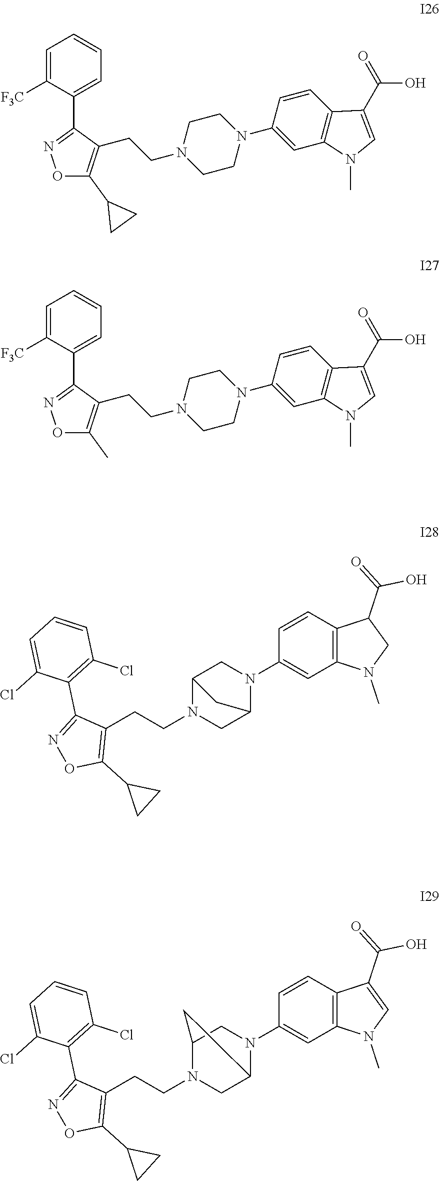 [SMILES: Cn1cc(C(=O)O)c2ccc(N3CCN(CCc4c(-c5ccccc5C(F)(F)F)noc4C4CC4)CC3)cc21 ; Cc1onc(-c2ccccc2C(F)(F)F)c1CCN1CCN(c2ccc3c(C(=O)O)cn(C)c3c2)CC1 ; CN1CC(C(=O)O)c2ccc(N3CC4CC3CN4CCc3c(-c4c(Cl)cccc4Cl)noc3C3CC3)cc21 ; Cn1cc(C(=O)O)c2ccc(N3CC4CC3CN4CCc3c(-c4c(Cl)cccc4Cl)noc3C3CC3)cc21] 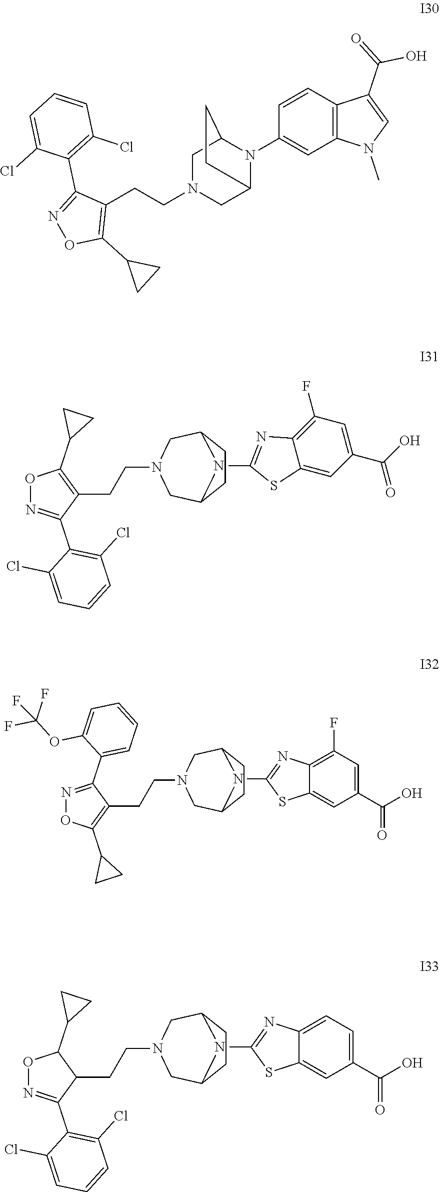 [SMILES: Cn1cc(C(=O)O)c2ccc(N3C4CCC3CN(CCc3c(-c5c(Cl)cccc5Cl)noc3C3CC3)C4)cc21 ; O=C(O)c1cc(F)c2nc(N3C4CCC3CN(CCc3c(-c5c(Cl)cccc5Cl)noc3C3CC3)C4)sc2c1 ; O=C(O)c1cc(F)c2nc(N3C4CCC3CN(CCc3c(-c5ccccc5OC(F)(F)F)noc3C3CC3)C4)sc2c1 ; O=C(O)c1ccc2nc(N3C4CCC3CN(CCC3C(c5c(Cl)cccc5Cl)=NOC3C3CC3)C4)sc2c1] 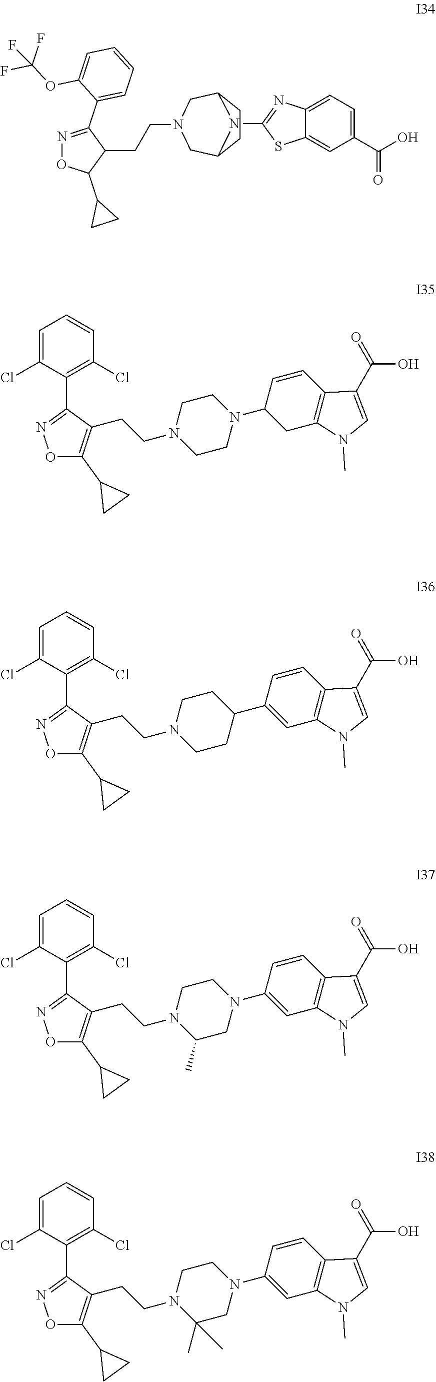 [SMILES: O=C(O)c1ccc2nc(N3C4CCC3CN(CCC3C(c5ccccc5OC(F)(F)F)=NOC3C3CC3)C4)sc2c1 ; Cn1cc(C(=O)O)c2c1CC(N1CCN(CCc3c(-c4c(Cl)cccc4Cl)noc3C3CC3)CC1)C=C2 ; Cn1cc(C(=O)O)c2ccc(C3CCN(CCc4c(-c5c(Cl)cccc5Cl)noc4C4CC4)CC3)cc21 ; C[C@H]1CN(c2ccc3c(C(=O)O)cn(C)c3c2)CCN1CCc1c(-c2c(Cl)cccc2Cl)noc1C1CC1 ; Cn1cc(C(=O)O)c2ccc(N3CCN(CCc4c(-c5c(Cl)cccc5Cl)noc4C4CC4)C(C)(C)C3)cc21] 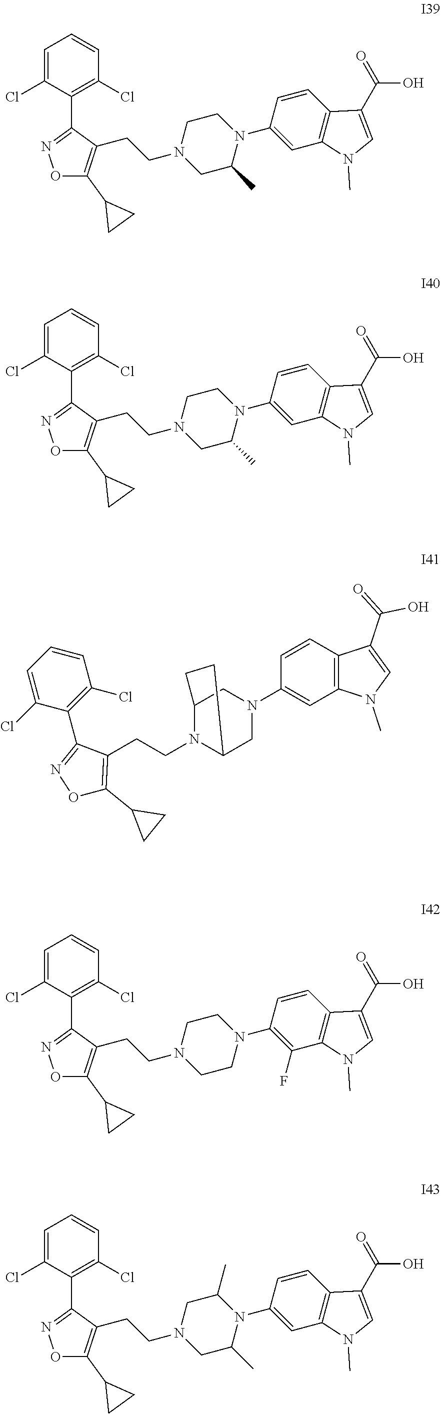 [SMILES: C[C@H]1CN(CCc2c(-c3c(Cl)cccc3Cl)noc2C2CC2)CCN1c1ccc2c(C(=O)O)cn(C)c2c1 ; C[C@@H]1CN(CCc2c(-c3c(Cl)cccc3Cl)noc2C2CC2)CCN1c1ccc2c(C(=O)O)cn(C)c2c1 ; Cn1cc(C(=O)O)c2ccc(N3CC4CCC(C3)N4CCc3c(-c4c(Cl)cccc4Cl)noc3C3CC3)cc21 ; Cn1cc(C(=O)O)c2ccc(N3CCN(CCc4c(-c5c(Cl)cccc5Cl)noc4C4CC4)CC3)c(F)c21 ; CC1CN(CCc2c(-c3c(Cl)cccc3Cl)noc2C2CC2)CC(C)N1c1ccc2c(C(=O)O)cn(C)c2c1] 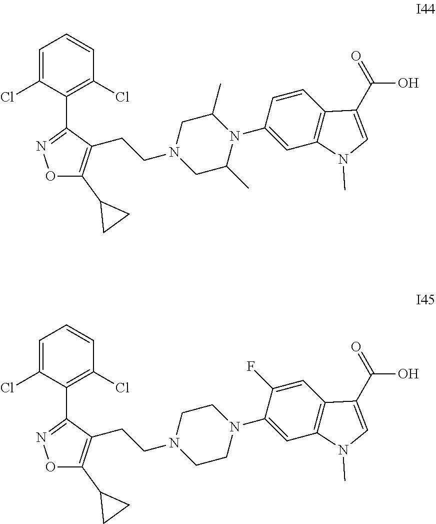 [SMILES: CC1CN(CCc2c(-c3c(Cl)cccc3Cl)noc2C2CC2)CC(C)N1c1ccc2c(C(=O)O)cn(C)c2c1 ; Cn1cc(C(=O)O)c2cc(F)c(N3CCN(CCc4c(-c5c(Cl)cccc5Cl)noc4C4CC4)CC3)cc21]
or a stereoisomer, enantiomer or a pharmaceutically acceptable salt thereof.

15. (canceled)

16. (canceled)

17. (canceled)

18. (canceled)

19. (canceled)

20. (canceled)

21. A compound having Formula I′: 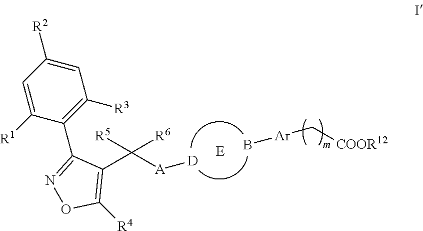
or a stereoisomer, enantiomer or a pharmaceutically acceptable salt thereof;
R¹, R² and R³ are independently selected from H, C₁₋₆alkyl, haloC₁₋₆alkyl, C₁₋₆ alkoxy, haloC₁₋₆alkoxy, or cyclopropyl;
R⁴ is selected from C₁₋₃alkyl, haloC₁₋₃alkyl or cyclopropyl optionally substituted with C₁₋₃ alkyl or haloC₁₋₃alkyl;
R⁵ and R⁶ are independently selected from H, C₁₋₃alkyl or haloC₁₋₃alkyl;
A is selected from C═O, CR⁷R⁸, O or NR⁹;
R⁷ and R⁸ are independently selected from H, C₁₋₃alkyl or C₁₋₃alkoxy;
R⁹ is selected from H, C₁₋₃alkyl or C₁₋₃alkoxy;
B is CR¹³ or N;
D is CR¹⁴ or N;
ring E is a substituted or unsubstituted 6-8 membered heteroring or bridged-heteroring;
Ar is phenylene, C₅₋₇ cycloalkylene or 5-14 membered monocyclic or bicyclic heteroaryl containing 1-2 heteroatoms selected from N, O and S; each of which is optionally substituted with R¹⁰ and R¹¹,
R¹⁰ and R¹¹ are independently selected from H, halogen, C₁₋₆ alkyl, haloC₁₋₆ alkyl, C₁₋₆ alkoxy, haloC₁₋₆ alkoxy, or cyclopropyl;
R¹² is selected from H, C₁₋₃alkyl or C₁₋₃alkoxy;
R¹³ is selected from H, OH, C₁₋₃alkyl or C₁₋₃alkoxy;
R¹⁴ is selected from H, OH, C₁₋₃alkyl or C₁₋₃alkoxy;
m is 0 or 1.

22. The compound of claim 21, wherein
A is selected from C═O or CR⁷R⁸ preferably, A is NMe;
B is CR¹³ or N;
D is N or CH;
R¹² is H or Me;
R¹³ is H or OH.

23. (canceled)

24. The compound of claim 21, wherein R¹, R² and R³ are independently selected from H, Cl, F, CH₃, OCF₃, CF₃ and OMe.

25. The compound of claim 21, wherein R⁴ is C₁₋₃ alkyl or cyclopropyl; preferably, R⁴ is methyl or i-Pr.

26. (canceled)

27. The compound of claim 21, wherein R⁵, R⁶, R⁷ and R⁸ are independently selected from hydrogen or Me.

28. (canceled)

29. The compound of claim 21, wherein R⁹ is selected from H, Me, Et, n-Pr or i-Pr; R¹² and R¹³ are independently selected from H, Me, Et, n-Pr or i-Pr.

30. The compound of claim 21, wherein Ar is selected from substituted or unsubstituted phenylene, pyridylene, pyrimidinylene, pyrazinylene, pyridazinylene, thiazolylene, benzothiazolyl, benzo[d]isothiazolyl, imidazo[1,2-a]pyridinyl, quinolinyl, 1H-indolyl, pyrrolo[1,2-b]pyridazinyl, benzofuranyl, benzo[b]thiophenyl, 1H-indazolyl, benzo[d]isoxazolyl, quinazolinyl, 1H-pyrrolo[3,2-c]pyridinyl, pyrazolo[1,5-a]pyrimidinyl, imidazo[1,2-b]pyridazinyl, pyrazolo[1,5-a]pyridinyl; each of which is optionally substituted with R¹⁰ and R¹¹ selected from H, halogen, C₁₋₆ alkyl, haloC₁₋₆ alkyl, C₁₋₆ alkoxy, haloC₁₋₆ alkoxy, or cyclopropyl, preferably Ar is selected from phenylene, benzothiazolyl, quinolinyl, 1H-indolyl, 1H-indazolyl, each of which is optionally substituted with 0˜2 groups of Me or F.

31. (canceled)

32. The compound of claim 30, wherein Ar is phenylene or selected from the following structure

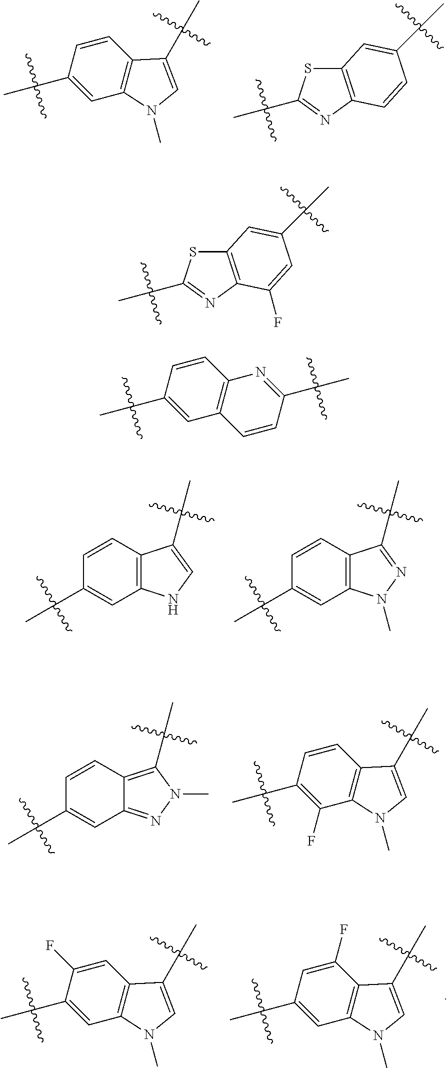

33. The compound of claim 21, wherein ring E is selected from the following structure, which is optionally substituted with 0˜2 groups of OH or Me 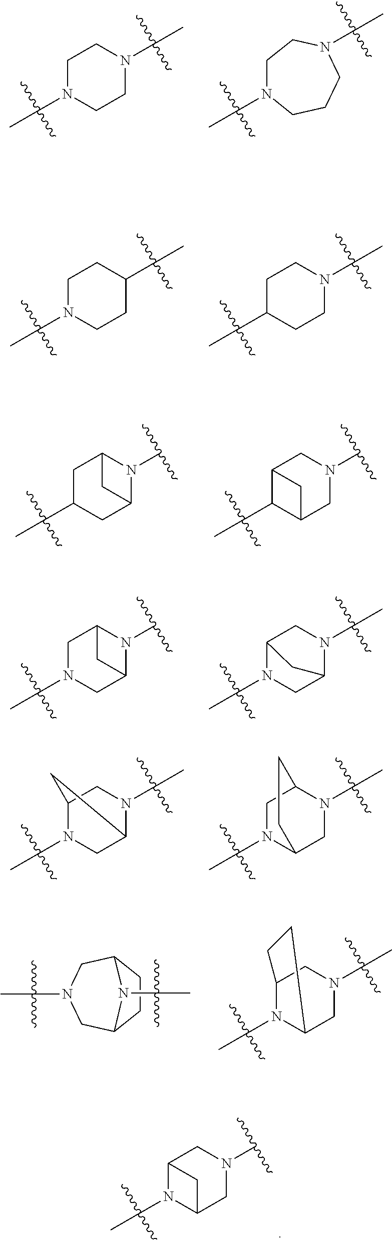
preferably, ring E is selected from the following structure:

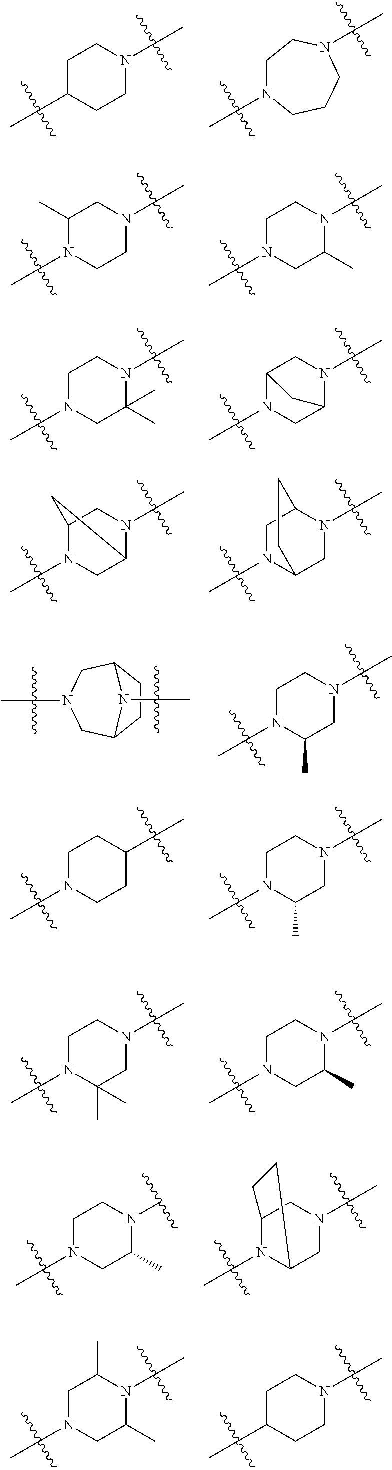

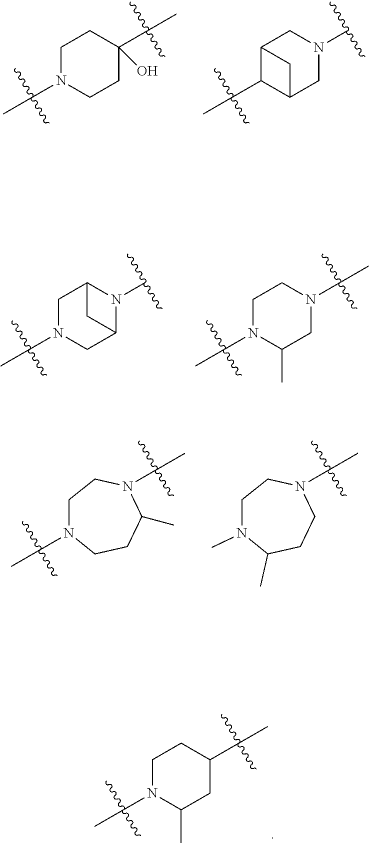

34. (canceled)

35. The compound of claim 21, wherein said compound is selected from the following structure 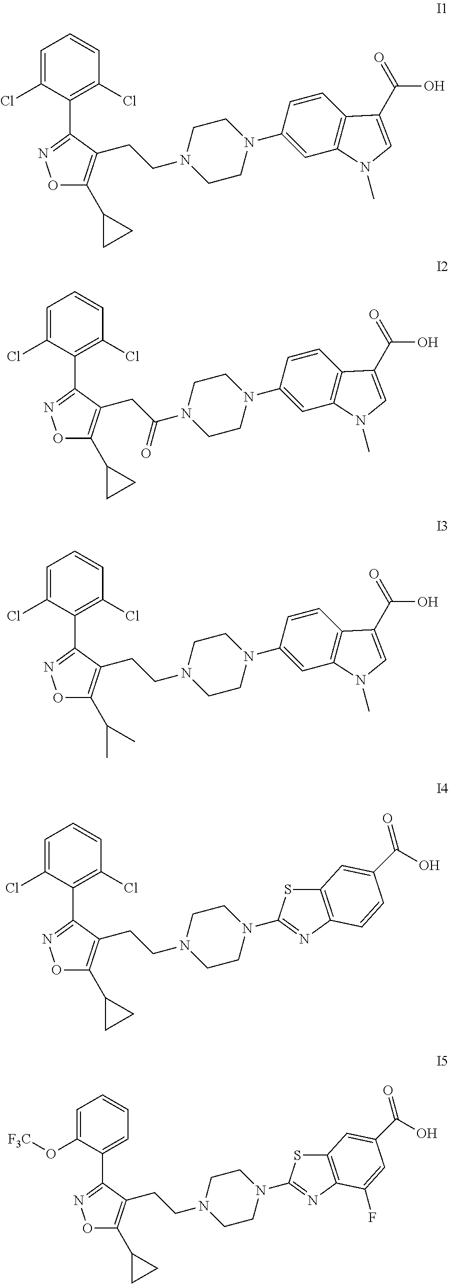 [SMILES: Cn1cc(C(=O)O)c2ccc(N3CCN(CCc4c(-c5c(Cl)cccc5Cl)noc4C4CC4)CC3)cc21 ; Cn1cc(C(=O)O)c2ccc(N3CCN(C(=O)Cc4c(-c5c(Cl)cccc5Cl)noc4C4CC4)CC3)cc21 ; CC(C)c1onc(-c2c(Cl)cccc2Cl)c1CCN1CCN(c2ccc3c(C(=O)O)cn(C)c3c2)CC1 ; O=C(O)c1ccc2nc(N3CCN(CCc4c(-c5c(Cl)cccc5Cl)noc4C4CC4)CC3)sc2c1 ; O=C(O)c1cc(F)c2nc(N3CCN(CCc4c(-c5ccccc5OC(F)(F)F)noc4C4CC4)CC3)sc2c1] 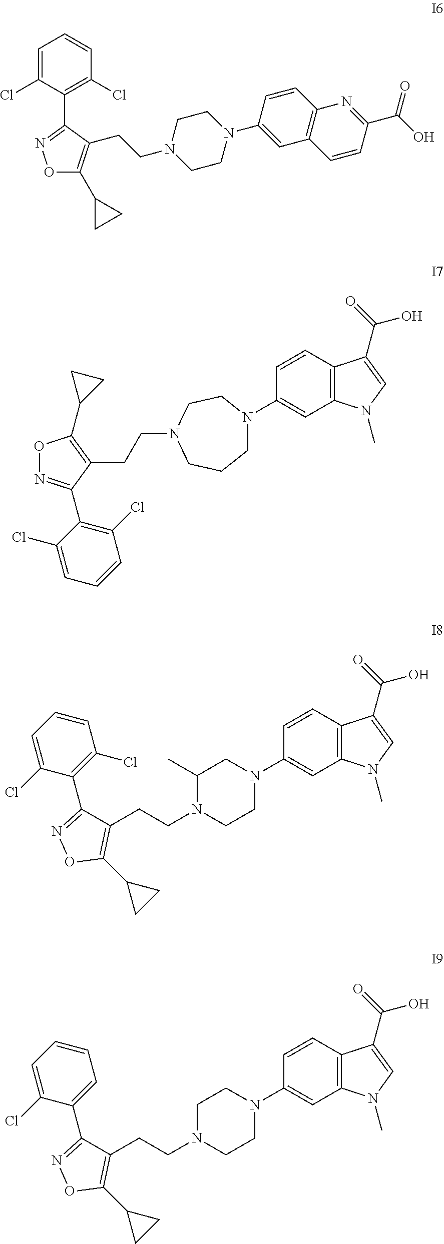 [SMILES: O=C(O)c1ccc2cc(N3CCN(CCc4c(-c5c(Cl)cccc5Cl)noc4C4CC4)CC3)ccc2n1 ; Cn1cc(C(=O)O)c2ccc(N3CCCN(CCc4c(-c5c(Cl)cccc5Cl)noc4C4CC4)CC3)cc21 ; CC1CN(c2ccc3c(C(=O)O)cn(C)c3c2)CCN1CCc1c(-c2c(Cl)cccc2Cl)noc1C1CC1 ; Cn1cc(C(=O)O)c2ccc(N3CCN(CCc4c(-c5ccccc5Cl)noc4C4CC4)CC3)cc21] 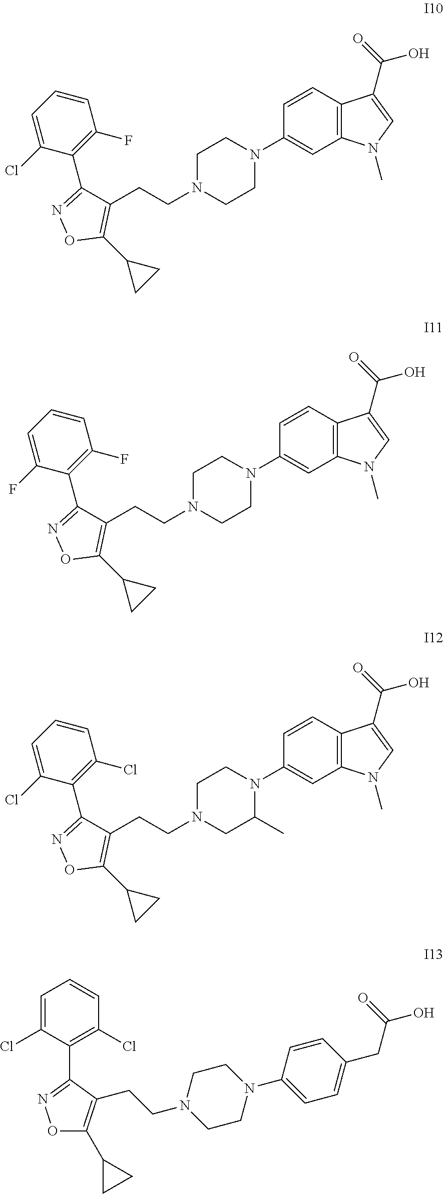 [SMILES: Cn1cc(C(=O)O)c2ccc(N3CCN(CCc4c(-c5c(F)cccc5Cl)noc4C4CC4)CC3)cc21 ; Cn1cc(C(=O)O)c2ccc(N3CCN(CCc4c(-c5c(F)cccc5F)noc4C4CC4)CC3)cc21 ; CC1CN(CCc2c(-c3c(Cl)cccc3Cl)noc2C2CC2)CCN1c1ccc2c(C(=O)O)cn(C)c2c1 ; O=C(O)Cc1ccc(N2CCN(CCc3c(-c4c(Cl)cccc4Cl)noc3C3CC3)CC2)cc1] 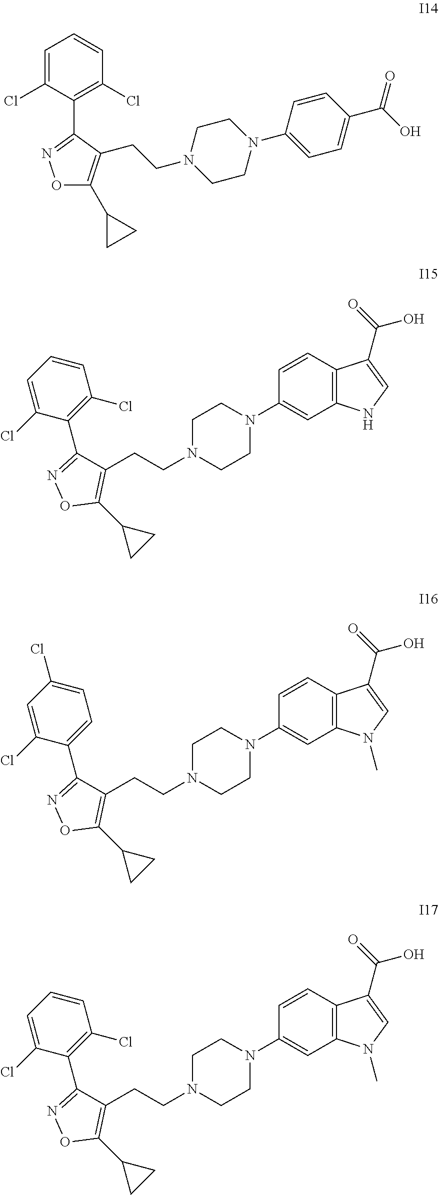 [SMILES: O=C(O)c1ccc(N2CCN(CCc3c(-c4c(Cl)cccc4Cl)noc3C3CC3)CC2)cc1 ; O=C(O)c1c[nH]c2cc(N3CCN(CCc4c(-c5c(Cl)cccc5Cl)noc4C4CC4)CC3)ccc12 ; Cn1cc(C(=O)O)c2ccc(N3CCN(CCc4c(-c5ccc(Cl)cc5Cl)noc4C4CC4)CC3)cc21 ; Cn1cc(C(=O)O)c2ccc(N3CCN(CCc4c(-c5c(Cl)cccc5Cl)noc4C4CC4)CC3)cc21] 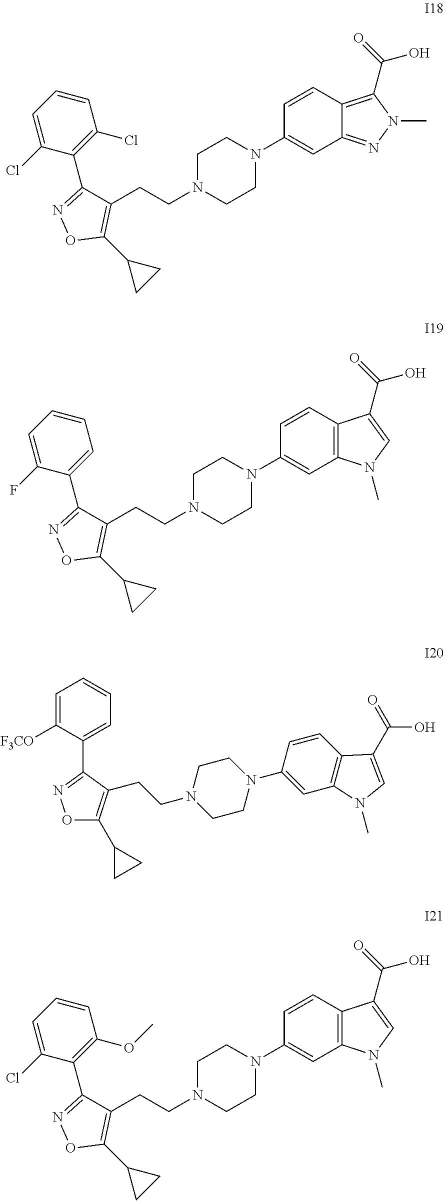 [SMILES: Cn1nc2cc(N3CCN(CCc4c(-c5c(Cl)cccc5Cl)noc4C4CC4)CC3)ccc2c1C(=O)O ; Cn1cc(C(=O)O)c2ccc(N3CCN(CCc4c(-c5ccccc5F)noc4C4CC4)CC3)cc21 ; Cn1cc(C(=O)O)c2ccc(N3CCN(CCc4c(-c5ccccc5OC(F)(F)F)noc4C4CC4)CC3)cc21 ; COc1cccc(Cl)c1-c1noc(C2CC2)c1CCN1CCN(c2ccc3c(C(=O)O)cn(C)c3c2)CC1] 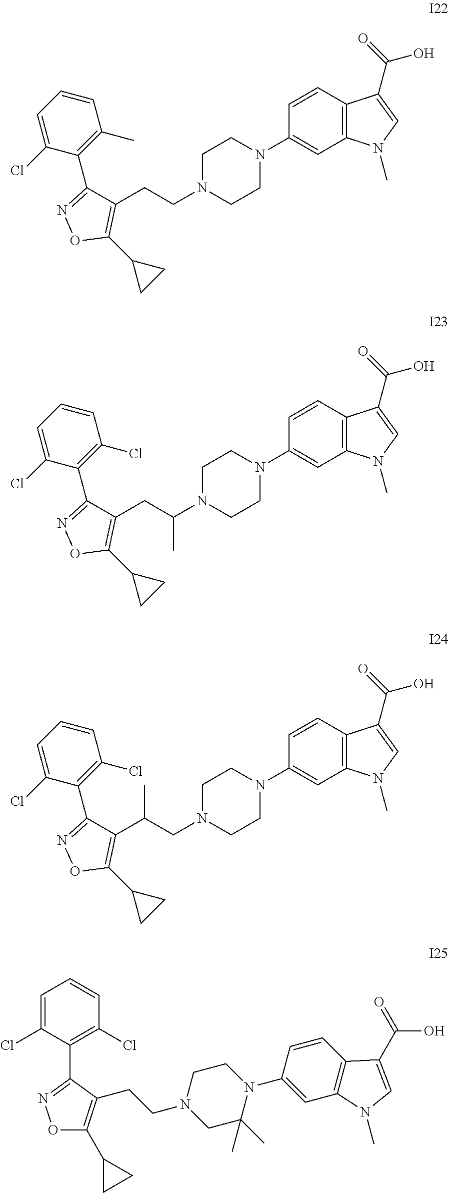 [SMILES: Cc1cccc(Cl)c1-c1noc(C2CC2)c1CCN1CCN(c2ccc3c(C(=O)O)cn(C)c3c2)CC1 ; CC(Cc1c(-c2c(Cl)cccc2Cl)noc1C1CC1)N1CCN(c2ccc3c(C(=O)O)cn(C)c3c2)CC1 ; CC(CN1CCN(c2ccc3c(C(=O)O)cn(C)c3c2)CC1)c1c(-c2c(Cl)cccc2Cl)noc1C1CC1 ; Cn1cc(C(=O)O)c2ccc(N3CCN(CCc4c(-c5c(Cl)cccc5Cl)noc4C4CC4)CC3(C)C)cc21] 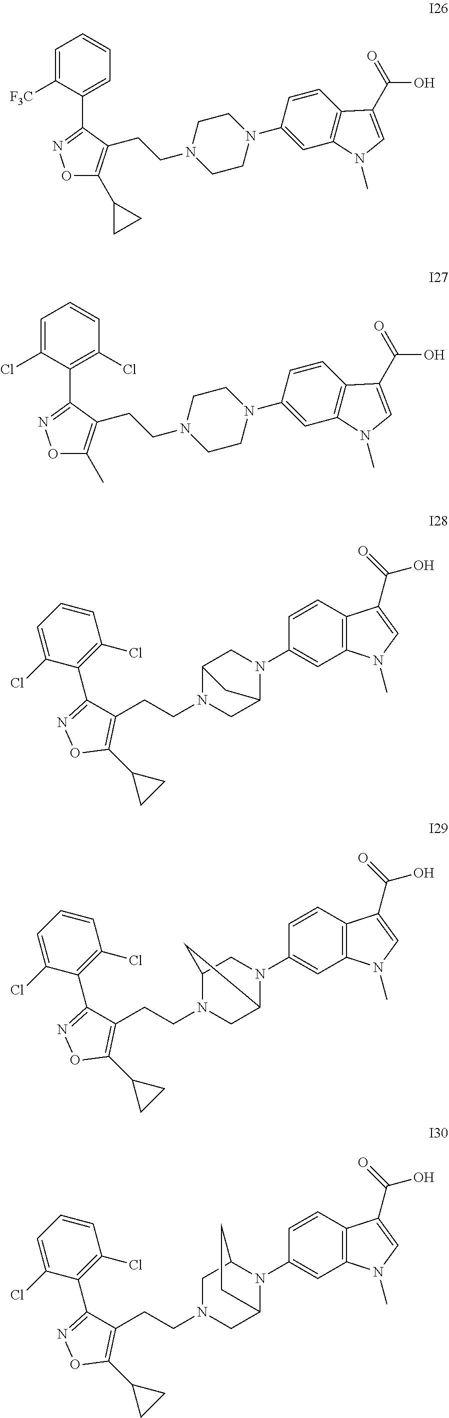 [SMILES: Cn1cc(C(=O)O)c2ccc(N3CCN(CCc4c(-c5ccccc5C(F)(F)F)noc4C4CC4)CC3)cc21 ; Cc1onc(-c2c(Cl)cccc2Cl)c1CCN1CCN(c2ccc3c(C(=O)O)cn(C)c3c2)CC1 ; Cn1cc(C(=O)O)c2ccc(N3CC4CC3CN4CCc3c(-c4c(Cl)cccc4Cl)noc3C3CC3)cc21 ; Cn1cc(C(=O)O)c2ccc(N3CC4CC3CN4CCc3c(-c4c(Cl)cccc4Cl)noc3C3CC3)cc21 ; Cn1cc(C(=O)O)c2ccc(N3C4CCC3CN(CCc3c(-c5c(Cl)cccc5Cl)noc3C3CC3)C4)cc21] 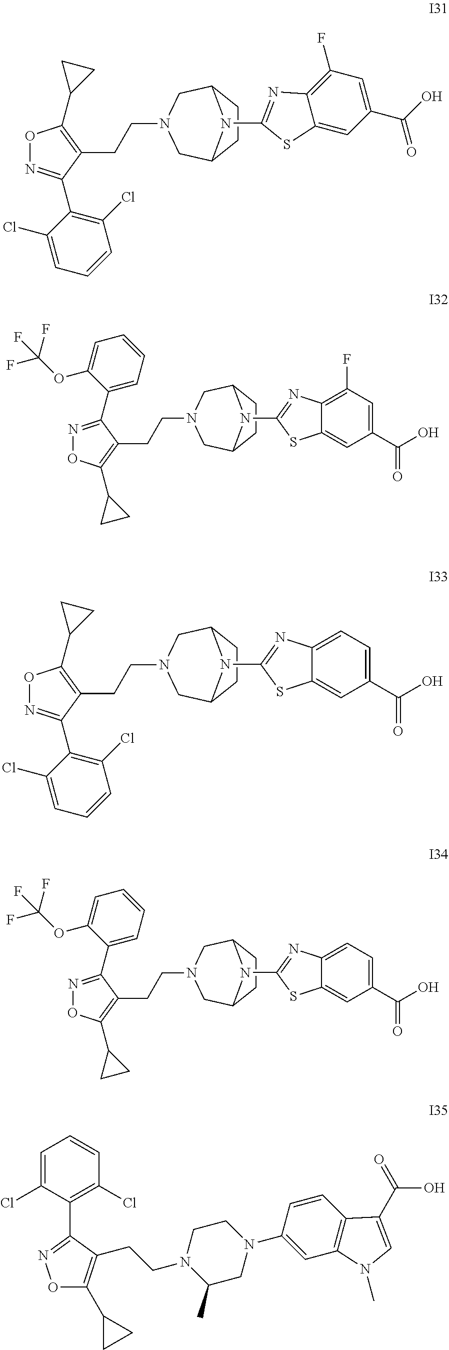 [SMILES: O=C(O)c1cc(F)c2nc(N3C4CCC3CN(CCc3c(-c5c(Cl)cccc5Cl)noc3C3CC3)C4)sc2c1 ; O=C(O)c1cc(F)c2nc(N3C4CCC3CN(CCc3c(-c5ccccc5OC(F)(F)F)noc3C3CC3)C4)sc2c1 ; O=C(O)c1ccc2nc(N3C4CCC3CN(CCc3c(-c5c(Cl)cccc5Cl)noc3C3CC3)C4)sc2c1 ; O=C(O)c1ccc2nc(N3C4CCC3CN(CCc3c(-c5ccccc5OC(F)(F)F)noc3C3CC3)C4)sc2c1 ; C[C@@H]1CN(c2ccc3c(C(=O)O)cn(C)c3c2)CCN1CCc1c(-c2c(Cl)cccc2Cl)noc1C1CC1] 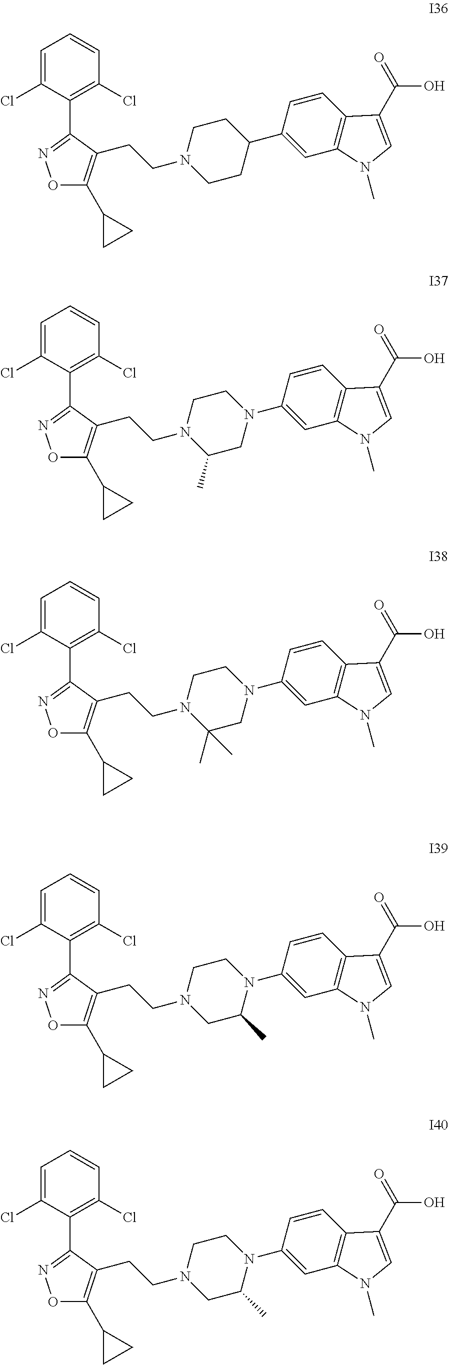 [SMILES: Cn1cc(C(=O)O)c2ccc(C3CCN(CCc4c(-c5c(Cl)cccc5Cl)noc4C4CC4)CC3)cc21 ; C[C@H]1CN(c2ccc3c(C(=O)O)cn(C)c3c2)CCN1CCc1c(-c2c(Cl)cccc2Cl)noc1C1CC1 ; Cn1cc(C(=O)O)c2ccc(N3CCN(CCc4c(-c5c(Cl)cccc5Cl)noc4C4CC4)C(C)(C)C3)cc21 ; C[C@H]1CN(CCc2c(-c3c(Cl)cccc3Cl)noc2C2CC2)CCN1c1ccc2c(C(=O)O)cn(C)c2c1 ; C[C@@H]1CN(CCc2c(-c3c(Cl)cccc3Cl)noc2C2CC2)CCN1c1ccc2c(C(=O)O)cn(C)c2c1] 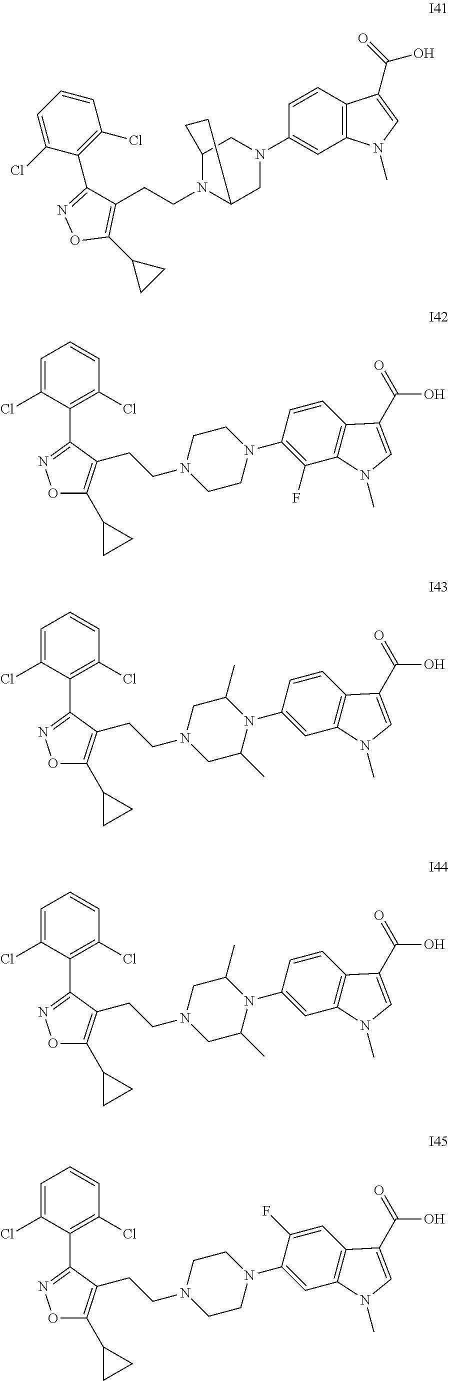 [SMILES: Cn1cc(C(=O)O)c2ccc(N3CC4CCC(C3)N4CCc3c(-c4c(Cl)cccc4Cl)noc3C3CC3)cc21 ; Cn1cc(C(=O)O)c2ccc(N3CCN(CCc4c(-c5c(Cl)cccc5Cl)noc4C4CC4)CC3)c(F)c21 ; CC1CN(CCc2c(-c3c(Cl)cccc3Cl)noc2C2CC2)CC(C)N1c1ccc2c(C(=O)O)cn(C)c2c1 ; CC1CN(CCc2c(-c3c(Cl)cccc3Cl)noc2C2CC2)CC(C)N1c1ccc2c(C(=O)O)cn(C)c2c1 ; Cn1cc(C(=O)O)c2cc(F)c(N3CCN(CCc4c(-c5c(Cl)cccc5Cl)noc4C4CC4)CC3)cc21] 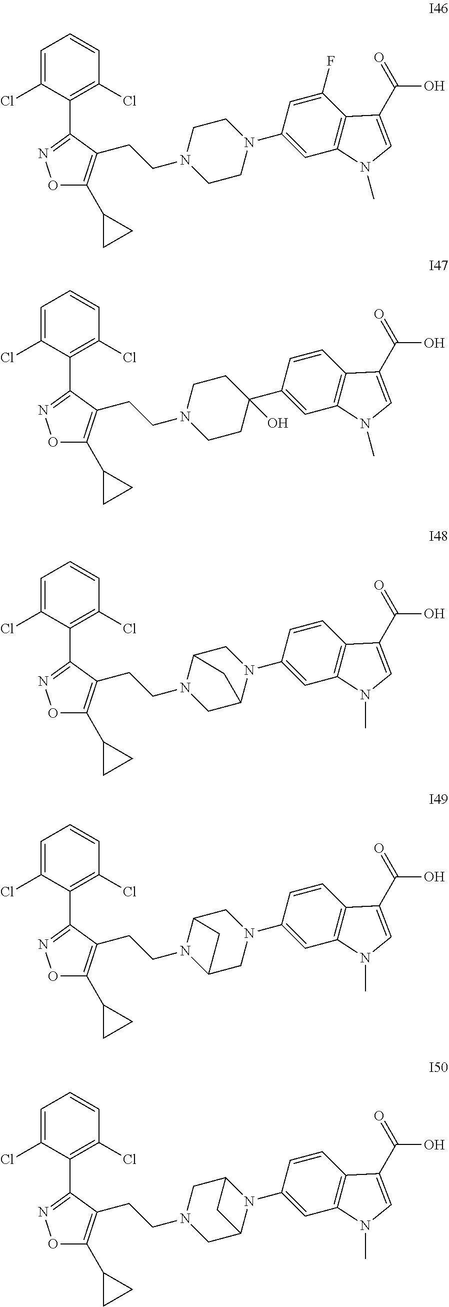 [SMILES: Cn1cc(C(=O)O)c2c(F)cc(N3CCN(CCc4c(-c5c(Cl)cccc5Cl)noc4C4CC4)CC3)cc21 ; Cn1cc(C(=O)O)c2ccc(C3(O)CCN(CCc4c(-c5c(Cl)cccc5Cl)noc4C4CC4)CC3)cc21 ; Cn1cc(C(=O)O)c2ccc(N3CC4CC3CN4CCc3c(-c4c(Cl)cccc4Cl)noc3C3CC3)cc21 ; Cn1cc(C(=O)O)c2ccc(N3CC4CC(C3)N4CCc3c(-c4c(Cl)cccc4Cl)noc3C3CC3)cc21 ; Cn1cc(C(=O)O)c2ccc(N3C4CC3CN(CCc3c(-c5c(Cl)cccc5Cl)noc3C3CC3)C4)cc21] 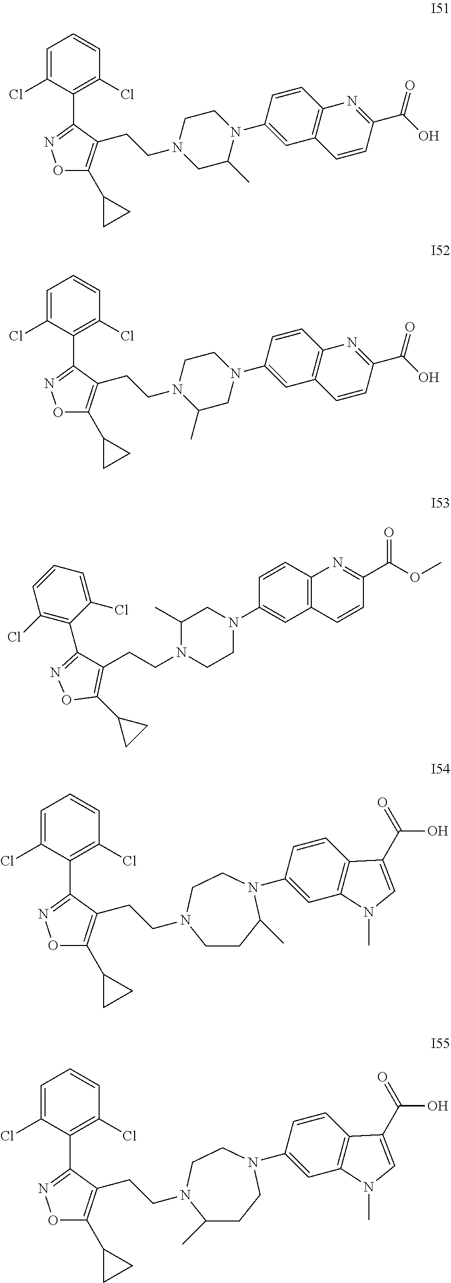 [SMILES: CC1CN(CCc2c(-c3c(Cl)cccc3Cl)noc2C2CC2)CCN1c1ccc2nc(C(=O)O)ccc2c1 ; CC1CN(c2ccc3nc(C(=O)O)ccc3c2)CCN1CCc1c(-c2c(Cl)cccc2Cl)noc1C1CC1 ; COC(=O)c1ccc2cc(N3CCN(CCc4c(-c5c(Cl)cccc5Cl)noc4C4CC4)C(C)C3)ccc2n1 ; CC1CCN(CCc2c(-c3c(Cl)cccc3Cl)noc2C2CC2)CCN1c1ccc2c(C(=O)O)cn(C)c2c1 ; CC1CCN(c2ccc3c(C(=O)O)cn(C)c3c2)CCN1CCc1c(-c2c(Cl)cccc2Cl)noc1C1CC1] 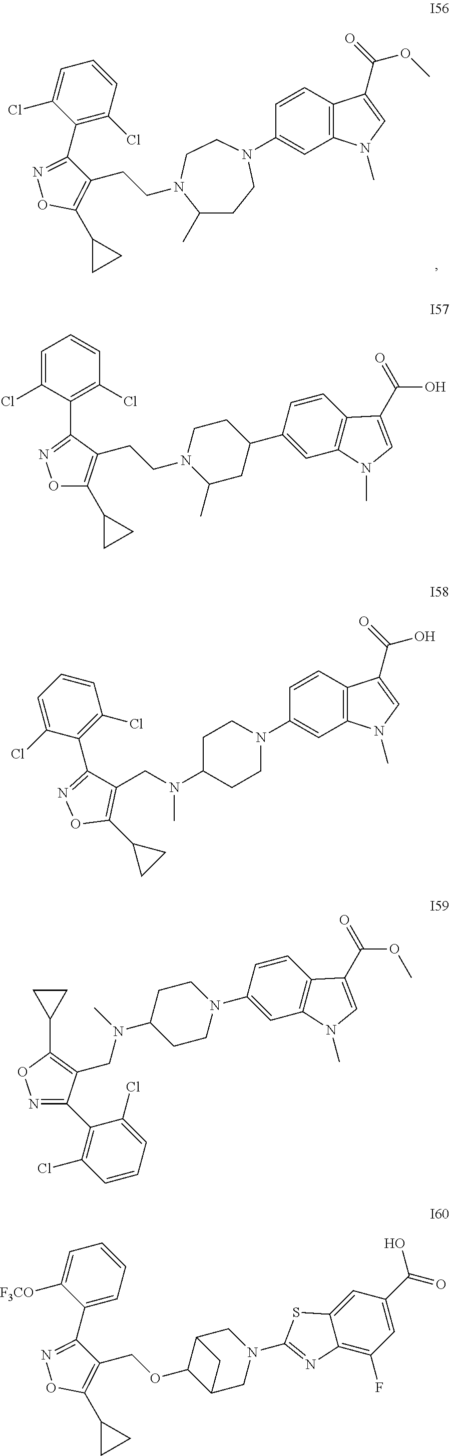 [SMILES: COC(=O)c1cn(C)c2cc(N3CCC(C)N(CCc4c(-c5c(Cl)cccc5Cl)noc4C4CC4)CC3)ccc12 ; CC1CC(c2ccc3c(C(=O)O)cn(C)c3c2)CCN1CCc1c(-c2c(Cl)cccc2Cl)noc1C1CC1 ; CN(Cc1c(-c2c(Cl)cccc2Cl)noc1C1CC1)C1CCN(c2ccc3c(C(=O)O)cn(C)c3c2)CC1 ; COC(=O)c1cn(C)c2cc(N3CCC(N(C)Cc4c(-c5c(Cl)cccc5Cl)noc4C4CC4)CC3)ccc12 ; O=C(O)c1cc(F)c2nc(N3CC4CC(C3)C4OCc3c(-c4ccccc4OC(F)(F)F)noc3C3CC3)sc2c1] 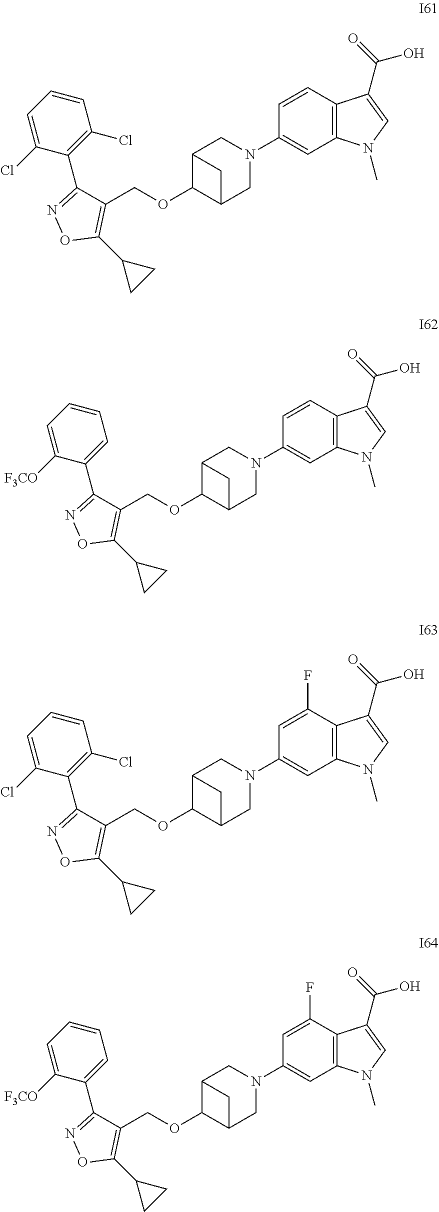 [SMILES: Cn1cc(C(=O)O)c2ccc(N3CC4CC(C3)C4OCc3c(-c4c(Cl)cccc4Cl)noc3C3CC3)cc21 ; Cn1cc(C(=O)O)c2ccc(N3CC4CC(C3)C4OCc3c(-c4ccccc4OC(F)(F)F)noc3C3CC3)cc21 ; Cn1cc(C(=O)O)c2c(F)cc(N3CC4CC(C3)C4OCc3c(-c4c(Cl)cccc4Cl)noc3C3CC3)cc21 ; Cn1cc(C(=O)O)c2c(F)cc(N3CC4CC(C3)C4OCc3c(-c4ccccc4OC(F)(F)F)noc3C3CC3)cc21] 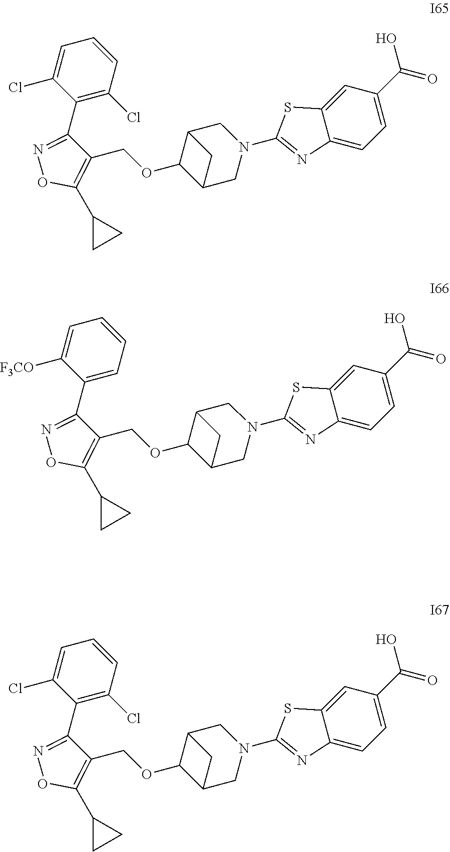 [SMILES: O=C(O)c1ccc2nc(N3CC4CC(C3)C4OCc3c(-c4c(Cl)cccc4Cl)noc3C3CC3)sc2c1 ; O=C(O)c1ccc2nc(N3CC4CC(C3)C4OCc3c(-c4ccccc4OC(F)(F)F)noc3C3CC3)sc2c1 ; O=C(O)c1ccc2nc(N3CC4CC(C3)C4OCc3c(-c4c(Cl)cccc4Cl)noc3C3CC3)sc2c1]
or a stereoisomer, enantiomer or a pharmaceutically acceptable salt thereof.

36. (canceled)

37. (canceled)

38. (canceled)

39. (canceled)

40. Use of a compound of claim 21, or a pharmaceutical composition thereof, for the preparation of a medicament for the treatment of a condition mediated by FXR in a subject.

41. The use of claim 40, wherein said condition is cholestasis, intrahepatic cholestatis, estrogen-induced cholestasis, drug-induced cholestasis, cholestasis of pregnancy, parenteral nutrition-associated cholestasis, primary biliary cirrhosis (PBC), primary sclerosing cholangistis (PSC), progressive familiar cholestatis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, dyslipidemia, atherosclerosis, diabetes, diabetic nephropathy, colitis, newborn jaundice, prevention of kernicterus, venocclusive disease, portal hypertension, metabolic syndrome, hypercholesterolemia, intestinal bacterial overgrowth, or erectile dysfunction.
